(19) United States
(12) Patent Application Publication
(10) Pub. No.: US 2007/0190063 A1
(43) Pub. Date: Aug. 16, 2007

(54) Antibody-mediated enhancement of immune response

(76) Inventors: Keith S. Bahjat, Concord, CA (US); Dirk G. Brockstedt, Oakland, CA (US); William M. Greenman, San Francisco, CA (US)

Correspondence Address: MORRISON & FOERSTER LLP, 755 PAGE MILL RD, PALO ALTO, CA, 94304-1018, US

(21) Appl. No.: 11/507,810

(22) Filed: Aug. 21, 2006

Related U.S. Application Data
(60) Provisional application No. 60/709,700, filed on Aug. 19, 2005.

(51) Int. Cl. A61K 39/42; A61K 39/395; A61K 39/02

(52) U.S. Cl. 424/155.1; 424/234.1; 424/160.1

(57) ABSTRACT

Provided are reagents and methods for administering an attenuated bacterium and a binding compound for treating a cancerous or infectious condition, where the binding compound comprises an antibody or an antigen binding site derived from an antibody.

### CLAIM OF PRIORITY AND BENEFIT

This application claims priority and benefit from the U.S. Provisional Application entitled, ANTIBODY-MEDIATED THERAPEUTIC EFFECTS, U.S. Ser. No. 60/709,700, filed Aug. 19, 2005, assigned to Cerus Corporation, which is hereby incorporated herein by reference in its entirety.

### STATEMENT REGARDING FEDERALLY SPONSORED RESEARCH OR DEVELOPMENT

This invention was made, in part, with U.S. government support under National Cancer Institute NHI 1 K23CA104160-01. The government may have certain rights in the invention.

### FIELD OF THE INVENTION

The present invention relates to compositions and methods for enhancing immunorecruitment for treating cancers, tumors, tumor metastases, precancerous disorders, and infections. The methods include the use of Listeria in combination with an antibody.

### BACKGROUND OF THE INVENTION

Cancer, tumors, and infections are treated with reagents that modulate the immune system. Reagents that modulate the immune system include vaccines, antibodies, adjuvants, cytokines, and small molecules such as CpG oligodeoxynucleotides and imidazoquinolines (see, e.g., Becker (2005) Virus Genes 30:251-266; Schetter and Vollmer (2004) Curr. Opin. Drug Devel. 7:204-210; Majewski, et al. (2005) Int. J. Dermatol. 44:14-19), Hofmann, et al. (2005) J. Clin. Virol. 32:86-91; Huber, et al. (2005) Infection 33:25-29; Carter (2001) Nature Revs. Cancer 1:118-129; Dechant and Valaerius (2001) Crit. Revs. Oncol. 39:69-77; O'Connor, et al. (2004) Neurology 62:2038-2043). Vaccines include classical vaccines (inactivated whole organisms, extracts, or antigens), T cell vaccines, dendritic cell (DC) vaccines, and nucleic acid-based vaccines (see, e.g., Robinson and Amara (2005) Nat. Med. Suppl. 11:S25-S32; Plotkin (2005) Nat. Med. Suppl. 11:S5-S11; Pashine, et al. (2005) Nat. Med. Suppl. 11:S63-S68; Larche and Wraith (2005) Nat. Med. Suppl. 11:S69-S76). Another reagent useful for modulating the immune system is Listeria monocytogenes (L. monocytogenes; Lm) and this reagent has proven to be useful in treating cancer and tumors (see, e.g., Brockstedt, et al. (2004) Proc. Natl. Acad. Sci. USA 101:13832-13837; Brockstedt, et al. (2005) Nat. Med. 11:853-860; Starks, et al. (2004) J. Immunol. 173:420-427; Shen, et al. (1995) Proc. Natl. Acad. Sci. USA 92:3987-3991).

There has been interest in using the Gram positive bacterium L. monocytogenes for treating experimental tumors in animals. Listeria has been administered by way of intratumoral injections (Bast, et al. (1975) J. Natl. Cancer Inst. 54:757-761). Listeria, both heat-killed or viable, administered as a mixture with an experimental tumor cell line, or injected directly into a tumor, inhibited subsequent growth of the tumor cells in vivo (see, e.g., Bast, et al. (1975) J. Natl. Cancer Inst. 54:757-761; Youdim (1976) J. Immunol. 116:579-584; Youdim (1977) Cancer Res. 37:572-577; Fulton, et al. (1979) Infection Immunity 25:708-716; Keller, et al. (1989) Int. J. Cancer 44:512-317; Keller, et al. (1990) Eur. J. Immunol. 20:695-698; Pace, et al. (1985) J. Immunol. 134:977-981). Related studies demonstrated that there was no inhibition of tumor growth where Listeria was systemically disseminated (or where the Listeria was administered at a different site from the site of the administered tumor cells) (Youdim, et al. (1974) J. Natl. Cancer Inst. 52:193-198). Mycobacterium bovis BCG has also been used to stimulate immune response, though this bacterium is unusually slow growing, and resists modification by genetic engineering or transduction.

L. monocytogenes (Lm) has a natural tropism for the liver and spleen and, to some extent, other tissues such as the small intestines (see, e.g., Dussurget, et al. (2004) Ann. Rev. Microbiol. 58:587-610; Gouin, et al. (2005) Curr. Opin. Microbiol. 8:35-45; Cossart (2002) Int. J. Med. Microbiol. 291:401-409; Vazquez-Boland, et al. (2001) Clin. Microbiol. Rev. 14:584-640; Schluter, et al. (1999) Immunobiol. 201:188-195; Kursar, et al. (2002) J. Immunol. 168:6382-6387; Nishikawa, et al. (1998) Microbiol. Immunol. 42:325-327). Where the bacterium resides in the intestines, passage to the bloodstream is mediated by listerial proteins, such as actA and internalin A (see, e.g., Manohar, et al. (2001) Infection Immunity 69:3542-3549; Lecuit, et al. (2004) Proc. Natl. Acad. Sci. USA 101:6152-6157; Lecuit and Cossart (2002) Trends Mol. Med. 8:537-542). Once the bacterium enters a host cell, the life cycle of L. monocytogenes involves escape from the phagolysosome and to the cytosol. This life cycle contrasts with that of Mycobacterium, which remains inside the phagolysosome (see, e.g., Clemens, et al. (2002) Infection Immunity 70:5800-5807; Schluter, et al. (1998) Infect. Immunity 66:5930-5938; Gutierrez, et al. (2004) Cell 119:753-766). L. monocytogenes' escape from the phagolysosome is mediated by listerial proteins, such as listeriolysin (LLO), PI-PLC, and PC-PLC (see, e.g., Portnoy, et al. (2002) J. Cell Biol. 158:409-414).

As both metabolically active L. monocytogenes and heat-killed L. monocytogenes have been used in studies of immune response, it should be noted that these two preparations do not stimulate the immune system in the same way. Regarding the differences between metabolically active Listeria, and heat-killed Listeria, and without limiting the present invention to any mechanism, and without excluding the present invention from any mechanism, it should be noted that heat-killed Listeria have been found to produce an immune response, but where protection is not long lasting; that heat-killed Listeria can induce CD8⁺ T cells, but the CD8⁺ T cells are functionally impaired; that Listeria blocked in metabolism generally can stimulate immune response by cross-presentation, but not cross-presentation of MHC Class I epitopes; that Listeria that cannot express listeriolysin (LLO) (e.g., heat-killed Listeria) fail to enter the cytoplasm and fail to efficiently induce, e.g., IL-12, MCP-1, CD40, and CD80 (see, e.g., Emoto, et al. (1997) Infection Immunity 65:5003-5009; Vazquez-Boland, et al. (2001) Clin. Microbiol. Revs. 14:584-640; Brzoza, et al. (2004) J. Immunol. 173:2641-2651; Serbina, et al. (2003) Immunity 19:891-901; Janda, et al. (2004) J. Immunol. 173:5644-5651; Kursar, et al. (2004) J. Immunol. 172:3167-3172; Brunt, et al. (1990) J. Immunol. 145:3540-3546; Lauvau, et al. (2001) Science 294:1735-1739).

The present application incorporates by reference, in its entirety, U.S. Provisional patent application IMMUNORECRUITMENTAND ACTIVATION FOR ANTI-TUMOR TREATMENT of Pardoll, et al., U.S. Ser. No. 60/709,699, filed Aug. 19, 2005, assigned to Cerus Corporation. Also incorporated by reference is the corresponding U.S. Basic application, LISTERIA-MEDIATED IMMUNORECRUITMENT AND ACTIVATION, AND METHODS OF USE THEREOF, filed concurrently herewith and owned by the same assignee. The application also incorporates by reference, in its entirety, ENGINEERED LISTERIA AND METHODS OF USE THEREOF, U.S. Ser. No. 11/395,197, filed Mar. 30, 2006, and assigned to Cerus Corporation.

Methods for treating tumors, cancers, precancerous disorders, dysplasias, angiogenesis of tumors, and infections, are often ineffective. The present invention fulfills this need by providing an antibody and a Listeria for use in enhancing immunorecruitment, immunoactivation, and antibody-mediated cell cytotoxicity (ADCC), for treatment of, for example, metastatic liver cancer.

### BRIEF DESCRIPTION OF THE DRAWINGS

FIG. 1A demonstrates increased killing of tumor cells by splenocytes from Listeria-treated mice, where the increase was stimulated by Erbitux®.

FIG. 1B shows increased killing of tumor cells by splenocytes prepared from poly(I:C)-treated mice, where the increase was stimulated by Erbitux®.

FIG. 1C demonstrates increased killing of tumor cells by splenocytes from Listeria-treated mice, where the increase was stimulated by C225 antibody.

FIG. 1D shows increased killing of tumor cells by splenocytes prepared from poly(I:C)-treated mice, where the increase was stimulated the C225 antibody.

FIG. 2 demonstrate the activation of NK cells, as assessed by CD69 expression, where splenocytes, the source of NK cells, were isolated from mice treated under three different conditions: (1) Hanks Buffered Salt Solution; (2) Lm ΔactAΔinlB; or (3) poly (I:C).

FIGS. 3A to 3E disclose survival data.

FIG. 3A demonstrates that administering L. monocytogenes ΔactA or L. monocytogenes ΔactAΔinlB improved survival to tumors, where the bacteria were not engineered to express any heterologous antigen. This figure shows the survival in response to different numbers of doses, that is, one dose, three doses, or three doses.

FIG. 3B also demonstrates that administering L. monocytogenes ΔactA or L. monocytogenes ΔactAΔinlB increased survival to tumors, where the bacteria were not engineered to express any heterologous antigen. This figure shows the survival in response to different numbers of doses, that is, doses at intervals of three days, or at intervals of one week.

FIG. 3C reveals that L. monocytogenes ΔactAΔinlB increased survival to tumors, where the bacteria were not engineered to express any heterologous antigen. Doses were provided at intervals of three days, and here one of three different levels of bacteria were administered. Also, doses were provided at weekly intervals, and here again, one of three different levels of bacteria was given.

FIG. 3D demonstrates that administering CTX (at t=4 days) alone results in some increase in survival, and that administering CTX (at t=4 days) plus Listeria (Listeria administered at days 5, 12, and 19; Listeria administered at days 6, 13, and 20; or Listeria at days 7, 14, and 21) results in even greater survival.

FIG. 3E discloses the results of progressively delaying combination therapy with CTX plus Listeria ΔactAΔinlB.

FIG. 3F reveals survival of mice to CT26 tumors, where CT26 tumor cell inoculated mice were treated with Lm ΔactAΔinlB or with no Lm ΔactAΔinlB, as indicated. Mice also received no antibody, or antibodies that specifically deplete CD4+ T cells; CD8+ T cells; or NK cells, as indicated.

FIG. 3G reveals survival of mice to CT26 tumors, where CT26-tumor cell inoculated mice were treated with Listeria ΔactA plus GM CSF vaccine (GVAX), along with agents that specifically deplete CD4+ T cells, CD8+ T cells, or NK cells.

FIG. 3H shows the percentage of mice that were tumor free at 60 days after tumor re-challenge. Results are shown for control mice (“Control”) and long term survivors that were previously injected with Lm ΔactAΔinlB following inoculation with CT26. The long term survivors were re-challenged without injection of depleting antibodies (“No antibody”), following injection of anti-CD4+ antibodies (“Anti-CD4+ antibody”), or following injection of anti-CD8+ antibodies (“Anti-CD8+ antibody”).

FIG. 4A demonstrates that administering attenuated Listeria resulted in a dose-dependent increase in hepatic NK cells.

FIG. 4B shows that.administering attenuated Listeria did not increase the percent of splenic NK cells.

FIG. 4C reveals that administering attenuated Listeria increased expression of CD69 by hepatic NK cells in a dose dependent manner.

FIG. 4D reveals that administering attenuated Listeria increased expression of CD69 by splenic NK cells.

FIG. 5A discloses that administering attenuated Listeria resulted in an increase in hepatic NKT cells.

FIG. 5B discloses that administering attenuated Listeria did not increase the percent of splenic NKT cells.

FIG. 5C demonstrates that administering attenuated Listeria increased the expression of CD69 by hepatic NKT cells.

FIG. 5D demonstrates that administering attenuated Listeria increased the expression of CD69 by splenic NKT cells.

FIGS. 6A and 6B show that administering attenuated Listeria did not result in an increase in total T cells, as a percent of leukocytes, in the liver or spleen.

FIGS. 6C and 6D disclose that administering attenuated Listeria did not result in an increase in CD4+ T cells, as a percent of leukocytes, in the liver or spleen.

FIG. 6E demonstrates that administering attenuated Listeria stimulated the dose-dependent expression of CD69 by hepatic CD4+ T cells.

FIG. 6F demonstrates that administering attenuated Listeria stimulated expression of CD69 by splenic CD4+ T cells.

FIGS. 7A and 7B show that administering attenuated Listeria did not result in an increase in CD8+ T cells, as a percent of leukocytes, in the liver or spleen.

FIG. 7C demonstrates that administering attenuated Listeria increased CD69 expression by hepatic CD8+ T cells.

FIG. 7D demonstrates that administering attenuated Listeria increased CD69 expression by splenic CD8+ T cells.

FIG. 8A reveals that administering attenuated Listeria increased the percent of total hepatic leukocytes occurring as GR-1+ neutrophils.

FIG. 8B reveals that administering attenuated Listeria increased the percent of total splenic leukocytes occurring as GR-1+ neutrophils..

FIG. 9A indicates that administering attenuated Listeria increased the percent of hepatic CD4+ T cells expressing CD25.

FIG. 9B shows that administering attenuated Listeria increased the median expression of CD25 by hepatic CD4+ T cells.

FIG. 9C indicates that administering attenuated Listeria had little or no influence on the percent of splenic CD4+ T cells expressing CD25.

FIG. 9D shows that administering attenuated Listeria had little or no influence on expression of CD25 by spleen CD4+ T cells.

FIGS. 10 and 11 disclose time course studies.

FIG. 10A shows that administering attenuated Listeria increased the percent of hepatic leukocytes that are NK cells.

FIG. 10B shows that administering attenuated Listeria had little or no influence on the percent of splenic leukocytes that are NK cells.

FIG. 11A shows that administering attenuated Listeria increased the percent of hepatic leukocytes that are neutrophils.

FIG. 11B shows that administering attenuated Listeria increased the percent of splenic leukocytes that are neutrophils.

FIGS. 12 to 13 disclose results with administration of a vaccine comprising an attenuated tumor cell engineered to express a cytokine (GM-CSF). This vaccine is called GVAX. The term “GVAX,” “GM vaccine,” and “GM-CSF vaccine” may be used interchangeably.

FIGS. 12A, 12B, 12C, 12D, 12E, 12F, 12G, 12H, and 12I disclose the immune responses in the liver following administration of L. monocytogenes ΔactA (the Listeria was not modified to contain a nucleic acid encoding a heterologous antigen.) Also shown are immune responses in the liver following administration of both the Listeria and the GVAX vaccine. The immune responses followed include NK cell number (FIG. 12A); NKT cell number (FIG. 12B); CD8⁺ T cell number (FIG. 12C); plasmacytoid DC number (FIG. 12D); myeloid DC number (FIG. 12E); tumor specific CD8⁺ T cell number (FIG. 12F); as well as cell activation as assessed by expression of mRNA encoding interferon-gamma (FIGS. 12G and 12H). FIG. 12I shows FACS analysis of CD8⁺ T cells from liver of CT26 tumor cell-treated mice, where mice had also been administered with, e.g., various therapeutic agents.

FIGS. 13A and 13B demonstrate that administering the vaccine alone resulted in some increase in survival, while administering an attenuated Listeria with the vaccine produced greater survival. The number of bacteria administered was 10⁷ colony forming units (1e7 colony forming units; CFU).

FIG. 14 demonstrates that giving the vaccine (GM) alone resulted in a slight improvement in survival, while giving vaccine plus an attenuated Listeria (GM+Lm actA or GM+Lm actA/inlB) resulted in greater survival, while giving the GM vaccine plus an attenuated Listeria and cyclophosphamide (CTX), resulted in even greater survival.

FIGS. 15A to C demonstrate survival to tumors, where animals were administered with the vaccine (GM) only, or vaccine (GM) plus different levels of an attenuated L. monocytogenes.

FIG. 15A shows survival data with L. monocytogenes ΔactA (deletion mutant) administered at 3×106 CFU, 1×107 CFU, or 3×107 CFU.

FIG. 15B discloses survival data with L. monocytogenes ΔactAΔinlB (deletion mutant) administered at 3×106 CFU, 1×107 CFU, or 3×107 CFU.

FIG. 15C reveals survival data with the vaccine only, or with L. monocytogenes ΔactAΔinlB administered at 3×10³ CFU, 3×10⁴ CFU, 3×10⁵ CFU, 3×10⁶ CFU, or 3×10⁷ CFU.

FIG. 16 discloses treatment of lung tumors with L. monocytogenes ΔactAΔinlB.

FIG. 17 shows memory response (Elispot assays) resulting from a re-challenge with CT26 tumor cells, where tumor-inoculated mice had initially been treated with no therapeutic agent, Listeria only, GM-CSF vaccine plus Listeria, or cyclophosphamide (CTX) only.

FIG. 18 shows tumor volume of tumors resulting from a re-challenge with CT26 tumor cells, where tumor-inoculated mice had initially been treated with no therapeutic agent, Listeria only, GM-CSF vaccine plus Listeria, or cyclophosphamide (CTX) only.

FIG. 19 shows cytokine expression.

FIG. 20 discloses NK cell activation and recruitment, and MCP-1 expression.

FIG. 21A discloses expression of IL-1Ralpha in monkeys, after administering Lm ΔactAΔinlB.

FIG. 21B discloses expression of interferon-gamma (IFNgamma) in monkeys, after administering Lm ΔactAΔinlB.

FIG. 21C reveals expression of tumor necrosis factor-alpha (TNFalpha) in monkeys, after administering Lm ΔactAΔinlB.

FIG. 21D discloses expression of MCP-1 in monkeys, after administering Lm ΔactAΔinlB.

FIG. 21 E demonstrates expression of MIP-1beta in monkeys, after administering Lm ΔactAΔinlB.

FIG. 21F discloses expression of interleukin-6 (IL-6) in monkeys, after administering Lm ΔactAΔinlB.

FIG. 21G discloses expression of various cytokines in monkeys, following administration of Lm ΔactAΔinlB.

FIG. 22 shows a comparison of the anti-tumor activity induced by Lm ΔactAΔinlB, heat-killed (HK) Lm ΔactAΔinlB, and Δhly Lm.

### SUMMARY OF THE INVENTION

The present invention is based, in part, on the recognition that administering an antibody and Listeria monocytogenes enhances an immune response against tumor cells and killing of tumor cells.

Aspects of the invention relate to stimulating and enhancing an immune response.

Provided is a method for stimulating an immune response against a cancerous or infectious condition in a mammal having the condition, comprising administering to the mammal effective amounts of a Listeria and one or both of: a. an antibody that specifically binds to an antigen of the condition; or b. a binding compound derived from the antigen-binding site of an antibody that specifically binds to an antigen of the condition and also specifically binds to an immune cell that mediates antibody-dependent cell cytotoxicity (ADCC), wherein the combination of the Listeria and the antibody, or binding compound, is effective in stimulating the response.

Also provided is the above method, wherein the Listeria and the antibody, or binding compound, are administered simultaneously. Moreover, what is provided is the above method wherein the Listeria and the antibody, or binding compound, are not administered simultaneously. Also supplied is the above method, wherein the Listeria is attenuated. In addition, what is supplied is the above method wherein the binding compound derived from the antigen-binding site of an antibody further comprises an Fc region, or an Fc region derivative. Note also, that what is supplied is the above method

wherein the derivative of the Fc region has one or both of: a. enhanced affinity for an activating receptor expressed by the cell that mediates ADCC; or b. decreased affinity for an inhibiting receptor expressed by the cell that mediates ADCC. In another aspect, what is provided is the above method, wherein the Fc region derivative comprises an IgG1 Fc region that contains one or more of the mutations: a. S298A; b. E333A; or c. K334A, wherein the mutation is useful in mediating increased activation of the cell that mediates ADCC.

In yet another aspect, what is provided is the above method wherein the binding compound comprises a bispecific antibody, and wherein the first binding site of the bispecific antibody specifically binds to the antigen of the condition and the second binding site of the bispecific antibody specifically binds to the immune cell that mediates ADCC. Also provided is the above method, wherein the binding compound is a peptide mimetic of an antibody that specifically binds to the antigen of the condition. Also contemplated is the above method, wherein the Listeria is metabolically active and is essentially incapable of one or more of: a. forming colonies; b. replicating; or c. dividing. In another aspect, what is contemplated is the above method wherein the Listeria is essentially metabolically inactive. Note also, that what is supplied is the above method wherein the attenuated Listeria is attenuated in one or more of: a. growth; b. cell-to-cell spread; c. binding to or entry into a cell; d. replication; or e. DNA repair.

Regarding attenuation, what is provided is the above method, wherein the Listeria is attenuated by one or more of: a. an actA mutation; b. an inlB mutation; c. a uvrA mutation; d. a uvrB mutation; e. a uvrC mutation; f. a nucleic acid targeted compound; or g. a uvrAB mutation and a nucleic acid targeting compound. In another aspect, provided is the above method wherein the nucleic acid targeting compound is a psoralen. Additionally, what is provided the above method, wherein the condition comprises one or more of a tumor, cancer, or pre-cancerous disorder. Moreover, what is contemplated is the above method wherein the condition comprises an infection. Note also that what is provided is the above method, wherein the condition comprises an infection by one or more of: a. hepatitis B; b. hepatitis C; c. cytomegalovirus (CMV); d. HIV; e. Epstein-Barr virus (EBV); or f. leishmaniasis. Furthermore, the present invention provides the above method, wherein the condition is of the liver. Also provided is the above method, wherein the immune response is against a cell of the condition. Additionally, what is provided is the above method wherein the immune response comprises an innate immune response. Note also that what is supplied is the above method, wherein the immune response comprises an adaptive immune response. Further, what is contemplated is the above method wherein the mammal is human. Added is the above method, wherein the Listeria is Listeria monocytogenes. Further provided is the above method, wherein the Listeria comprises a nucleic acid encoding a heterologous antigen. Also, provided is the above method wherein the Listeria is a first reagent, and the antibody, or the binding compound, is a second reagent, further comprising administering a third reagent to the mammal. And also, provided is the above method wherein the third reagent comprises one or more of: a. an agonist or antagonist of a cytokine; b. an inhibitor of a T regulatory cell (Treg); or c. cyclophosphamide (CTX). And what is supplied is the above method, wherein the immune response comprises activation of, or an inflammation by, one or any combination of: a. an NK cell; b. an NKT cell; c. a dendritic cell (DC); d. a monocyte or macrophage; e. a neutrophil; f. a toll-like receptor (TLR), or g. nucleotide-binding oligomerization domain protein (NOD protein), as compared with immune response in the absense of the administering of the effective amount of the Listeria. Another aspect is the above method, wherein the immune response comprises increased expression of one or any combination of: a. CD69; b. interferon-gamma (IFNgamma); c. interferon-alpha (IFNalpha) or interferon-beta (IFNbeta); d. interleukin-12 (IL-12), e. monocyte chemoattractant protein (MCP-1), or interleukin-6 (IL-6), as compared with expression in the absence of the administering of the effective amount of the Listeria. The invention also embraces the above method, wherein the stimulating results in: a. an increase in the percent of NK cells in hepatic leukocytes of the mammal compared to the percent without the administering of the Listeria; or b. an increase in expression of an activation marker by a hepatic NK cell, compared to the expression without the administering of the Listeria. Furthermore, what is supplied is the above method, wherein the increase in the percent of NK cells in the population of hepatic leukocytes is at least: a. 5%; b. 10%; c. 15%; d. 20%; or e. 25%, greater than compared to the percent without the administering of the Listeria. Another aspect of the present invention is the above method, wherein the administered Listeria is one or both of: a. not administered orally to the mammal; or b. administered to the mammal as a composition that is at least 99% free of other types of bacteria.

The following aspects relate to methods of treating.

The present invention provides a method for treating a cancerous or infectious condition in a mammal having the condition, comprising administering to the mammal effective amounts of a Listeria with one or both of: a. an antibody that specifically binds to an antigen of the condition; or b. a binding compound derived from an antibody that specifically binds to an antigen of the condition and also specifically binds to an immune cell that mediates ADCC, wherein the combination of the Listeria and the antibody, or binding compound, is effective in ameliorating or reducing the condition. Provided also is the above method, wherein the Listeria and the antibody, or binding compound, are administered simultaneously. And also provided is the above method, wherein the Listeria and the antibody, or binding compound, are not administered simultaneously. Moreover, what is provided is the above method, wherein the Listeria is attenuated. And also provided is the above method, wherein the binding compound derived from the antigen-binding site of an antibody further comprises an Fc region, or an Fc region derivative. Another aspect provides the above method, wherein the derivative of the Fc region has one or both of: a. enhanced affinity for an activating receptor expressed by the cell that mediates ADCC; or b. decreased affinity for an inhibiting receptor expressed by the cell that mediates ADCC. Yet another aspect of the present invention provides the above method, wherein the Fc region derivative comprises an IgG1 Fc region that contains one or more of the mutations: a. S298A; b. E333A; or c. K334A,

wherein the mutation is useful in mediating increased activation of the cell that mediates ADCC. And what is contemplated is the above method, wherein the binding compound comprises a bispecific antibody, wherein the first binding site of the bispecific antibody specifically binds to the antigen of the condition and the second binding site of the bispecific antibody specifically binds to the immune cell that mediates ADCC. Also provided is the above method, wherein the binding compound is a peptide mimetic of an antibody that specifically binds to the antigen of the condition. Note also, that what is provided in the present invention, is the above method wherein the Listeria is metabolically active and is essentially incapable of one or more of: a. forming colonies; b. replicating; or c. dividing. Further, the invention contemplates the above method, wherein the Listeria is essentially metabolically inactive. Note also, that the invention embraces the above method, wherein the attenuated Listeria is attenuated in one or more of: a. growth; b. cell-to-cell spread; c. binding to or entry into a cell; d. replication; or e. DNA repair. Also encompassed, is the above method, wherein the Listeria is attenuated by one or more of: a. an actA mutation; b. an inlB mutation; c. a uvrA mutation; d. a uvrB mutation; e. a uvrC mutation; f. a nucleic acid targeting compound; or g. a uvrAB mutation and a nucleic acid targeting compound. Furthermore, what is contemplated is the above method, wherein the nucleic acid targeting compound is a psoralen. Also available, is the above method wherein the condition comprises a cancer, tumor, or pre-cancerous disorder. The invention further encompasses the above method, wherein the condition comprises an infection. In yet another aspect, the invention embraces the above method, wherein the condition comprises an infection by one or more of: a. hepatitis B; b. hepatitis C; c. CMV; d. HIV; e. EBV; or f. leishmaniasis. And the invention supplies the above method, wherein the condition is of the liver. Also, it provides the above method wherein the immune response is against a cell of the condition. In yet an additional aspect, the invention provides the above method, wherein the immune response comprises an innate immune response. Note also, that what is supplied is the above method wherein the immune response comprises an adaptive immune response. Also embraced is the above method, wherein the mammal is human. Encompassed by the present invention is the above method, wherein the Listeria is Listeria monocytogenes. Contemplated by the present invention, is the above method, wherein the Listeria comprises a nucleic acid encoding a heterologous antigen. Furthermore, what is supplied is the above method, wherein the Listeria is a first reagent, and the antibody, or the binding compound, is a second reagent, further comprising administering a third reagent to the mammal. And what is supplied is the above method,

wherein the third reagent comprises one or more of: a. an agonist or antagonist of a cytokine; b. an inhibitor of a T regulatory cell (Treg); or c. cyclophosphamide (CTX). Note also, that the invention additionally provides the above method, wherein the immune response comprises activation of, or inflammation by, one or any combination, of: a. an NK cell; b. an NKT cell; c. a dendritic cell (DC); d. a monocyte or macrophage; e. a neutrophil; or f. a toll-like receptor (TLR) or nucleotide-binding oligomerization domain (NOD) protein, as compared with immune response in the absence of the administering of the effective amount of the Listeria. Another aspect that is contemplated is the above method, wherein the immune response comprises increased expression of one or any combination of: a. CD69; b. interferon-gamma (IFNgamma); c. interferon-alpha (IFNalpha) or interferon-beta (IFNbeta); d. interleukin-12 (IL-12), e. monocyte chemoattractant protein (MCP-1), or f. interleukin-6 (IL-6), as compared with expression in the absence of the administering of the effective amount of the attenuated Listeria. Additionally, what is supplied is the above method, wherein the stimulating results in: a. an increase in the percent of NK cells in hepatic leukocytes, compared to the percent without the administering of the Listeria; or b. an increase in expression of an activation marker by a hepatic NK cell, compared to the expression without the administering the Listeria. And also embraced is the above method, wherein the increase in the percent of NK cells in the hepatic leukocytes is at least: a. 5%; b. 10%; c. 15%; d. 20%; or e. 25% greater than compared to the percent without the administering of the Listeria. And also embraced, is the above method, wherein the treating increases survival of the mammal to the condition, as determined by comparison to a suitable control mammal having the condition not administered with the Listeria, antibody, or binding compound. Moreover, also provided is the above method, wherein the treating increases survival of the mammal by at least: a. five days; b. ten days; c. fifteen days; or d. twenty days. Encompassed by the invention is the above method, wherein the condition comprises one or more of cancer cells, tumors, or an infectious agent, and wherein the treating reduces one or more of the: a. number of tumors or cancer cells; b. tumor mass; or c. titer of the infectious agent, in the mammal. Another aspect of the present invention is the above method, wherein the administered Listeria is one or both of: a. not administered orally to the mammal; or b. administered to the mammal as a composition that is at least 99% free of other types of bacteria.

The following aspects relate to the individual aspects disclosed above.

The present invention comprises a method of stimulating the immune system against an infectious disorder, where the infectious disorder is a Listeria infection. Also comprised, is a method of stimulating the immune system against an infectious disorder, where the infectious disorder is not a Listeria infection, that is, excludes listerial infections.

Each of the aspects disclosed herein encompasses methods using a Listeria that is not attenuated. Also, each of the aspects encompasses methods using a Listeria that is attenuated.

Each of the aspects disclosed herein encompasses methods and reagents using a Listeria that comprises a nucleic acid encoding at least one tumor antigen, a Listeria that comprises a nucleic acid encoding at least one cancer antigen, a Listeria that comprises a nucleic acid encoding at least one heterologous antigen, as well as a Listeria that expresses at least one tumor antigen, cancer antigen, and/or heterologous antigen.

Each of the aspects disclosed herein encompasses methods and reagents using an a Listeria that does not comprise a nucleic acid encoding a tumor antigen, an a Listeria that does not comprise a nucleic acid encoding a cancer antigen, a Listeria that does not comprise a nucleic acid encoding a heterologous antigen, as well as an a Listeria that does not express a tumor antigen, cancer antigen, and/or a heterologous antigen.

Each of the aspects disclosed herein encompasses methods and reagents using a Listeria that comprises a nucleic acid encoding an antigen from a non-listerial infectious organism. Each of the above-disclosed aspects encompasses methods and reagents using a Listeria that does comprises a nucleic acid encoding an antigen from a virus or parasite.

Each of the aspects disclosed herein encompasses methods and reagents using a Listeria that does not comprise a nucleic acid encoding an antigen from a non-listerial infectious organism. Each of the above-disclosed aspects encompasses methods and reagents using a Listeria that does not comprise a nucleic acid encoding an antigen from a virus or parasite.

Each of the aspects disclosed herein also encompasses a Listeria that is not prepared by growing on a medium based on animal protein, but is prepared by growing on a different type of medium. Each of the above-disclosed aspects also encompasses a Listeria that is not prepared by growing on a medium containing peptides derived from animal protein, but is prepared by growing on a different type of medium. Moreover, each of the above-disclosed aspects encompasses administration of a Listeria by a route that is not oral or that is not enteral. Additionally, each of the above-disclosed aspects includes administration of a Listeria by a route that does not require movement from the gut lumen to the lymphatics or bloodstream.

Each of the aspects disclosed herein further comprises a method wherein the Listeria are not injected directly into the tumor or are not directly injected into a site that is affected by the cancer, precancerous disorder, tumor, or infection.

Additionally, each of the aspects disclosed herein encompasses administering the Listeria by direct injection into a tumor, by direct injection into a cancerous lesion, and/or by direct injection into a lesion of infection. Also, the invention includes each of the above aspects, where administration is not by direct injection into a tumor, not by direct injection into a cancerous lesion, and/or not by direct injection into a lesion of infection.

Provided is a vaccine where the heterologous antigen, as in any of the aspects disclosed herein, is a tumor antigen or is derived from a tumor antigen. Also provided is a vaccine where the heterologous antigen, as in any of the aspects disclosed herein, is a cancer antigen, or is derived from a cancer antigen. Moreover, what is provided is a vaccine where the heterologous antigen, as in any of the aspects disclosed herein, is an antigen of an infectious organism, or is derived from an antigen of an infectious organism, e.g., a virus, bacterium, or multi-cellular organism.

A further aspect provides a nucleic acid where the heterologous antigen, as in any of the aspects disclosed herein, is a tumor antigen or derived from a tumor antigen. Also provided is a nucleic acid where the heterologous antigen, as in any of the aspects disclosed herein, is a cancer antigen, or is derived from a cancer antigen. Moreover, what is provided is a nucleic acid, where the heterologous antigen, as in any of the aspects disclosed herein, is an antigen of an infectious organism, or is derived from an antigen of an infectious organism, e.g., a virus, bacterium, or multi-cellular organism.

In another aspect, what is provided is a Listeria where the heterologous antigen, as in any of the aspects disclosed herein, is a tumor antigen or derived from a tumor antigen. Also provided is a Listeria where the heterologous antigen, as in any of the aspects disclosed herein, is a cancer antigen, or is derived from a cancer antigen. Moreover, what is provided is a Listeria, where the heterologous antigen, as in any of the aspects disclosed herein, is an antigen of an infectious organism, or is derived from an antigen of an infectious organism, e.g., a virus, bacterium, or multi-cellular organism.

Each of the above-disclosed aspects also encompasses an attenuated Listeria that is not prepared by growing on a medium based on animal or meat protein, but is prepared by growing on a different type of medium. Provided is an attenuated Listeria not prepared by growing on a medium based on meat or animal protein, but is prepared by growing on a medium based on yeast and/or vegetable derived protein.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual publication, sequences accessed by a GenBank Accession No., patent application, patent, Sequence Listing, nucleotide or oligo- or polypeptide sequence in the Sequence Listing, as well as figures and drawings in said publications and patent documents, was specifically and individually indicated to be incorporated by reference.

I. Definitions.

Abbreviations used to indicate a mutation in a gene, or in a bacterium encoding a gene, are as follows. By way of example, the abbreviation “Listeria ΔactA” means that part, or all, of the actA gene was deleted. The delta symbol (Δ) means deletion. Lm means “Listeria monocytogenes.” An abbreviation including a superscripted minus sign (Listeria actA⁻) means that the actA gene was mutated, e.g., by way of a deletion, point mutation, or frameshift mutation, but not limited to these types of mutations. Exponentials may be abbreviated. For example “3e7” means 3×10⁷.

“Administration,” “administering,” and “treatment,” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration,” “administering,” and “treatment” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration,” “administration,” and “treatment” also encompass in vitro and ex vivo treatments, e.g., of a cell, by a reagent, diagnostic, binding composition, or by another cell. Depending on the context, “treatment” of a subject can imply that the subject is in need of treatment, e.g., in the situation where the subject comprises a disorder expected to be ameliorated by administration of a reagent.

The administered antibody, or binding compound derived from an antibody, that is administered to a mammal does not include an antibody that is generated in its entirety, by the subject or mammal. In other words, the administered antibody of the present invention does not encompass antibodies generated as follows: (1) A mammal with a cancerous disorder biosynthesizes a tumor antigen, or a mammal with an infection biosynthesizes a bacterial antigen, viral antigen, and the like, and; (2) the antigen stimulates the immune system of the mammal to biosynthesize an antibody. The immune system of the mammal may produce an antibody, and the antibody may contribute to ADCC, however, this antibody is not encompassed by the administered antibody or binding composition of the invention.

An agonist, as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor. An antagonist, as it relates to a ligand and receptor, comprises a molecule, combination of molecules, or a complex, that antagonizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of the receptor. By way of example, an antagonist of GM-CSF receptor includes, without implying any limitation, an antibody that binds to GM-CSF and prevents GM-CSF from binding to GM-CSF receptor, or an antibody that binds to GM-CSF receptor and prevents GM-CSF from binding to the receptor, or where the antibody locks the receptor in an inactive conformation.

“Antibody” refers to a polypeptide comprising a framework region from an immunoglobulin gene or fragments thereof that specifically recognizes and binds an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A “partially humanized” or “chimeric” antibody contains heavy and light chain variable regions of, e.g., murine origin, joined onto human heavy and light chain constant regions. A “humanized” or “fully humanized” antibody contains the amino acid sequences from the six complementarity-determining regions (CDRs) of the parent antibody, e.g., a mouse antibody, grafted to a human antibody framework. “Human” antibodies are antibodies containing amino acid sequences that are of 100% human origin, where the antibodies may be expressed, e.g., in a human, animal, bacterial, or viral host (Baca, et al. (1997) J. Biol. Chem. 272:10678-10684; Clark (2000) Immunol. Today 21:397-402).

Antibody fragments can be produced by digestion with various peptidases or by recombinant techniques. For example, pepsin digests an antibody below the disulfide linkages in the hinge region to produce F(ab′)₂, a dimer of Fab which itself is a light chain joined to V_{H}-C_{H}1 by a disulfide bond. The F(ab′)₂ can be reduced under mild conditions to break the disulfide linkage in the hinge region, thereby converting the F(ab′)₂ dimer into an Fa′ monomer. The Fa′ monomer is essentially Fab with part of the hinge region. “Fv” fragment comprises a dimer of one heavy chain and one light chain variable domain in tight association with each other. A single variable domain (or half of an Fv comprising only three CDRs specific for an antigen) has the ability to recognize and bind antigen, although at a lower affinity than the entire binding site. “Antibody” can refer to an antibody fragment produced by the modification of an intact antibody, to antibody compositions synthesized de novo using recombinant DNA methodologies, to single chain antibodies, to antibodies produced by phage display methods, and to monoclonal antibodies (U.S. Pat. No. 4,816,567 issued to Cabilly, et al.; U.S. Pat. No. 4,642,334 issued to Moore, et al.; Queen, et al. (1989) Proc. Natl Acad. Sci. USA 86:10029-10033; Kohler, et al. (1975) Nature 256:495-497).

“Monoclonal antibody” (mAb) refers to an antibody obtained from a population of substantially homogeneous antibodies, i.e., the individual antibody polypeptides comprising the population are identical except for possible naturally occurring mutations in the polypeptide chain that may be present in minor amounts, or to heterogeneity in glycosylation, disulfide formation, or folding, and the like. “Monoclonal antibody” does not suggest or limit any characteristic of the oligosaccharide component, or that there is homogeneity or heterogeneity with regard to oligosaccharide component. Monoclonal antibodies are highly specific, being directed against a single antigenic site or epitope. Polyclonal antibody preparations typically include different antibodies directed against different epitopes.. In addition to their specificity, monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. “Monoclonal antibodies” also include clones of antigen-recognition and binding-site containing antibody fragments, such as those derived from phage antibody libraries. “Diabody” refers to a fragment comprising a heavy chain variable domain (V_{H}) connected to a light chain variable domain (V_{L}) (Hollinger, et al. (1993) Proc. Natl. Acad. Sci. USA 90:6444-6448).

“Antigen presenting cells” (APCs) are cells of the immune system used for presenting antigen to T cells. APCs include dendritic cells, monocytes, macrophages, marginal zone Kupffer cells, microglia, Langerhans cells, T cells, and B cells (see, e.g., Rodriguez-Pinto and Moreno (2005) Eur. J. Immunol. 35:1097-1105). Dendritic cells occur in at least two lineages. The first lineage encompasses pre-DC1, myeloid DC1, and mature DC1. The second lineage encompasses CD34⁺⁺CD45RA⁻ early progenitor multipotent cells, CD34⁺⁺CD45RA⁺ cells, CD34⁺⁺CD45RA⁺⁺CD4⁺ IL-3Ralpha⁺⁺ pro-DC2 cells, CD4⁺CD11c⁻ plasmacytoid pre-DC2 cells, lymphoid human DC2 plasmacytoid-derived DC2s, and mature DC2s (see, e.g., Gilliet and Liu (2002) J. Exp. Med. 195:695-704; Bauer, et al. (2001) J. Immunol. 166:5000-5007; Arpinati, et al. (2000) Blood 95:2484-2490; Kadowaki, et al. (2001) J. Exp. Med. 194:863-869; Liu (2002) Human Immunology 63:1067-1071).

“Attenuation” and “attenuated” encompasses a bacterium, virus, parasite, tumor cell, and the like, that is modified to reduce toxicity to a host. The host can be a human or animal host, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and conventionally by at least 100,000-fold.

“Attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

Attenuation can be effected by, e.g., heat-treatment or chemical modification. Attenuation can also be effected by genetic modification of a nucleic acid that modulates, e.g., metabolism, extracellular growth, or intracellular growth, genetic modification of a nucleic acid encoding a virulence factor, such as listerial prfA, actA, listeriolysin (LLO), an adhesion mediating factor (e.g., an internalin), mpl, phosphatidylcholine phospholipase C (PC-PLC), phosphatidylinositol-specific phospholipase C (PI-PLC; plcA gene), any combination of the above, and the like. Attenuation can be assessed by comparing a biological function of an attenuated Listeria with the corresponding biological function shown by an appropriate parent Listeria.

The present invention includes the use of a Listeria that is attenuated by treating with a nucleic acid targeting agent or a nucleic acid targeted compound, such as a cross-linking agent, a psoralen, a nitrogen mustard, cis-platin, a bulky adduct, ultraviolet light, gamma irradiation, any combination therof, and the like. The Listeria can also be attenuated by mutating at least one nucleic acid repair gene, e.g., uvrA, uvrB, uvrAB, uvrC, uvrD, uvrAB, phrA, and/or recA. Moreover, the invention includes the use of a Listeria attenuated by both a nucleic acid targeting agent and by mutating a nucleic acid repair gene. Additionally, the invention includes the use of Listeria treated with a light sensitive nucleic acid targeting agent, such as a psoralen, or a light sensitive nucleic acid cross-linking agent, such as a psoralen, followed by exposure to ultraviolet light (see, e.g., U.S. Pat. Publication Nos. U.S. 2004/0228877 of Dubensky, et al. and U.S. 2004/0197343 of Dubensky, et al.).

“Cancerous condition” and “cancerous disorder” encompass, without implying any limitation, a cancer, a tumor, a metastasis, an angiogenesis of a tumor, and precancerous disorders such as dysplasias.

“Conservatively modified variants” applies to both amino acid and nucleic acid sequences. With respect to particular nucleic acid sequences, a conservatively modified variant refers to nucleic acids encoding identical amino acid sequences, or amino acid sequences that have one or more conservative substitutions. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).
(1) Hydrophobic: Norleucine, Ile, Val, Leu, Phe, Cys, Met;
(2) Neutral hydrophilic: Cys, Ser, Thr;
(3) Acidic: Asp, Glu;
(4) Basic: Asn, Gln, His, Lys, Arg;
(5) Residues that influence chain orientation: Gly, Pro;
(6) Aromatic: Trp, Tyr, Phe; and
(7) Small amino acids: Gly, Ala, Ser.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. An effective amount also encompasses an amount that results in a desired immune response.

An “extracellular fluid” encompasses, e.g., serum, plasma, blood, interstitial fluid, cerebrospinal fluid, secreted fluids, lymph, bile, sweat, and urine. An “extracelluar fluid” can comprise a colloid or a suspension, e.g., whole blood or coagulated blood.

“Gene” refers to a nucleic acid sequence encoding an oligopeptide or polypeptide. The oligopeptide or polypeptide can be biologically active, antigenically active, biologically inactive, or antigenically inactive, and the like. The term gene encompasses, e.g., the sum of the open reading frames (ORFs) encoding a specific oligopeptide or polypeptide; the sum of the ORFs plus the nucleic acids encoding introns; the sum of the ORFs and the operably linked promoter(s); the sum of the ORFS and the operably linked promoter(s) and any introns, the sum of the ORFS and the operably linked promoter(s), intron(s), and promoter(s), and other regulatory elements, such as enhancer(s). The term gene can also refer to a nucleic acid that encodes a peptide encompassing an antigen or an antigenically active fragment of a peptide, oligopeptide, polypeptide, or protein. The term gene does not necessarily imply that the encoded gene has any biological activity, aside from antigenic stimulation of innate and/or adaptive immune response. A nucleic acid sequence encoding a non-expressible sequence is generally considered a pseudogene. The term gene also encompasses nucleic acid sequences encoding a ribonucleic acid such as rRNA, tRNA, or a ribozyme.

“Growth” of a bacterium encompasses, without limitation, functions of bacterial physiology and bacterial nucleic acids relating to colonization, replication, increase in listerial protein content, increase in listerial lipid content. Unless specified otherwise explicitly or by context, growth of a bacterium encompasses growth of the bacterium outside a host cell, and also growth inside a host cell. Growth related genes include, without implying any limitation, those that mediate energy production (e.g., glycolysis), nutrient transport, transcription, translation, and replication.

“Growth”, as a term used in the listerial art, refers to bacterial growth and multiplication in the cytoplasm of an infected host cell and generally does not refer to in vitro growth. For example, a gene that is highly specific for “growth” is one which encodes a protein that does not contribute to growth in vitro, and does not appreciably contribute to growth in conventional bacterial broth or agar, but does contribute to some extent or to a large extent to intracellular growth and multiplication in the cytoplasm of an infected cell.

Conventionally, growth of attenuated Listeria used in the present invention is at most 80% that of the parent Listeria strain, more conventionally growth of the attenuated Listeria is at most 70% that of the parent Listeria strain, most conventionally growth of the attenuated Listeria is at most 60% that of the parent Listeria strain, normally, growth of the attenuated Listeria of the present invention is at most 50% that of the parent Listeria strain; more normally growth is at most 45% that of the parent strain; most normally growth is 40% that of the parent strain; often growth is at most 35% that of the parent strain, more often growth is at most 30% that of the parent strain; and most often growth is at most 25% that of the parent strain; usually growth is at most 20% that of the parent strain; more usually growth is at most 15% that of the parent strain; most usually growth is at most 10% that of the parent strain; typically growth is at most 5% that of the parent strain; more typically growth of the attenuated Listeria used in the present invention is at most 1% that of the parent strain; and often growth is not detectable. Growth of the parent and the attenuated strain can be compared by measuring extracellular growth of both organisms. Growth of the parent and the attenuated strain can also be compared by measuring intracellular growth of both organisms.

The term “growth related gene” includes a gene that stimulates the rate of intracellular growth by the same amount that stimulates the rate of extracellular growth, by at least 20% greater than it stimulates the rate of extracellular growth; more normally by at least 30% greater than the rate it stimulates extracellular growth; most normally at least 40% greater than the rate it stimulates extracellular growth; usually at least 60% greater than the rate it stimulates extracellular growth; more usually at least 80% greater than the rate it stimulates extracellular growth; most usually it stimulates the rate of intracellular growth at least 100% (2-fold) greater than the rate it stimulates extracellular growth; often at least 3-fold greater than the rate it stimulates extracellular growth; more often at least 4-fold greater than the rate it stimulates extracellular growth; and most often at least 10-fold greater than the rate it stimulates extracellular growth; typically at least 50-fold greater than the rate it stimulates extracellular growth; and most typically at least 100-fold greater than the rate it stimulates extracellular growth.

“Immune condition” or “immune disorder” encompasses a disorder, condition, syndrome, or disease resulting from ineffective, inappropriate, or pathological response of the immune system, e.g., to a persistent infection or to a persistent cancer (see, e.g., Jacobson, et al. (1997) Clin. Immunol. Immunopathol. 84:223-243). “Immune condition” or “immune disorder” encompasses, e.g., pathological inflammation, an inflammatory disorder, and an autoimmune disorder or disease. “Immune condition” or “immune disorder” also can refer to infections, persistent infections, and proliferative conditions, such as cancer, tumors, and angiogenesis, including infections, tumors, and cancers that resist irradication by the immune system. “Immune condition” or “immune disorder” also encompasses cancers induced by an infective agent, including the non-limiting examples of cancers induced by hepatitis B virus, hepatitis C virus, simian virus 40 (SV40), Epstein-Barr virus, papillomaviruses, polyomaviruses, Kaposi's sarcoma herpesvirus, human T-cell leukemia virus, and Helicobacter pylori (see, e.g., Young and Rickinson (2004) Nat. Rev. Cancer 4:757-768; Pagano, et al. (2004) Semin. Cancer Biol. 14:453-471; Li, et al. (2005) Cell Res. 15:262-271).

“Innate immunity,” “innate response,” and “innate immune response” encompasses, without limitation, a response resulting from recognition of a pathogen-associated molecular pattern (PAMP). “Innate response” can encompass a response mediated by a toll-like receptor (TLR), mediated by a NOD protein (nucleotide-binding oligomerization domain protein), or mediated by scavenger receptors, mannose receptors, or beta-glucan receptors (see, e.g., Pashine, et al. (2005) Nat. Med. Suppl. 11:S63-S68). “Innate response” is characterized by the fact that a TLR can be stimulated by any member of a family of ligands (not merely by one ligand having a distinct structure). Moreover, “innate response” is distinguished in that a ligand that stimulates a TLR can promote a response against an antigen, where the ligand need not have any structural identity or structural similarity to the antigen. Innate response also encompasses physiological activities mediated by opsons or lectins (see, e.g., Doherty and Arditi (2004) J. Clin. Invest. 114:1699-1703; Tvinnereim, et al. (2004) J. Immunol. 173:1994-2002; Vankayalapati, et al. (2004) J. Immunol. 172:130-137; Kelly, et al. (2002) Nat. Immunol. 3:83-90; Alvarez-Dominguez, et al. (1993) Infection Immunity 61:3664-3672; Alvarez-Dominguez, et al. (2000) Immunology 101:83-89; Roos, et al. (2004) Eur. J. Immunol. 34:2589-2598; Takeda and Akira (2005) International Immunity 17:1-14; Weiss, et al. (2004) J. Immunol. 172:4463-4469; Chamaillard, et al. (2003) Cell Microbiol. 5:581-592; Philpott and Girardin (2004) Mol. Immunol. 41:1099-1108).

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

“Ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity, or it may have a different identity, as the first cell. A ligand or receptor may be entirely intracellular, that is, it may reside in the cytosol, nucleus, or in some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

As used herein, the term “kit” refers to components packaged and/or marked for use with each other, although not necessarily simultaneously. A kit may contain the antibody containing composition and the Listeria containing composition in separate containers. A kit may also contain the pharmaceutically acceptable excipients in separate containers. A kit may also contain instructions for combining the components so as to formulate immunogenic compositions suitable for administration to a mammal.

A bacterium that is “metabolically active” encompasses a bacterium, including a L. monocytogenes, where colony formation is impaired or substantially prevented but where transcription is essentially not impaired; where replication is impaired or substantially prevented but where transcription is essentially not impaired; or where cell division is impaired or substantially prevented but where transcription is essentially not impaired. A bacterium that is “metabolically active” also encompasses a bacterium, including a L. monocytogenes, where colony formation, replication, and/or cell division, is impaired or substantially prevented but where an indication of metabolism, e.g., translation, secretion, transport, respiration, fermentation, glycolysis, motility is not impaired or is essentially not impaired. Various indicia of metabolism for L. monocytogenes are disclosed (see, e.g., Karlin, et al. (2004) Proc. Natl. Acad. Sci. USA 101:6182-6187; Gilbreth, et al. (2004) Curr. Microbiol. 49:95-98).

The metabolically active bacterium of the present invention encompasses a bacterium where colony formation, replication, and/or cell division, is under 5% that of a suitable parent (or control) bacterium but where metabolism as compared to that of a suitable parent (or control) bacterium, is normally at least 20% that of the parent, more normally at least 30% that of the parent, most normally at least 40% that of the parent, typically at least 50% that of the parent, more typically at least 60% that of the parent, most typically at least 70% that of the parent, usually at least 80% that of the parent, more usually at least 90% that of the parent, and most usually indistinguishable from that of the parent bacterium, and in another aspect, greater than that of the parent.

The metabolically active bacterium of the present invention encompasses a bacterium where colony formation, replication, and/or cell division, is under 0.5% that of a suitable parent (or control) bacterium and where metabolism, as compared to that of a suitable parent (or control) bacterium, is normally at least 20% that of the parent, more normally at least 30% that of the parent, most normally at least 40% that of the parent, typically at least 50% that of the parent, more typically at least 60% that of the parent, most typically at least 70% that of the parent, usually at least 80% that of the parent, more usually at least 90% that of the parent, and most usually indistinguishable from that of the parent bacterium, and in another aspect, greater than that of the parent. Colony formation, replication, and/or cell division is measured under conditions that facilitate replication (e.g., not frozen). A bacterium that is essentially metabolically inactive includes, without limitation, a bacterium that is heat-killed. Residual metabolic activity of an essentially metabolically inactive bacterium can be due to, for example, oxidation of lipids, oxidation of sulfhydryls, reactions catalyzed by heavy metals, or to enzymes that are stable to heat-treatment.

“Nucleic acid” refers to deoxyribonucleotides or ribonucleotides and polymers thereof in either single stranded, double-stranded form, or multi-stranded form. The term nucleic acid may be used interchangeably with gene, cDNA, mRNA, oligonucleotide, and polynucleotide, depending on the context. A particular nucleic acid sequence can also implicitly encompasses “allelic variants” and “splice variants.”

“Operably linked” in the context of a promoter and a nucleic acid encoding a mRNA means that the promoter can be used to initiate transcription of that nucleic acid.

“Peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length and generally less than about 25 amino acids in length, where the maximal length is a function of custom or context. The terms “peptide” and “oligopeptide” may be used interchangeably.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses methods using polypeptide variants, e.g., involving glycosylation, phosphorylation, sulfation, disulfide bond formation, deamidation, isomerization, cleavage points in signal or leader sequence processing, covalent and non-covalently bound cofactors, oxidized variants, and the like. The formation of disulfide linked proteins is described (see, e.g., Woycechowsky and Raines (2000) Curr. Opin. Chem. Biol. 4:533-539; Creighton, et al. (1995) Trends Biotechnol. 13:18-23).

“Precancerous condition” encompasses, without limitation, dysplasias, preneoplastic nodules; macroregenerative nodules (MRN); low-grade dysplastic nodules (LG-DN); high-grade dysplastic nodules (HG-DN); biliary epithelial dysplasia; foci of altered hepatocytes (FAH); nodules of altered hepatocytes (NAH); chromosomal imbalances; aberrant activation of telomerase; re-expression of the catalytic subunit of telomerase; expression of endothelial cell markers such as CD31, CD34, and BNH9, as they might effect a tissue, organ, or cell (see, e.g., Terracciano and Tomillo (2003) Pathologica 95:71-82; Su and Bannasch (2003) Toxicol. Pathol. 31:126-133; Rocken and Carl-McGrath (2001) Dig. Dis. 19:269-278; Kotoula, et al. (2002) Liver 22:57-69; Frachon, et al. (2001) J. Hepatol. 34:850-857; Shimonishi, et al. (2000) J. Hepatobiliary Pancreat. Surg. 7:542-550; Nakanuma, et al. (2003) J. Hepatobiliary Pancreat. Surg. 10:265-281). Methods for diagnosing cancer and dysplasia are disclosed (see, e.g., Riegler (1996) Semin. Gastrointest. Dis. 7:74-87; Benvegnu, et al. (1992) Liver 12:80-83; Giannini, et al. (1987) Hepatogastroenterol. 34:95-97; Anthony (1976) Cancer Res. 36:2579-2583).

“Recombinant” when used with reference, e.g., to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived, e.g., from a recombinant nucleic acid, virus, plasmid, vector, or the like. “Recombinant bacterium” encompasses a bacterium where the genome is engineered by recombinant methods, e.g., by way of a mutation, deletion, insertion, and/or a rearrangement. “Recombinant bacterium” also encompasses a bacterium modified to include a recombinant extra-genomic nucleic acid, e.g., a plasmid or a second chromosome.

“Sample” refers to a sample from a human, animal, placebo, or research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a preferred aspect an antibody will have an affinity that is greater than about 10⁹ liters/mol, as determined, e.g., by Scatchard analysis (Munsen, et al. (1980) Analyt. Biochem. 107:220-239). It is recognized by the skilled artisan that some binding compounds can specifically bind to more than one target, e.g., an antibody specifically binds to its antigen as well as to an Fc receptor.

“Spread” of a bacterium encompasses “cell to cell spread,” that is, transmission of the bacterium from a first host cell to a second host cell, as mediated, for example, by a vesicle. Functions relating to spread include, but are not limited to, e.g., formation of an actin tail, formation of a pseudopod-like extension, and formation of a double-membraned vacuole.

Normally, spread of an attenuated Listeria of the present invention is at most 90% that of the parent Listeria strain; more normally spread is at most 80% that of the parent strain; most normally spread is at most 70% that of the parent strain; often spread is at most 60% that of the parent strain; more often spread is at most 50% that of the parent strain; and most often spread is at most 40% that of the parent strain; usually spread is at most 30% that of the parent strain; more usually spread is at most 20% that of the parent strain; most usually spread is at most 10% that of the parent strain; conventionally spread is at most 5% that of the parent strain; more conventionally spread of the attenuated Listeria of the present invention is at most 1% that of the parent strain; and most conventionally spread is not detectable.

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530 issued to Netti, et al.)

“Vaccine” encompasses preventative vaccines. Vaccine also encompasses therapeutic vaccines, e.g., a vaccine administered to a mammal that comprises a condition or disorder associated with the antigen or epitope provided by the vaccine.

II. General.

The invention, in some aspects, provides methods that include administering as one of the reagents Listeria, e.g., Listeria monocytogenes, or other listerial species, for the treatment or prevention of an immune disorder, tumor, cancer, precancerous disorder, or infection, e.g., of the liver, pancreas, gastrointestinal tract, lung, brain, metastasis, metastases, and the like. In the methods of the invention, the Listeria serves as a general immunorecruiting agent, resulting in increased inflammation or in immune cell activation at at one or more sites where the Listeria accumulates. As the Listeria need not be engineered to express a heterologous antigen (e.g., a tumor antigen), any one aspect of the present invention can stimulate immune response to a plurality of tumor types (each tumor type expressing a different antigenic profile), not merely to one tumor type. The Listeria of the invention can also be modified to contain a nucleic acid that encodes at least one heterologous antigen, e.g., an antigen of a tumor cell, virus, or pathogen.

Provided are methods and reagents for treating metastases to the liver from another tissue, e.g., from the colon to the liver, as well as for treating metastases from the liver to another tissue (see, e.g., Yasui and Shimizu (2005) Int. J. Clin. Oncol. 10:86-96; Rashidi, et al. (2000) Clin. Cancer Res. 6:2464-2468; Stoeltzing, et al. (2003) Ann. Surg. Oncol. 10:722-733; Amemiya, et al. (2002) Ophthalmic Epidemiol. 9:35-47).

The invention, in certain aspects, can treat liver tumors arising from de novo tumorigenesis in the liver, or from metastases to the liver from another part of the liver, or from metastases to the liver from the gasterointestinal tract, colon, rectum, ovary, nervous system, endocrine tissues, neuroendocrine tissues, breast, lung, or other part of the body (see, e.g., Liu, et al. (2003) World J. Gastroenterol. 9:193-200; Cormio, et al. (2003) Int. J. Gynecol. Cancer 13:125-129; Sarmiento and Que (2003) Surg. Oncol. Clin. N. Am. 12:231-242; Athanbasakis, et al. (2003) Eur. J. Gastroenterol. Hepatol. 15:1235-1240; Diaz, et al. (2004) Breast 13:254-258).

The pathways of immune response, including NK cell response, generally parallel each other in mice and primates, including humans. Immune response to L. monocytogenes involves an innate response, as well as adaptive response. Innate response is usually identified with increased activity of neutrophils, NK cells, NKT cells, DCs, monocyte/macrophages, and toll-like receptors (TLRs). Innate response to Listeria involves early recruitment of cells such as neutrophils, NK cells, and monocytes, in the mouse and human. Activity of a TLR can be assessed, e.g., by measuring activity of IL-1-R associated kinase (IRAK), NF-kappaB, JNK, caspase-1 dependent cleavage of IL-18 precursor, or activation of IRF-3 (see, e.g., Takeda, et al. (2003) Ann. Rev. Immunol. 21:335-376).

The pathways of adaptive immunity also generally parallel each other in mice and primates, including humans.

Mouse and human NK cells occur as two subsets, one subset high in expression of IL-12 receptor subunit (IL-12Rbeta2) and one low in this receptor subunit. With respect to inhibitory receptors expressed by NK cells, mouse NK cells express gp49B, similar to KIR of human NK cells and mouse NK cells express Ly-49A, which is similar to CD94/NKG2A on human NK cells. With respect to activating receptors on NK cells, both mouse and human NK cells express NKG2D (see, e.g., Chakir, et al. (2000) J. Immunol. 165:4985-4993; Smith, et al. (2000) J. Exp. Med. 191:1341-1354; Ehrlich, et al. (2005) J. Immunol. 174:1922-1931; Peritt, et al. (1998) J. Immunol. 161:5821-5824).

NKT cells occur in both humans and mice. NKT cells of humans and mice show the same reactivity against glyceramides. Human and murine NKT cells express TLRs and show phenotypic and functional similarities. NKT cells mediate immune response to tumors, where IL-12 produced by a DC acts on an NKT cell, stimulating the NKT cell to produce IFNgamma which, in turn, activates NK cells and CD8⁺ T cells to kill tumors (see, e.g., Couedel, et al. (1998) Eur. J. Immunol. 28:4391-4397; Sakamoto, et al. (1999) J. Allergy Clin. Immunol. 103:S445-S451; Saikh, et al. (2003) J. Infect. Dis. 188:1562-1570). NKT cells play a role in response to Listeria (see, e.g., Emoto, et al. (1997) Infection Immunity 65:5003-5009; Taniguchi, et al. (2003) Annu. Rev. Immunol. 21:483-513; Sidobre, et al. (2004) Proc. Natl. Acad. Sci. 101: 12254-12259).

In both the mouse and humans, monocytes serve as precursors to macrophages and dendritic cells. The CX₃CR1^{low} monocytes of mice correspond to the CD14^{high}CD16⁻ monocytes of humans. The CX₃CR1^{high} monocytes of mice correspond to CD14^{low}CD16^{high} of humans (Sunderkotter, et al. (2004) J. Immunol. 172:4410-4417).

Both mice and humans have two lineages of dendritic cells, where the dendritic cells have their origins in pre-dendritic cells (pre-DC1 and pre-DC2). Both humans and mice have pre-DC1 cells and pre-DC2 cells. The pre-DC1 cells mature into CD11c⁺CD8alpha⁺CD11b⁻DCs, which have the property of inducing TH1-type immune response. The pre-DC2 cells mature into CD11c⁺CD8alpha⁻CD11b⁺DCs, which have the property of inducing TH2-type immune response (Boonstra, et al. (2003) J. Exp. Med. 197:101-109; Donnenberg, et al. (2001) Transplantation 72:1946-1951; Becker (2003) Virus Genes 26:119-130). Mice and humans both have plasmacytoid dendritic cells (pDCs), where both mouse and human pDCs express interferon-alpha in response to viral stimulation (Carine, et al. (2003) J. Immunol. 171:6466-6477). Moreover, both the mouse and humans have myeloid DC where, for example, both mouse and human myeloid DCs can express CCL17 (Penna, et al. (2002) J. Immunol. 169:6673-6676; Alferink, et al. (2003) J. Exp. Med. 197:585-599).

Both mice and humans have CD8⁺ T cells. Both mouse and human CD8⁺ T cells comprise similar subsets, that is, central memory T cells and effector memory T cells (see, e.g., Walzer, et al. (2002) J. Immunol. 168:2704-2711). Immune response of CD8⁺ T cells are similar for both mouse and human CD8⁺ T cells as it applies, for example, to expression of CD127 and IL-2 (Fuller, et al. (2005) J. Immunol. 174:5926-5930).

Listeria induces maturation of DCs. L. monocytogenes stimulates the maturation of both human and murine dendritic cells, as measured by listerial-stimulated expression of, e.g., CD86 (see, e.g., Kolb-maurer, et al. (2000) Infection Immunity 68:3680-3688; Brzoza, et al. (2004) J. Immunol. 173:2641-2651; Esplugues, et al. (2005) Blood 105:4399-4406; Paschen, et al. (2000) Eur. J. Immunol. 30:3447-3456).

Neutrophils of both the mouse and human are stimulated by Listeria (see, e.g., Kobayashi, et al. (2003) Proc. Natl. Acad. Sci. USA 100:10948-10953; Torres, et al. (2004) 72:2131-2139; Sibelius, et al. (1999) Infection Immunity 67:1125-1130; Tvinnereim, et al. (2004) J. Immunol. 173:1994-2002). Neutrophils can be detected or characterized by the marker Gr-1 (also known as Gr1 and Ly-6G). Methods for measuring Gr-1 are available (see, e.g., Dumortier, et al. (2003) Blood 101:2219-2226; Bliss, et al. (2000) J. Immunol. 165:4515-4521).

Toll-like receptors (TLRs) comprise a family of about ten receptors, mediating innate response to bacterial components, viral components, and analogues thereof, including lipopolysaccharide (LPS), lipoteichoic acids, peptidoglycan components, lipoprotein, nucleic acids, flagellin, and CpG-DNA. Both humans and mice express the following toll-like receptors: TLR1, TLR2, TLR3, TLR4, TLR5, TLR6, TLR7, TLR8, and TLR9 (Janssens and Beyaert (2003) Clinical Microb. Revs. 16:637-646).

Response to L. monocytogenes, by mouse and human systems, involves expression of IFN-gamma (see, e.g., Way and Wilson (2004) J. Immunol. 173:5918-5922; Ouadrhiri, et al. (1999) J. Infectious Diseases 180:1195-1204; Neighbors, et al. (2001) J. Exp. Med. 194:343-354; Calorini, et al. (2002) Clin. Exp. Metastasis 19:259-264; Andersson, et al. (1998) J. Immunol. 161:5600-5606).

Response to L. monocytogenes, by both mouse and human systems, involves expression of tumor necrosis factor (TNF) (see, e.g., Flo, et al. (2000) J. Immunol. 164:2064-2069; Calorini, et al. (2002) Clin. Exp. Metastasis 19:259-264; Brzoza, et al. (2004) J. Immunol. 173:2641-2651).

Response to L. monocytogenes, as shown by murine and human studies, involves expression of interleukin-12 (IL- 12) (see, e.g., Brzoza, et al. (2004) J. Immunol. 173:2641-2651; Cleveland, et al. (1996) Infection Immunity 64:1906-1912; Andersson, et al. (1998) J. Immunol. 161:5600-5606).

CD69 is an activation marker of immune cells, as determined in studies of murine and human immune cells (see, e.g., Pisegna, et al. (2002) J. Immunol. 169:68-74; Gerosa, et al. (2002) J. Exp. Med. 195:327-333; Borrego, et al. (1999) Immunology 97:159-165).

The following concerns cytokines, e.g., interferon-gamma and MCP-1. Interferon-gamma (IFN-gamma) is expressed by both humans and mice. IFN-gamma is a key cytokine in the immune system's response against tumors and microbial pathogens, as well as against tumor angiogenesis. IFN-gamma mediates immune response against liver tumors and viral hepatitis, for example, by studies administering vaccines against hepatitis virus, administration of IFN-gamma, or administering anti-IFN antibodies (see, e.g., Grassegger and Hopfl (2004) Clin. Exp. Dermatol. 29:584-588; Tannenbaum and Hamilton (2000) Semin. Cancer Biol. 10:113-123; Blankensetein and Qin (2003) Curr. Opin. Immunol. 15:148-154; Fidler, et al. (1985) J. Immunol. 135:4289-4296; Okuse, et al. (2005) Antiviral Res. 65:23-34; Piazzolla, et al. (2005) J. Clin. Immunol. 25:142-152; Xu, et al. (2005) Vaccine 23:2658-2664; Irie, et al. (2004) Int. J. Cancer 111:238-245).

Monocyte chemoattractant protein (MCP-1; CCL2) is expressed by humans and mice. MCP-1 promotes macrophage infiltration of tumors. MCP-1 is mediates immune response to viral hepatitis infections. Moreover, administered MCP-1 promotes tumors eradication by macrophages. In other studies, MCP-1 was correlated with efficiency of drug therapy against viral hepatitis (See, e.g., Nakamura, et al. (2004) Cancer Gene Ther. 11:1-7; Luo, et al. (1994) J. Immunol. 153:3708-3716; Panasiuk, et al. (2004) World J. Gastroenterol. 10:36639-3642).

Immune response can involve response to proteins, peptides, cells expressing proteins or peptides, as well as against other entities such as nucleic acids, oligosaccharides, glycolipids, and lipids. For example, immune response against a virus can include immune response against a peptide of the virus, a nucleic acid of the virus, a glycolipid of the virus, or an oligosaccharide of the virus (see, e.g., Rekvig, et al. (1995) Scand. J. Immunol. 41:593-602; Waisman, et al. (1996) Cell Immunol. 173:7-14; Cerutti, et al. (2005) Mol. Immunol. 42:327-333; Oschmann, et al. (1997) Infection 25:292-297; Paradiso and Lindberg (1996) Dev. Biol. Stand. 87:269-275).

A broad spectrum of tumors, viruses, bacteria, and other pathogens, are attacked by NK cells and NKT cells. The targets of NK cells and NKT cells include, e.g., colon adenocarcinomas, neuroblastomas, sarcomas, lymphomas, breast cancers, melanomas, erythroleukemic tumors, leukemias, mastocytomas, colon carcinomas, breast adenocarcinomas, ovarian adenocarcinomas, fibrosarcomas, melanomas, lung carcinomas, rhabdomyosarcomas, gliomas, renal cell cancers, gastric cancers, lung small cell carcinomas, cancers arising from metastasis to the liver, as well as a range of viruses, including, hepatitis A virus, hepatitis B virus, hepatitis C virus, herpes simplex virus, gamma herpes viruses, Epstein-Barr virus (EBV), HIV, dengue virus, and a range of bacteria, such as Mycoplasma, and Brucella (see, e.g., Vujanovic, et al. (1996) J. Immunol. 157:1117-1126; Kashii, et al. (1999) J. Immunol. 163:5358-5366; Giezeman-Smits, et al. (1999) J. Immunol. 163:71-76; Turner, et al. (2001) J. Immunol. 166:89-94; Kawarada, et al. (2001) J. Immunol. 167:5247-5253; Scott-Algara and Paul (2002) Curr. Mol. Med. 2:757-768; Kambach, et al. (2001) J. Immunol. 167:2569-2576; Westwood, et al. (2003) J. Immunol. 171:757-761; Roda, et al. (2005) J. Immunol. 175:1619-1627; Poggi, et al. (2005) J. Immunol. 174:2653-2660; Metelitsa, et al. (2001) J. Immunol. 167:3114-3122; Wei, et al. (2000) J. Immunol. 165:3811-3819; Bakker, et al. (1998) J. Immunol. 160:5239-5245; Makrigiannis, et al. (2004) J. Immunol. 172:1414-1425; Golding, et al. (2001) Microbes Infect. 3:43-48; Lai, et al. (1990) J. Infect. Dis. 161:1269-1275; Ohga, et al. (2002) Crit. Rev. Oncol. Hematol. 44:203-215; Wakimoto, et al. (2003) Gene Ther. 10:983-990; Chen, et al. (2005) J. Viral Hepat. 12:38-45; Baba, et al. (1993) J. Clin. Lab Immunol. 40:47-60; Li, et al. (2004) J. Leukoc. Biol. 76:1171-1179; Scalzo (2002) Trends Microbiol. 10:470-474; Ahlenstiel and Rehermann (2005) Hepatology 41:675-677; Chen, et al. (2005) J. Viral Hepat. 12:38-45; Sun and Gao (2004) Gasteroenterol. 127:1525-1539; Li, et al. (2004) J. Leukoc. Biol. 76:1171-1179; Ahmad and Alvarez (2004) J. Leukoc. Biol. 76:743-759; Cook (1997) Eur. J. Gasteroenterol. Hepatol. 9:1239-1247; Williams and Riordan (2000) J. Gasteroenterol. Hepatol. 15 (Suppl.) G17-G25; Varani and Landini (2002) Clin. Lab. 48:39-44; Rubin (1997) Clin. Liver Dis. 1:439-452; Loh, et al. (2005) J. Virol. 79:661-667; Shresta, et al. (2004) Virology 319:262-273; Fjaer, et al. (2005) Pediatr. Transplant 9:68-73; Li, et al. (2004) World J. Gasteroenterol. 10:3409-3413; Collin, et al. (2004) J. Hepatol. 41:174-175; Ohga, et al. (2002) Crit. Rev. Oncol. Hematol. 44:203-215).

NK cells can eliminate a broad range of parasitic organisms and protozoans, such as those responsible for toxoplasmosis, trypanosomiasis, leishmaniasis, and malaria (see, e.g., Korbel, et al. (2004) Int. J. Parasitol. 34:1517-1528; Mavoungou, et al. (2003) Eur. Cytokine Netw. 14:134-142; Doolan and Hoffman (1999) J. Immunol. 163:884-892).

III. Antibody-Mediated Therapeutic Effects.

The methods of the invention can stimulate immune response by way of antibody dependent cell cytotoxicity (ADCC), as well as other mechanisms. ADCC can be mediated by NK cells, macrophages, and neutrophils. The invention provides methods that comprise administering a Listeria and an antibody to stimulate immune response against a tumor, cancer, pre-cancerous disorder, and/or an infection. Without limiting the invention to any mechanism of action, antibody mediated cell cytotoxicity can involve antibody dependent cell cytotoxicity (ADCC), where an administered antibody binds to a cytotoxic cell via its Fc region and to a target cell via its variable region, resulting in the lysis or phagocytosis of the target cell. In another scenario of antibody action, the Fc region of an administered antibody binds to a dendritic cell, while the variable region of the antibody binds to a moribund target cell, where the immediate result is enhanced uptake of the target cell by the dendritic cell, and the downstream result is increased presentation (cross-presentation) of epitopes derived from the target cell (see, e.g., Brady (2005) Infect. Immun. 73:671-678; Dhodapkar, et al. (2005) Proc. Natl. Acad. Sci. USA 102:2910-2915; Dhodapkar and Dhodapkar (2005) Proc. Natl. Acad. Sci. USA 102:6243-6244; Groh, et al. (2005) Proc. Natl. Acad. Sci. USA 102:6461-6466).

The present invention provides methods that utilize antibodies, as well as binding compounds containing an antigen binding site of an antibody; the Fc receptor binding site of an antibody; both the antigen binding site of an antibody and the Fc receptor binding site of an antibody, for use in mediated cell cytotoxicity. “Antigen binding site” encompasses compositions and molecules derived from the antigen binding site of an antibody. “Fc receptor binding site of an antibody” encompasses compositions and molecules derived from an Fc receptor binding site of an antibody.

The present invention provides methods that utilize peptide mimetics, including peptide mimetics of an antibody that specifically binds to an antigen of a tumor, cancer, infectious agent, virus, bacterium, protozoan, and the like. Peptide mimetics, including peptide mimetic of antibodies, are designed and prepared by established methods (see, e.g., Casset, et al. (2003) Biochem. Biophys. Res. Commun. 18:198-205; Casset, et al. (2003) Biochem. Biophys. Res. Commun. 307:198-205; [no authors listed] (2000) Nat. Biotechnol. 18:137; Andrade-Gordon, et al. (1999) Proc. Natl. Acad. Sci. USA 96:12257-12262; Sato and Sone (2003) Biochem. J. 371:603-608; Park, et al. (2000) Nat. Biotechnol. 18:194-198; Engleman, et al. (1997) J. Clin. Invest. 99:2284-2292; Martin-Moe, et al. (1995) Peptide Res. 8:70-76; Venkatesh, et al. (2002) Peptides 23:573-580; Muyldermans and Lauwereys (1999) J. Mol. Recognit. 12:131-140; Maryanoff, et al. (2003) Curr. Med. Chem. Cardiovasc. Hematol. Agents 1:13-36; Yoshimori, et al. (2005) Apeptosis 10:323-329; Kadono, et al. (2005) Biochem. Biophys. Res. Commun. 326:859-865).

Methods for using antibodies to mediate immune response against tumors, cancers, and infections or infective agents, are available (see, e.g., Presta (2002) Curr. Pharm. Biotechnol. 3:237-256; Presta, et al. (2002) Biochem. Soc. Trans. 30:487-490; Clynes, et al. (2000) Nat. Med. 6:443-446; Green, et al. (2002) Cancer Res. 62:6891-6900; Dechant and Valerius (2001) Crit. Rev. Oncol. Hematol. 39:69-77; Sondel and Hank (2001) Hematol. Oncol. Clin. North Am. 15:703-721; Sulica, et al. (2001) Int. Rev. Immunol. 20:371-414; Carter (2001) Nature Rev. Cancer 1:118-129; Sun (2003) Immunol. Res. 27:539-548; Daeron (1997) Annu. Rev. Immunol. 15:203-234; Ward and Ghetie (1995) Therapeutic Immunol. 2:77-94; Ravetch and Kinet (1991) Annu. Rev. Immunol. 9:457-492).

Without limiting the present invention to any particular mechanism, the following discussion concerns antibody dependent cell cytotoxicity (ADCC). ADCC can be mediated by the Fc region of the administered antibody (Prange, et al, supra; Yokayama and Plougastel (2003) Nat. Rev. Immunol. 3:304-316; Trinchieri and Valiante (1993) Nat. Immunol. 12:218-234). However, the present invention is not necessarily limited to an administered antibody that comprises an Fc region, or a binding compound derived from an antibody that comprises an Fc region. Instead of comprising an antigen binding site and an Fc region, the contemplated binding compound can comprise a bifunctional antibody. The contemplated bifimctional antibody, or multifunctional antibody, can contain a first binding site that specifically binds a tumor antigen and a second binding site that specifically binds an Fc receptor.

Also provided for use in the methods is a bifunctional antibody comprising a first antigen binding site derived from a first antibody that specifically binds an antigen of a tumor cell, cancer cell, or infectious agent, and a second antigen binding site derived from a second antibody that specifically binds to an NK cell, monocyte, or other cell that mediates ADCC. The second antibody can specifically bind to marker or membrane-associate protein of, for example, an NK cell, an NKT cell, a monocyte, or a gammadelta T cell. The second antibody can specifically bind to, e.g., activating KIR-L (2DS1 to 5; 3DS1); inhibiting KIR-L (2DL1 to 2DL5; 3DL1-3DL3); CD94/CD159a (NKG2A); CD85j (ILT-2/LIR-1); CD56; CD57; CD62 (L-selectin); CD162R (PEN5); CD122 (subunit of IL-2 receptor); NKp80; NKp46; NKp30; CD161 (NKRP-1 expression); NK1.1; DX5 (see, e.g., Pascal, et al. (2004) Eur. J. Immunol. 34:2930-2940; Sivori, et al. (2003) Eur. J. Immunol. 33:3439-3447; Takayama, et al. (2003) Immunology 108:211-219; Vemeris, et al. (2001) Biol. Blood Marrow Transplant. 7:532-542; Rischer, et al. (2004) Br. J. Haematol. 126:583-592).

The present invention encompasses methods for administering a Listeria bacterium, including a Listeria monocytogenes bacterium, with an antibody or a binding compound derived from an antibody. The Listeria can be attenuated. Without limitation, the Listeria can be attenuated in growth, spread, entry into a cell, growth and spread, growth and entry into a cell, spread and entry into a host cell, or all three (growth, spread, and entry into a host cell). Moreover, the present invention provides reagents and methods for administering a Listeria bacterium, including a Listeria monocytogenes bacterium, with an antibody or a binding composition (or compound) derived from an antibody, where the Listeria is engineered to comprise a nucleic acid encoding an antigen. The antigen can be from, or derived from, a tumor antigen, cancer antigen, infectious organism antigen, pathogen antigen, viral antigen, bacterial antigen, antigen from a parasite, a listerial antigen, an antigen heterologous to the Listeria bacterium, or an antigen from the Listeria bacterium.

Where the Listeria bacterium is engineered to comprise a nucleic acid encoding an antigen, the antigen can be one specifically bound by the administered antibody, or the antigen can be one that is not specifically bound by the administered antibody.

Additionally, the present invention encompasses reagents and methods where more than one antibody is administered, for example, where a first administered antibody can specifically bind a first antigen and where a second administered antibody can specifically bind a second antigen. Moroever, the invention provides a Listeria comprising a polynucleotide encoding more than one antigen, for example, where the polynucleotide comprises a first nucleic acid encoding a first antigen and a second nucleic acid encoding a second antigen. Provided is any and all combinations of the above reagents and methods.

The Listeria of the invention can be engineered to express enzymes required for the biosynthesis of an antigen such as, e.g., a lipid, phosopholipid, glycolipid, oligosaccharide, glycopeptide, or glycoprotein.

Provided are reagents and methods of modulating expression and/or activity of an Fc receptor. The present invention encompasses reagents and methods for inhibiting or reducing an inhibiting Fc receptor, e.g., Fc gammaRIIB, and for increasing, stimulating, or activating an activating Fc receptor, e.g., FcgammaRIII.

Where an antibody is administered, complement-dependent cytotoxicity (CDC) can also contribute to immune response against a tumor, cancer, pre-cancerous disorder, or infection. Therapeutic antibodies that work, at least in part, by CDC include Rituxan®, Herceptin®, Campath®, MT201 (anti-Ep-CAM IgG1), and an anti-Ep-CAM (IgG2a) (see, e.g., Prang, et al. (2005) Br. J. Cancer 92:342-349). The invention provides reagents and methods to administer a Listeria, antibody, along with a stimulant of CDC, such as beta-glucan (Hong, et al. (2003) Cancer Res. 63:9023-9031). Fungal beta-glucans, and analogues thereof, can enhance CDC (see, e.g., Hong, et al. (2003) Cancer Res. 63:9023-9031).

Once a tumor cell is killed or rendered moribund, e.g., by the action of a cytotoxic T cell, the moribund tumor cell can be taken up by a dendritic cell (DC), where the DC then presents tumor antigens (cross-presentation). Uptake of a killed or moribund cell can be enhanced by administering an antibody specific to that tumor cell, resulting in a complex of antitumor antibodies and the tumor cell. This complex is bound by Fc receptors of the DC. Once bound, the antibody/tumor cell complex (or antibody/antigen complex) is taken up by the DC (see, e.g., Dhodapkar, et al. (2005) Proc. Natl. Acad. Sci. USA 102:2910-2915; Dhodapkar and Dhodapkar (2005) Proc. Natl. Acad. Sci. USA 102:6243-6244; Groh, et al. (2005) Proc. Natl. Acad. Sci. USA 102:6461-6466). What is available, for use in the invention, are anti-tumor antibodies, anti-infective agent antibodies, anti-pathogen antibodies, and the like, for used in enhancing enhancing uptake by DCs and/or for use in enhancing cross-presentation by DCs. Provided are engineered modified to enhance binding to activating Fc receptors, reducing binding to inhibiting Fc receptors, or to both. One goal of the present invention is to inhibit or knock out one or more inhibiting Fc receptors.

Also provided is a first antibody that specifically binds to an inhibiting Fc receptor, and related methods, for use in administering to a patient experiencing a tumor, infection, pathogen, and the like, and for reducing or preventing binding of a second antibody (anti-tumor antibody; anti-pathogen antibody) to said inhibiting Fc receptor (see, e.g., Dhodapkar, et al. (2005) Proc. Natl. Acad. Sci. USA 102:2910-2915).

The reagents and methods of the present invention are not limited, and are not to be limited, by the mechanism of action (e.g., ADCC or CDC) of the administered antibody or binding compound derived from the antibody.

IV. Antibodies and Derivatives thereof.

Monoclonal, polyclonal, and humanized antibodies useful for the invention can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205-6213; He, et al. (1998) J. Immunol. 160:1029-1035; Tang, et al. (1 999) J. Biol. Chem. 274:27371-27378). A humanized antibody, to give a non-limiting example, can contain the amino acid sequences from six complementarity determining regions (CDRs) of the parent mouse antibody, which are grafted on a human antibody framework.

Reagents and methods to humanize an antibody (or a binding compound derived from an antibody), to alter binding of complement to an antibody (or to a binding compound to an antibody), to modify binding of tissue factor to an antibody (or to a binding compound derived from an antibody), to modify binding of the antibody to an Fc receptor, and to modify an an antibody (or a binding compound derived from an antibody) with polyethyleneglycol (PEG) are available (see, e.g., Idusogie, et al. (2001) J. Immunol. 166:2571-2575; Presta, et al. (2001) Thromb. Haemost. 85:379-389; Leong, et al. (2001) Cytokine 16:106-119; Presta (2002) Curr. Pharm. Biotechnol. 3:237-256; Presta, et al. (2002) Biochem. Soc. Trans. 30:487-490; Presta (2003) Curr. Opin. Struct. Biol. 13:519-525; U.S. Pat. Pub. No. US 2004/0236078 of Carter and Presta; Rasmussen, et al. (2001) Proc. Natl. Acad. Sci. USA 98:10296-10301).

Alternatives to humanization include use of fully human antibodies, as well as human antibody libraries displayed on phage or human antibody libraries contained in transgenic mice (see, e.g., Vaughan, et al. (1996) Nat. Biotechnol. 14:309-314; Barbas (1995) Nat. Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nat. Biotechnol. 17:397-399).

Fv fragments, Fab fragments, single chain antibodies, single domain antibodies, and bispecific antibodies for use in the present invention are described (see, e.g., Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) New Engl. J. Med. 148:1547-1553; Willuda, et al. (1999) Cancer Res. 59:5758-5767; U.S. Pat. Applic. No. 2005/0136050 of Kufer, et al.).

What is available is a bifunctional antibody comprising a first binding site (from or derived from an antibody) specific for a tumor antigen and a second binding site (from or derived from an antibody) specific for an Fc receptor. Contemplated is a bifunctional antibody comprising a binding site specific for an activating Fc receptor (e.g., FcgammaRIII) and a binding site specific for a tumor antigen or antigen of an infectious agent. Also encompassed is a multifunctional antibody comprising more than one binding site specific for an Fc receptor and more than one binding site specific for a tumor antigen or antigen of an infectious agent (see, e.g., Renner, et al. (2001) Cancer Immunol. Immunother. 50:102-108; Kudo, et al. (1999) Tohoku J. Exp. Med. 188:275-288; Fanger, et al. (1994) Immunomethods 4:72-81; Bruenke, et al. (2004) Br. J. Haematol. 125:167-179).

What is also available is a variety of Fc regions for use with the antigen-binding site of an antibody. Antibodies occur in a number of classes and subclasses, and each has a characteristic Fc region, where each Fc region may bind with differing relative specificities to various Fc receptors. For example, Fc gamma RIII (activating receptor) binds preferentially to IgG1 and IgG3 (to the Fc regions of these antibody classes) while Fc gamma RIIb (inhibiting receptor) binds less to IgG1. Hence, an antibody of the IgG1 class can have a greater effect in stimulating ADCC than an antibody of the IgG3 class. Along a similar vein, a number of mutations in the Fc region can increase binding to Fc gamma RIIIa (activating receptor) and decrease binding to Fc gamma RIIb (inhibiting receptor). What is available are mutations, such as S298A; E333A; K334A; and/or D264A, as well as alterations of the oligosaccharide bound to the antibody that improve ADCC, e.g., fucose-deficient IgG1 shows improved ADCC. The reagents of the present invention encompass antibodies with increased binding to an activating Fc receptor and/or decreased binding to an inhibiting Fc receptor (see, e.g., Gessner, et al. (1998) Ann. Hematol. 76:231; Shields, et al. (2001) 276:6591-6604; Shields, et al. (2002) J. Biol. Chem. 277:26733-26740; Presta, et al. (2002) Biochem. Soc. Trans. 30:487-490; Clynes, et al. (2000) Nature 4:443-446).

Antigen fragments can be joined to other materials, such as fused or covalently joined polypeptides, to be used as immunogens. An antigen and its fragments may be fused or covalently linked to a variety of immunogens, such as keyhole limpet hemocyanin, bovine serum albumin, or ovalbumin (see, e.g., Coligan, et al. (1994) Current Protocols in Immunol., Vol. 2, 9.3-9.4, John Wiley and Sons, New York, N.Y.). Peptides of suitable antigenicity can be selected from the polypeptide target, using an algorithm, such as those of Parker, et al. (1986) Biochemistry 25:5425-5432; Jameson and Wolf (1988) Cabios 4:181-186; or Hopp and Woods (1983) Mol. Immunol. 20:483-489).

Purification of an antigen is not necessary for the generation of antibodies. Immunization can be performed by DNA vector immunization (see, e.g., Wang, et al. (1997) Virology 228: 278-284). Alternatively, animals can be immunized with cells bearing the antigen of interest. Splenocytes can then be isolated from the immunized animals, and the splenocytes can fused with a myeloma cell line to produce a hybridoma. Resultant hybridomas can be screened for production of the desired antibody by functional assays or biological assays, that is, assays not dependent on possession of the purified antigen. Immunization with cells can prove superior for antibody generation than immunization with purified antigen (see, e.g., Meyaard, et al. (1997) Immunity 7:283-290; Wright, et al. (2000) Immunity 13:233-242; Preston, et al. (1997) Eur. J. Immunol. 27:1911-1918; Kaithamana, et al. (1999) New Engl. J. Med. 163:5157-5164).

Antibody screening and antigen binding properties can be measured, e.g., by surface plasmon resonance or enzyme linked immunosorbent assay (ELISA). The antibodies of this invention can be used for affinity chromatography in isolating the antibody's target antigen and associated bound proteins. The present invention provides high, moderate, and low antibodies for anti-tumor therapy. In tumor therapy, a high affinity antibody may bind only to the surface, while a moderate affinity antibody may diffuse throughout the tumor, resulting in higher therapeutic efficiency (see, e.g., Anderson, et al. (2004) J. Proteome Res. 3:228-234; Santala and Saviranta (2004) J. Immunol. Methods 284:159-163; Leuking, et al. (2003) Mol. Cell Proteomics 2:1342-1349; Seideman and Peritt (2002) J. Immunol. Methods 267:165-171; Neri, et al. (1997) Nat. Biotechnol. 15:1271-1275; Jonsson, et al. (1991) Biotechniques 11:620-627; Hubble (1997) Immunol. Today 18:305-306; Wilchek, et al. (1984) Meth. Enzymol. 104:3-55; Adams, et al. (1998) Cancer Res. 58:485: Adams, et al. (2001) Cancer Res. 61:4750).

Antigens, antigenic fragments, and epitopes, are available for use in generating the antibodies of the present invention (Table 3). Also available are nucleic acids for use in expressing the antigens, e.g., for generating the antibodies, and also for preparing a recombinant bacterium that expresses the antigen (Table 3).

IV. Fc Region Variants.

Several antibody effector functions are mediated by Fc receptors (FcRs). Fc receptors bind the Fc region of an antibody. FcRs are defined by their specificity for immunoglobulin isotypes; Fc receptors for IgG antibodies are referred to as Fc gamma R, for IgE as Fc epsilon R, for IgA as Fc alpha R and so on. Three subclasses of Fc gamma R have been identified: Fc gamma RI (CD64), Fc gamma RII (CD32) and Fc gamma RIII (CD16). Because each Fc gamma R subclass is encoded by two or three genes, and alternative RNA spicing leads to multiple transcripts, a broad diversity in Fc gamma R isoforms exists. The three genes encoding the Fc gamma RI subclass (Fc gamma RIA, Fc gamma RIB and Fc gamma RIC) are clustered in region 1q21.1 of the long arm of chromosome 1; the genes encoding Fc gamma RII isoforms (Fc gamma RIIA, Fc gamma RIIB and Fc gamma RIIC) and the two genes encoding Fc gamma RIII (Fc gamma RIIIA and Fc gamma RIIIB) are all clustered in region 1q22. These different FcR subtypes are expressed on different cell types (see, e.g., Ravetch and Kinet (1991) Annu. Rev. Immunol. 9:457-492). For example, in humans, Fc gamma RIIIB is found only on neutrophils, whereas Fc gamma RIIIA is found on macrophages, monocytes, natural killer (NK) cells, and a subpopulation of T-cells. Notably, Fc gamma RIIIA is the only FcR present on NK cells, one of the cell types implicated in ADCC (see, U.S. Pat. No. 6,737,056 issued to Presta).

Fc gamma RI, Fc gamma RII and Fc gamma RIII are immunoglobulin superfamily (IgSF) receptors; Fc gamma RI has three IgSF domains in its extracellular domain, while Fc gamma RII and Fc gamma RIII have only two IgSF domains in their extracellular domains (U.S. Pat. No. 6,737,056 issued to Presta).

What is available for use in the invention is a variant of a parent polypeptide comprising an Fc region, which variant mediates ADCC in the presence of human effector cells more effectively or binds an Fc gamma receptor (Fc gamma R) with better affinity, than the parent polypeptide and comprises at least one amino acid modification in the Fc region. The Fc region of the parent polypeptide typically comprises a human Fc region; e.g., a human IgG1, IgG2, IgG3 or IgG4 Fc region. The polypeptide variant also typically comprises an amino acid modification (e.g. a substitution) at any one or more of amino acid positions 256, 290, 298, 312, 326, 330, 333, 334, 360, 378 or 430 of the Fc region, wherein the numbering of the residues in the Fc region is that of the EU index as in Kabat (U.S. Pat. No. 6,737,056 issued to Presta).

In addition, what is available is a polypeptide comprising a variant Fc region with altered Fc gamma receptor (Fc gamma R) binding affinity, which polypeptide comprises an amino acid modification at any one or more of amino acid positions 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439 of the Fc region, wherein the numbering of the residues in the Fc region is that of the EU index as in Kabat. The variant Fc region quite often comprises a variant human IgG Fc region, e.g., a variant human IgG1, IgG2, IgG3 or IgG4 Fc region. Where the parent polypeptide had a non-human murine Fc region, different residues from those identified herein may impact FcR binding. For example, in the murine IgG2b/murine Fc gamma RII system, IgG E318 was found to be important for binding (Lund et al. (1992) Molec. Immunol. 27:53-59), whereas E318A had no effect in the human IgG/human Fc gamma RII system (see U.S. Pat. No. 6,737,056 issued to Presta).

The polypeptide variant may display reduced binding to an Fc gamma RI and comprise an amino acid modification at any one or more of amino acid positions 238, 265, 269, 270, 327 or 329 of the Fc region, wherein the numbering of the residues in the Fc region is that of the EU index as in Kabat (U.S. Pat. No. 6,737,056 issued to Presta).

The polypeptide variant may display reduced binding to an Fc gamma RII and comprise an amino acid modification at any one or more of amino acid positions 238, 265, 269, 270, 292, 294, 295, 298, 303, 324, 327, 329, 333, 335, 338, 373, 376, 414, 416, 419, 435, 438 or 439 of the Fc region, wherein the numbering of the residues in the Fc region is that of the EU index as in Kabat (U.S. Pat. No. 6,737,056 issued to Presta).

The polypeptide variant of interest may display reduced binding to an Fc gamma RIII and comprise an amino acid modification at one or more of amino acid positions 238, 239, 248, 249, 252, 254, 265, 268, 269, 270, 272, 278, 289, 293, 294, 295, 296, 301, 303, 322, 327, 329, 338, 340, 373, 376, 382, 388, 389, 416, 434, 435 or 437 of the Fc region, wherein the numbering of the residues in the Fc region is that of the EU index as in Kabat (U.S. Pat. No. 6,737,056 issued to Presta).

In another aspect, the polypeptide variant with altered Fc gamma R binding affinity displays improved binding to the Fc gamma R and comprises an amino acid modification at any one or more of amino acid positions 255, 256, 258, 267, 268, 272, 276, 280, 283, 285, 286, 290, 298, 301, 305, 307, 309, 312, 315, 320, 322, 326, 330, 331, 333, 334, 337, 340, 360, 378, 398 or 430 of the Fc region, wherein the numbering of the residues in the Fc region is that of the EU index as in Kabat (U.S. Pat. No. 6,737,056 issued to Presta).

For example, the polypeptide variant may display increased binding to an Fc gamma RIII and, optionally, may further display decreased binding to an Fc gamma RII. An exemplary such variant comprises amino acid modification(s) at position(s) 298 and/or 333 of the Fc region, wherein the numbering of the residues in the Fc region is that of the EU Index as in Kabat (U.S. Pat. No. 6,737,056 issued to Presta).

The polypeptide variant may display increased binding to an Fc gamma RII and comprise an amino acid modification at any one or more of amino acid positions 255, 256, 258, 267, 268, 272, 276, 280, 283, 285, 286, 290, 301, 305, 307, 309, 312, 315, 320, 322, 326, 330, 331, 337, 340, 378, 398 or 430 of the Fc region, wherein the numbering of the residues in the Fc region is that of the EU index as in Kabat. Such polypeptide variants with increased binding to an Fc gamma RII may optionally further display decreased binding to an Fc gamma RIII and may, for example, comprise an amino acid modification at any one or more of amino acid positions 268, 272, 298, 301, 322 or 340 of the Fc region, wherein the numbering of the residues in the Fc region is that of the EU index as in Kabat (U.S. Pat. No. 6,737,056 issued to Presta).

Also, what is available is a polypeptide comprising a variant Fc region with altered neonatal Fc receptor (FcRn) binding affinity, which polypeptide comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 382, 386, 388, 400, 413, 415, 424, 433, 434, 435, 436, 439 or 447 of the Fc region, wherein the numbering of the residues in the Fc region is that of the EU index as in Kabat. Such polypeptide variants with reduced binding to an FcRn may comprise an amino acid modification at any one or more of amino acid positions 252, 253, 254, 255, 288, 309, 386, 388, 400, 415, 433, 435, 436, 439 or 447 of the Fc region, wherein the numbering of the residues in the Fc region is that of the EU index as in Kabat. The above-mentioned polypeptide variants may, alternatively, display increased binding to FcRn and comprise an amino acid modification at any one or more of amino acid positions 238, 256, 265, 272, 286, 303, 305, 307, 311, 312, 317, 340, 356, 360, 362, 376, 378, 380, 382, 413, 424 or 434 of the Fc region, wherein the numbering of the residues in the Fc region is that of the EU index as in Kabat (U.S. Pat. No. 6,737,056 issued to Presta).

Fc region variants can be classified as follows. Listed is the binding property and the position of the substitutions in the Fc region:
Class 1A. Reduced binding to all Fc gamma R (238, 265, 269, 270, 297*, 327, 329). The asterisk * refers to the deglycosylated version.
Class 1B. Reduced binding to both Fc gamma RII and Fc gamma RIII (239, 294, 295, 303, 338) 373, 376, 416, 435).
Class 2. Improved binding to both Fc gamma RII and Fc gamma RIII (256, 290, 312, 326, 330, 339*, 378, 430). The asterisk* means preferably combined with other Fc modifications, as described (U.S. Pat. No. 6,737,056 issued to Presta).
Class 3. Improved binding to Fc gamma RII and no effect on Fc gamma RIII binding (255, 258, 267, 276, 280, 283, 285, 286, 305, 307, 309, 315, 320, 331, 337, 398).
Class 4. Improved binding to Fc gamma RII and reduced binding to Fc gamma RIII (268, 272, 301, 322, 340).
Class 5. Reduced binding to Fc gamma RII and no effect on Fc gamma RIII binding (292, 324, 335, 414, 419, 438, 439).
Class 6. Reduced binding to Fc gamma RII and improved binding to Fc gamma RIII (298, 333).
Class 7. No effect on Fc gamma RII binding and reduced binding to Fc gamma RIII (248, 249, 252, 254, 278, 289, 293, 296, 338, 382, 388, 389, 434, 437).
Class 8. No effect on Fc gamma RII binding and improved binding to Fc gamma RIII (334, 360).

To generate an Fc region with improved ADCC activity, the parent polypeptide preferably has pre-existing ADCC activity, e.g., it comprises a human IgG1 or human IgG3 Fc region. In one aspect, the variant with improved ADCC mediates ADCC substantially more effectively than an antibody with a native sequence IgG1 or IgG3 Fc region and the antigen-binding region of the variant. An an alternate aspect, the variant comprises, or consists essentially of, substitutions of two or three of the residues at positions 298, 333 and 334 of the Fc region. Most usually, residues at positions 298, 333 and 334 are substituted (e.g. with alanine residues). Moreover, in order to generate the Fc region variant with improved ADCC activity, one will generally engineer an Fc region variant with improved binding affinity for Fc gamma RIII, which is thought to be an important FcR for mediating ADCC. For example, one may introduce an amino acid modification (e.g. a substitution) into the parent Fc region at any one or more of amino acid positions 256, 290, 298, 312, 326, 330, 333, 334, 360, 378 or 430 to generate such a variant. The variant with improved binding affinity for Fc gamma RIII may further have reduced binding affinity for Fc gamma RII, especially reduced affinity for the inhibiting Fc gamma RIIB receptor (U.S. Pat. No. 6,737,056 issued to Presta).

The amino acid modification(s) can be introduced into the CH2 domain of a Fc region. The CH2 domain is important for FcR binding activity, but also into a part of the Fc region other than in the lower hinge region thereof.

Useful amino acid positions for modification in order to generate a variant IgG Fc region with altered Fc gamma receptor (Fc gamma R) binding affinity or activity include any one or more of amino acid positions 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439 of the Fc region. Normally, the parent Fc region used as the template to generate such variants comprises a human IgG Fc region. Where residue 331 is substituted, the parent Fc region is preferably not human native sequence IgG3, or the variant Fc region comprising a substitution at position 331 preferably displays increased FcR binding, e.g. to Fc gamma RII (U.S. Pat. No. 6,737,056 issued to Presta).

To generate an Fc region variant with reduced binding to the Fc gamma R one may introduce an amino acid modification at any one or more of amino acid positions 238, 239, 248, 249, 252, 254, 265, 268, 269, 270, 272, 278, 289, 292, 293, 294, 295, 296, 298, 301, 303, 322, 324, 327, 329, 333, 335, 338, 340, 373, 376, 382, 388, 389, 414, 416, 419, 434, 435, 437, 438 or 439 of the Fc region.

Variants which display reduced binding to Fc gamma RI, include those comprising an Fc region amino acid modification at any one or more of amino acid positions 238, 265, 269, 270, 327 or 329.

Variants which display reduced binding to Fc gamma RII include those comprising an Fc region amino acid modification at any one or more of amino acid positions 238, 265, 269, 270, 292, 294, 295, 298, 303, 324, 327, 329, 333, 335, 338, 373, 376, 414, 416, 419, 435, 438 or 439.

Fc region variants which display reduced binding to Fc gamma RIII include those comprising an Fc region amino acid modification at any one or more of amino acid positions 238, 239, 248, 249, 252, 254, 265, 268, 269, 270, 272, 278, 289, 293, 294, 295, 296, 301, 303, 322, 327, 329, 338, 340, 373, 376, 382, 388, 389, 416, 434, 435 or 437 (U.S. Pat. No. 6,737,056 issued to Presta).

Variants with improved binding to one or more Fc gamma Rs may also be made. Such Fc region variants may comprise an amino acid modification at any one or more of amino acid positions 255, 256, 258, 267, 268, 272, 276, 280, 283, 285, 286, 290, 298, 301, 305, 307, 309, 312, 315, 320, 322, 326, 330, 331, 333, 334, 337, 340, 360, 378, 398 or 430 of the Fc region.

For example, the variant with improved Fc gamma R binding activity may display increased binding to Fc gamma RIII, and optionally may further display decreased binding to Fc gamma RII; e.g. the variant may comprise an amino acid modification at position 298 and/or 333 of an Fc region.

Variants with increased binding to Fc gamma RII include those comprising an amino acid modification at any one or more of amino acid positions 255, 256, 258, 267, 268, 272, 276, 280, 283, 285, 286, 290, 301, 305, 307, 309, 312, 315, 320, 322, 326, 330, 331, 337, 340, 378, 398 or 430 of an Fc region. Such variants may further display decreased binding to Fc gamma RIII. For example, they may include an Fc region amino acid modification at any one or more of amino acid positions 268, 272, 298, 301, 322 or 340 (U.S. Pat. No. 6,737,056 issued to Presta).

While it is preferred to alter binding to a Fc gamma R, Fc region variants with altered binding affinity for the neonatal receptor (FcRn) are also contemplated. Fc region variants with improved affinity for FcRn are anticipated to have longer serum half-lives, and such molecules will have useful applications in methods of treating mammals where long half-life of the administered polypeptide is desired, e.g., to treat a chronic disease or disorder. Fc region variants with decreased FcRn binding affinity, on the contrary, are expected to have shorter half-lives, and such molecules may, for example, be administered to a mammal where a shortened circulation time may be advantageous, e.g. for in vivo diagnostic imaging or for polypeptides which have toxic side effects when left circulating in the blood stream for extended periods, etc. Fc region variants with decreased Fcln binding affinity are anticipated to be less likely to cross the placenta, and thus may be utilized in the treatment of diseases or disorders in pregnant women (U.S. Pat. No. 6,737,056 issued to Presta).

Fc region variants with altered binding affinity for FcRn include those comprising an Fc region amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 382, 386, 388, 400, 413, 415, 424, 433, 434, 435, 436, 439 or 447. Those which display reduced binding to FcRn will generally comprise an Fc region amino acid modification at any one or more of amino acid positions 252, 253, 254, 255, 288, 309, 386, 388, 400, 415, 433, 435, 436, 439 or 447; and those with increased binding to FcRn will usually comprise an Fc region amino acid modification at any one or more of amino acid positions 238, 256, 265, 272, 286, 303, 305, 307, 311, 312, 317, 340, 356, 360, 362, 376, 378, 380, 382, 413, 424 or 434 (U.S. Pat. No. 6,737,056 issued to Presta).

Furthermore, the present invention comprises the use of antibodies in which one or more alterations have been made in the Fc region in order to change functional or pharmacokinetic properties of the antibodies. Such alterations may result in a decrease or increase of C1q binding and CDC (complement dependent cytotoxicity) or of Fc gamma R binding and antibody-dependent cellular cytotoxicity (ADCC). Substitutions can for example be made in one or more of the amino acid positions 234, 235, 236, 237, 297, 318, 320, and 322 of the heavy chain constant region, thereby causing an alteration in an effector function while retaining binding to antigen as compared with the unmodified antibody (see, e.g., U.S. Pat. Nos. 5,624,821 and 5,648,260, both issued to Winter, et al.). Further reference may be had to WO 00/42072 disclosing antibodies with altered Fc regions that increase ADCC, and WO 94/29351 disclosing antibodies having mutations in the N-terminal region of the CH2 domain that alter the ability of the antibodies to bind to FcRI and thereby decreases the ability of the antibodies to bind to C1q which in turn decreases the ability of the antibodies to fix complement. Shields teaches combination variants, e.g., T256A/S298A, S298A/E333A, and S298A/E333A/K334A, that improve Fc gamma RIII binding (Shields et al. (2001) J. Biol. Chem. 276:6591-6604) (U.S. Pat. Applic. 2004/0208873 of Teeling, et al.).

The different IgG subclasses have different affinities for the Fc gamma Rs, with IgG 1 and IgG3 typically binding substantially better to the receptors than IgG2 and IgG4 (see, e.g., Presta, et al. (2002) Biochem. Soc. Trans. 30:487-490; Jefferis, et al. (2002) Immunol Lett 82:57-65). All Fc gamma Rs bind the same region on IgG Fc, yet with different affinities: the high affinity binder Fc gamma RI has a Kd for IgG1 of 10⁻⁸ M⁻¹, whereas the low affinity receptors Fc gamma RII and Fc gamma RIII generally bind at 10⁻⁶ and 10⁻⁵ respectively. The extracellular domains of Fc gamma RIIIa and Fc gamma RIIIb are 96% identical, however Fc gamma RIIIb does not have a intracellular signaling domain (U.S. Pat. Applic. 2004/0208873 of Teeling, et al.). Furthermore, whereas Fc gamma RI, Fc gamma RIIa/c, and Fc gamma RIIIa are positive regulators of immune complex-triggered activation, characterized by having an intracellular domain that has an immunoreceptor tyrosine-based activation motif (ITAM), Fc gamma RIIb has an immunoreceptor tyrosine-based inhibition motif (ITIM) and is therefore inhibitory. Thus the former are referred to as activation receptors, and Fc gamma RIIb is referred to as an inhibitory receptor. The receptors also differ in expression pattern and levels on different immune cells. Yet another level of complexity is the existence of a number of Fc gamma R polymorphisms in the human proteome. A particularly relevant polymorphism with clinical significance is V158/F158 Fc gamma RIIIa. Human IgG 1 binds with greater affinity to the V158 allotype than to the F 158 allotype. This difference in affinity, and presumably its effect on ADCC and/or ADCP, has been shown to be a significant determinant of the efficacy of the anti-CD20 antibody rituximab. Patients with the V158 allotype respond favorably to rituximab treatment; however, patients with the lower affinity F158 allotype respond poorly (Cartron etal. (2002) Blood 99:754-758). Approximately 10-20% of humans are V1581V158 homozygous, 45% are V158/F158 heterozygous, and 35-45% of humans are F158/F158 homozygous (Lehrnbecher etal. (1999) Blood 94:4220-4232; Cartron et al. (2002) Blood 99:754-758). Thus 80-90% of humans are poor responders, that is they have at least one allele of the F158 Fc gamma RIIa (U.S. Pat. Applic. 2005/0054832 of Lazar, et al.)

Also available for use in the invention are Fc variants that have been characterized using one or more of the experimental methods described herein. In one aspect, said Fc variants comprise at least one amino acid substitution at a position selected from the group consisting of: 230, 233, 234, 235, 239, 240, 241, 243, 244, 245, 247, 262, 263, 264, 265, 266, 267, 269, 270, 272, 273, 274, 275, 276, 278, 283, 296, 297, 298, 299, 302, 313, 318, 320, 323, 324, 325, 326, 327, 328, 329, 330, 331, 332, 333, 334, and 335, wherein the numbering of the residues in the Fc region is that of the EU index as in Kabat. In one aspect, said Fc variants comprise at least one amino acid substitution at a position selected from the group consisting of: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and 428, wherein the numbering of the residues in the Fc region is that of the EU index as in Kabat. In a preferred aspect, said Fc variants comprise at least one substitution selected from the group consisting of P230A, E233D, L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, S267T, S267H, S267D, S267N, E269H, E269Y, E269F, E269R, E269T, E269L, E269N, D270Q, D270T, D270H, E272S, E272K, E272I, E272Y, V273I, K274T, K274E, K274R, K274L, K274Y, F275W, N276S, N276E, N276R, N276L, N276Y, Y278T, Y278E, Y278K, Y278W, E283R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, V302I, W313F, E318R, K320T, K320D, K320I, K322T, K322H, V323I, S324T, S324D, S324R, S324I, S324V, S324L, S324Y, N325Q, N325L, N325I, N325D, N325E, N325A, N325T, N325V, N325H, K326L, K326I, K326T, A327N, A327L, A327D, A327T, L328M, L328D, L328E, L328N, L328Q, L328F, L328I, L328V, L328T, L328H, L328A, P329F, A330L, A330Y, A330V, A330I, A330F, A330R, A330H, A330S, A330W, A330M, P331V, P331H, I332D, I332E, I332N, I332Q, I332T, I332H, I332Y, I332A, E333T, E333H, E333I, E333Y, K334I, K334T, K334F, T335D, T335R, and T335Y, wherein the numbering of the residues in the Fc region is that of the EU index as in Kabat. In a variety of alternate aspects, said Fc variants are selected from the group consisting of V264L, V264I, F241W, F241L, F243W, F243L, F241L/F243L/V262I/V264I, F241 W/F243W, F241 W/F243W/V262A/V264A, F241 L/V262I, F243L/V264I, F243L/V262I/V264W, F241 Y/F243Y/N262T/V264T, F241BE/F243R/V262E/V264R, F241 E/F243Q/V262T/V264E, F241 R/F243 Q/V262T/V264R-, F241 E/F243Y/V262T/V264R, L328M, L328E, L328F I332E, L328M/I332E, P244H, P245A, P247V, W313F, P244H/P245A/P247V, P247G, V264I/I332E, F241 E/F243R/V262E/V264R/I332E, F241 E/F243Q/V262T/V264E/I332E, F24 lR/F243Q/V262T/V264R/I332E, F241 E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241 Y/F243 Y/V262T/V264T/N297D/I33-2E, A330Y/I332E, V264I/A330Y/I332E, A330L/I332E, V264I/A330L/I332E, L234D, L234D, L234E, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239EIN297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A33 OL/I332E, L328N, L328H, S239D/I332E/A330I, N297D/I332E/S239D/A330L, P230A, E233D, P230A/E233D, P230A/E233D/I332E, S267T, S267H, S267D, S267N, E269T, E269L, E269N, D270Q, D270T, D270H, E272S, E272K, E272I, E272Y, V273I, K274T, K274E, K274R, K274L, K274Y, F275W, N276S, N276E, N276R, N276L, N276Y, Y278T, Y278E, Y278K, Y278W, E283R, V302I, E318R, K320T, K320D, K320I, K322T, K322H, V323I, S324T, S324D, S324R, S324I, S324V, S324L, S324Y, K326L, K326I, K326T, A327D, A327T, A330S, A330W, A330M, P331V, P331H, E333T, E333H, E333I, E333Y, K334I, K334T, K334F, T335D, T335R, T335Y, L234I/L235D, V240I/266I, S239D/A330Y/I332E/L234I, S239D/A330Y/I332E/L235D, S239D/A330Y/I332E/V240I, S239D/A330Y/I332E/V264T-, S239D/A330Y/I332E/V266I, S239D/A330Y/I332E/K326E, S239D/A330Y/I332E/K326T, S239D/N297D/I332E/A330Y, S239D/N297D/I332E/A330Y- /F241 S/F243H/V262T/V264T, S239D/N297D/I332E/L235D, and S239D/N297D/I332E/K326E, wherein the numbering of the residues in the Fc region is that of the EU index as in Kabat (U.S. Pat. Applic. 2005/0054832 of Lazar, et al.).

What is also available for the invention are Fc variants that are selected from the group consisting of D221K, D221Y, K222E, K222Y, T223E, T223K, H224E, H224Y, T225E, T225, T225K, T225W, P227E, P227K, P227Y, P227G, P228E, P228K, P228Y, P228G, P230E, P230Y, P230G, A231E, A231K, A231Y, A231P, A231G, P232E, P232K, P232Y, P232G, E233N, E233Q, E233K, E233R, E233S, E233T, E233H, E233A, E233V, E233L, E233I, E233F, E233M, E233Y, E233W, E233G, L234K, L234R, L234S, L234A, L234M, L234W, L234P, L234G, L235E, L235K, L235R, L235A, L235M, L235W, L235P, L235G, G236D, G236E, G236N, G236Q, G236K, G236R, G236S, G236T, G236H, G236A, G236V, G236L, G236I, G236F, G236M, G236Y, G236W, G236P, G237D, G237E, G237N, G237Q, G237K, G237R, G237S, G237T, G237H, G237V, G237L, G237I, G237F, G237M, G237Y, G237W, G237P, P238D, P238E, P238N, P238Q, P238K, P238R, P238S, P238T, P238H, P238V, P238L, P238I, P238F, P238M, P238Y, P238W, P238G, S239Q, S239K, S239R, S239V, S239L, S239I, S239M, S239W, S239P, S239G, F241D, F241E, F241Y, F243E, K246D, K246E, K246H, K246Y, D249Q, D249H, D249Y, R255E, R255Y, E258S, E258H, E258Y, T260D, T260E, T260H, T260Y, V262E, V262F, V264D, V264E, V264N, V264Q, V264K, V264R, V264S, V264H, V264W, V264P, V264G, D265Q, D265K, D265R, D265S, D265T, D265H, D265V, D265L, D265I, D265F, D265M, D265Y, D265W, D265P, S267E, S267Q, S267K, S267R, S267V, S267L, S267I, S267F, S267M, S267Y, S267W, S267P, H268D, H268E, H268Q, H268K, H268R, H268T, H268V, H268L, H268I, H268F, H268M, H268W, H268P, H268G, E269K, E269S, E269V, E269I, E269M, E269W, E269P, E269G, D270R, D270S, D270L, D270I, D270F, D270M, D270Y, D270W, D270P, D270G, P271D, P271E, P271N, P271Q, P271K, P271R, P271S, P271T, P271H, P271A, P271V, P271L, P271I, P271F, P271M, P271Y, P271W, P271G, E272D, E272R, E272T, E272H, E272V, E272L, E272F, E272M, E272W, E272P, E272G, K274D, K274N, K274S, K274H, K274V, K274I, K274F, K274M, K274W, K274P, K274G, F275L, N276D, N276T, N276H, N276V, N276I, N276F, N276M, N276W, N276P, N276G, Y278D, Y278N, Y278Q, Y278R, Y278S, Y278H, Y278V, Y278L, Y278I, Y278M, Y278P, Y278G, D280K, D280L, D280W, D280P, D280G, G281D, G281K, G281Y, G281P, V282E, V282K, V282Y, V282P, V282G, E283K, E283H, E283L, E283Y, E283P, E283G, V284E, V284N, V284T, V284L, V284Y, H285D, H285E, H285Q, H285K, H285Y, H285W, N286E, N286Y, N286P, N286G, K288D, K288E, K288Y, K290D, K290N, K290H, K290L, K290W, P291D, P291E, P291Q, P291T, P291H, P291I, P291G, R292D, R292E, R292T, R292Y, E293N, E293R, E293S, E293T, E293H, E293V, E293L, E293I, E293F, E293M, E293Y, E293W, E293P, E293G, E294K, E294R, E294S, E294T, E294H, E294V, E294L, E294I, E294F, E294M, E294Y, E294W, E294P, E294G, Q295D, Q295E, Q295N, Q295R, Q295S, Q295T, Q295H, Q295V, Q295I, Q295F, Q295M, Q295Y, Q295W, Q295P, Q295G, Y296K, Y296R, Y296A, Y296V, Y296M, Y296G, N297Q, N297K, N297R, N297T, N297H, N297V, N297L, N297I, N297F, N297M, N297Y, N297W, N297P, N297G, S298D, S298E, S298Q, S298K, S298R, S298I, S298F, S298M, S298Y, S298W, T299D, T299E, T299N, T299Q, T299K, T299R, T299L, T299F, T299M, T299Y, T299W, T299P, T299G, Y300D, Y300E, Y300N, Y300Q, Y300K, Y300R, Y300S, Y300T, Y300H, Y300A, Y300V, Y300M, Y300W, Y300P, Y300G, R301D, R301E, R301H, R301Y, V303D, V303E, V303Y, S304D, S304N, S304T, S304H, S304L, V305E, V305T, V305Y, K317E, K317Q, E318Q, E318H, E318L, E318Y, K320N, K320S, K320H, K320V, K320L, K320F, K320Y, K320W, K320P, K320G, K322D, K322S, K322V, K322I, K322F, K322Y, K322W, K322P, K322G, S324H, S324F, S324M, S324W, S324P, S324G, N325K, N325R, N325S, N325F, N325M, N325Y, N325W, N325P, N325G, K326P, A327E, A327K, A327R, A327H, A327V, A327I, A327F, A327M, A327Y, A327W, A327P, L328D, L328Q, L328K, L328R, L328S, L328T, L328V, L328I, L328Y, L328W, L328P, L328G, P329D, P329E, P329N, P329Q, P329K, P329R, P329S, P329T, P329H, P329V, P329L, P329I, P329M, P329Y, P329W, P329G, A330E, A330N, A330T, A330P, A330G, P331D, P331Q, P331R, P331T, P331L, P3311, P331F, P331M, P331Y, P331W, I332K, I332R, I332S, I332V, I332L, I332F, I332M, I332W, I332P, I332G, E333L, E333F, E333M, E333P, K334P, T335N, T335S, T335H, T335V, T335L, T335I, T335F, T335M, T335W, T335P, T335G, I336E, I336K, I336Y, S337E, S337N, and S337H, wherein the numbering of the residues in the Fc region is that of the EU index as in Kabat (U.S. Pat. Applic. 2005/0054832 of Lazar, et al.).

What is also available for use in the invention is an Fc variant that binds with greater affinity to one or more Fc gamma Rs. In one aspect, said Fc variants have affinity for an Fc gamma R that is more than 1-fold greater than that of the parent Fc polypeptide. In an alternate aspect, said Fc variants have affinity for an Fc gamma R that is more than 5-fold greater than that of the parent Fc polypeptide. In a preferred aspect, said Fc variants have affinity for an Fc gamma R that is between 5-fold and 300-fold greater than that of the parent Fc polypeptide. In one aspect, said Fc variants comprise at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, wherein the numbering of the residues in the Fc region is that of the EU index as in Kabat. In a preferred aspect, said Fc variants comprise at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V2641, V264T, V264Y, V266I, 272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, wherein the numbering of the residues in the Fc region is that of the EU index as in Kabat. In a mostly preferred aspect, said Fc variants are selected from the group consisting of V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L3281/1332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/V332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T-, S239D/A330Y/I332E/K326E, and S239D/A330Y/I332E/K326T, wherein the numbering of the residues in the Fc region is that of the EU index as in Kabat (U.S. Pat. Applic. 2005/0054832 of Lazar, et al.).

In one aspect, said Fc variants comprise at least one amino acid substitution at a position selected from the group consisting of: 234, 235, 239, 240, 264, 296, 330, and I332, wherein the numbering of the residues in the Fc region is that of the EU index as in Kabat. In a typical aspect, the Fc variants comprise at least one amino acid substitution selected from the group consisting of: L234Y, L234I, L235I, S239D, S239E, S239N, S239Q, V240A, V240M, V264I, V264Y, Y296Q, A330L, A330Y, A330I, I332D, and I332E, wherein the numbering of the residues in the Fc region is that of the EU index as in Kabat. In another typical aspect, said Fc variants are selected from the group consisting of: I332E, V264I/I332E, S239E/I332E, S239Q/I332E, Y296Q, A330L, A330Y, I332D, S239D, S239D/I332E, A330Y/I332E, V264I/A330Y/I332E, A330L/I332E, V264I/A330L/I332E, L234Y, L234I, L235I, V240A, V240M, V264Y, A330I, S239D/A330L/I332E, S239D/S298A/I332E, S239N/S298A/I332E, S239D/V264I/I332E, S239D/V264I/S298A/I332E, and S239D/V264I/A330L/I332E, wherein the numbering of the residues in the Fc region is that of the EU index as in Kabat (U.S. Pat. Applic. 2005/0054832 of Lazar, et al.).

What is available for use in the invention are Fc variants that mediate effector function more effectively in the presence of effector cells. In one aspect, said Fc variants mediate ADCC that is greater than that mediated by the parent Fc polypeptide. In a typical aspect, said Fc variants mediate ADCC that is more than 5-fold greater than that mediated by the parent Fc polypeptide. In a more typical aspect, said Fc variants mediate ADCC that is between 5-fold and 1000-fold greater than that mediated by the parent Fc polypeptide. In one aspect, said Fc variants comprise at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, wherein the numbering of the residues in the Fc region is that of the EU index as in Kabat. In a normal aspect, said Fc variants comprise at least one amino acid substitutions selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, wherein the numbering of the residues in the Fc region is that of the EU index as in Kabat. In a more normal aspect, said Fc variants are selected from the group consisting of: V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A33CL/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, wherein the numbering of the residues in the Fc region is that of the EU index as in Kabat (U.S. Pat. Applic. 2005/0054832 of Lazar, et al.).

Also provided for use in the methods are Fc variants that bind with weaker affinity to one or more Fc gamma Rs. In one aspect, said Fc variants comprise at least one amino acid substitution at a position selected from the group consisting of: 230, 233, 234, 235, 239, 240, 241, 243, 244, 245, 247, 262, 263, 264, 265, 266, 267, 269, 270, 273, 276, 278, 283, 296, 297, 298, 299, 313, 323, 324, 325, 327, 328, 329, 330, 332, and 333, wherein the numbering of the residues in the Fc region is that of the EU index as in Kabat. In a usual aspect, said Fc variants comprise an amino acid substitution at a position selected from the group consisting of: P230A, E233D, L234D, L234N, L234Q, L234T, L234H, L234V, L234F, L234I, L235N, L235Q, L235T, L235H, L235V, L235F, L235D, S239E, S239N, S239Q, S239F, S239H, S239Y, V240A, V240T, F241W, F241L, F241Y, F241E, F241R, F243W, F243L F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264E, D265G, D265N, D265Q, D265Y, D265F, D265V, D265I, D265L, D265H, D265T, V266A, V266T, V266M, S267Q, S267L, E269H, E269Y, E269F, E269R, E269T, E269L, E269N, D270Q, D270T, D270H, V273I, N276S, N276E, N276R, N276Y, Y278E, Y278W, E283R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, V323I, S324R, S324L, S324Y, N325Q, N325L, N325I, N325D, N325E, N325A, N325V, N325H, A327N, A327L, L328M, 328E, L328N, L328Q, A327D, A327T, L328F, L328H, L328A, L328N, L328H, P329F, A330L, A330V, A330F, A330R, A330H, I332N, I332Q, I332T, I332H, I332Y, I332A, E333T, and E333H, wherein the numbering of the residues in the Fc region is that of the EU index as in Kabat. In a more usual aspect, said Fc variants are selected from the group consisting of: V264L, F241W, F241L, F243W, F243L, F241L/F243L/V262I/V264I, F241W/F243W, F241 W/F243W/V262A/V264A, F241L/V262I, F243L/V262I/L264W, F241 Y/F243Y/V262T/V264T, F241 E/F243R/V262E/V264R, F241 E/F243Q/V262T/V264E, F241 R/F243Q/V262T/V264R, F241E/F243Y/V262T/V264R, L328M, L328E, L328F, P244H, P245A, P247V, W313F, P244H/P245A/P247V, P247G, F241E/F243R/V262E/V264R/I332E, F241E/F243Y/V262T/V264R/I332E, D265G, D265N, S239E/D265G, S239E/D265N, S239E/D265Q, Y296E, Y296Q, T299I, A327N, S267Q/A327S, S267L/A327S, A327L, P329F, A330L, N297S, N297D, N297S/I332E, I332N, I332Q, V264F, V263I, T299A, T299S, T299V, N325Q, N325L, N325I, S239N, S239F, S239N/I332N, S239N/I332Q, S239Q/I332N, S239Q/I332Q, Y296D, Y296N, L234D, L234N, L234Q, L234T, L234H, L234V, L234F, L235N, L235Q, L235T, L235H, L235V, L235F, S239H, S239Y, V240A, V263T, V263M, V264M, V266A, V266T, V266M, E269H, E269Y, E269F, E269R, Y296S, Y296T, Y296L, Y296I, A298H, T299H, A330V, A330F, A330R, A330H, N325D, N325E,-N325A, N325V, N325H, L328E/I332E, L328N/I332E, L328Q/I332E, L328H/I332E, L328A, I332T, I332H, I332Y, I332A, L328N, L328H, E233D, P230A/E233D, E269T, E269L, E269N, D270Q, D270T, D270H, V273I, N276S, N276E, N276R, N276Y, Y278E, Y278W, E283R, V323I, S324R, S324L, S324Y, A327D, A327T, E333T, E333H, and L234I/L235D, wherein the numbering of the residues in the Fc region is that of the EU index as in Kabat (U.S. Pat. Applic. 2005/0054832 of Lazar, et al.).

Fc variants may be used that mediate ADCC in the presence of effector cells less effectively.-In one aspect, said Fc variants comprise at least one amino acid substitution at a position selected from the group consisting of: 230, 233, 234, 235, 239, 240, 241, 243, 244, 245, 247, 262, 263, 264, 265, 266, 267, 269, 270, 273, 276, 278, 283, 296, 297, 298, 299, 313, 323, 324, 325, 327, 328, 329, 330, 332, and 333, wherein the numbering of the residues in the Fc region is that of the EU index as in Kabat. In a usual aspect, said Fc variants comprise at least one amino acid substitution at a position selected from the group consisting of: P230A, E233D, L234D, L234N, L234Q, L234T, L234H, L234V, L234F, L234I, L235N, L235Q, L235T, L235H, L235V, L235F, L235D, S239E, S239N, S239Q, S239F, S239H, S239Y, V240A, V240T, F241W, F241L, F241Y, F241E, F241R, F243W, F243L F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264E, D265G, D265N, D265Q, D265Y, D265F, D265V, D265I, D265L, D265H, D265T, V266A, V266T, V266M, S267Q, S267L, E269H, E269Y, E269F, E269R, E269T, E269L, E269N, D270Q, D270T, D270H, V273I, N276S, N276E, N276R, N276Y, Y278E, Y278W, E283R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, V323I, S324R, S324L, S324Y, N325Q, N325L, N325I, N325D, N325E, N325A, N325V, N325H, A327N, A327L, L328M, 328E, L328N, L328Q, A327D, A327T, L328F, L328H, L328A, L328N, L328H, P329F, A330L, A330V, A330F, A330R, A330H, I332N, I332Q, I332T, I332H, I332Y, I332A, E333T, and E333H, wherein the numbering of the residues in the Fc region is that of the EU index as in Kabat. In a conventional aspect, said Fc variants are selected from the group consisting of: V264L, F241W, F241L, F243W, F243L, F241L/F243I/V262I/V264I, F241W/F243W, F241W/F243W/V262A/V264A, F241L/V262I, F243L/V262I/V264W, F241 Y/F243Y/V262T/V264T, F241 E/F243R/V262E/V264R, F241 E/F243Q/V262T/V264E, F241 R/F243Q/V262T/V264R-, F241E/F243Y/V262T/V264R, L328M, L328E, L328F, P244H, P245A, P247V, W313F, P244H/P245A/P247V, P247G, F241 E/F243R[V262E/N264R/I332E, F241 E/F243Y/V262T/V264R/I332E, D265G, D265N, S239E/D265G, S239E/D265N, S239E/D265Q, Y296E, Y296Q, T299I, A327N, S267Q/A327S, S267L/A327S, A327L, P329F, A330L, N297S, N297D, N297S/I332E, I332N, I332Q, V264F, V263I, T299A, T299S, T299V, N325Q, N325L, N325I, S239N, S239F, S239N/I332N, S239N/I332Q, S239Q/I332N, S239Q/I332Q, Y296D, Y296N, L234D, L234N, L234Q, L234T, L234H, L234V, L234F, L235N, L235Q, L235T, L235H, L235V, L235F, S239H, S239Y, V240A, V263T, V263M, V264M, V266A, V266T, V266M, E269H, E269Y, E269F, E269R, Y296S, Y296T, Y296L, Y296I, A298H, T299H, A330V, A330F, A330R, A330H, N325D, N325E, N325A, N325V, N325H, L328E/I332E, L328N/I332E, L328Q/I332E, L328H/I332E, L328A, I332T, I332H, I332Y, I332A, L328N, L328H, E233D, P230A/E233D, E269T, E269L, E269N, D270Q, D270T, D270H, V273I, N276S, N276E, N276R, N276Y, Y278E, Y278W, E283R, V323I, S324R, S324L, S324Y, A327D, A327T, E333T, E333H, and L234I/L235D, wherein the numbering of the residues in the Fc region is that of the EU index as in Kabat (U.S. Pat. Applic. 2005/0054832 of Lazar, et al.).

Fc variants may be used that have improved function and/or solution properties as compared to the aglycosylated form of the parent Fc polypeptide. Improved functionality herein includes but is not limited to binding affinity to an Fc ligand. Improved solution properties herein includes but is not limited to stability and solubility. In one aspect, said aglycosylated Fc variants bind to an Fc gamma R with an affinity that is comparable to or better than the glycosylated parent Fc polypeptide. In an alternate aspect, said Fc variants bind to an Fc gamma R with an affinity that is within 0.4-fold of the glycosylated form of the parent Fc polypeptide. In one aspect, said Fc variants comprise at least one amino acid substitution at a position selected from the group consisting of: 239, 241, 243, 262, 264, 265, 296, 297, 330, and 332, wherein the numbering of the residues in the Fc region is that of the EU index as in Kabat. In a usual aspect, said Fc variants comprise an amino acid substitution selected from the group consisting of: S239D, S239E, F241Y, F243Y, V262T, V264T, V264E, D265Y, D265H, D265V, D265I, Y296N, N297D, A330Y, and I332E, wherein the numbering of the residues in the Fc region is that of the EU index as in Kabat. In a mostly preferred aspect, said Fc variants are selected from the group consisting of: N297D/I332E, F241Y/F243Y/V262T/V264T/N297D/I332E, S239D/N297D/I332E, S239E/N297D/I332E, S239D/D265Y/N297D/I332E, S239D/D265H/N297D/I332E, V264E/N297D/I332E, Y296N/N297D/I332E, N297D/A330Y/I332E, S239D/D265V/N297D/I332E, S239D/D265II/N297D/I332E, and N297D/S298A/A330Y/I332E, wherein the numbering of the residues in the Fc region is that of the EU index as in Kabat (U.S. Pat. Applic. 2005/0054832 of Lazar, et al.).

Provided also are mutants with enhanced altered affinities for Fc gamma RIIIA and/or Fc gamma RIIa. Also supplied are mutants with enhanced affinity for Fc gamma RIIIA and reduced or no affinity for Fc gamma RIIB. Further provided are mutants with enhanced affinity to Fc gamma RIIIA and Fc gamma RIIB (U.S. Pat. Applic. 2005/00064514 of Stavenhagen, et al.).

V. Antibodies to Antigens of Tumor Cells, Infectious Agents, and the like.

The present invention can utilize an antibody, or binding compound derived from an antibody, that specifically binds a protein, or oligopeptide or epitope derived from a protein, of Table 3. It can also utilize a bacterial genome, e.g., a listerial genome, or a bacterium, e.g., L. monocytogenes, comprising a nucleic acid encoding at least one protein, or oligopeptide or epitope derived from a protein, of Table 3. The nucleic acid can be plasmid-based or chromosomal, that is, the nucleic acid can be integrated into the bacterial genome. The encoded protein can be engineered to be intracellular (within the bacterium), secreted from the bacterium, bound to the cell wall of the bacterium, and/or bound to the cell membrane of the bacterium.

TABLE 3
Antigens and nucleic acids encoding antigens.

| | |
|---|---|
| Antigen | Reference |

Tumor antigens

| | |
|---|---|
| Mesothelin | GenBank Acc. No. NM_005823; U40434; NM_013404; BC003512 (see |
| | also, e.g., Hassan, et al. (2004) Clin. Cancer Res. 10: 3937-3942; |
| | Muminova, et al. (2004) BMC Cancer 4:19; Iacobuzio-Donahue, et al. |
| | (2003) Cancer Res. 63: 8614-8622). |
| Prostate stem cell antigen | GenBank Acc. No. AF043498; AR026974; AR302232 (see also, e.g., |
| (PSCA). | Argani, et al. (2001) Cancer Res. 61: 4320-4324; Christiansen, et al. (2003) |
| | Prostate 55: 9-19; Fuessel, et al. (2003) 23: 221-228). |
| Prostate acid phosphatase | Small, et al. (2000) J. Clin. Oncol. 18: 3894-3903; Altwein and Luboldt |
| (PAP); prostate-specific | (1999) Urol. Int. 63: 62-71; Chan, et al. (1999) Prostate 41: 99-109; Ito, et |
| antigen (PSA); PSM; | al. (2005) Cancer 103: 242-250; Schmittgen, et al. (2003) Int. J. Cancer |
| PSMA. | 107: 323-329; Million, et al. (1999) Eur. Urol. 36: 278-285. |
| Six-transmembrane | See, e.g., Machlenkin, et al. (2005) Cancer Res. 65: 6435-6442; GenBank |
| epithelial antigen of | Acc. No. NM_018234; NM_001008410; NM_182915; NM_024636; |
| prostate (STEAP). | NM_012449; BC011802. |
| Prostate carcinoma tumor | See, e.g., Machlenkin, et al. (2005) Cancer Res. 65: 6435-6442; GenBank |
| antigen-1 (PCTA-1). | Acc. No. L78132. |
| Prostate tumor-inducing | See, e.g., Machlenkin, et al. (2005) Cancer Res. 65: 6435-6442). |
| gene-1 (PTI-1). | |
| Prostate-specific gene | See, e.g., Machlenkin, et al. (2005) Cancer Res. 65: 6435-6442). |
| with homology to | |
| G protein-coupled | |
| receptor. | |
| Prostase (an antrogen | See, e.g., Machlenkin, et al. (2005) Cancer Res. 65: 6435-6442; GenBank |
| regulated serine | Acc. No. BC096178; BC096176; BC096175. |
| protease). | |
| Proteinase 3. | GenBank Acc. No. X55668. |
| Cancer-testis antigens, | GenBank Acc. No. NM_001327 (NY-ESO-1) (see also, e.g., Li, et al. |
| e.g., NY-ESO-1; SCP-1; | (2005) Clin. Cancer Res. 11: 1809-1814; Chen, et al. (2004) Proc. Natl. |
| SSX-1; SSX-2; SSX-4; | Acad. Sci. USA. 101(25): 9363-9368; Kubuschok, et al. (2004) Int. J. |
| GAGE, CT7; CT8; CT10; | Cancer. 109: 568-575; Scanlan, et al. (2004) Cancer Immun. 4:1; Scanlan, |
| MAGE-1; MAGE-2; | et al. (2002) Cancer Res. 62: 4041-4047; Scanlan, et al. (2000) Cancer |
| MAGE-3; MAGE-4; | Lett. 150: 155-164; Dalerba, et al. (2001) Int. J. Cancer 93: 85-90; Ries, et |
| MAGE-6; LAGE-1. | al. (2005) Int. J. Oncol. 26: 817-824. |
| MAGE-A1, MAGE-A2; | Otte, et al. (2001) Cancer Res. 61: 6682-6687; Lee, et al. (2003) Proc. Natl. |
| MAGE-A3; MAGE-A4; | Acad. Sci. USA 100: 2651-2656; Sarcevic, et al. (2003) Oncology 64: 443-449; |
| MAGE-A6; MAGE-A9; | Lin, et al. (2004) Clin. Cancer Res. 10: 5708-5716. |
| MAGE-A10; | |
| MAGE-A12; GAGE-3/6; | |
| NT-SAR-35; BAGE; | |
| CA125. | |
| GAGE-1; GAGE-2; | De Backer, et al. (1999) Cancer Res. 59: 3157-3165; Scarcella, et al. |
| GAGE-3; GAGE-4; | (1999) Clin. Cancer Res. 5: 335-341. |
| GAGE-5; GAGE-6; | |
| GAGE-7; GAGE-8; | |
| GAGE-65; GAGE-11; | |
| GAGE-13; GAGE-7B. | |
| HIP1R; LMNA; | Scanlan, et al. (2002) Cancer Res. 62: 4041-4047. |
| KIAA1416; Seb4D; | |
| KNSL6; TRIP4; MBD2; | |
| HCAC5; MAGEA3. | |
| DAM family of genes, | Fleishhauer, et al. (1998) Cancer Res. 58: 2969-2972. |
| e.g., DAM-1; DAM-6. | |
| RCAS1. | Enjoji, et al. (2004) Dig. Dis. Sci. 49: 1654-1656. |
| RU2. | Van Den Eynde, et al. (1999) J. Exp. Med. 190: 1793-1800. |
| CAMEL. | Slager, et al. (2004) J. Immunol. 172: 5095-5102; Slager, et al. (2004) |
| | Cancer Gene Ther. 11: 227-236. |
| Colon cancer associated | Scanlan, et al. (2002) Cancer Res. 62: 4041-4047. |
| antigens, e.g., NY-CO-8; | |
| NY-CO-9; NY-CO-13; | |
| NY-CO-16; NY-CO-20; | |
| NY-CO-38; NY-CO-45; | |
| NY-CO-9/HDAC5; | |
| NY-CO-41/MBD2; | |
| NY-CO-42/TRIP4; | |
| NY-CO-95/KIAA1416; | |
| KNSL6; seb4D. | |
| N-Acetylglucosaminyl- | Dosaka-Akita, et al. (2004) Clin. Cancer Res. 10: 1773-1779. |
| tranferase V (GnT-V). | |
| Elongation factor 2 | Renkvist, et al. (2001) Cancer Immunol Immunother. 50: 3-15. |
| mutated (ELF2M). | |
| HOM-MEL-40/SSX2 | Neumann, et al. (2004) Int. J. Cancer 112: 661-668; Scanlan, et al. (2000) |
| | Cancer Lett. 150: 155-164. |
| BRDT. | Scanlan, et al. (2000) Cancer Lett. 150: 155-164. |
| SAGE; HAGE. | Sasaki, et al. (2003) Eur. J. Surg. Oncol. 29: 900-903. |
| RAGE. | See, e.g., Li, et al. (2004) Am. J. Pathol. 164: 1389-1397; Shirasawa, et al. |
| | (2004) Genes to Cells 9: 165-174. |
| MUM-1 (melanoma | Gueguen, et al. (1998) J. Immunol. 160: 6188-6194; Hirose, et al. (2005) |
| ubiquitous mutated); | Int. J. Hematol. 81: 48-57; Baurain, et al. (2000) J. Immunol. 164: 6057-6066; |
| MUM-2; MUM-2 Arg- | Chiari, et al. (1999) Cancer Res. 59: 5785-5792. |
| Gly mutation; MUM-3. | |
| LDLR/FUT fusion | Wang, et al. (1999) J. Exp. Med. 189: 1659-1667. |
| protein antigen of | |
| melanoma. | |
| NY-REN series of renal | Scanlan, et al. (2002) Cancer Res. 62: 4041-4047; Scanlan, et al. (1999) |
| cancer antigens. | Cancer Res. 83: 456-464. |
| NY-BR series of breast | Scanlan, et al. (2002) Cancer Res. 62: 4041-4047; Scanlan, et al. (2001) |
| cancer antigens, e.g., | Cancer Immunity 1:4. |
| NY-BR-62; NY-BR-75; | |
| NY-BR-85; NY-BR-62; | |
| NY-BR-85. | |
| BRCA-1; BRCA-2. | Stolier, et al. (2004) Breast J. 10: 475-480; Nicoletto, et al. (2001) Cancer |
| | Treat Rev. 27: 295-304. |
| DEK/CAN fusion | von Lindern, et al. (1992) Mol. Cell. Biol. 12: 1687-1697. |
| protein. | |
| Ras, e.g., wild type ras, | GenBank Acc. No. P01112; P01116; M54969; M54968; P01111; P01112; |
| ras with mutations at | K00654. |
| codon 12, 13, 59, or 61, | |
| e.g., mutations G12C; | |
| G12D; G12R; G12S; | |
| G12V; G13D; A59T; | |
| Q61H. K-RAS; H-RAS; | |
| N-RAS. | |
| BRAF (an isoform of | Tannapfel, et al. (2005) Am. J. Clin. Pathol. 123: 256-2601; Tsao and Sober |
| RAF). | (2005) Dermatol. Clin. 23: 323-333. |
| Melanoma antigens, | GenBank Acc. No. NM_206956; NM_206955; NM_206954; |
| including HST-2 | NM_206953; NM_006115; NM_005367; NM_004988; AY148486; |
| melanoma cell antigens. | U10340; U10339; M77481. See, e g., Suzuki, et al. (1999) J. Immunol. |
| | 163: 2783-2791. |
| Survivin | GenBank Acc. No. AB028869; U75285 (see also, e.g., Tsuruma, et al. |
| | (2004) J. Translational Med. 2:19 (11 pages); Pisarev, et al. (2003) Clin. |
| | Cancer Res. 9: 6523-6533; Siegel, et al. (2003) Br. J. Haematol. 122: 911-914; |
| | Andersen, et al. (2002) Histol. Histopathol. 17: 669-675). |
| MDM-2 | NM_002392; NM_006878 (see also, e.g., Mayo, et al. (1997) Cancer Res. |
| | 57: 5013-5016; Demidenko and Blagosklonny (2004) Cancer Res. |
| | 64: 3653-3660). |
| Methyl-CpG-binding | Muller, et al. (2003) Br. J. Cancer 89: 1934-1939; Fang, et al. (2004) |
| proteins (MeCP2; | World J. Gastreenterol. 10: 3394-3398. |
| MBD2). | |
| NA88-A. | Moreau-Aubry, et al. (2000) J. Exp. Med. 191: 1617-1624. |
| Histone deacetylases | Waltregny, et al. (2004) Eur. J. Histochem. 48: 273-290; Scanlan, et al. |
| (HDAC), e.g., HDAC5. | (2002) Cancer Res. 62: 4041-4047. |
| Cyclophilin B (Cyp-B). | Tamura, et al. (2001) Jpn. J. Cancer Res. 92: 762-767. |
| CA 15-3; CA 27.29. | Clinton, et al. (2003) Biomed. Sci. Instrum. 39: 408-414. |
| Heat shock protein | Faure, et al. (2004) Int. J. Cancer 108: 863-870. |
| Hsp70. | |
| GAGE/PAGE family, | Brinkmann, et al. (1999) Cancer Res. 59: 1445-1448. |
| e.g., PAGE-1; PAGE-2; | |
| PAGE-3; PAGE-4; | |
| XAGE-1; XAGE-2; | |
| XAGE-3. | |
| MAGE-A, B, C, and D | Lucas, et al. (2000) Int. J. Cancer 87: 55-60; Scanlan, et al. (2001) Cancer |
| families. MAGE-B5; | Immun. 1:4. |
| MAGE-B6; MAGE-C2; | |
| MAGE-C3; MAGE-3; | |
| MAGE-6. | |
| Kinesin 2; TATA element | Scanlan, et al. (2001) Cancer Immun. 30: 1-4. |
| modulatory factor 1; | |
| tumor protein D53; NY | |
| Alpha-fetoprotein (AFP) | Grimm, et al. (2000) Gastroenterol. 119: 1104-1112. |
| SART1; SART2; | Kumamuru, et al. (2004) Int. J. Cancer 108: 686-695; Sasatomi, et al. |
| SART3; ART4. | (2002) Cancer 94: 1636-1641; Matsumoto, et al. (1998) Jpn. J. Cancer Res. |
| | 89: 1292-1295; Tanaka, et al. (2000) Jpn. J. Cancer Res. 91: 1177-1184. |
| Preferentially expressed | Matsushita, et al. (2003) Leuk. Lymphoma 44: 439-444; Oberthuer, et al. |
| antigen of melanoma | (2004) Clin. Cancer Res. 10: 4307-4313. |
| (PRAME). | |
| Carcinoembryonic | GenBank Acc. No. M29540; E03352; X98311; M17303 (see also, e.g., |
| antigen (CEA), CAP1-6D | Zaremba (1997) Cancer Res. 57: 4570-4577; Sarobe, et al. (2004) Curr. |
| enhancer agonist peptide. | Cancer Drug Targets 4: 443-454; Tsang, et al. (1997) Clin. Cancer Res. |
| | 3: 2439-2449; Fong, et al. (2001) Proc. Natl. Acad. Sci. USA 98: 8809-8814). |
| HER-2/neu. | Disis, et al. (2004) J. Clin. Immunol. 24: 571-578; Disis and Cheever |
| | (1997) Adv. Cancer Res. 71: 343-371. |
| cdk4; cdk6; p16 (INK4); | Ghazizadeh, et al. (2005) Respiration 72: 68-73; Ericson, et al. (2003) Mol. |
| Rb protein. | Cancer Res. 1: 654-664. |
| TEL; AML1; | Stams, et al. (2005) Clin. Cancer Res. 11: 2974-2980. |
| TEL/AML1. | |
| Telomerase (TERT). | Nair, et al. (2000) Nat. Med. 6: 1011-1017. |
| 707-AP. | Takahashi, et al. (1997) Clin. Cancer Res. 3: 1363-1370. |
| Annexin, e.g., | Zimmerman, et al. (2004) Virchows Arch. 445: 368-374. |
| Annexin II. | |
| BCR/ABL; BCR/ABL | Cobaldda, et al. (2000) Blood 95: 1007-1013; Hakansson, et al. (2004) |
| p210; BCR/ABL p190; | Leukemia 18: 538-547; Schwartz, et al. (2003) Semin. Hematol. 40: 87-96; |
| CML-66; CML-28. | Lim, et al. (1999) Int. J. Mol. Med. 4: 665-667. |
| BCL2; BLC6; | Iqbal, et al. (2004) Am. J. Pathol. 165: 159-166. |
| CD10 protein. | |
| CDC27 (this is a | Wang, et al. (1999) Science 284: 1351-1354. |
| melanoma antigen). | |
| Sperm protein 17 (SP17); | Arora, et al. (2005) Mol. Carcinog. 42: 97-108. |
| 14-3-3-zeta; MEMD; | |
| KIAA0471; TC21. | |
| Tyrosinase-related | GenBank Acc. No. NM_001922. (see also, e.g., Bronte, et al. (2000) |
| proteins 1 and 2 (TRP-1 | Cancer Res. 60: 253-258). |
| and TRP-2). | |
| gp100/pmel-17. | GenBank Acc. Nos. AH003567; U31798; U31799; U31807; U31799 (see |
| | also, e.g., Bronte, et al. (2000) Cancer Res. 60: 253-258). |
| TARP. | See, e.g., Clifton, et al. (2004) Proc. Natl. Acad. Sci. USA 101: 10166-10171; |
| | Virok, et al. (2005) Infection Immunity 73: 1939-1946. |
| Tyrosinase-related | GenBank Acc. No. NM_001922. (see also, e.g., Bronte, et al. (2000) |
| proteins 1 and 2 (TRP-1 | Cancer Res. 60: 253-258). |
| and TRP-2). | |
| Melanocortin 1 receptor | Salazar-Onfray, et al. (1997) Cancer Res. 57: 4348-4355; Reynolds, et al. |
| (MC1R); MAGE-3; | (1998) J. Immunol. 161: 6970-6976; Chang, et al. (2002) Clin. Cancer Res. |
| gp100; tyrosinase; | 8: 1021-1032. |
| dopachrome tautomerase | |
| (TRP-2); MART-1. | |
| MUC-1; MUC-2. | See, e.g., Davies, et al. (1994) Cancer Lett. 82: 179-184; Gambus, et al. |
| | (1995) Int. J. Cancer 60: 146-148; McCool, et al. (1999) Biochem. J. |
| | 341: 593-600. |
| Spas-1. | U.S. Published Pat. Appl. No. 20020150588 of Allison, et al. |
| CASP-8; FLICE; MACH. | Mandruzzato, et al. (1997) J. Exp. Med. 186: 785-793. |
| CEACAM6; CAP-1. | Duxbury, et al. (2004) Biochem. Biophys. Res. Commun. 317: 837-843; |
| | Morse, et al. (1999) Clin. Cancer Res. 5: 1331-1338. |
| HMGB1 (a DNA binding | Brezniceanu, et al. (2003) FASEB J. 17: 1295-1297. |
| protein and cytokine). | |
| ETV6/AML1. | Codrington, et al. (2000) Br. J. Haematol. 111: 1071-1079. |
| Mutant and wild type | Clements, et al. (2003) Clin. Colorectal Cancer 3: 113-120; Gulmann, et al. |
| forms of adenomatous | (2003) Appl. Immunohistochem. Mol. Morphol. 11: 230-237; Jungck, et al. |
| polyposis coli (APC); | (2004) Int. J. Colorectal. Dis. 19: 438-445; Wang, et al. (2004) J. Surg. |
| beta-catenin; c-met; p53; | Res. 120: 242-248; Abutaily, et al. (2003) J. Pathol. 201: 355-362; Liang, et |
| E-cadherin; | al. (2004) Br. J. Surg. 91: 355-361; Shirakawa, et al. (2004) Clin. Cancer |
| cyclooxygenase-2 | Res. 10: 4342-4348. |
| (COX-2). | |
| Renal cell carcinoma | Mulders, et al. (2003) Urol. Clin. North Am. 30: 455-465; Steffens, et al. |
| antigen bound by mAB | (1999) Anticancer Res. 19: 1197-1200. |
| G250. | |

Francisella tularensis antigens

| | |
|---|---|
| Francisella tularensis | Complete genome of subspecies Schu S4 (GenBank Acc. No. AJ749949); |
| A and B. | of subspecies Schu 4 (GenBank Acc. No. NC_006570). Outer membrane |
| | protein (43 kDa) Bevanger, et al. (1988) J. Clin. Microbiol. 27: 922-926; |
| | Porsch-Ozcurumez, et al. (2004) Clin. Diagnostic. Lab. Immunol. |
| | 11: 1008-1015). Antigenic components of F. tularensis include, e.g., 80 |
| | antigens, including 10 kDa and 60 kDa chaperonins (Havlasova, et al. |
| | (2002) Proteomics 2: 857-86), nucleoside diphosphate kinase, isocitrate |
| | dehydrogenase, RNA-binding protein Hfq, the chaperone ClpB |
| | (Havlasova, et al. (2005) Proteomics 5: 2090-2103). See also, e.g., Oyston |
| | and Quarry (2005) Antonie Van Leeuwenhoek 87: 277-281; Isherwood, et |
| | al. (2005) Adv. Drug Deliv. Rev. 57: 1403-1414; Biagini, et al. (2005) |
| | Anal. Bioanal. Chem. 382: 1027-1034. |

Malarial antigens

| | |
|---|---|
| Circumsporozoite protein | See, e.g., Haddad, et al. (2004) Infection Immunity 72: 1594-1602; |
| (CSP); SSP2; HEP17; | Hoffman, et al. (1997) Vaccine 15: 842-845; Oliveira-Ferreira and |
| Exp-1 orthologs found in | Daniel-Ribeiro (2001) Mem. Inst. Oswaldo Cruz, Rio de Janeiro 96: 221-227. |
| P. falciparum; and | CSP (see, e.g., GenBank Acc. No. AB121024). SSP2 (see, e.g., |
| LSA-1. | GenBank Acc. No. AF249739). LSA-1 (see, e.g., GenBank Acc. No. |
| | Z30319). |
| Ring-infected erythrocyte | See, e.g., Stirnadel, et al. (2000) Int. J. Epidemiol. 29: 579-586; Krzych, et |
| survace protein (RESA); | al. (1995) J. Immunol. 155: 4072-4077. See also, Good, et al. (2004) |
| merozoite surface | Immunol. Rev. 201: 254-267; Good, et al. (2004) Ann. Rev. Immunol. |
| protein 2 (MSP2); Spf66; | 23: 69-99. MSP2 (see, e.g., GenBank Acc. No. X96399; X96397). MSP1 |
| merozoite surface | (see, e.g., GenBank Acc. No. X03371). RESA (see, e.g., GenBank Acc. |
| protein 1(MSP1); 195A; | No. X05181; X05182). |
| BVp42. | |
| Apical membrane | See, e.g., Gupta, et al. (2005) Protein Expr. Purif. 41: 186-198. AMA1 |
| antigen 1 (AMA1). | (see, e.g., GenBank Acc. No. AJ494913; AJ494905; AJ490565). |

Viruses

| Hepatitis A | GenBank Acc. Nos., e.g., NC_001489; AY644670; X83302; K02990; |
|---|---|
| | M14707. |
| Hepatitis B | Complete genome (see, e.g., GenBank Acc. Nos. AB214516; NC_003977; |
| | AB205192; AB205191; AB205190; AJ748098; AB198079; AB198078; |
| | AB198076; AB074756). |
| Hepatitis C | Complete genome (see, e.g., GenBank Acc. Nos. NC_004102; AJ238800; |
| | AJ238799; AJ132997; AJ132996; AJ000009; D84263). |
| Hepatitis D | GenBank Acc. Nos, e.g. NC_001653; AB118847; AY261457. |
| Human papillomavirus, | See, e.g., Trimble, et al. (2003) Vaccine 21: 4036-4042; Kim, et al. (2004) |
| including all 200+ | Gene Ther. 11: 1011-1018; Simon, et al. (2003) Eur. J. Obstet. Gynecol. |
| subtypes (classed in | Reprod. Biol. 109: 219-223; Jung, et al. (2004) J. Microbiol. 42: 255-266; |
| 16 groups), such as the | Damasus-Awatai and Freeman-Wang (2003) Curr. Opin. Obstet. Gynecol. |
| high risk subtypes 16, 18, | 15: 473-477; Jansen and Shaw (2004) Annu. Rev. Med. 55: 319-331; |
| 30, 31, 33, 45. | Roden and Wu (2003) Expert Rev. Vaccines 2: 495-516; de Villiers, et al. |
| | (2004) Virology 324: 17-24; Hussain and Paterson (2005) Cancer |
| | Immunol. Immunother. 54: 577-586; Molijn, et al. (2005) J. Clin. Virol. 32 |
| | (Suppl. 1) S43-S51. GenBank Acc. Nos. AY686584; AY686583; |
| | AY686582; NC_006169; NC_006168; NC_006164; NC_001355; |
| | NC_001349; NC_005351; NC_001596). |
| Human T-cell | See, e.g., Capdepont, et al. (2005) AIDS Res. Hum. Retrovirus 21: 28-42; |
| lymphotropic virus | Bhigjee, et al. (1999) AIDS Res. Hum. Restrovirus 15: 1229-1233; |
| (HTLV) types I and II, | Vandamme, et al. (1998) J. Virol. 72: 4327-4340; Vallejo, et al. (1996) J. |
| including the | Aquir. Immune Defic. Syndr. Hum. Retrovirol. 13: 384-391. HTLV type I |
| HTLV type I subtypes | (see, e.g., GenBank Acc. Nos. AY563954; AY563953. HTLV type II |
| Cosmopolitan, Central | (see, e.g., GenBank Acc. Nos. L03561; Y13051; AF139382). |
| African, and | |
| Austro-Melanesian, and | |
| the HTLV type II | |
| subtypes IIa, IIb, IIc, and | |
| IId. | |
| Coronaviridae, including | See, e.g., Brian and Baric (2005) Curr. Top. Microbiol. Immunol. 287: 1-30; |
| Coronaviruses, such as | Gonzalez, et al. (2003) Arch. Virol. 148: 2207-2235; Smits, et al. |
| SARS-coronavirus | (2003) J. Virol. 77: 9567-9577; Jamieson, et al. (1998) J. Infect. Dis. |
| (SARS-CoV), and | 178: 1263-1269 (GenBank Acc. Nos. AY348314; NC_004718; |
| Toroviruses. | AY394850). |
| Rubella virus. | GenBank Acc. Nos. NC_001545; AF435866. |
| Mumps virus, including | See, e.g., Orvell, eta l. (2002) J. Gen. Virol. 83: 2489-2496. See, e.g., |
| the genotypes A, C, D, G, | GenBank Acc. Nos. AY681495; NC_002200; AY685921; AF201473. |
| H, and I. | |
| Coxsackie virus A | See, e.g., Brown, et al. (2003) J. Virol. 77: 8973-8984. GenBank Acc. |
| including the serotypes 1, | Nos. AY421768; AY790926: X67706. |
| 11, 13, 15, 17, 18, 19, 20, | |
| 21, 22, and 24 (also | |
| known as Human | |
| enterovirus C; HEV-C). | |
| Coxsackie virus B, | See, e.g., Ahn, et al. (2005) J. Med. Virol. 75: 290-294; Patel, et al. (2004) |
| including subtypes 1-6. | J. Virol. Methods 120: 167-172; Rezig, et al. (2004) J. Med. Virol. 72: 268-274. |
| | GenBank Acc. No. X05690. |
| Human enteroviruses | See, e.g., Oberste, et al. (2004) J. Virol. 78: 855-867. Human enterovirus A |
| including, e.g., human | (GenBank Acc. Nos. NC_001612); human enterovirus B (NC_001472); |
| enterovirus A (HEV-A, | human enterovirus C (NC_001428); human enterovirus D (NC_001430). |
| CAV2 to CAV8, CAV10, | Simian enterovirus A (GenBank Acc. No. NC_003988). |
| CAV12, CAV14, | |
| CAV16, and EV71) and | |
| also including HEV-B | |
| (CAV9, CBV1 to CBV6, | |
| E1 to E7, E9, E11 to E21, | |
| E24 to E27, E29 to E33, | |
| and EV69 and E73), as | |
| well as HEV. | |
| Polioviruses including | See, e.g., He, et al. (2003) J. Virol. 77: 4827-4835; Hahsido, et al. (1999) |
| PV1, PV2, and PV3. | Microbiol. Immunol. 43: 73-77. GenBank Acc. No. AJ132961 (type 1); |
| | AY278550 (type 2); X04468 (type 3). |
| Viral encephalitides | See, e.g., Hoke (2005) Mil. Med. 170: 92-105; Estrada-Franco, et al. |
| viruses, including equine | (2004) Emerg. Infect. Dis. 10: 2113-2121; Das, et al. (2004) Antiviral Res. |
| encephalitis, Venezuelan | 64: 85-92; Aguilar, et al. (2004) Emerg. Infect. Dis. 10: 880-888; Weaver, |
| equine encephalitis | et al. (2004) Arch. Virol. Suppl. 18: 43-64; Weaver, et al. (2004) Annu. |
| (VEE) (including | Rev. Entomol. 49: 141-174. Eastern equine encephalitis (GenBank Acc. |
| subtypes IA, IB, IC, ID, | No. NC_003899; AY722102); Western equine encephalitis (NC_003908). |
| IIIC, IIID), Eastern | |
| equine encephalitis | |
| (EEE), Western equine | |
| encephalitis (WEE), | |
| St. Louis encephalitis, | |
| Murray Valley | |
| (Australian) encephalitis, | |
| Japanese encephalitis, | |
| and tick-born | |
| encephalitis. | |
| Human herpesviruses, | See, e.g., Studahl, et al. (2000) Scand. J. Infect. Dis. 32: 237-248; Padilla, |
| including | et al. (2003) J. Med. Virol. 70 (Suppl. 1) S103-S110; Jainkittivong and |
| cytomegalovirus (CMV), | Langlais (1998) Oral Surg. Oral Med. 85: 399-403. GenBank Nos. |
| Epstein-Barr virus | NC_001806 (herpesvirus 1); NC_001798 (herpesvirus 2); X04370 and |
| (EBV), human | NC_001348 (herpesvirus 3); NC_001345 (herpesvirus 4); NC_001347 |
| herpesvirus-1 (HHV-1), | (herpesvirus 5); X83413 and NC_000898 (herpesvirus 6); NC_001716 |
| HHV-2, HHV-3, HHV-4, | (herpesvirus 7). |
| HHV-5, HHV-6, HHV-7, | Human herpesviruses types 6 and 7 (HHV-6; HHV-7) are disclosed by, |
| HHV-8, herpes B virus, | e.g., Padilla, et al. (2003) J. Med. Virol. 70 (Suppl. 1)S103-S110. Human |
| herpes simplex virus | herpesvirus 8 (HHV-8), including subtypes A-E, are disclosed in, e.g., |
| types 1 and 2 (HSV-1, | Treurnicht, et al. (2002) J. Med. Virul. 66: 235-240. |
| HSV-2), and varicella | |
| zoster virus (VZV). | |
| HIV-1 including group M | See, e.g., Smith, et al. (1998) J. Med. Virol. 56: 264-268. See also, e.g., |
| (including subtypes A to | GenBank Acc. Nos. DQ054367; NC_001802; AY968312; DQ011180; |
| J) and group O (including | DQ011179; DQ011178; DQ011177; AY588971; AY588970; AY781127; |
| any distinguishable | AY781126; AY970950; AY970949; AY970948; X61240; AJ006287; |
| subtypes) (HIV-2, | AJ508597; and AJ508596. |
| including subtypes A-E. | |
| Epstein-Barr virus | See, e.g., Peh, et al. (2002) Pathology 34: 446-450. Epstein-Barr virus |
| (EBV), including | strain B95-8 (GenBank Acc. No. V01555). |
| subtypes A and B. | |
| Reovirus, including | See, e.g., Barthold, et al. (1993) Lab. Anim. Sci. 43: 425-430; Roner, et al. |
| serotypes and strains 1, 2, | (1995) Proc. Natl. Acad. Sci. USA 92: 12362-12366; Kedl, et al. (1995) J. |
| and 3, type 1 Lang, type 2 | Virol. 69: 552-559. GenBank Acc. No. K02739 (sigma-3 gene surface |
| Jones, and type 3 | protein). |
| Dearing. | |
| Cytomegalovirus (CMV) | See, e.g., Chern, et al. (1998) J. Infect. Dis. 178: 1149-1153; Vilas Boas, et |
| subtypes include | al. (2003) J. Med. Virol. 71: 404-407; Trincado, et al. (2000) J. Med. Virol. |
| CMV subtypes I-VII. | 61: 481-487. GenBank Acc. No. X17403. |
| Rhinovirus, including all | Human rhinovirus 2 (GenBank Acc. No. X02316); Human rhinovirus B |
| serotypes. | (GenBank Acc. No. NC_001490); Human rhinovirus 89 (GenBank Acc. |
| | No. NC_001617); Human rhinovirus 39 (GenBank Acc. No. AY751783). |
| Adenovirus, including all | — |
| serotypes. | |
| Varicella-zoster virus, | See, e.g., Loparev, et al. (2004) J. Virol. 78: 8349-8358; Carr, et al. (2004) |
| including strains and | J. Med. Virol. 73: 131-136; Takayama and Takayama (2004) J. Clin. Virol. |
| genotypes Oka, Dumas, | 29: 113-119. |
| European, Japanese, and | |
| Mosaic. | |
| Filoviruses, including | See, e.g., Geisbert and Jahrling (1995) Virus Res. 39: 129-150; Hutchinson, |
| Marburg virus and Ebola | et al. (2001) J. Med. Virol. 65: 561-566. Marburg virus (see, e.g., |
| virus, and strains such as | GenBank Acc. No. NC_001608). Ebola virus (see, e.g., |
| Ebola-Sudan (EBO-S), | GenBank Acc. Nos. NC_006432; AY769362; NC_002549; AF272001; |
| Ebola-Zaire (EBO-Z), | AF086833). |
| and Ebola-Reston | |
| (EBO-R). | |
| Arenaviruses, including | Junin virus, segment S (GenBank Acc. No. NC_005081); Junin virus, |
| lymphocytic | segment L (GenBank Acc. No. NC_005080). |
| choriomeningitis (LCM) | |
| virus, Lassa virus, Junin | |
| virus, and Machupo virus. | |
| Rabies virus. | See, e.g., GenBank Acc. Nos. NC_001542; AY956319; AY705373; |
| | AF499686; AB128149; AB085828; AB009663. |
| Arboviruses, including | Dengue virus type 1 (see, e.g., GenBank Acc. Nos. AB195673; |
| West Nile virus, Dengue | AY762084). Dengue virus type 2 (see, e.g., GenBank Acc. Nos. |
| viruses 1 to 4, Colorado | NC_001474; AY702040; AY702039; AY702037). Dengue virus type 3 |
| tick fever virus, Sindbis | (see, e.g., GenBank Acc. Nos. AY923865; AT858043). Dengue virus |
| virus, hantavirus, | type 4 (see, e.g., GenBank Acc. Nos. AY947539; AY947539; AF326573). |
| Togaviraidae, | Sindbis virus (see, e.g., GenBank Acc. Nos. NC_001547; AF429428; |
| Flaviviridae, | J02363; AF103728). |
| Bunyaviridae, | |
| Reoviridae, | |
| Rhabdoviridae, | |
| Orthomyxoviridae, and | |
| Poxviridae. | |
| Poxvirus including | Viriola virus (see, e.g., GenBank Acc. Nos. NC_001611; Y16780; |
| orthopoxvirus (variola | X72086; X69198). |
| virus, monkeypox virus, | |
| vaccinia virus, cowpox | |
| virus), yatapoxvirus | |
| (tanapox virus, Yaba | |
| monkey tumor virus), | |
| parapoxvirus, and | |
| molluscipoxvirus. | |
| Yellow fever. | See, e.g., GenBank Acc. No. NC_002031; AY640589; X03700. |
| Hantaviruses, including | See, e.g., Elgh, et al. (1997) J. Clin. Microbiol. 35: 1122-1130; Sjolander, |
| serotypes Hantaan | et al. (2002) Epidemiol. Infect. 128: 99-103; Zeier, et al. (2005) Virus |
| (HTN), Seoul (SEO), | Genes 30: 157-180. GenBank Acc. No. NC_005222 and NC_005219 |
| Dobrava (DOB), Sin | (Hantavirus). See also, e.g., GenBank Acc. Nos. NC_005218; |
| Nombre (SN), Puumala | NC_005222; NC_005219. |
| (PUU), and Dobrava-like | |
| Saaremaa (SAAV). | |
| Flaviviruses, including | See, e.g., Mukhopadhyay, et al. (2005) Nature Rev. Microbiol. 3: 13-22. |
| Dengue virus, Japanese | GenBank Acc. Nos NC_001474 and AY702040 (Dengue). GenBank Acc. |
| encephalitis virus, West | Nos. NC_001563 and AY603654. |
| Nile virus, and yellow | |
| fever virus. | |
| Measles virus. | See, e.g., GenBank Acc. Nos. AB040874 and AY486084. |
| Human | Human parainfluenza virus 2 (see, e.g., GenBank Acc. Nos. AB176531; |
| parainfluenzaviruses | NC003443). Human parainfluenza virus 3 (see, e.g., GenBank Acc. No. |
| (HPV), including HPV | NC_001796). |
| types 1-56. | |
| Influenza virus, including | Influenza nucleocapsid (see, e.g., GenBank Acc. No. AY626145). |
| influenza virus types A, | Influenza hemagglutinin (see, e.g., GenBank Acc. Nos. AY627885; |
| B, and C. | AY555153). Influenza neuraminidase (see, e.g., GenBank Acc. Nos. |
| | AY555151; AY577316). Influenza matrix protein 2 (see, e.g., GenBank |
| | Acc. Nos. AY626144(. Influenza basic protein 1 (see, e.g., GenBank Acc. |
| | No. AY627897). Influenza polymerase acid protein (see, e.g., GenBank |
| | Acc. No. AY627896). Influenza nucleoprotein (see, e.g., GenBank Acc. |
| | No. AY627895). |
| Influenza A virus | Hemagglutinin of H1N1 (GenBank Acc. No. S67220). Influenza A virus |
| subtypes, e.g., swine | matrix protein (GenBank Acc. No. AY700216). Influenza virus A H5H1 |
| viruses (SIV): H1N1 | nucleoprotein (GenBank Acc. No. AY646426). H1N1 haemagglutinin |
| influenzaA and swine | (GenBank Acc. No. D00837). See also, GenBank Acc. Nos. BD006058; |
| influenza virus. | BD006055; BD006052. See also, e.g., Wentworth, et al. (1994) J. Virol. |
| | 68: 2051-2058; Wells, et al. (1991) J.A.M.A. 265: 478-481. |
| Respiratory syncytial | Respiratory syncytial virus (RSV) (see, e.g., GenBank Acc. Nos. |
| virus (RSV), including | AY353550; NC_001803; NC001781). |
| subgroup A and subgroup | |
| B. | |
| Rotaviruses, including | Human rotavirus C segment 8 (GenBank Acc. No. AJ549087); Human |
| human rotaviruses A to E, | rotavirus G9 strain outer capsid protein (see, e.g., GenBank Acc. No. |
| bovine rotavirus, rhesus | DQ056300); Human rotavirus B strain non-structural protein 4 (see, e.g., |
| monkey rotavirus, and | GenBank Acc. No. AY548957); human rotavirus A strain major inner |
| human-RVV | capsid protein (see, e.g., GenBank Acc. No. AY601554). |
| reassortments. | |
| Polyomavirus, including | See, e.g., Engels, et al. (2004) J. Infect. Dis. 190: 2065-2069; Vilchez and |
| simian virus 40 (SV40), | Butel (2004) Clin. Microbiol. Rev. 17: 495-508; Shivapurkar, et al. (2004) |
| JC virus (JCV) and BK | Cancer Res. 64: 3757-3760; Carbone, et al. (2003) Oncogene 2: 5173-5180; |
| virus (BKV). | Barbanti-Brodano, et al. (2004) Virology 318: 1-9) (SV40 complete |
| | genome in, e.g., GenBank Acc. Nos. NC_001669; AF168994; AY271817; |
| | AY271816; AY120890; AF345344; AF332562). |
| Coltiviruses, including | Attoui, et al. (1998) J. Gen. Virol. 79: 2481-2489. Segments of Eyach |
| Colorado tick fever virus, | virus (see, e.g., GenBank Acc. Nos. AF282475; AF282472; AF282473; |
| Eyach virus. | AF282478; AF282476; NC_003707; NC_003702; |
| | NC_003703; NC_003704; NC_003705; NC_003696; NC_003697; |
| | NC_003698; NC_003699; NC_003701; NC_003706; NC_003700; |
| | AF282471; AF282477). |
| Calciviruses, including | Snow Mountain virus (see, e.g., GenBank Acc. No. AY134748). |
| the genogroups Norwalk, | |
| Snow Mountain group | |
| (SMA), and Saaporo. | |
| Parvoviridae, including | See, e.g., Brown (2004) Dev. Biol. (Basel) 118: 71-77; Alvarez-Lafuente, |
| dependovirus, parvovirus | et al. (2005) Ann. Rheum. Dis. 64: 780-782; Ziyaeyan, et al. (2005) Jpn. J. |
| (including | Infect. Dis. 58: 95-97; Kaufman, et al. (2005) Virology 332: 189-198. |
| parvovirus B19), and | |
| erythrovirus. | |
| The cited references and the nucleic acids, peptides, and polypeptides disclosed therein, are all incorporated herein by reference in their entirety. The list of antigens and their nucleic acids, and the list of methods of administration, are not intended to be limiting to the present invention. The invention encompasses the use of, but is not limited to, nucleic acids encoding mutants, muteins, splice variants, fragments, truncated variants, soluble variants, extracellular domains, | |
| # intracellular domains, mature sequences, and the like, of the disclosed antigens. Provided are nucleic acids encoding epitopes, oligo- and polypeptides of these antigens. Also provided are codon optimized aspects, i.e., optimized for expression in Listeria. | |

In a further aspect, the non-Listerial antigens used in the present invention may be derived from Human Immunodeficiency Virus (HIV), e.g., gp120; gp160; gp41; gag antigens such as p24 gag or p55 gag, as well as protein derived from the pol, env, tat, vir, rev, nef, vpr, vpu, and LTR regions of HIV. The heterologous antigens contemplated include those from herpes simplex virus (HSV) types 1 and 2, from cytomegalovirus, from Epstein-Barr virus, or Varicella Zoster Virus. Also encompassed are antigens derived from a heptatis virus, e.g., hepatitis A, B, C, delta, E, or G. Moreover, the antigens also encompass antigens from Picornaviridae (poliovirus; rhinovirus); Caliciviridae; Togaviridae (rubella; dengue); Flaviviridiae; Coronaviridae; Reoviridae; Birnaviridae; Rhabdoviridae; Orthomyxoviridae; Filoviridae; Paramyxoviridae (mumps; measle); Bunyviridae; Arenaviridae; Retroviradae (HTLV-I; HIV-1); Papillovirus, tick-borne encephalitis viruses, and the like.

In yet another aspect, the present invention provides reagents and methods for the prevention and treatment of bacterial and parasitic infections, e.g., Salmonella, Neisseria, Borrelia, Chlamydia, Bordetella, plasmodium, Toxoplasma, Mycobacterium tuberculosis, Bacillus anthracis, Yersinia pestis, Diphtheria, Pertussis, Tetanus, bacterial or fungal pneumonia, Otitis Media, Gonorrhea, Cholera, Typhoid, Meningitis, Mononucleosis, Plague, Shigellosis, Salmonellosis, Legionaire's Disease, Lyme disease, Leprosy, Malaria, Hookworm, Onchocerciasis, Schistosomiasis, Trypanasomes, Leshmania, Giardia, Amoebiasis, Filariasis, Borelia, and Trichinosis (see, e.g., Despommier, et al. (2000) Parasitic Dieases, 4^{th} ed., Apple Trees Productions, New York, N.Y.; U.S. Governnent (2002) 21st Century Collection Centers for Disease Control (CDC) Emerging Infectious Diseases (EID)—Comprehensive Collection from 1995 to 2002 with Accurate and Detailed Information on Dozens of Serious Virus and Bacteria Illnesses—Hantavirus, Influenza, AIDS, Malaria, TB, Pox, Bioterrorism, Smallpox, Anthrax, Vaccines, Lyme Disease, Rabies, West Nile Virus, Hemorrhagic Fevers, Ebola, Encephalitis (Core Federal Information Series).

Antibodies for use in the present invention for mediating the ADCC are available, including antibodies that bind specifically to, e.g., mesothelin, PSCA, proteinase-3, wt-1, RAS, or other antigens (Table 4). The present invention also provides bispecific antibodies comprising a first binding site (derived from a first antibody) that specifically binds to a tumor antigen and a second binding site (derived from a second antibody) that specifically binds to an Fc receptor, e.g., FcgammaRIII (CD16); FcgammaRI (CD64); or FcalphaRI (CD89) (see, e.g., Peipp and Valerius (2002) Biochem. Soc. Trans. 30:507-511). Moreover, the invention also provides bispecific antibodies comprising a first binding site (derived from a first antibody) that specifically binds to a tumor antigen and a second binding site (derived from a second antibody) that specifically binds to any membrane-associated or membrane-bound antigen of an immune cell, e.g., of an NK cell or monocyte.

TABLE 4
Antibodies useful for the methods of the present invention.

| | |
|---|---|
| Antigen | Reference and/or supplier of antibody |

Antibodies to tumors, tumor antigens, tumor-associated antigens,
self antigens, angiogenesis antigens, and the like.

| | |
|---|---|
| Proteinase-3 | See, e.g., Rooney, et al. (2001) Am. J. Respir. Cell Mol. Biol. |
| | 24: 747-754; CLB, Amsterdam, Holland. |
| Wt-1 | See, e.g., Shigehara, et al. (1998) J. Am. Soc. Nephrol. 14: 1998-2003; |
| | Sasaki, et al. (2004) Kidney International 65: 469; Bowles, et |
| | al. (2001) Transplantation 72: 330-333; Silberstein, et al. (1997) |
| | Proc. Natl. Acad. Sci. USA 94: 8132-8137; Scharnhorst, et al. (1999) |
| | J. Biol. Chem. 274: 23456-23462. |
| Survivin | See, e.g., Stratagene, La Jolla, CA; Jaskoll, et al. (2001) BMC |
| | Developmental Biol. 1:5. |
| CEA | See, e.g., Ryser, et al. (1992) J. Nuclear Med. 33: 1766-1773. |
| RAS | See, e.g., Rubio, et al. (2003) Biochem. J. 376: 571-576; Oncogene |
| | Science (Cambridge, MA); ATCC (Manassas, VA); Calbiochem |
| | (San Diego, CA); Upstate (Waltham, MA); Werge, et al. (1994) |
| | FEBS Lett. 351: 393-396. |
| Mesothelin and CA125 (a.k.a. | See, e.g., Chowdhury, et al. (1998) Proc. Natl. Acad. Sci. USA |
| MUC16), e.g., the monoclonal | 95: 669-674; Hassan, et al. (2000) J. Immunother. 23: 473-479; |
| antibody K1. (Mesothelin and | Chowdhury, et al. (1999) J. Immunol. Methods 231: 83-91; |
| CA125/MUC16 bind to each | Brinkmann, et al. (1997) Int. J. Cancer 71: 638-644; Rump, et al. |
| other.) | (2004) J. Biol. Chem. 279: 9190-9198; Nelson and Ordonez (2003) |
| | Mod. Pathol. 16: 192-197. |
| NY-ESO-1 | See, e.g., Mischo, et al. (2003) Cancer Immunity 3:5. |
| LAGE | Mandic, et al. (2003) Cancer Res. 63: 6506-6515. |
| HOM-MEL-40/SSX2 | See, e.g., Wagner, et al. (2003) Cancer Immunity 3:18; Neumann, et |
| | al. (2004) Int. J. Cancer 112: 661-668. |
| MUM-1. | Hirose, et al. (2005) Int. J. Hematol. 81: 48-57. |
| Melanoma associated antigens, | See, e.g., Rimoldi, et al. (1999) Int. J. Cancer 82: 901-997; Rimoldi, |
| including the various MAGE | et al. (2000) Int. J. Cancer 86: 749-751; Bai, et al. (2005) Mol. Cell |
| antigens (e.g., MAGE-1, 3, 4, | Biol. 25: 1238-1257; Schichijo, et al. (1995) J. Immunol. Methods |
| 10, and 11), tyrosinase, gp-100, | 186: 137-149; Murer, et al. (2004) Melanoma Res. 14: 257-262; |
| and Melan-A/MART-1. | Jungbluth, et al. (2001) Applied Immunohistochem. Molecular |
| | Morphol. 9:1; Jaanson, et al. (2003) Melanoma Res. 13: 473-482; |
| | Heegaard, et al. (2000) Melanoma Res. 10: 350-354. |
| Papillomavirus E7. | See, e.g., Felton-Edkins, et al. (2003) EMBO J. 22: 2422-2432; |
| | Smahel, et al. 6: 1092-1101. |
| BRCA-1; BRCA-2. | See, e.g., Korhonen, et al. (2003) J. Neuroscience Res. 71: 769-776; |
| | Tutt, et al. (2001) EMBO J. 20: 4704-4716; Bethyl Laboratories |
| | (Montgomery, TX); Sigma-Aldrich (St. Louis, MO). |
| PAP. | See, e.g., Seki, et al. (2004) Am. J. Surgical Pathol. 28:10; Lin, et al. |
| | (1983) Cancer Res. 43: 3841-3846. |
| RCAS1. | See, e.g., Enjoji, et al. (2004) Dig Liver Dis. 36: 622-627. |
| RAGE. | See, e.g., Li, et al. (2004) Am. J. Pathol. 164: 1389-1397; Shirasawa, |
| | et al. (2004) Genes to Cells 9: 165-174. |
| SART-1; SART-2; SART-3. | See, e.g., Takedatsu, et al. (2004) J. Immunotherapy 27: 289-297; |
| | Murayama, et al. (2000) J. Immunotherapy 23: 511-518. |
| SP-17. | See, e.g., Lim, et al. (2001) Blood 97: 1508-1510. |
| CAP-1 | See, e.g., Bertling, et al. (2004) J. Biol. Chem. 15: 2324-2334; Fling, |
| | et al. (2001) Proc. Natl. Acad. Sci. USA 98: 1160-1165. |
| TARP. | See, e.g., Clifton, et al. (2004) Proc. Natl. Acad. Sci. USA |
| | 101: 10166-10171; Virok, et al. (2005) Infection Immunity 73: 1939-1946. |
| Tyrosinase-related protein-1 | See, e.g., Okamoto, et al. (1998) J. Invest. Dermatol.111: 1034-1039; |
| and 2 (TRP-1 and TRP-2). | Huang, et al. (1998) J. Invest. Dermatol. 111: 662-667; Negroiu, et |
| | al. (2005) Biochem. Biophys. Res. Commun. 328: 914-921. |
| gp100. | See, e.g., Huang, et al. (1998) J Invest Dermatol. 111: 662-667; |
| | Busam, et al. (2001) Am. J. Surg. Pathol. 25: 197-204; Zymed, Inc. |
| | (South San Francisco, CA). |
| MUC-1; MUC-2. | See, e.g., Davies, et al. (1994) Cancer Lett. 82: 179-184; Gambus, et |
| | al. (1995) Int. J. Cancer 60: 146-148; McCool, et al. (1999) |
| | Biochem. J. 341: 593-600. |
| TERT. | See, e.g., Liu, et al. (2002) Am. J. Respir. Cell Mol. Biol. 26: 534-540; |
| | Hashimoto, et al. (2004) J. Clin. Invest. 113: 243-252; |
| | Minamino, et al. (2001) Mol. Cell. Biol. 21: 3336-3342. |
| G250. | See, e.g., Belumer, et al. (2004) Br. J. Cancer 90: 985-990. |
| Beta-catenin. | See, e.g., Schwartz, et al. (1999) J. Am. Soc. Nephrol. 10: 2297-2305. |
| BCL-2 family of proteins. | See, e.g., Sigma-Aldrich (St. Louis, MO). |
| p53. | See, e.g., Sigma-Aldrich (St. Louis, MO). |
| EGP-2 (a.k.a. GA733-A). | See, e.g., Willuda, et al. (1999) Cancer Res. 59: 5758-5767. |
| c-erbB-2 (a.k.a. p185; HER-2). | See, e.g., Dean, et al. (1998) Clin. Cancer Res. 4: 2545-2550; |
| Prostate Stem Cell Antigen | See, e.g., Wente, et al. (2002) Pancreatology 2: 217-361; Zhigang |
| (PSCA). | and Wenlv (2004) World J. Surgical Oncol. 2:13. |
| Prostate-specific membrane | See, e.g., Pinto, et al. (1996) Clin. Cancer Res. 2: 1445-1451. |
| antigen (PSM). | |
| HER-2 (e.g., Trastuzumab, | See, e.g., Cersosimo (2003) Am. J. Health Syst. Pharm. 60: 1531-1548 |
| Herceptin ®). | and 1631-1641. |
| 1D10 (e.g., Remitogen ®). | See, e.g., Shi, et al. (2002) Leuk. Lymphoma 43: 1303-1312. |
| Epidermal growth factor | Caponigro, et al. (2005) Curr. Opin. Oncol. 17: 212-217. |
| receptor (e.g., Cetuximab; | |
| Erbitux ®). | |
| Vascular endothelial growth | See, e.g., Hurwitz, et al. (2004) New Engl. J. Med. 350: 2335-2342; |
| factor receptor (e.g., | Venook (2005) Oncologist 10: 250-261; Kabbinavar, et al. |
| Bevacizumab, Avastin ®). | (2005) J. Clin. Oncol. May 2 [epub ahead of print]; Wang, et al. |
| | (2004) Angiogenesis 7: 335-345. |
| CD20 (e.g., Tositumomab; | See, e.g., Cersosimo (2003) Am. J. Health Syst. Pharm. 60: 1531-1548; |
| Bexxar ®; Ibritumomab | Vose (2004) Oncologist 9: 160-172 |
| tiuxetan; Zevalin ®; rituximab; | |
| Rituxan ®). | |
| CD22 (e.g., Epratuzumab). | See, e.g., Leonard, et al. (2004) Clin. Cancer Res. 10: 5327-5334. |
| CD25. | See, e.g., Zhang, et al. (2004) Cancer Res. 64: 5825-5829. |
| CD33 (e.g., Gemtuzumab; | See, e.g., Cersosimo (2003) Am. J. Health Syst. Pharm. 60: 1531-1548 |
| Mylotarg ®). | and 1631-1641; Golay, et al. (2005) Br. J. Haematol. 128: 310-317; |
| | Linenberger (2005) Leukemia 19: 176-182 |
| CD52 (e.g., Alemtuzumab; | See, e.g., Cersosimo (2003) Am. J. Health Syst. Pharm. 60: 1531-1548 |
| Campath ®). | and 1631-1641. |
| Anti-alpha-upsilon-beta3 | See, e.g., Gutheil, et al. (2000) Clin. Cancer Res. 6: 3056-3061. |
| antibody (integrin used in | |
| angiogenesis). | |

Antibodies to viruses, bacteria, parasites, and the like, and to antigens thereof.

| | |
|---|---|
| Papillomavirus, e.g., E6, E7. | See, e.g., Kanduc, et al. (2001) Peptides 22: 1981-1985; Lagrange, et |
| | al. (2005) J. Gen. Virol. 86: 1001-1007; Kanduc, et al. (2004) |
| | Peptides 25: 243-250; McLaughlin-Drubin, et al. (2004) Virology |
| | 322: 213-219; Wang, et al. (2003) Virology 311: 213-221. |
| Tularensis. | See, e.g., Porsch-Ozcurumez, et al. (2004) Clin. Diagnostic Lab. |
| | Immunol. 11: 1008-1015; Stenmark, et al. (2003) Microb. Pathog. |
| | 35: 73-80; Fulop, et al. (2001) Vaccine 19: 4465-4472. |
| Hepatitis A, B, C, D, E., e.g., | See, e.g., Keck, et al. (2004) J. Virol. 78: 7257-7263; Zhang, et al. |
| E1 protein, E2 protein, pre-S1 | (2005) Vaccine 23: 2881-2892; Keck, et al. (2004) J. Virol. 78: 9224-9232; |
| protein; capsid protein. | Hong, et al. (2004) Virology 318: 134-141; Kim, et al. (2004) |
| | Virology 318: 598-607; Cao, et al. (2004) Zhonghua Shi Yan He Lin |
| | Chuang Bing Du Xue Za Zhi 18: 20-23; Bose, et al. (2003) Mol. |
| | Immunol. 40: 617-631; Ramirez, et al. (2003) Biotechnol. Appl. |
| | Biochem. 38: 223-230; Burioni, et al. (2002) J. Virol. 76: 11775-11779; |
| | Heijtink, et al. (2001) J. Med. Virol. 64: 427-434. |
| Hepatitis A-neutralizing | See, e.g., Stapleton, et al. (1993) J. Virol. 67: 1080-1085. |
| antibodies, e.g., B5B3; K2-4F2; | |
| K3-4C8; K3-2F2; and 3.2.4. | |
| HIV, e.g., HRG214; gp160; | See, e.g., Pett, et al. (2004) HIV Clin. Trials 5: 91-98; Rollman, et al. |
| gp120; viral infectivity factor | (2004) Gene Ther. 11: 1146-1154; Fessel (2005) Med. Hypotheses |
| (vif). | 64: 261-263; Dezube, et al. (2004) J. Clin. Virol. 31 (Suppl. 1): S45-S47. |
| Adenovirus, e.g., hexon coat | See, e.g., Varghese, et al. (2004) 78: 12320-12332. |
| protein. | |
| Influenza viruses A and B, | See, e.g., Rovida, et al. (2005) J. Med. Virol. 75: 336-347; Varich |
| paraiinfluenza viruses, and | and Kaverin (2004) Arch. Virol. 149: 2271-2276; Liu, et al. (2004) |
| human respiratory syncytial | Immunol. Lett. 93: 131-136. |
| virus, and adenovirus. | |
| SARS-coronavirus. | See, e.g., Gubbins, et al. (2005) Mol. Immunol. 42: 125-136. |
| Hantaan virus, e.g., G1 and G2 | See, e.g., Ogino, et al. (2004) J. Virol. 78: 10776-10782. |
| envelope glycoproteins. | |
| Poliovirus. | See, e.g., Yanagiya, et al. (2005) J. Virol. 79: 1523-1532. |
| Dengue viruses. | See, e.g., Goncalvez, et al. (2004) J. Virol. 78: 12910-12918. |
| Japanese encephalitis virus. | See, e.g., Wu, et al. (2004) Vaccine 23: 163-171. |
| HSV-2, e.g., HSV-2 | See, e.g., Parr, et al. (2005) Arch. Virol., April 14 [epub ahead of |
| glycoprotein D. | print]. |
| West Nile virus, e.g., envelope | See, e.g., Li, et al. (2005) Virology 335: 99-105. |
| protein. | |
| RSV (bronchiolitis virus), e.g., | See, e.g., Domachowske and Rosenburg (2005) Pediatr. Ann. |
| palivizumab antibody. | 34: 35-41. |
| Cytogam ® (Cytomegalovirus). | See, e.g., Forthal, et al. (2001) Transpl. Infect. Dis. 3 (Suppl. 2) |
| | 31-34). |
| Mycobacterium tuberculosis. | See, e.g., Shoenfeld, et al. (1986) Clin. Exp. Immunol. 66: 255-261. |
| Plasmodium falciparum, e.g., | See, e.g., Bouharoun-Tayoun, et al. (2004) Exp. Parasitol. 108: 47-52. |
| MSP-3 protein. | |
| Papillomavirus E7. | See, e.g., Kanduc, et al. (2001) Peptides 22: 1981-1985. |

Anti-Fc receptor antibodies.

| | |
|---|---|
| Fcgamma RI (a.k.a. CD64). | See, e.g., Kepley, et al. (2004) J. Biol. Chem. 279: 35139-35149; |
| | Kudo, et al. (1999) Tohoku J. Exp. Med. 188: 275-288. |
| Fcgamma RII (a.k.a. CD32). | See, e.g., Kepley, et al. (2004) J. Biol. Chem. 279: 35139-35149; |
| | Scott-Zaki, et al. (2000) Cell Immunol. 201: 89-93. |
| FcgammaRIIIa; FcgammaRIIIb | See, e.g., Shahied, et al. (2004) J. Biol. Chem. 279: 53907-53914; |
| (a.k.a. CD16) (activating | Kepley, et al. (2004) J. Biol. Chem. 279: 35139-35149; Durand, et |
| receptor on NK cells; | al. (2001) J. Immunol. 167: 3996-4007; Renner, et al. (2001) Cancer |
| monocytes; neutrophils). | Immunol. Immunother. 50: 102-108; Kudo, et al. (1999) Tohoku J. |
| | Exp. Med. 188: 275-288; Scott-Zaki, et al. (2000) Cell Immunol. |
| | 201: 89-93; Bruenke, et al. (2004) Br. J. Haematol. 125: 167-179. |
| FcalphaRI (IgA Fc receptor) | See, e.g., Peipp and Valerius (2002) Biochem. Soc. Trans. 30: 507-511; |
| (CD89). | Shen (1992) J. Leukoc. Biol. 51: 373-378; Mota, et al. (2003) |
| | Eur. J. Immunol. 33: 2197-2205. |

Antibodies to components of immune cells, e.g., NK cells, monocytes, neutrophils.

| CD16; CD56; CD57; CD69; | See, e.g., Kenna, et al. (2003) J. Immunol. 171: 1775-1779; |
|---|---|
| CD94; CD158a; CD161. | Cameron, et al. (2003) Br. J. Dermatol. 149: 160-164; Cameron, et |
| | al. (2002) Arch. Dermatol. Res. 294: 363-369; Mitsui, et al. (2004) |
| | Br. J. Haematol. 126: 55-62. |
| CD122 (IL-2R subunit). | See, e.g., Harada, et al. (2004) Exp. Hematol. 32: 614-621; |
| | Takayama, et al. (2003) Immunology 108: 211-219. |
| NK1; NK1.1; DX5. | See, e.g., Verneris, et al. (2001) Biol. Blood Marrow Transplant. |
| | 7: 532-542; Gloeckner-Hofmann, et al. (2000) Ann. Hematol. |
| | 79: 635-639. |
| NKp46; NKp30; CD16; | See, e.g., Sivori, et al. (2003) Eur. J. Immunol. 33: 3439-3447. |
| CD94/NKG2A. | |
| KIR2DL1/2DS1; | See, e.g., Pascal, et al. (2004) Eur. J. Immunol. 34: 2930-2940. |
| KIR2DL2/2DL3/2DS2; | |
| KIR3DL1; KIR2DS4; CD94; | |
| CD161; CD162R. | |
| General suppliers of antibodies | Sigma-Aldrich (St. Louis, MO); Acris Antibodies (Hiddenhausen, |
| | Germany); Zymed Laboratories (South San Francisco, CA); and |
| | Calbiochem (San Diego, CA); Santa Cruz Biotechnology (Santa |
| | Cruz, CA). |
| The antigens, antibodies, and binding compositions derived from an antibody, and nucleic acids encoding said antigens, antibodies, and binding compositions, for use in the present invention, are not limited to or by the listed references and suppliers. The listed references also disclose nucleic acids encoding the antigens that are specifically bound by the identified antibodies. The present invention encompasses the use of a bispecific antibody comprising a first binding site | |
| # derived from an anti-NK cell marker antibody and a second binding site derived from an anti-tumor antigen antibody. | |

VI. Treating Infections.

What is available for the invention, in some aspects, are methods and reagents for stimulating immune response to infections, e.g., infections of the liver. These include infections from hepatotropic viruses and viruses that mediate hepatitis, e.g., hepatitis B virus, hepatitis C virus, and cytomegalovirus. The invention contemplates methods to treat other hepatotropic viruses, such as herpes simplex virus, Epstein-Barr virus, and dengue virus. NK cells, for example, have been shown to mediate immune response against these viruses (see, e.g., Ahlenstiel and Rehermann (2005) Hepatology 41:675-677; Chen, et al. (2005) J. Viral Hepat. 12:38-45; Sun and Gao (2004) Gasteroenterol. 127:1525-1539; Li, et al. (2004) J. Leukoc. Biol. 76:1171-1179; Ahmad and Alvarez (2004) J. Leukoc. Biol. 76:743-759; Cook (1997) Eur. J. Gasteroenterol. Hepatol. 9:1239-1247; Williams and Riordan (2000) J. Gasteroenterol. Hepatol. 15 (Suppl.)G17-G25; Varani and Landini (2002) Clin. Lab. 48:39-44; Rubin (1997) Clin. Liver Dis. 1:439-452; Loh, et al. (2005) J. Virol. 79:661-667; Shresta, et al. (2004) Virology 319:262-273; Fjaer, et al. (2005) Pediatr. Transplant 9:68-73; Li, et al. (2004) World J. Gasteroenterol. 10:3409-3413; Collin, et al. (2004) J. Hepatol. 41:174-175; Ohga, et al. (2002) Crit. Rev. Oncol. Hematol. 44:203-215).

In another aspect, the invention provides methods and reagents for treating parasitic infections, e.g., parasitic infections of the liver. These include, without limitation, liver flukes (e.g., Clonorchis, Fasciola hepatica, Opisthorchis), Leishmania, Ascaris lumbricoides, Schistosoma, and helminths. Helminths include, e.g., nematodes (roundworms), cestodes (tapeworms), and trematodes (flatworms or flukes). NK cells, as well as other immune cells, respond to these infections (see, e.g., Tliba, et al. (2002) Vet. Res. 33:327-332; Keiser and Utzinger (2004) Expert Opin. Pharmacother. 5:1711-1726; Kaewkes (2003) Acta Trop. 88:177-186; Srivatanakul, et al. (2004) Asian Pac. J. Cancer Prev. 5:118-125; Stuaffer, et al. (2004) J. Travel Med. 11:157-159; Nylen, et al. (2003) Clin. Exp. Immunol. 131:457-467; Bukte, et al. (2004) Abdom. Imaging 29:82-84; Singh and Sivakumar (2003) 49:55-60; Wyler (1992) Parisitol. Today 8:277-279; Wynn, et al. (2004) Immunol. Rev. 201:156-167; Asseman, et al. (1996) Immunol. Lett. 54:11-20; Becker, et al. (2003) Mol. Biochem. Parasitol. 130:65-74; Pockros and Capozza (2005) Curr. Infect. Dis. Rep. 7:61-70; Hsieh, et al. (2004) J. Immunol. 173:2699-2704; Korten, et al. (2002) J. Immunol. 168:5199-5206; Pockros and Capozza (2004) Curr. Gastroenterol. Rep. 6:287-296).

Yet another aspect of invention provides methods and reagents for treating bacterial infections, e.g., by hepatotropic bacteria. Provided are methods and reagents for treating, e.g., Mycobacterium tuberculosis, Treponemapallidum, and Salmonella spp. NK cells, as well as other cells of the immune system, respond to these bacterial infections (see, e.g., Cook (1997) Eur. J. Gasteroenterol. Hepatol. 9:1239-1247; Vankayalapati, et al. (2004) J. Immunol. 172:130-137; Sellati, et al. (2001) J. Immunol. 166:4131-4140; Jason, et al. (2000) J. Infectious Dis. 182:474-481; Kirby, et al. (2002) J. Immunol. 169:4450-4459; Johansson and Wick (2004) J. Immunol. 172:2496-2503; Hayashi, et al. (2004) Intern. Med. 43:521-523; Akcay, et al. (2004) Int. J. Clin. Pract. 58:625-627; de la Barrera, et al. (2004) Clin. Exp. Immunol. 135:105-113).

VII. Listerial Genes and Proteins, Including Virulence Factors.

L. monocytogenes expresses various genes and gene products that contribute to growth or colonization in the host (Table 5). Some of these genes and gene products are classed as “virulence factors.” The virulence factors facilitate bacterial infection of host cells. These virulence factors include actA, listeriolysin (LLO), protein 60 (p60), internalin A (inlA), internalin B (inIB), phosphatidylcholine phospholipase C (PC-PLC), phosphatidylinositol-specific phospholipase C (PI-PLC; picA gene). A number of other internalins have been characterized, e.g., InlC2, InlD, InlE, and InIF (Dramsi, et al. (1997) Infect. Immunity 65:1615-1625). Mpl, a metalloprotease that processes proPL-PLC to active PL-PLC, is also a virulence factor (Chakraborty, et al. (2000) Int. J. Med. Microbiol. 290:167-174; Williams, et al. (2000) J. Bact. 182:837-841). Nucleic acid sequences encoding these virulence factors, as well as a number of other factors that contribute to growth or to spread, are available (Table 5). Without implying any limitation, what is available for use in the invention, is a Listeria bacterium altered, mutated, or attenuated in one or more of the genes or sequences of Table 5.

Table 5 enables one of ordinary skill in the art to identify corresponding genes or coding sequences in various strains of L monocytogenes, and to prepare an attenuated L. monocytogenes for use in the methods of the invention.

TABLE 5
Sequences of L. monocytogenes nucleic acids and proteins.

| Protein/Gene | Nucleotides | GenBank Acc. No. |
|---|---|---|
| Actin assembly inducing | 209470-211389 (coding | NC_003210 |
| protein precursor (ActA | sequence) | |
| gene) | 209456-211389 (gene) | |
| actA in various | — | AF497169; AF497170; |
| L. monocytogenes subtypes. | | AF497171; AF497172; |
| | | AF497173; AF497174; |
| | | AF497175; AF497176; |
| | | AF497177; AF497178; |
| | | AF497179; AF497180; |
| | | AF497181; AF497182; |
| | | AF497183 (Lasa, et al. |
| | | (1995) Mol. Microbiol. |
| | | 18: 425-436). |
| Listeriolysin O precursor | 205819-207408 | NC_003210 |
| (LLO) (hly gene) | | |
| Internalin A (InlA) | 454534-456936 | NC_003210 |
| Internalin B (InlB) | 457021-458913 | NC_003210 |
| SvpA | — | Bierne, et al. (2004) J. |
| | | Bacteriol. 186: 1972-1982; |
| | | Borezee, et al. (2000) |
| | | Microbiology 147: 2913-2923. |
| p104 (a.k.a. LAP) | | Pandiripally, et al. (1999) J. |
| | | Med. Microbiol. 48: 117-124; |
| | | Jaradat, et al. (2003) |
| | | Med. Microbiol. Immunol. |
| | | 192: 85-91. |
| Phosphatidylinositol- | 204624-205577 | NC_003210 |
| specific phospholipase C | | |
| (PI-PLC) (plcA gene) | | |
| Phosphatidylcholine- | 1-3031 | X59723 |
| specific phospholipase C | | |
| (PC-PLC) (plcB gene) | | |
| Zinc metalloprotease | 207739-209271 | NC_003210 |
| precursor (Mpl) | | |
| p60 (protein 60; invasion | Complement of | NC_003210 (Lenz, et al. |
| associated protein (iap)). | 618932-620380 | (2003) Proc. Natl. |
| | | Acad. Sci. |
| | | USA 100: 12432-12437). |
| Sortase | 966245-966913 | NC_003210 |
| Listeriolysin positive | 203607-203642 | NC_003210 |
| regulatory protein (PrfA | | |
| gene) | | |
| Listeriolysin positive | 1-801 | AY318750 |
| regulatory protein (PrfA | | |
| gene) | | |
| PrfB gene | 2586114-2587097 | NC_003210 |
| FbpA gene | 570 amino acids | Dramsi, et al. (2004) Mol. |
| | | Microbiol. 53: 639-649. |
| Auto gene | — | Cabanes, et al. (2004) Mol. |
| | | Microbiol. 51: 1601-1614. |
| Ami (amidase that mediates | — | Dussurget, et al. (2004) |
| adhesion) | | Annu. Rev. Microbiol. |
| | | 58: 587-610. |
| dlt operon (dltA; dltB; dltC; | 487-2034 (dltA) | GenBank Acc. No: |
| dltD). | | AJ012255 (Abachin, et al. |
| | | (2002) Mol. Microbiol. |
| | | 43: 1-14.) |
| prfA boxes | — | Table 1 of Dussurget, et al. |
| | | (2002) Mol. Microbiol. |
| | | 45: 1095-1106. |
| Htp (sugar-P transporter) | 1-1386 | GenBank Acc. No. |
| | | AJ315765 (see, e.g., |
| | | Milohanic, et al. (2003) |
| | | Mol. Microbiol. 47: 1613-1625). |

The referenced nucleic acid sequences, and corresponding translated amino acid sequences, and the cited amino acid sequences, and the corresponding nucleic acid sequences associated with or cited in that reference, are incorporated by reference herein in their entirety.

Listeriolysin (LLO), encoded by the hly gene, mediates escape of the bacterium from the phagolysosome and into the cytoplasm of the host cell. LLO also mediates effective transfer of the bacterium from one host cell to a neighboring host cell. During spread, LLO mediates escape of the bacterium from a double membrane vesicle into the cytoplasm of the neighboring cell (see, e.g., Glomski, et al. (2003) Infect. Immun. 71:6754-6765; Gedde, et al. (2000) Infect. Immun. 68:999-1003; Glomski, et al. (2002) J. Cell Biol. 156:1029-1038; Dubail, et al. (2001) Microbiol. 147:2679-2688; Dramsi and Cosssart (2002) J. Cell Biol. 156:943-946).

ActA is a protein of Listeria's surface that recruits the host cell's actin. In other words, Act A serves as a scaffold to assemble host cell actin and other proteins of the cytoskeleton, where assembly occurs at the surface of the bacterium. ActA mediates propulsion of the Listeria through the host cell's cytoplasm. ActA mutants are able to escape from the phagocytic vacuole, but grow inside the host cytosol as “microcolonies” and do not spread from cell to cell (see, e.g., Machner, et al. (2001) J. Biol. Chem. 276:40096-40103; Lauer, et al. (2001) Mol. Microbiol. 42:1163-1177; Portnoy, et al. (2002) J. Cell Biol. 158:409-414).

Internalin A is a ligand for the mammalian membrane-bound protein, E-cadherin. Internalin B is a ligand for a small number of mammalian membrane-bound proteins, e.g., Met receptor (also known as HGF-R/Met) and gClq-R, and proteoglycans. L. monocytogenes can express about 24 members of the internalin-related protein family, including, e.g., an internalin encoded by the irpA gene (see, e.g., Bierne and Cossart (2000) J. Cell Sci. 115:3357-3367; Schluter, et al. (1998) Infect. Immun. 66:5930-5938; Dormann, et al. (1997) Infect. Immun. 65:101-109).

Sortase proteins catalyze the processing and maturation of internalin A. Two sortases have been identified in L. monocytogenes, srtA and srtB. The srtA mutant is defective in bacterial internalization, as determined in studies with human enterocytes and hepatocytes. Hence, mature internalin A is needed for uptake by enterocytes and hepatocytes. The srtA mutant can still be taken up by cells that are able to utilize other mechanisms of uptake, such as the internalin, e.g., In1B (see, e.g., Bierne, et al. (2002) Mol. Microbiol. 43:869-881).

Two phospholipases, PI-PLC (encoded by pIcA gene) and PC-PLC (encoded by plcB gene), are also among the virulence factors. PI-PLC mediates lysis of the host phagosome, allowing escape of the bacterium into the cytosol. Bacterial mutants in PC-PLC show reduced virulence and are found to accumulate within the double-membrane vesicles that mediate cell-to-cell transmission (see, e.g., Camilli, et al. (1993) Mol. Microbiol. 8:143-157; Schulter, et al. (1998) Infect. Immun. 66:5930-5938).

Protein p60, encoded by the iap gene, mediates intracellular movement and cell-to-cell spread. Intracellular movement and spread in iap gene mutants are much reduced (Pilgrim, et al. (2003) Infect. Immun. 71:3473-3484).

What is available is a Listeria attenuated in at least one regulatory factor, e.g., a promoter or a transcription factor. ActA expression is regulated by two different promoters, one immediately upstream of actA and the second in front of the mpl gene, upstream of actA (Lauer, et al. (2002) J. Bacteriol. 184:4177-4186). The present invention provides a nucleic acid encoding inactivated, mutated, or deleted in at least one actA promoter. The transcription factor prfA is required for transcription of a number of L. monocytogenes genes, e.g., hly, pIcA, actA, mpl, prfA, and iap. PrfA's regulatory properties are mediated by, e.g., the PrfA-dependent promoter (PinIC) and the PrfA-box. The present invention provides a nucleic acid encoding inactivated, mutated, or deleted in at least one of PrfA, PinIC, PrfA-box, and the like (see, e.g., Lalic-Mullthaler, et al. (2001) Mol. Microbiol. 42:111-120; Shetron-Rama, et al. (2003) Mol. Microbiol. 48:1537-1551; Luo, et al. (2004) Mol. Microbiol. 52:39-52). Together, inlA and inIB are regulated by five promoters (Lingnau, et al. (1995) Infect. Immun. 63:3896-3903). The invention provides a Listeria attenuated in one or more of these promoters.

What is available for the invention is a Listeria bacterium that is attenuated by treatment with a DNA cross-linking agent (e.g., psoralen) and by inactivating at least one gene that mediates DNA repair, e.g., a recombinational repair gene (e.g., recA) or an ultraviolet light damage repair gene (e.g., uvrA, uvrB, uvrAB, uvrC, uvrD, phrA, phrB) (see, e.g., U.S. Pat. Publication No. 2004/0228877 of Dubensky, et al. and U.S. Pat. Publication No. 2004/0197343 of Dubensky, et al.).

VIII. Listeria Strains.

What is available for the invention are a number of Listeria strains for making or engineering an attenuated Listeria of the present invention (Table 6). The Listeria of the present invention is not to be limited by the strains disclosed in this table.

TABLE 6
Strains of Listeria for use in the present invention.

| L. monocytogenes 10403S wild type. | Bishop and Hinrichs (1987) J. Immunol. 139: 2005-2009; |
|---|---|
| | Lauer, et al. (2002) J. Bact. 184: 4177-4186. |
| L. monocytogenes DP-L4056 (phage cured). The | Lauer, et al. (2002) J. Bact. 184: 4177-4186. |
| prophage-cured 10403S strain is designated | |
| DP-L4056. | |
| L. monocytogenes DP-L4027, which is DP-L2161, | Lauer, et al. (2002) J. Bact. 184: 4177-4186; Jones and |
| phage cured, deleted in hly gene. | Portnoy (1994) Infect. Immunity 65: 5608-5613. |
| L. monocytogenes DP-L4029, which is DP-L3078, | Lauer, et al. (2002) J. Bact. 184: 4177-4186; Skoble, |
| phage cured, deleted in actA. | et al. (2000) J. Cell Biol. 150: 527-538. |
| L. monocytogenes DP-L4042 (delta PEST) | Brockstedt, et al. (2004) Proc. Natl. Acad. Sci. USA |
| | 101: 13832-13837; supporting information. |
| L. monocytogenes DP-L4097 (LLO-S44A). | Brockstedt, et al. (2004) Proc. Natl. Acad. Sci. USA |
| | 101: 13832-13837; supporting information. |
| L. monocytogenes DP-L4364 (delta lplA; lipoate | Brockstedt, et al. (2004) Proc. Natl. Acad. Sci. USA |
| protein ligase). | 101: 13832-13837; supporting information. |
| L. monocytogenes DP-L4405 (delta inlA). | Brockstedt, et al. (2004) Proc. Natl. Acad. Sci. USA |
| | 101: 13832-13837; supporting information. |
| L. monocytogenes DP-L4406 (delta inlB). | Brockstedt, et al. (2004) Proc. Natl. Acad. Sci. USA |
| | 101: 13832-13837; supporting information. |
| L. monocytogenes CS-L0001 (delta actA-delta inlB). | Brockstedt, et al. (2004) Proc. Natl. Acad. Sci. USA |
| | 101: 13832-13837; supporting information. |
| L. monocytogenes CS-L0002 (delta actA-delta lplA). | Brockstedt, et al. (2004) Proc. Natl. Acad. Sci. USA |
| | 101: 13832-13837; supporting information. |
| L. monocytogenes CS-L0003 (L461T-delta lplA). | Brockstedt, et al. (2004) Proc. Natl. Acad. Sci. USA |
| | 101: 13832-13837; supporting information. |
| L. monocytogenes DP-L4038 (delta actA-LLO | Brockstedt, et al. (2004) Proc. Natl. Acad. Sci. USA |
| L461T). | 101: 13832-13837; supporting information. |
| L. monocytogenes DP-L4384 (S44A-LLO L461T). | Brockstedt, et al. (2004) Proc. Natl. Acad. Sci. USA |
| | 101: 13832-13837; supporting information. |
| L. monocytogenes. Mutation in lipoate protein | O'Riordan, et al. (2003) Science 302: 462-464. |
| ligase (LplA1). | |
| L. monocytogenes DP-L4017 (10403S hly (L461T) | U.S. Provisional Pat. Appl. Ser. No. 60/490,089 filed |
| point mutation in hemolysin gene. | Jul. 24, 2003. |
| L. monocytogenes EGD. | GenBank Acc. No. AL591824. |
| L. monocytogenes EGD-e. | GenBank Acc. No. NC_003210. ATCC Acc. No. |
| | BAA-679. |
| L. monocytogenes strain EGD, complete genome, | GenBank Acc. No. AL591975 |
| segment 3/12 | |
| L. monocytogenes. | ATCC Nos. 13932; 15313; 19111-19120; 43248-43251; |
| | 51772-51782. |
| L. monocytogenes DP-L4029 deleted in uvrAB. | U.S. Provisional Pat. Appl. Ser. No. 60/541,515 filed |
| | Feb. 2, 2004; U.S. Provisional Pat. Appl. Ser. No. |
| | 60/490,080 filed Jul. 24, 2003. |
| L. monocytogenes DP-L4029 deleted in uvrAB | U.S. Provisional Pat. Appl. Ser. No. 60/541,515 filed |
| treated with a psoralen. | Feb. 2, 2004. |
| L. monocytogenes actA-/inlB-double mutant. | Deposited with ATCC on Oct. 3, 2003. Acc. No. |
| | PTA-5562. |
| L. monocytogenes lplA mutant or hly mutant. | U.S. Pat. Applic. No. 20040013690 of Portnoy, et al. |
| L. monocytogenes DAL/DAT double mutant. | U.S. Pat. Applic. No. 20050048081 of Frankel and |
| | Portnoy. |
| L. monocytogenes str. 4b F2365. | GenBank Acc. No. NC_002973. |
| Listeria ivanovii | ATCC No. 49954 |
| Listeria innocua Clip11262. | GenBank Acc. No. NC_003212; AL592022. |
| Listeria innocua, a naturally occurring hemolytic | Johnson, et al. (2004) Appl. Environ. Microbiol. |
| strain containing the PrfA-regulated virulence gene | 70: 4256-4266. |
| cluster. | |
| Listeria seeligeri. | Howard, et al. (1992) Appl. Eviron. Microbiol. |
| | 58: 709-712. |
| Listeria innocua with L. monocytogenes | Johnson, et al. (2004) Appl. Environ. Microbiol. |
| pathogenicity island genes. | 70: 4256-4266. |
| Listeria innocua with L. monocytogenes internalin A | See, e.g., Lingnau, et al. (1995) Infection Immunity |
| gene, e.g., as a plasmid or as a genomic nucleic acid. | 63: 3896-3903; Gaillard, et al. (1991) Cell 65: 1127-1141). |
| The present invention encompasses reagents and methods that comprise the above listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); daaA (dat; D-amino acid aminotransferase); plcA; plcB; actA; or any nucleic acid that mediates growth, spread, breakdown of a single | |
| # walled vesicle, breakdown of a double walled vesicle, binding to a host cell, uptake by a host cell. The present invention is not to be limited by the particular strains disclosed above. | |

IX. Combinations of an Administered Listeria and Administered Antibody.

The Listeria and the antibody, or binding composition derived from an antibody, can be administered concomitantly, that is, where the administering for each of these reagents can occur at time intervals that partially or fully overlap each other. The Listeria and the antibody, or binding composition derived from an antibody, can be administered during time intervals that do not overlap each other. For example, the first reagent (Listeria or antibody) can be administered within the time frame of t=0 to 1 hours, while the second reagent (antibody or Listeria) can be administered within the time frame of t=1 to 2 hours. Also, the first reagent can be administered within the time frame of t=0 to 1 hours, while the second reagent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the second reagent can be administered somewhere within the time frame of, e.g., t=minus 2-3 hours, t=minus 3-4 hours, t=minus 4-5 hours, t=minus 5-6 hours, t=minus 6-7 hours, t=minus 7-8 hours, t=minus 8-9 hours, t=minus 9-10 hours, and the like.

To provide another example, the first reagent (Listeria or antibody) can be administered within the time frame of t=0 to 1 days, while the second reagent (antibody or Listeria) can be administered within the time frame of t=1 to 2 days. Also, the first reagent can be administered within the time frame of t=0 to 1 days, while the second reagent can be administered somewhere within the time frame of t=2-3 days, t=3-4 days, t=4-5 days, t=5-6 days, t=6-7 days, t=7-8 days, t=8-9 days, t=9-10 days, and the like. Moreover, the second reagent can be administered, e.g., somewhere in the time frame of t=minus 2-3 days, t=minus 3-4 days, t=minus 4-5 days, t=minus 5-6 days, t=minus 6-7 days, t=minus 7-8 days, t=minus 8-9 days, t=minus 9-10 days, and the like.

In another aspect, administration of the Listeria can begin at t=0 hours, where the administration results in a peak (or maximal plateau) in plasma concentration of the Listeria, and where administration of the antibody is initiated at about the time that the concentration of plasma Listeria reaches said peak concentration, at about the time that the concentration of plasma Listeria is 95% said peak concentration, at about the time that the concentration of plasma Listeria is 90% said peak concentration, at about the time that the concentration of plasma Listeria is 85% said peak concentration, at about the time that the concentration of plasma Listeria is 80% said peak concentration, at about the time that the concentration of plasma Listeria is 75% said peak concentration, at about the time that the concentration of plasma Listeria is 70% said peak concentration, at about the time that the concentration of plasma Listeria is 65% said peak concentration, at about the time that the concentration of plasma Listeria is 60% said peak concentration, at about the time that the concentration of plasma Listeria is 55% said peak concentration, at about the time that the concentration of plasma Listeria is 50% said peak concentration, at about the time that the. concentration of plasma Listeria is 45% said peak concentration, at about the time that the concentration of plasma Listeria is 40% said peak concentration, at about the time that the concentration of plasma Listeria is 35% said peak concentration, at about the time that the concentration of plasma Listeria is 30% said peak concentration, at about the time that the concentration of plasma Listeria is 25% said peak concentration, at about the time that the concentration of plasma Listeria is 20% said peak concentration, at about the time that the concentration of plasma Listeria is 15% said peak concentration, at about the time that the concentration of plasma Listeria is 10% said peak concentration, at about the time that the concentration of plasma Listeria is 5% said peak concentration, at about the time that the concentration of plasma Listeria is 2.0% said peak concentration, at about the time that the concentration of plasma Listeria is 0.5% said peak concentration, at about the time that the concentration of plasma Listeria is 0.2% said peak concentration, or at about the time that the concentration of plasma Listeria is 0.1%, or less than, said peak concentration.

In another aspect, administration of the antibody, or binding compound, can begin at t=0 hours, where the administration results in a peak (or maximal plateau) in plasma concentration of the antibody, or binding compound, and where administration of the Listeria is initiated at about the time that the concentration of plasma antibody reaches said peak concentration, at about the time that the concentration of plasma antibody is 95% said peak concentration, at about the time that the concentration of plasma antibody is 90% said peak concentration, at about the time that the concentration of plasma antibody is 85% said peak concentration, at about the time that the concentration of plasma antibody is 80% said peak concentration, at about the time that the concentration of plasma antibody is 75% said peak concentration, at about the time that the concentration of plasma antibody is 70% said peak concentration, at about the time that the concentration of plasma antibody is 65% said peak concentration, at about the time that the concentration of plasma antibody is 60% said peak concentration, at about the time that the concentration of plasma antibody is 55% said peak concentration, at about the time that the concentration of plasma antibody is 50% said peak concentration, at about the time that the concentration of plasma antibody is 45% said peak concentration, at about the time that the concentration of plasma antibody is 40% said peak concentration, at about the time that the concentration of plasma antibody is 35% said peak concentration, at about the time that the concentration of plasma antibody is 30% said peak concentration, at about the time that the concentration of plasma antibody is 25% said peak concentration, at about the time that the concentration of plasma antibody is 20% said peak concentration, at about the time that the concentration of plasma antibody is 15% said peak concentration, at about the time that the concentration of plasma antibody is 10% said peak concentration, at about the time that the concentration of plasma antibody is 5% said peak concentration, at about the time that the concentration of plasma antibody is 2.0% said peak concentration, at about the time that the concentration of plasma antibody is 0.5% said peak concentration, at about the time that the concentration of plasma antibody is 0.2% said peak concentration, or at about the time that the concentration of plasma antibody is 0. 1%, or less than, said peak concentration. As it is recognized that alteration of the Listeria or antibody may occur in vivo, the above concentrations can be assessed after measurement of intact reagent, or after measurement of an identifiable degradation product of the intact reagent.

The above-disclosed administration schedules apply to the administered antibody relative to the administered Listeria , and to an administered additional reagent (e.g., cytokine, attenuated tumor cell, attenuated tumor cell expressing a cytokine, or small molecule) relative to the Listeria.

The skilled artisan will recognize that biological compartments other than plasma, e.g., whole blood, serum, urine, bile, liver biopsies, can be used for the timing of reagent administration.

The Listeria can be administered in multiple doses, e.g., one dose, two doses, three doses, four doses, five doses, six doses, seven doses, eight doses, nine doses, ten doses, and so on. The antibody, or binding composition, can also be administeredin multiple doses, e.g., one dose, two doses, three doses, four doses, five doses, six doses, seven doses, eight doses, nine doses, ten doses, and so on. Where multiple doses are used, the Listeria can be administered in multiple doses, while only one dose of antibody is used. Also, the Listeria can be administered as one dose, while multiple doses of antibody are used.

X. Reagents Administered with an Administered Listeria.

What is available for use in the present invention are reagents for administering in conjunction with a Listeria, e.g., an attenuated Listeria. These reagents include biological reagents such as cytokines, dendritic cells, antibody/epitope complexes, vaccines, as well as small molecule reagents such as 5-fluorouracil and, in addition, reagents that modulate regulatory T cells, such as cyclophosphamide or anti-CTLA4 antibody. The reagents can be administered with the Listeria or independently (before or after) the Listeria. For example, the reagent can be administered immediately before (or after) the Listeria, on the same day as, one day before (or after), one week before (or after), one month before (or after), or two months before (or after) the Listeria, and the like.

i. Biological reagents. Biological reagents or macromolecules of the present invention encompass an agonist or antagonist of a cytokine, a nucleic acid encoding an agonist or antagonist of a cytokine, a cell expressing a cytokine, or an agonistic or antagonistic antibody. Biological reagents include, without limitation, a TH-1 cytokine, a TH-2 cytokine, IL-2, IL-12, FLT3-ligand, GM-CSF, IFNgamma, a cytokine receptor, a soluble cytokine receptor, a chemokine, tumor necrosis factor (TNF), CD40 ligand, or a reagent that stimulates replacement of a proteasome subunit with an immunoproteasome subunit.

What is available for use with the invention, is a biological reagent, such as GM-CSF, IL-2, IL-3, IL-4, IL-12, IL-18, tumor necrosis factor-alpha (TNF-alpha), or inducing protein-10, or a cell engineered to express the biological reagent. Other contemplated reagents include agonists of B7-1, B7-2, CD28, CD40 ligand, or OX40 ligand (OX40L), and novel forms engineered to be soluble or engineered to be membrane-bound (see, e.g., Kambach, et al. (2001) J. Immunol. 167:2569-2576; Greenfield, et al. (1998) Crit. Rev. Immunol. 18:389-418; Pamey and Chang (2003) J. Biomed. Sci. 10:37-43; Gri, et al. (2003) J. Immunol. 170:99-106; Chiodoni, et al. (1999) J. Exp. Med. 190:125-133; Enzler, et al. (2003) J. Exp. Med. 197:1213-1219; Soo Hoo, et al. (1999) J. Immunol 162:7343-7349; Mihalyo, et al. (2004) J. Immunol. 172:5338-5345; Chapoval, et al. (1998) J. Immunol. 161:6977-6984).

Without implying any limitation, the present invention provides the following biologicals. MCP-1, MIP 1-alpha, TNF-alpha, and interleukin-2, for example, are effective in treating a number of tumor types (see, e.g., Nakamoto, et al. (2000) Anticancer Res. 20(6A):4087-4096; Kamada, et al. (2000) Cancer Res. 60:6416-6420; Li, et al. (2002) Cancer Res. 62:4023-4028; Yang, et al. (2002) Zhonghua Wai Ke Za Zhi 40:789-791; Hoving, et al. (2005) Cancer Res. 65:4300-4308; Tsuchiyama, et al. (2003) Cancer Gene Ther. 10:260-269; Sakai, et al. (2001) Cancer Gene Ther. 8:695-704).

The present invention provides reagents and methods encompassing a Flt3-ligand agonist, and an Flt3-ligand agonist in combination with Listeria. Flt3-ligand (Fms-like tyrosine kinase 3 ligand) is a cytokine that can generate an antitumor immune response (see, e.g., Dranoff (2002) Immunol. Revs. 188:147-154; Mach, et al. (2000) Cancer Res. 60:3239-3246; Furumoto, et al. (2004) J. Clin. Invest. 113:774-783; Freedman, et al. (2003) Clin. Cancer Res. 9:5228-5237; Mach, et al. (2000) Cancer Res. 60:3239-3246).

In another aspect, the present invention contemplates administration of a dendritic cell (DC) that expresses at least one tumor antigen, or infectious agent antigen. Expression by the DC of an antigen can be mediated by way of, e.g., peptide loading, tumor cell extracts, fusion with tumor cells, transduction with mRNA, or transfection by a vector. Relevant methods are described (see, e.g., Klein, et al. (2000) J. Exp. Med. 191:1699-1708; Conrad and Nestle (2003) Curr. Opin. Mol. Ther. 5:405-412; Gilboa and Vieweg (2004) Immunol. Rev. 199:251-263; Paczesny, et al. (2003) Semin. Cancer Biol. 13:439-447; Westermann, et al. (1998) Gene Ther. 5:264-271).

ii. Small molecule reagents. The methods and reagents of the present invention also encompass small molecule reagents, such as 5-fluorouracil, methotrexate, irinotecan, doxorubicin, prednisone, dolostatin-10 (D10), combretastatin A-4, mitomycin C (MMC), vincristine, colchicines, vinblastine, fungal beta-glucans, cyclophosphamide, and the like (see, e.g., Hurwitz, et al. (2004) New Engl. J. Med. 350:2335-2342; Pelaez, et al. (2001) J. Immunol. 166:6608-6615; Havas, et al. (1990) J. Biol. Response Modifiers 9:194-204; Turk, et al. (2004) J. Exp. Med. 200:771-782; Ghiringhelli, et al. (2004) Eur. J. Immunol. 34:336-344; Andrade-Mena (1994) Int. J. Tissue React. 16:95-103; Chrischilles, et al. (2003) Cancer Control 10:396-403). Also encompassed are compositions that are not molecules, e.g., salts and ions.

Provided are analogues of cyclophosphamide (see, e.g., Jain, et al. (2004) J. Med. Chem. 47:3843-3852; Andersson, et al. (1994) Cancer Res. 54:5394-5400; Borch and Canute (1991) J. Med. Chem. 34:3044-3052; Ludeman, et al. (1979) J. Med. Chem. 22:151-158; Zon (1982) Prog. Med. Chem. 19:205-246).

Also embraced by the invention are small molecule reagents that stimulate innate immune response, e.g., CpG oligonucleotides, imiquimod, and alphaGalCer. CpG oligonucleotides mediate immune response via TLR9 (see, e.g., Chagnon, et al. (2005) Clin. Cancer Res. 11:1302-1311; Speiser, et al. (2005) J. Clin. Invest. February 3 (epub ahead of print); Mason, et al. (2005) Clin. Cancer Res. 11:361-369; Suzuki, et al. (2004) Cancer Res. 64:8754-8760; Taniguchi, et al. (2003) Arnu. Rev. Immunol. 21:483-513; Takeda, et al. (2003) Annu. Rev. Immunol. 21:335-376; Metelitsa, et al. (2001) J. Immunol. 167:3114-3122).

Other useful small molecule reagents include those derived from bacterial peptidoglycan, such as certain NOD 1 ligands and/or NOD2 ligands, such as diaminopimelate-containing muropeptides (see, e.g., McCaffrey, et al. (2004) Proc. Natl. Acad. Sci. USA 101:11386-11391; Royet and Reighhart (2003) Trends Cell Biol. 13:610-614; Chamaillard, et al. (2003) Nature Immunol. 4:702-707; Inohara and Nunez (2003) Nature Rev. Immunol. 3:371-382; Inohara, et al. (2004) Annu. Rev. Biochem. November 19 [epub ahead of print]).

iii. Regulatory T cells. The invention includes reagents and methods for modulating activity of T regulatory cells (Tregs; suppressor T cells). Attenuation or inhibition of Treg cell activity can enhance the immune system's killing of tumor cells. A number of reagents have been identified that inhibit Treg cell activity. These reagents include, e.g., cyclophosphamide (a.k.a. Cytoxan®; CTX), anti-CD25 antibody, modulators of GITR-L or GITR, a modulator of Forkhead-box transcription factor (Fox), a modulator of LAG-3, anti-IL-2R, and anti-CTLA4 (see, e.g., Pardoll (2003) Annu. Rev. Immunol. 21:807-839; Ercolini, et al. (2005) J. Exp. Med. 201:1591-1602; Haeryfar, et al. (2005) J. Immunol. 174:3344-3351; Ercolini, et al. (2005) J. Exp. Med. 201:1591-1602; Mihalyo, et al. (2004) J. Immunol. 172:5338-5345; Stephens, et al. (2004) J. Immunol. 173:5008-5020; Schiavoni, et al. (2000) Blood 95:2024-2030; Calmels, et al. (2004) Cancer Gene Ther. October 08 (epub ahead of print); Mincheff, et al. (2004) Cancer Gene Ther. Sept.17 [epub ahead of print]; Muriglan, et al. (2004) J. Exp. Med. 200:149-157; Stephens, et al. (2004) J. Immunol. 173:5008-5020; Coffer and Burgering (2004) Nat. Rev. Immunol. 4:889-899; Kalinichenko, et al. (2004) Genes Dev. 18:830-850; Cobbold, et al. (2004) J. Immunol. 172:6003-6010; Huang, et al. (2004) Immunity 21:503-513). CTX shows a bimodal effect on the immune system, where low doses of CTX inhibit Tregs (see, e.g., Lutsiak, et al. (2005) Blood 105:2862-2868).

CTLA4-blocking agents, such as anti-CTLA4 blocking antibodies, can enhance immune response to proliferative disorders, such as cancer and infections (see, e.g., Zubairi, et al. (2004) Eur. J. Immunol. 34:1433-1440; Espenschied, et al. (2003) J. Immunol. 170:3401-3407; Davila, et al. (2003) Cancer Res. 63:3281-3288; Hodi, et al. (2003) Proc. Natl. Acad. Sci. USA 100:4712-4717). Where the present invention uses anti-CTLA4 antibodies, and the like, the invention is not necessarily limited to use for inhibiting Tregs, and also does not necessarily always encompass inhibition of Tregs.

Lymphocyte activation gene-3 (LAG-3) blocking agents, such as anti-LAG-3 antibodies or soluble LAG-3 (e.g., LAG-3 Ig), can enhance immune response to proliferative disorders. Anti-LAG-3 antibodies reduce the activity of Tregs (see, e.g., Huang, et al. (2004) Immunity 21:503-513; Triebel (2003) Trends Immunol. 24:619-622; Workman and Vignali (2003) Eur. J. Immunol. 33:970-979; Cappello, et al. (2003) Cancer Res. 63:2518-2525; Workman, et al. (2004) J. Immunol. 172:5450-5455; Macon-Lemaitre and Triebel (2005) Immunology 115:170-178).

iv. Vaccines. The use of vaccines comprising a tumor antigen, a nucleic acid encoding a tumor antigen, a vector comprising a nucleic acid encoding a tumor antigen, a cell comprising a tumor antigen, a tumor cell, or an attenuated tumor cell, are encompassed by the invention. Provided are reagents derived from a nucleic acid encoding a tumor antigen, e.g., a codon optimized nucleic acid, or a nucleic acid encoding two or more different tumor antigens, or a nucleic acid expressing rearranged epitopes of a tumor antigen, e.g., where the natural order of epitopes is ABCD and the engineered order is ADBC, or a nucleic acid encoding a fusion protein comprising at least two different tumor antigens. The invention encompasses, but is not limited to, the use of nucleic acids encoding mutants, muteins, splice variants, fragments, truncated variants, soluble variants, extracellular domains, intracellular domains, mature sequences, and the like, of the disclosed antigens. Provided are nucleic acids encoding epitopes, oligo- and polypeptides of these antigens. Also provided are codon optimized aspects, i.e., optimized for expression in Listeria.

Vaccines comprising a tumor cell, an attenuated tumor cell, or a recombinant tumor cell engineered to express a cytokine or other immune modulating agent, are provided for use in the present invention. For example, a tumor cell can be engineered to express an agent that modulates immune response, e.g., GM-CSF, IL-2, IL-4, or IFNgamma (see, e.g., U.S. Pat. Nos. 6,033,674 and 6,350,445 issued to Jaffee, et al.; Golumbek, et al. (1991) Science 254:713-716; Ewend, et al. (2000) J. Immunother. 23:438-448; Zhou, et al. (2005) Cancer Res. 65:1079-1088; Porgador, et al. (1993) J. Immunol. 150:1458-1470; Poloso, et al. (2001) Front. Biosci. 6:D760-D775). The vaccine can be administered by a gel matrix (see, e.g., Salem, et al. (2004) J. Immunol. 172:5159-5167).

The present invention may also use a vaccine comprising a dendritic cell (or other APC) engineered to express a tumor antigen (see, e.g., Avigan (1999) Blood Rev. 13:51-64; Kirk and Mule (2000) Hum. Gene Ther. 11:797-806). Also provided for use are, e.g., synthetic peptides, purified antigens, oligosaccharides, and tumor cell lysates, as a source of tumor antigen (see, e.g., Lewis, et al. (2003) Int. Rev. Immunol. 22:81-112; Razzaque, et al. (2000) Vaccine 19:644-647; Meng and Butterfield (2002) Pharm. Res. 19:926-932; Le Poole, et al. (2002) Curr. Opin. Oncol. 14:641-648). Moreover, the present invention may use a heat shock protein, where the heat shock protein elicits tumor-specific immunity (see, e.g., Udono, et al. (1994) Proc. Natl. Acad. Sci. USA 91:3077-3081; Wang, et al. (2000) Immunol. Invest. 29:131-137).

The Listeria used in the invention can be, but are not necessarily, engineered to contain a nucleic acid encoding at least one heterologous antigen, for example, at least one tumor antigen. The Listeria can be modified by non-recombinant or recombinant methods, e.g., by a plasmid, a recombinant plasmid, by chemical mutagenesis of the genome, or by recombinant modification of the genome. The Listeria can be modified, without limitation, by a plasmid comprising a nucleic acid encoding at least one antigen, by a transposon comprising a nucleic acid encoding at least one antigen, by site-directed integration with a nucleic acid encoding at least one antigen, or by homologous recombination with a nucleic acid encoding at least one antigen (see, e.g., Camilli, et al. (1993) Mol. Microbiol. 8:143-157; Camilli (1992) Genetic analysis of Listeria monocytogenes Determinants of Pathogenesis, Univ. of Pennsylvania, Doctoral thesis; Thompson, et al. (1998) Infect. Immunity 66:3552-3561; Skoble, et al. (2000) J. Cell Biol. 150:527-537; Smith and Youngman (1992) Biochimie 74:705-711; Lei, et al. (2001) J. Bact. 183:1133-1139; Li and Kathariou (2003) Appl. Environ. Microbiol. 69:3020-3023; Lauer, et al. (2002) J. Bacteriol. 184:4177-4186).

Alternatively, or in addition, the vaccine can be administered as a nucleic acid vaccine, liposome, soluble antigen, particulate antigen, colloidal antigen, conjugated antigen, an engineered tumor cell, or an attenuated tumor cell. The vaccine can take the form of a nucleic acid vaccine, liposome, soluble antigen, particulate antigen, colloidal antigen, conjugated antigen, an engineered tumor cell, or an attenuated tumor cell. The list of methods of administration, are not intended to be limiting to the present invention.

XI. Therapeutic Compositions.

The Listeria and an antibody, or binding compound derived from the binding site of an antibody, as well as vaccines, small molecules, biological reagents, and adjuvants that are provided herein can be administered to a host, either alone or in combination with a pharmaceutically acceptable excipient, in an amount sufficient to induce an appropriate immune response to an immune disorder, a proliferative disorder, a cancer, or an infectious disorder. The immune response can comprise, without limitation, specific response, non-specific response, innate response, adaptive immunity, primary immune response, secondary immune response, memory immune response, immune cell activation, immune cell proliferation, immune cell differentiation, and any combination thereof.

A “pharmaceutically acceptable excipient” or “diagnostically acceptable excipient” is meant to include, but is not limited to, sterile distilled water, saline, phosphate buffered solutions, amino acid-based buffers, or bicarbonate buffered solutions. An excipient selected and the amount of excipient used will depend upon the mode of administration. Administration may be oral, intravenous, subcutaneous, dermal, intradermal, intramuscular, parenteral, intraorgan, intralesional, intranasal, inhalation, intraocular, intramuscular, intravascular, intrarectal, intraperitoneal, or any one of a variety of well-known routes of administration. The administration can comprise an injection, infusion, or a combination thereof.

The Listeria of the present invention can be stored, e.g., frozen, lyophilized, as a suspension, as a cell paste, or complexed with a solid matrix or gel matrix.

An effective amount for a particular patient may vary depending on factors such as the condition being treated, the overall health of the patient, the method route and dose of administration and the severity of side affects. An effective amount for a particular patient may vary depending on factors such as the condition being treated, the overall health of the patient, the method route and dose of administration and the severity of side affects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The Listeria used in the invention, in some aspects, can be administered in a dose, or dosages, where each dose comprises at least 1000 Listeria cells/kg body weight; normally at least 10,000 cells; more normally at least 100,000 cells; most normally at least 1 million cells; often at least 10 million cells; more often at least 100 million cells; most often at least 1 billion cells; usually at least 10 billion cells; Listeria cells/kg body weight, or greater. The present invention provides the above doses where the units of Listeria administration is colony forming units (CFU), the equivalent of CFU prior to psoralen-treatment, or where the units are number of Listeria cells.

The Listeria used in the present invention, in other aspects, can be administered in a dose, or dosages, where each dose comprises between 10⁷ and 10⁸ Listeria per 70 kg body weight (or per 1.7 square meters surface area; or per 1.5 kg liver weight); 2×10⁷ and 2×10⁸ Listeria per 70 kg body weight (or per 1.7 square meters surface area; or per 1.5 kg liver weight); 5×10⁷ and 5×10⁸ Listeria per 70 kg body weight (or per 1.7 square meters surface area; or per 1.5 kg liver weight); 10⁸ and 10⁹ Listeria per 70 kg body weight (or per 1.7 square meters surface area; or per 1.5 kg liver weight); between 2.0×10⁸ and 2.0×10⁹ Listeria per 70 kg (or per 1.7 square meters surface area, or per 1.5 kg liver weight); between 5.0×10⁸ to 5.0×10⁹ Listeria per 70 kg (or per 1.7 square meters surface area, or per 1.5 kg liver weight); between 10⁹ and 10¹⁰ Listeria per 70 kg (or per 1.7 square meters surface area, or per 1.5 kg liver weight); between 2×10⁹ and 2×10¹⁰ Listeria per 70 kg (or per 1.7 square meters surface area, or per 1.5 kg liver weight); between 5×10⁹ and 5×10¹⁰ Listeria per 70 kg (or per 1.7 square meters surface area, or per 1.5 kg liver weight); between 10¹¹ and 10¹² Listeria per 70 kg (or per 1.7 square meters surface area, or per 1.5 kg liver weight); between 2×10¹¹ and 2×10¹² Listeria per 70 kg (or per 1.7 square meters surface area, or per 1.5 kg liver weight); between 5×10¹¹ and 5×10¹² Listeria per 70 kg (or per 1.7 square meters surface area, or per 1.5 kg liver weight); between 10¹² and 10¹³ Listeria per 70 kg (or per 1.7 square meters surface area); between 2×10¹² and 2×10¹³ Listeria per 70 kg (or per 1.7 square meters surface area, or per 1.5 kg liver weight); between 5×10¹² and 5×10¹³ Listeria per 70 kg (or per 1.7 square meters surface area, or per 1.5 kg liver weight); or greater.

The number of Listeria can be determined by, e.g., counting individual bacteria under a microscope or by counting colony forming units (CFUs). The mouse liver, at the time of administering the Listeria of the present invention, weighs about 1.5 grams. Human liver weighs about 1.5 kilograms.

Also provided is the use of one or more of the above doses, where the dose is administered by way of one injection every day, one injection every two days, one injection every three days, one injection every four days, one injection every five days, one injection every six days, or one injection every seven days, where the injection schedule is maintained for, e.g., one day only, two days, three days, four days, five days, six days, seven days, two weeks, three weeks, four weeks, five weeks, or longer. The invention also embraces combinations of the above doses and schedules, e.g., a relatively large initial dose of Listeria, followed by relatively small subsequent doses of Listeria.

Antibodies, monoclonal antibodies, binding compounds, or binding compositions derived from the antigen binding site of an antibody, and/or from the Fc receptor binding site of an antibody, cytokines, and mediators of immune response, are administered. The present invention provides, without limitation, doses, e.g., 0.001-0.005 mg/kg body weight; 0.005-0.01 mg/kg; 0.01-0.5 mg/kg; 0.5-1.0 mg/kg; 1.0-5.0 mg/kg; 5.0-10.0 mg/kg; 10-50 mg/kg; 50-100 mg/kg; 100-500 mg/kg; 500-1000 mg/kg; and 1000-5000 mg/kg body weight.

Moreover, the present invention provides, without limitation, doses of at least 0.001 mg/kg body weight; at least 0.005 mg/kg; at least 0.01 mg/kg; at least 0.5 mg/kg; at least 1.0 mg/kg; at least 5.0 mg/kg; at least 10-50 mg/kg; at least 50 mg/kg; at least 100 mg/kg; at least 500 mg/kg; and at least 1000 mg/kg body weight.

The present invention provides doses of, e.g., at least 1.0 mg/m²; at least 2.5 mg/m²; at least 5.0 mg/m²; at least 10 mg/m²; at least 25 mg/m²; at least 50 mg/m²; at least 100 mg/m²; at least 250 mg/m²; at least 1000 mg/m², and at least 2500 mg/m².

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The present invention encompasses a method of administering Listeria that is oral. Also provided is a method of administering Listeria that is intravenous. Moreover, what is provided is a method of administering Listeria that is intramuscular. The invention supplies a Listeria bacterium, or culture or suspension of Listeria bacteria, prepared by growing in a medium that is meat based, or that contains polypeptides derived from a meat or animal product. Also supplied by the present invention is a Listeria bacterium, or culture or suspension of Listeria bacteria, prepared by growing in a medium that does not contain meat or animal products, prepared by growing on a medium that contains vegetable polypeptides, prepared by growing on a medium that is not based on yeast products, or prepared by growing on a medium that contains yeast polypeptides.

The present invention encompasses a method of administering Listeria that is not oral. Also provided is a method of administering Listeria that is not intravenous. Moreover, what is provided is a method of administering Listeria that is not intramuscular. The invention supplies a Listeria bacterium, or culture or suspension of Listeria bacteria, prepared by growing in a medium that is not meat based, or that does not contain polypeptides derived from a meat or animal product. Also supplied by the present invention is a Listeria bacterium, or culture or suspension of Listeria bacteria, prepared by growing in a medium based on vegetable products, that contains vegetable polypeptides, that is based on yeast products, or that contains yeast polypeptides.

Methods for co-administration or treatment with an additional therapeutic agent, e.g., a cytokine, chemotherapeutic agent, antibiotic, or radiation, are well known in the art (Hardman, et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

Where an administered antibody, cytokine, or other therapeutic agent produces toxicity, an appropriate dose can be one where the therapeutic effect outweighs the toxic effect. Generally, an optimal dosage of the present invention is one that maximizes therapeutic effect, while limiting any toxic effect to a level that does not threaten the life of the patient or reduce the efficacy of the therapeutic agent. Signs of toxic effect, or anti-therapeutic effect include, without limitation, e.g., anti-idiotypic response, immune response to a therapeutic antibody, allergic reaction; hematologic and platelet toxicity, elevations of aminotransferases, alkaline phosphatase, creatine kinase, neurotoxicity, nausea, and vomiting (see, e.g., Huang, et al. (1990) Clin. Chem. 36:431-434).

Doses, dosing schedules, and methods for assessing toxicity, for therapeutic antibodies are disclosed (see, e.g., Jayson, et al. (2002) J. Natl. Cancer Inst. 94:1484-1493; Welt, et al. (2003) Clin. Cancer Res. 9:1338-1346; Kips, et al. (2003) Am. J. Resp. Crit. Care Med. 167:1655-1659; Tolcher, et al. (2003) J. Clin. Oncol. 21:211-222; Maciejewski, et al. (2003) Blood 102:3584-3586; Nishimoto, et al. (2003) J. Rheumatol. 30:1426-1435; Leonard, et al. (2003) J. Clin. Oncol. 21:3051-3059; Tobinai (2003) Cancer Chemother. Pharmacol. 52:Suppl.1:S90-S96; Scott, et al. (2003) Clin. Cancer Res. 9:1639-1647; Ghosh, et al. (2003) New Engl. J. Med. 348:24-32; Hassan, et al. (2004) Clin. Cancer Res. 10:16-18; Lebwohl, et al. (2003) New Engl. J. Med. 349:2004-2013; O'Brien, et al. (2003) Cancer 98:2657-2663).

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The reagents and methods of the present invention provide a vaccination method comprising only one vaccination; or comprising a first vaccination; or comprising at least one booster vaccination; at least two booster vaccinations; or at least three booster vaccinations. Guidance in parameters for booster vaccinations is available (see, e.g., Marth (1997) Biologicals 25:199-203; Ramsay, et al. (1997) Immunol. Cell Biol. 75:382-388; Gherardi, et al. (2001) Histol. Histopathol. 16:655-667; Leroux-Roels, et al. (2001) Acta Clin. Belg. 56:209-219; Greiner, et al. (2002) Cancer Res. 62:6944-6951; Smith, et al. (2003) J. Med. Virol. 70:Suppl.1:S38-S41; Sepulveda-Amor, et al. (2002) Vaccine 20:2790-2795).

Formulations of therapeutic and diagnostic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The invention also provides a kit comprising a Listeria cell, a listerial cell culture, or a lyophilized cell preparation, and a compartment. In addition, the present invention provides a kit comprising a Listeria cell, listerial cell culture, or a lyophilized cell preparation and a reagent. Also provided is a kit comprising a Listeria cell, a listerial cell culture, or a lyophilized cell preparation and instructions for use or disposal. Moreover, the present invention provides a kit comprising a Listeria cell, a listerial cell culture, or lyophilized cell preparation, and compartment and a reagent.

Furthermore, what is provided is a kit comprising Listeria cells and an antibody (or a binding compound derived from an antibody). Also available is a kit comprising instructions for use, and an antibody (or a binding compound derived from an antibody). Moreover, what is provided is a kit comprising a compartment, and Listeria cells and an antibody (or a binding compound derived from an antibody). Also supplied is a kit comprising a Listeria bacterium and instructions for using the Listeria bacterium with an antibody. Moreover, provided is a kit comprising an antibody and instructions for using the antibody with Listeria bacteria.

The present invention provides kits and methods for assessing inflammation of a tissue or organ in response to an administered Listeria. Inflammation encompasses an increase in the number (found within a biological compartment) of immune cells, leukocytes, lymphocytes, neutrophils, NK cells, CD4⁺T cells, CD8⁺T cells, B cells, pre-dendritic cells, dendritic cells, monocytes, macrophages, eosinophils, basophils, and/or mast cells, or any combination of the above, and the like. The kits of the present invention also provide for assessing the maturation state or activation state of one or more of the above cells. For identifying the cells and their number, an organ, tissue, or tumor can be pressed through a mesh filter to disperse the immune cells, purified using Percoll®, and identified by Fluorescence Activated Cell Sorting (FACS) (see, e.g., Woo, et al. (1994) Transplantation 58:484-491; Goossens, et al. (1990) J. Immunol. Methods 132:137-144). Inflammation can be measured as number of cells per gram tissue, or an increase in cells per gram tissue as compared with numbers from a non-inflammed state. Also available are methods for assessing Listeria-induced tissue damage, e.g., assays for leukocytosis, lymphopenia, and/or serum transaminases (Angelakopoulos, et al. (2002) Infection Immunity 70:3592-3601; Rochling (2001) Clin. Cornerstone 3:1-12; Roe (1993) Clin. Intensive Care 4:174-182).

Provided is a kit comprising a Listeria and one or more of: (a) an antibody that specifically binds to an antigen of a cancerous or infectious disorder or condition; or (b) a binding compound derived from the antigen-binding site of an antibody that specifically binds to an antigen of the condition and also specifically binds to an immune cell that mediates antibody-dependent cell cytotoxicity (ADCC). Also provided is a kit comprising a Listeria and instructions for administering the Listeria at one or both of: (a) concomitantly with; or (b) at a different time, or during a different time interval than, an antibody that specifically binds to an antigen of a cancerous or infectious condition or a binding compound derived from the antigen-binding site of an antibody that specifically binds to an antigen of the condition and also specifically binds to an immune cell that mediates antibody-dependent cell cytotoxicity (ADCC).

XII. Uses.

The present invention provides methods to administer a Listeria in conjunction with at least one other reagent for use in the recruitment and/or activation of immune cells for treating a proliferative condition or disorder. The second reagent is preferably an antibody that specifically binds to an antigen of a condition for which the individual is being treated, or alternatively, is a binding compound derived from the antigen-binding site of the antibody. The antigen-binding compound also specifically binds to an immune cell that mediates ADCC. The methods are provided for treating a condition or disorder in a tissue or organ where the Listeria naturally accumulates, e.g., the liver. Without limiting the invention to treating liver disorders, it should be noted that L. monocytogenes is a hepatotropic bacterium. Methods are available for administration of Listeria, e.g., intravenously, subcutaneously, intramuscularly, intraperitoneally, orally, by way of the urinary tract, by way of a genital tract, by way of the gastrointestinal tract, or by inhalation (Dustoor, et al. (1977) Infection Immunity 15:916-924; Gregory and Wing (2002) J. Leukoc. Biol. 72:239-248; Hof, et al. (1997) Clin. Microbiol. Revs. 10:345-357; Schluter, et al. (1999) Immunobiol. 201:188-195; Hof (2004) Expert Opin. Pharmacother. 5:1727-1735; Heymer, et al. (1988) Infection 16(Suppl. 2):S106-S111; Yin, et al. (2003) Environ. Health Perspectives 111:524-530).

The present invention contemplates methods of preventing and/or treating cancer of the breast, ovary, cervix, vulva, endometrial cancer, prostate, testes, lung, bronchus, oral cavity, pharynx, hypopharynx, nasopharynx, larynx, esophagus, stomach, small intestines, colon, rectum, gastrointestinal carcinoid tumors, bladder, lymphomas, non-Hodgkin's lymphoma, Hodgkin's lymphoma, melanomas of the skin, skin cancer (non-melanoma), kidney, Wilms' tumor, ureter, pancreas, head, neck, thyroid, brain, eye and orbit, retinoblastoma, multiple myeloma, liver, biliary tree, gall bladder, bile duct, leukemia, acute and chronic lymphoblastic leukemia, acute and chronic myeloid leukemia, soft tissues including the heart, soft tissue sarcoma, pleura, malignant mesothelioma, bones, joints, nose, nasal cavity, middle ear, peritoneum, omentum, mesentery (see, e.g., Devita, et al. (eds) (2004) Cancer: Principles and Practice of Oncology, 7^{th} ed., Lippincott, Williams, & Wilkins, Phila., PA; Casciato (ed.) (2004) Manual of Clinical Oncology, 5^{th} ed., Lippincott, Williams, & Wilkins, Phila., Pa.; Pizzo and Poplack (eds.) (2001) Principles and Practice of Pediatric Oncology, 4^{th} ed., Lippincott, Williams, & Wilkins, Phila., Pa.; Rubin, et al. (eds.) (2001) Clinical Oncology:A Multi-Disciplinary Approach for Physicians and Students, 8^{th} ed., W.B.Saunders, Co., Phila., Pa.; Scheinberg and Jurcic (2004) Treatment of Leukemia and Lymphoma, Academic Press, San Diego, Calif.).

The present invention results, without implying any limitation, in the reduction of the number of abnormally proliferating cells, reduction in the number of cancer cells, reduction in the number of tumor cells, reduction in the tumor volume, reduction of the number of infectious organisms or pathogens per unit of biological fluid or tissue (e.g., serum), reduction in viral titer (e.g., serum), where it is normally reduced by at least 5%, more normally reduced by at least 10%, most normally reduced by at least 15%, preferably reduced by at least 20%, more preferably reduced by at least 25%, most normally reduced by at least 30%, usually reduced by at least 40%, more usually reduced by at least 50%, most usually reduced by at least 60%, conventionally reduced by at least 70%, more conventionally reduced by at least 80%, most conventionally reduced by at least 90%, and still most conventionally reduced by at least 99%. The unit of reduction can be, without limitation, number of tumor cells/mammalian subject; number of tumor cells/liver; number of tumor cells/spleen; mass of tumor cells/mammalian subject; mass of tumor cells/liver; mass of tumor cells/spleen; number of viral particles or viruses or titer per gram of liver; number of viral particles or viruses or titer per cell; number of viral particles or viruses or titer per ml of blood; and the like.

The present invention provides reagents and methods for stimulating innate response as mediated by, e.g., NK cells, NKT cells, dendritic cells and other APCs, CD4⁺T cells, CD8⁺T cells, and gammadelta T cells.

Provided are reagents and methods for stimulating innate response mediated by, e.g., an APC, an APC that migrates to the liver, an APC that is generated to mature in the liver, or an APC that is located in the liver, such as a dendritic cell (DC), Kupfer cell, or liver sinusoidal endothelial cell (LSEC). The present invention is not limited, unless specified explicitly or by context, to the receptors, signaling molecules, or cells that mediate the innate response.

The growth medium used to prepare a Listeria can be characterized by chemical analysis, high pressure liquid chromatography (HPLC), mass spectroscopy, gas chromatography, spectroscopic methods, and the like. The growth medium can also be characterized by way of antibodies specific for components of that medium, where the component occurs as a contaminant with the Listeria, e.g., a contaminant in the listerial powder, frozen preparation, or cell paste. Antibodies, specific for peptide or protein antigens, or glycolipid, glycopeptide, or lipopeptide antigens, can be used in ELISA assays formulated for detecting animal-origin contaminants. Antibodies for use in detecting antigens, or antigenic fragments, of animal origin are available (see, e.g., Fukuta, et al. (1977) Jpn. Heart J. 18:696-704; DeVay and Adler (1976) Ann. Rev. Microbiol. 30:147-168; Cunningham, et al. (1984) Infection Immunity 46:34-41; Kawakita, et al. (1979) Jpn. Cir. J. 43:452-457; Hanly, et al. (1994) Lupus 3:193-199; Huppi, et al. (1987) Neurochem. Res. 12:659-665; Quackenbush, et al. (1985) Biochem. J. 225:291-299). The invention supplies kits and diagnostic methods that facilitate testing the Listeria's influence on the immune system. Testing can involve comparing one strain of Listeria with another strain of Listeria, or a parent Listeria strain with a mutated Listeria strain. Methods of testing comprise, e.g., phagocytosis, spreading, antigen presentation, T cell stimulation, cytokine response, host toxicity, LD₅₀, and efficacy in ameliorating a pathological condition.

The present invention provides methods to increase survival of a subject, host, patient, test subject, experimental subject, veterinary subject, and the like, to a proliferative disorder, a tumor, a cancer, immune disorder, and/or an infectious agent. The infectious agent can be a virus, bacterium, or parasite, or any combination thereof. The method comprises administering a Listeria, for example, as a suspension, bolus, gel, matrix, injection, or infusion, and the like. The administered Listeria increases survival, as compared to an appropriate control (e.g., nothing administered or an administered placebo, and the like) by usually at least one day; more usually at least four days; most usually at least eight days, normally at least 12 days; more normally at least 16 days; most normally at least 20 days, often at least 24 days; more often at least 28 days; most often at least 32 days, conventionally at least 40 days, more conventionally at least 48 days; most conventionally at least 56 days; typically by at least 64 days; more typically by at least 72 days; most typically at least 80 days; generally at least six months; more generally at least eight months; most generally at least ten months; commonly at least 12 months; more commonly at least 16 months; and most commonly at least 20 months, or more.

The invention provides each of the above-disclosed aspects, where the administered Listeria are administered as a composition that is at least 90% free of other types of bacteria, that is at least 95% free of other types of bacteria, that is at least 99% free of other types of bacteria, or that is at least 99.9% free of other types of bacteria. Other types of bacteria include, e.g., a serotype of L. monocytogenes other than that disclosed above. Other types of bacteria also include, e.g., L. welshimeri, L. seeligeri, L. innocua, L. grayi, S. typhimurium (Silva, et al. (2003) Int. J. Food Microbiol. 81:241-248; Pini and Gilbert (1988) Int. J. Food Microbiol. 6:317-326; Council of Experts (2003) Microbiological Tests in The United States Pharmacopeia, The National Formulary, Board of Trustees, pp. 2148-2162).

The invention provides a Listeria bacterium, or a Listeria strain, that is killed but metabolically active (KBMA) (see, e.g., Brockstedt, et al. (2005) Nat. Med. [July 24 epub ahead of print]). A KBMA Listeria bacterium is metabolically active, but cannot form a colony, e.g., on agar. An inactivating mutation in at least one DNA repair gene, e.g., ΔuvrAB, enables killing of Listeria using concentrations of a nucleic acid cross-linking agent (e.g., psoralen) at low concentrations, where these concentrations are sufficient to prevent colony formation but not sufficient to substantially impair metabolism. The result of limited treatment with psoralen/UVA light, and/or of treatment with a nucleic acid cross-linking agent that is highly specific for making interstrand genomic cross links, is that the bacterial cells are killed but remain metabolically active.

Each of the above disclosed methods contemplates admininstering a composition comprising a Listeria and an excipient, a Listeria and a carrier, a Listeria and buffer, a Listeria and a reagent, a Listeria and a pharmaceutically acceptable carrier, a Listeria and an agriculturally acceptable carrier, a Listeria and a veterinarily acceptable carrier, a Listeria and a stabilizer, a Listeria and a preservative, and the like.

The present invention provides reagents and methods for treating conditions that are both cancerous (neoplasms, malignancies, cancers, tumors, and/or precancerous disorders, dysplasias, and the like) and infectious (infections). Provided are reagents and methods for treating disorders that are both cancerous (neoplasms, malignancies, cancers, tumors, and/or precancerous disorders, dysplasias, and the like) and infectious. With infection with certain viruses, such as papillomavirus and polyoma virus, the result can be a cancerous condition, and here the condition is both cancerous and infectious. A condition that is both cancerous and infectious can be detected, as a non-limiting example, where a viral infection results in a cancerous cell, and where the cancerous cell expresses a viral-encoded antigen. As another non-limiting example, a condition that is both cancerous and infectious is one where immune response against a tumor cell involves specific recognition against a viral-encoded antigen (See, e.g., Montesano, et al. (1990) Cell 62:435-445; Ichaso and Dilworth (2001) Oncogene 20:7908-7916; Wilson, et al. (1999) J. Immunol. 162:3933-3941; Daemen, et al. (2004) Antivir. Ther. 9:733-742; Boudewijn, et al. (2004) J. Natl. Cancer Inst. 96:998-1006; Liu, et al. (2004) Proc. Natl. Acad. Sci. USA 101:14567-14571).

The present invention provides a method of administering an attenuated Listeria, e.g., Lm ΔactA or Lm ΔactAΔin1B, by way of a plurality of doses, and an attenuated tumor vaccine, by way of a plurality of doses. In one aspect, the attenuated tumor is engineered to contain a nucleic acid encoding a cytokine, e.g., GM-CSF. In another aspect, the attenuated tumor is not engineered to contain a nucleic acid encoding a cytokine.

The present invention provides a method comprising administration of a metabolically active Listeria for stimulating adaptive immunity (including long-term adaptive immunity; memory response; and recall response), e.g., to a tumor, cancer, infectious agent, viral, parasitic, or bacterial antigen. The invention encompasses the above method, further comprising administration of one or more of a cytokine, e.g., GM-CSF, an attenuated tumor, an attenuated tumor expressing the cytokine, or an inhibitor of Tregs, such as cyclophosphamide (CTX). In another aspect, the above invention comprises the above method, where the Listeria is not engineered to express a heterologous antigen, e.g., an antigen derived from a tumor cell, cancer cell, or infective agent.

The present invention provides a method comprising administering an attenuated Listeria, e.g., Lm ΔactA or Lm ΔactAΔin1B, with attenuated tumor cells (e.g. irradiated metastatic cells), where the cells had been engineered to express a cytokine, e.g., GM-CSF. In the present invention, the Listeria are not engineered to comprise any nucleic acid encoding any heterologous antigen, e.g., a tumor or infectious agent antigen. In another aspect of the present invention, the Listeria are engineered to comprise a nucleic acid encoding a heterologous antigen. In another aspect, the administration also includes an antibody, or binding composition derived from an antibody, that specifically recognizes a tumor antigen, e.g., a tumor antigen of the administered attenuated tumor cell.

XIII. General Methods.

Standard methods of biochemistry and molecular biology are described (see, e.g., Maniatis, et al. (1982) Molecular Cloning, A Laboratory Manual, Cold Spring Harbor Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols:A Guide to Methods and Applications, Academic Press, N.Y. Standard methods are also found in Ausbel, et al. (2001) Curr. Protocols in Mol. Biol., Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). Methods for producing fusion proteins are described (see, e.g., Invitrogen (2005) Catalogue, Carlsbad, Calif.; Amersham Pharmacia Biotech. (2005) Catalogue, Piscataway, N.J.; Liu, et al. (2001) Curr. Protein Pept. Sci. 2:107-121; Graddis, et al. (2002) Curr. Pharm. Biotechnol. 3:285-297). Splice overlap extension PCR, and related methods, are described (see, e.g., Horton, et al. (1990) Biotechniques 8:528-535; Horton, et al. (1989) Gene 77:61-68; Horton (1995) Mol Biotechnol. 3:93-99; Warrens, et al. (1997) Gene 186:29-35; Guo and Bi (2002) Methods Mol. Biol. 192:111-119; Johnson (2000) J. Microbiol. Methods 41:201-209; Lantz, et al. (2000) Biotechnol. Annu. Rev. 5:87-130; Gustin and Burk (2000) Methods Mol. Biol. 130:85-90; QuikChange® Mutagenesis Kit, Stratagene, La Jolla, Calif.). Engineering codon preferences of signal peptides, secretory proteins, and heterologous antigens, to fit the optimal codons of a host are described (Sharp, et al. (1987) Nucl. Acids Res. 15:1281-1295; Uchijima, et al. (1998) J. Immunol. 161:5594-5599).

Methods for protein purification such as immunoprecipitation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described. See, e.g., Coligan, et al. (2000) Current Protocols in Protein Science, Vol. 2, John Wiley and Sons, Inc., New York; Walker (ed.) (2002) Protein Protocols Handbook, Humana Press, Towota, N.J.; Lundblad (1995) Techniques in Protein Modification, CRC Press, Boca Raton, Fla. Techniques for characterizing binding interactions are described (Coligan, et al. (2001) Current Protocols in Immunology, Vol. 4, John Wiley and Sons, Inc., New York; Parker, et al. (2000) J. Biomol. Screen. 5: 77-88; Karlsson, et al. (1991) J. Immunol. Methods 145:229-240; Neri, et al. (1997) Nat. Biotechnol. 15:1271-1275; Jonsson, et al. (1991) Biotechniques 11:620-627; Friguet, et al. (1985) J. Immunol. Methods 77: 305-319; Hubble (1997) Immunol. Today 18:305-306; Shen, et al. (2001) J. Biol. Chem. 276:47311-47319).

Software packages for determining, e.g., antigenic fragments, leader sequences, protein folding, functional domains, glycosylation sites, and sequence alignments, are available (see, e.g., Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690). Methods for determining coding sequences (CDS) are available (Furono, et al. (2003) Genome Res. 13:1478-1487).

Computer algorithms (e.g., BIMAS; SYFPEITHI) for identifying peptides that bind to MHC Class I and/or MHC Class II are available (Thomas, et al. (2004) J. Exp. Med. 200:297-306). These algorithms can provide nucleic acids of the present invention that encode proteins comprising the identified peptides.

Sequences of listerial proteins and nucleic acids can be found on the world wide web at: (1) ncbi.nlm.nih.gov; (2) genolist.Pasteur (with clicking on “listilist”); and (3) tigr.org (with clicking on “comprehensive microbial resource”).

Methods are available for assessing internalization of a Listeria by an APC, and for assessing presentation of listerial-encoded antigens by the APC. Methods are also available for presentation of these antigens to T cell, and for assessing antigen-dependent priming of the T cell. A suitable APC is murine DC 2.4 cell line, while suitable T cell is the B3Z T cell hybridoma (see, e.g., U.S. Provisional Pat. Appl. Ser. No. 60/490,089 filed Jul. 24, 2003; Shen, et al. (1997) J. Immunol. 158:2723-2730; Kawamura, et al. (2002 J. Immunol. 168:5709-5715; Geginat, et al. (2001) J. Immunol. 166:1877-1884; Skoberne, et al. (2001) J. Immunol. 167:2209-2218; Wang, et al. (1998) J. Immunol. 160:1091-1097; Bullock, et al. (2000) J. Immunol. 164:2354-2361; Lippolis, et al. (2002) J. Immunol. 169:5089-5097). Methods for preparing dendritic cells (DCs), ex vivo modification of the DCs, and administration of the modified DCs, e.g., for the treatment of a cancer, pathogen, or infective agent, are available (see, e.g., Ribas, et al. (2004) J. Immunother. 27:354-367; Gilboa and Vieweg (2004) Immunol. Rev. 199:251-263; Dees, et al. (2004) Cancer Immunol. Immunother. 53:777-785; Eriksson, et al. (2004) Eur. J. Immunol. 34:1272-1281; Goldszmid, et al. (2003) J. Immunol. 171:5940-5947; Coughlin and Vonderheide (2003) Cancer Biol. Ther. 2:466-470; Colino and Snapper (2003) Microbes Infect. 5:311-319).

Elispot assays and intracellular cytokine staining (ICS) for characterizing immune cells are available (see, e.g., Lalvani, et al. (1997) J. Exp. Med. 186:859-865; Waldrop, et al. (1997) J. Clin. Invest. 99:1739-1750; Hudgens, et al. (2004) J. Immunol. Methods 288:19-34; Goulder, et al. (2001) J. Virol. 75:1339-1347; Goulder, et al. (2000) J. Exp. Med. 192:1819-1831; Anthony and Lehman (2003) Methods 29:260-269; Badovinac and Harty (2000) J. Immunol. Methods 238:107-117).

Methods for using animals in the study of cancer, metastasis, and angiogenesis, and for using animal tumor data for extrapolating human treatments are available (see, e.g., Hirst and Balmain (2004) Eur J Cancer 40:1974-1980; Griswold, et al. (1991) Cancer Metastasis Rev. 10:255-261; Hoffman (1999) Invest. New Drugs 17:343-359; Boone, et al. (1990) Cancer Res. 50:2-9; Moulder, et al. (1988) Int. J. Radiat. Oncol. Biol. Phys. 14:913-927; Tuveson and Jacks (2002) Curr. Opin. Genet. Dev. 12:105-110; Jackson-Grusby (2002) Oncogene 21:5504-5514; Teicher, B. A. (2001) Tumor Models in Cancer Research, Humana Press, Totowa, N.J.; Hasan, et al. (2004) Angiogenesis 7:1-16; Radovanovic, et al. (2004) Cancer Treat. Res. 117:97-114; Khanna and Hunter (2004) Carcinogenesis 26:513-523; Crnic and Christofori (2004) Int. J. Dev. Biol. 48:573-581).

Colorectal cancer hepatic metastases can be generated using primary hepatic injection, portal vein injection, or whole spleen injection of tumor cells (see, e.g., Suh, et al. (1999) J. Surgical Oncology 72:218-224; Dent and Finley-Jones (1985) Br. J. Cancer 51:533-541; Young, et al. (1986) J. Natl. Cancer Inst. 76:745-750; Watson, et al. (1991) J. Leukoc. Biol. 49:126-138).

The broad scope of this invention is best understood with reference to the following examples, which are not intended to limit the invention to any specific aspects.

### EXAMPLES

I. Standard Methods Used in the Examples.

The Listeria monocytogenes strains used in the present work are described (see, e.g., Brockstedt, et al. (2004) Proc. Natl. Acad. Sci. USA 101: 13832-13837). L. monocytogenes ΔactAΔin1B is available from American Type Culture Collection (ATCC) at PTA-5562. L. monocytogenes ΔactAΔuvrAB is available from ATCC at PTA-5563.

A number of animal tumor models were used, where these models utilized BALB/c mice and the syngeneic colorectal cancer line CT26 (ATCC CRL-2638). The models used in the present invention included: (1) Subcutaneous CT26 tumors; and (2) Injection of tumor cells into half of a surgically bisected spleen, followed by immediate excision of the injected half (hemi-spleen model). The hemi-spleen model established colorectal cancer hepatic metastases without producing a primary tumor in the spleen. The hemi-spleen method allows seeding of the liver with tumor cells through the portal circulation without the presence of a primary tumor in the injected spleen. Where indicated, mice were treated with GM-CSF secreting tumor vaccines, where vaccination was initiated three days after tumor challenge.

CT26, an immortal mouse colorectal cancer cell line (generated by exposure of BALB/c background mice rectal tissue to methylcholanthrine) was used to establish tumors used in the present study (Corbett, et al. (1975) Cancer Res. 35:2434-2439). The vaccine cell line was derived from CT26 cells transduced to secrete murine GM-CSF using a replication defective MFG retroviral vector (Dranoff, et al. (1993) Proc. Natl. Acad. Sci. USA 90:3539-3543). Tumor cell lines were grown in tumor media containing (vol/vol) 900 ml RPMI media, 100 ml 10% heat inactivated fetal calf serum, 10 ml penicillin/streptomycin (10,000 U/ml), 10 ml MEM non-essential amino acids (10 mM), 10 ml HEPES buffer (1 M), 10 ml sodium pyruvate (100 mM), and 10 ml L-glutamate (200 mM).

For subcutaneous tumor model studies, BALB/c mice were injected with 0.1 million CT26 colorectal cancer cells suspended in 0.05 ml HBSS below the left lower nipple. Tumors were allowed to grow for 28 days in control mice. Tumors were measured bi-weekly in three dimensions using calipers. Treated mice were vaccinated with GM-CSF secreting tumor cells on a bi-weekly basis.

Hemi-spleen injections were as follows. BALB/c mice were anaesthetized and the spleen exposed. The spleen was divided into two hemi-spleens, leaving the vascular pedicles intact. Using a 27 gauge needle, about 0.1 million viable CT26 cells in 0.4 ml HBSS buffer were injected into the spleen, thus allowing cells to flow to the liver. The vascular pedicle draining the cancer-contaminated hemi-spleen was ligated with a clip, and the CT26-contaminated hemi-spleen was excised, leaving a functional hemi-spleen free of tumor cells.

In all studies, except for one study as indicated, the vaccine (tumor cell vaccine) was prepared by treating the tumor cells with gamma-rays. In this one study, the vaccine was prepared by photochemical treatment (psoralen and UV light). In all studies, except where indicated, the number of pathologic CT26 tumor cells used in the innoculum (not the attenuated CT26 cells used in the vaccine) administered was about 0.1 million cells. Subjecting tumor cells with gamma-rays or photochemical treatment results in attenuated tumor cells that can provide an antigen or antigens, and can express an immunomodulating agent such as GM-CSF, but cannot grow and/or replicate. Where a nucleic acid encoding GM-CSF is used as part of a vaccine, the terms “GM vaccine” and “GM-CSF vaccine” may be used interchangeably.

In general, mice receiving Listeria weighed 20-25 grams, and had a surface area of about 0.0066 square meters.

Anti-CD16/32, anti-CD69, anti-CD25, and anti-CD3 were from eBioscience (San Diego, Calif.). Total numbers of NK cells and NK-T cells was determined using the following cocktail: CD45 to stain all leukocytes, to separate these from residual liver cells, and CD 19 to eliminate B cells from the analysis. Then, the two parameter plot of CD3 versus DX-5 was used to identify T cells (CD3⁺DX-5⁻), NK cells (DX-5⁺CD3⁻), and NK-T cells (CD3⁺DX-5⁺). Cyclophosphamide was from Sigma (St. Louis, Mo.), and dissolved in HBSS before injecting in animals.

Preparation of attenuated Listeria monocytogenes (e.g., Lm ΔactA and Lm ΔactAΔin1B), reagents and methods for engineering a nucleic acid encoding the tumor antigen AH 1-A5, or a nucleic acid encoding ovalbumin, in Listeria monocytogenes (Lm), methods for measuring hepatic aminotransferases, equipment for flow cytometry (FACS®), and methods for measuring tumor metastases, were as described (Brockstedt, et al. (2004) Proc. Natl. Acad. Sci. USA 101:13832-13837).

II. Antibody-Dependent Cytotoxicity (ADCC).

ADCC was demonstrated according to the following study design. Listeria monocytogenes (Lm) was administered to mice, followed by an interval of time to allow interaction with the immune system, followed by removal of stimulated splenocytes, exposure of the splenocytes to target cells, and incubation allowing lysis by the splenocytes of the target cells. Target cell lysis was enhanced where the origin of the splenocytes was Lm-treated mice, where further enhancement occurred with added antibody.

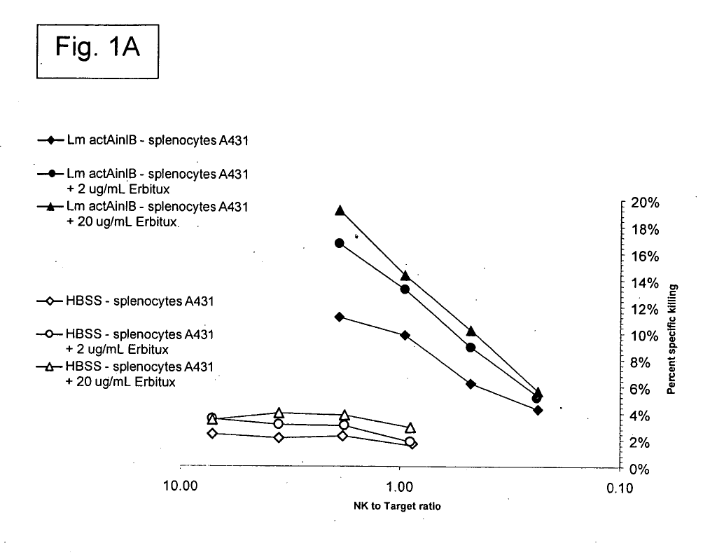

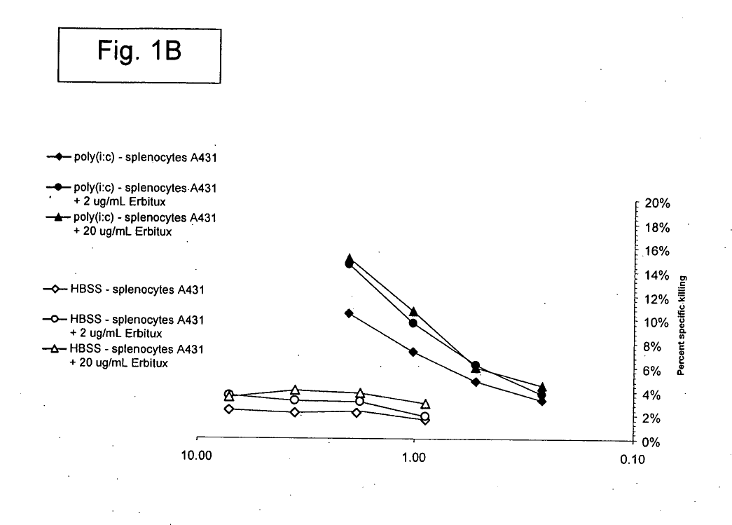

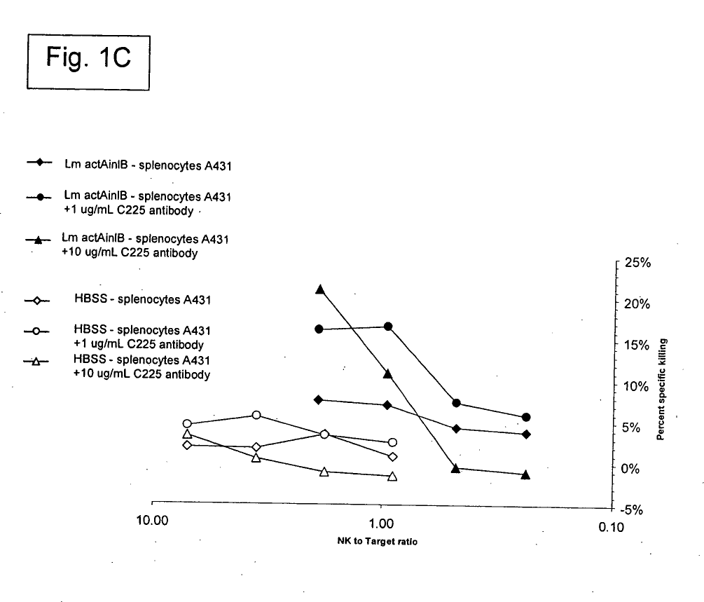

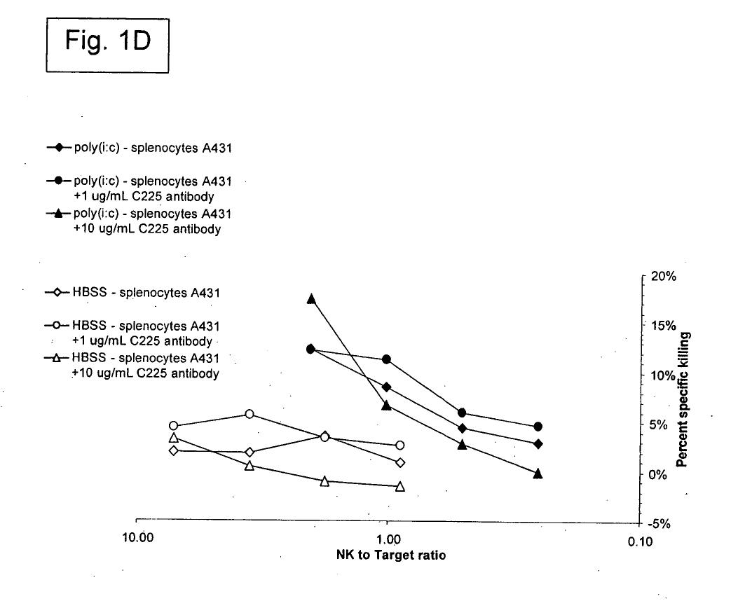

FIGS. 1A to 1D demonstrate synergy between Lm and antibodies, in ADCC.

Synergistic effects resulted, as demonstrated by comparing the percent killing with administration of Listeria only, antibody only, or Listeria plus antibody. To view the numbers, FIG. 1A demonstrates that killing with antibody only was about 3%. With Listeria only was about 11%, but with the combination of antibody plus Listeria, killing was about 19%. FIG. 1C demonstrates the same effect, that is, killing with antibody only was about 3%, killing with Listeria only was about 8%, where a synergistic effect was shown after administration of both antibody and Listeria (killing of about 20%) (FIG. 1C).

i. Methods.

In detail, Lm ΔactAΔinlB was administered to mice followed, 24 hours later, by removing the splenocytes and determining in vitro splenocyte-mediated lysis of target tumor cells, in the presence and absence of added antibody. The antibody was a humanized antibody, derived from a mouse hybridoma, where the antibody was specific for epidermal growth factor receptor (EGFR).

Mice were prepared in three ways. C57Bl/6 mice were treated with: (1) Lm ΔactAΔinlB (1×10⁷ cfu) (experimental mice); (2) 100 micrograms poly(I:C) (positive control mice); or (3) Hanks buffered salt solution (HBSS) (negative control mice).

Poly(I:C) activates immune cells when administered in vivo or in vitro (see, e.g., Laskay, et al. (1993) Eur. J. Immunol. 23:2237-2241; Schmidt, et al. (2004) J. Immunol. 172:138-143; Gerosa, et al. (2005) J. Immunol. 174:727-734).

Twenty-four hours after administering the bacteria, poly(I:C), or HBSS, spleens were harvested, single cell suspensions were prepared, and the suspensions were mixed with target cells (⁵¹Cr loaded A431 cells). After mixing the splenocyte suspensions with the target cells, target cell lysis was assessed.

A431 cells are an epidermal squamous cell carcinoma cell line (CRL-1555, American Type Culture Collection, Manassas, Va.). The target cells had been pre-mixed with one of:
(1) HBSS;
(2) The anti-epidermal growth factor receptor antibody, Erbitux® (2 or 20 micrograms/ml; final concentration); or
(3) The anti-epidermal growth factor receptor antibody, C225 antibody (1 or 10 micrograms/ml; final concentration), followed by a 30 minute incubation.

Erbitux® was from Imclone (New York, N.Y.). The C225 antibody was ATCC Number HB-8508 (also known as 225) from American Type Culture Collection (ATCC) (Manassas, Va.).

Once the target cells were incubated with antibody, the cells were then mixed with splenocytes, where splenocyte-mediated lysis of target cells was permitted to occur for four hours. Lysis was assessed by measuring release of ⁵¹ Cr. Lysis assays contained a constant number of target cells (5,000 cells) in 0.2 ml. The splenocyte population contained a variety of immune cells, including NK cells. In the incubation mixtures, the ratios of NK cells to target cells was controlled so that the ratio would occur at specific ratios, from about 0.10 to about 10.0 (FIGS. 1A to 1D).

ii. Results.

The following concerns splenocytes isolated from mice treated with L. monocytogenes ΔactAΔinlB. With these Listeria-exposed splenocytes, killing of the target cells increased with increasing ratios of NK cells/target cells, where killing was found to be stimulated by Erbitux® (FIG. 1A, upper three curves) as well as by the C225 antibody (FIG. 1C, upper three curves). At the highest NK cell/target cell ratio, the percent killing by Listeria-exposed splenocytes of the target cells was about 11% (no antibody) and about 19% (20 microgram/ml Erbitux®) (FIG. 1A). With the other source of antibody (C225), the results were as follows (FIG. 1C). At the highest NK cell/target cell ratio, the percent killing of Listeria-exposed splenocytes of target cells was about 8% (no antibody) and about 21% (10 micrograms/ml C225) (FIG. 1C). In positive control incubations, where the source of splenocytes was poly(I:C)-treated mice, the poly(I:C)-treatment was found to enhance killing by splenocytes of target cells, where this killing was further enhanced by adding antibody (FIGS. 1B, upper three curves, and 1D, upper three curves).

The results were as follows (FIGS. 1 to 1D). The lower three curves in each of FIGS. 1A to 1D show controls, where all the controls involved splencytes isolated from HBSS-treated mice. With splenocytes from control mice (no Listeria and no poly(I:C)), about 2 to 4% of the target cells were killed, with little or no change in the percent killed at different ratios of NK cells/target cells.

The three sources of splenocytes used for the above studies were examined in some detail. To repeat, the three sources of splenocytes were those isolated 24 hours post-injection of: (1) L monocytogenes ΔactAΔinlB (1×10⁷ cfu); (2) 100 micrograms poly(I:C); or (3) HBSS. The isolated splenocytes were analyzed by flow cytometry, utilizing anti-NK1.1 antibody and anti-CD69 antibody as probes. The anti-NK1.1 antibody was used to determine if the cell was an NK cell, and the anti-CD69 antibody was used to assess activation state of each NK, cell. The antibodies were from eBioscience, San Diego, Calif.

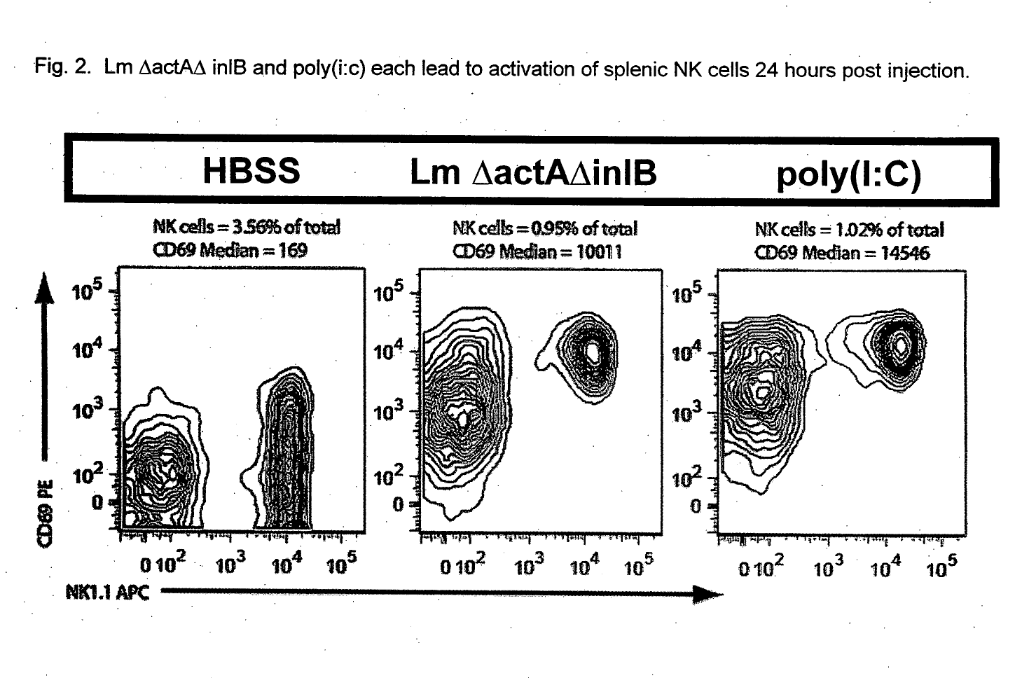

The results were as follows (FIG. 2). With the negative control splenocytes (HBSS treatment), NK cells comprised about 3.5% of the splenocytes, and median phycoerythrin (PE) fluorescence intensity, reflecting CD69 expression by the NK cells, was 169 (FIG. 2).

With Listeria treatment, NK cells comprised about 0.95% of the splenocytes, and the median phycoerythrin (PE) fluorescence intensity, reflecting CD69 expression, was 10,011 (FIG. 2).

With positive control splenocytes (poly I:C-treatment), NK cells comprised about 1.0% of the total splenocytes, and the median phycoerythrin (PE) fluorescence intensity, reflecting CD69 expression, was 14,546 (FIG. 2).

The activation of the splenocyte NK cells occurring after treatment with the Listeria or with poly(I:C) (FIG. 2) is consistent with the increases in splenocyte-dependent target cell lysis, after treatment with Listeria or poly(I:C) (FIGS. 1A to 1D).

III. Administration of Attenuated Listeria (with No Vaccine) Enhanced Survival to Liver Tumors (Generated via Hemispleen Injection Model).

Hepatic tumors were induced in mice as follows. CT26 tumor cells were administered to all mice on day zero (t=0 days) to initiate hepatic tumor formation. Mice were treated intravenously (i.v.) with no Listeria (-▪-lower curve of small squares), with the indicated amount of Listeria ΔactA (-⋄-open diamonds; -▴-triangles; -●-filled circles); or with the indicated amount of Listeria ΔactAΔinlB (-∇-inverted triangles; -▪-upper curve of large squares; -♦-filled diamonds) (FIG. 1A).

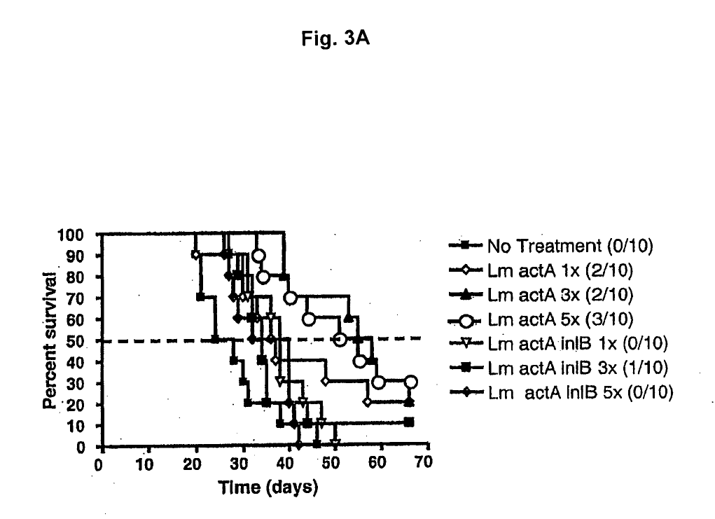

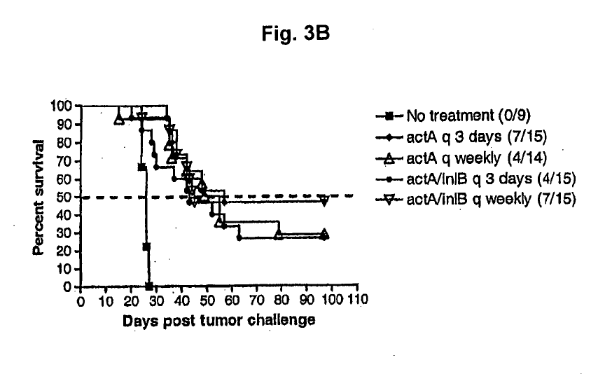

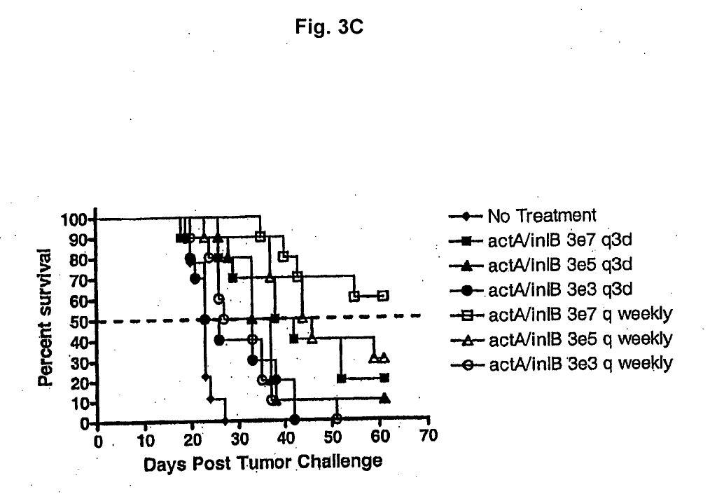

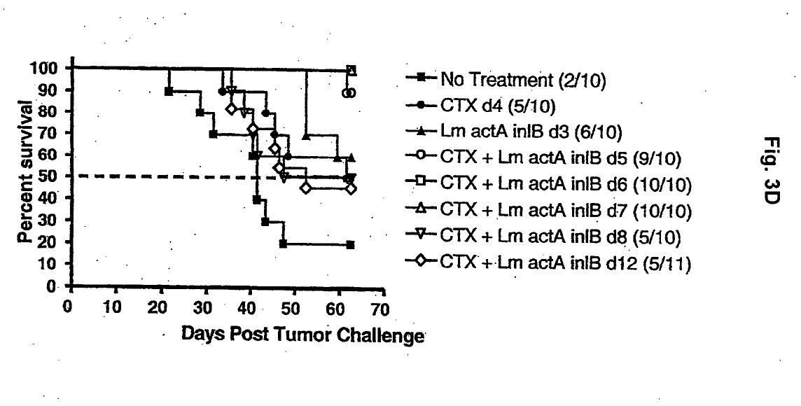

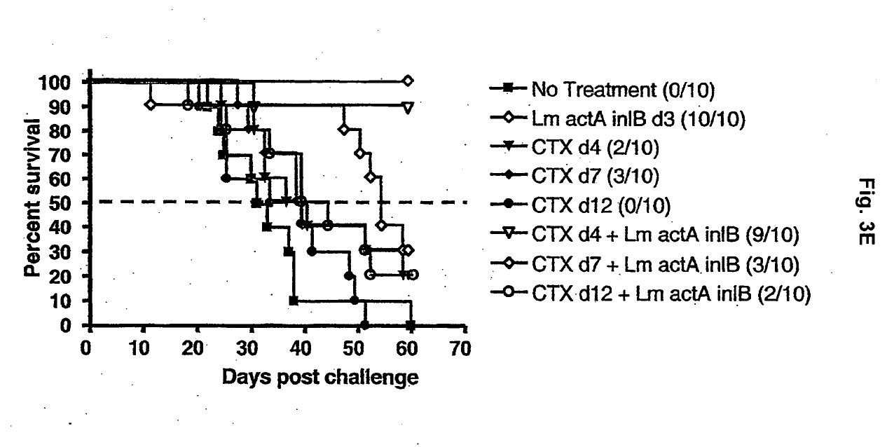

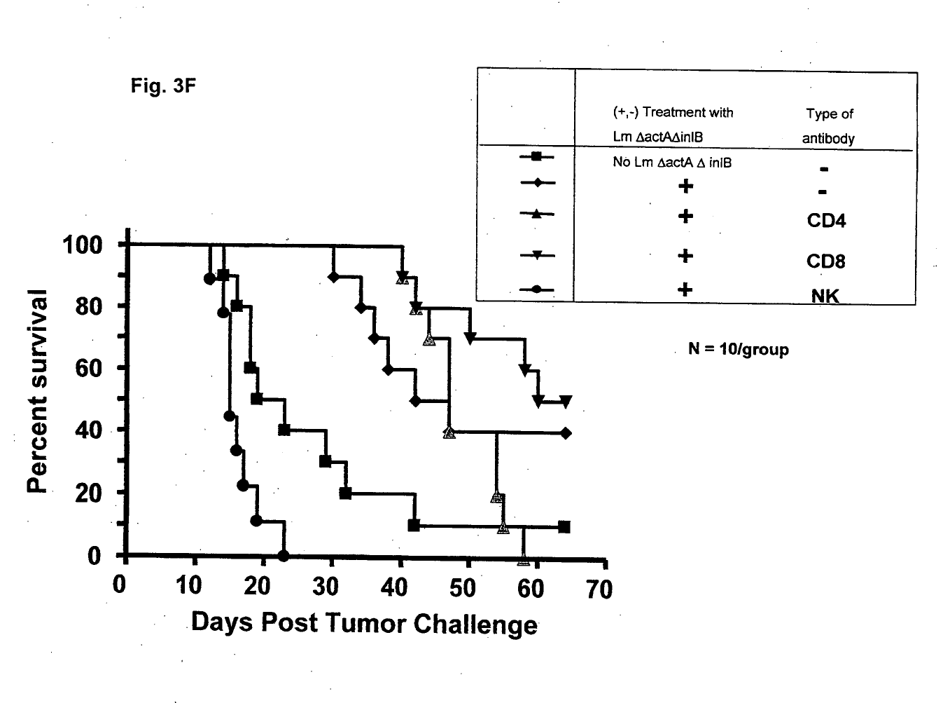

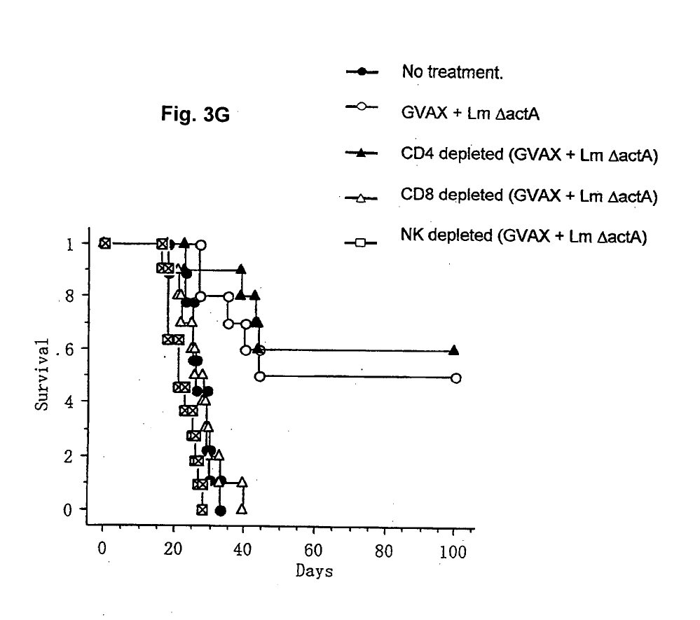

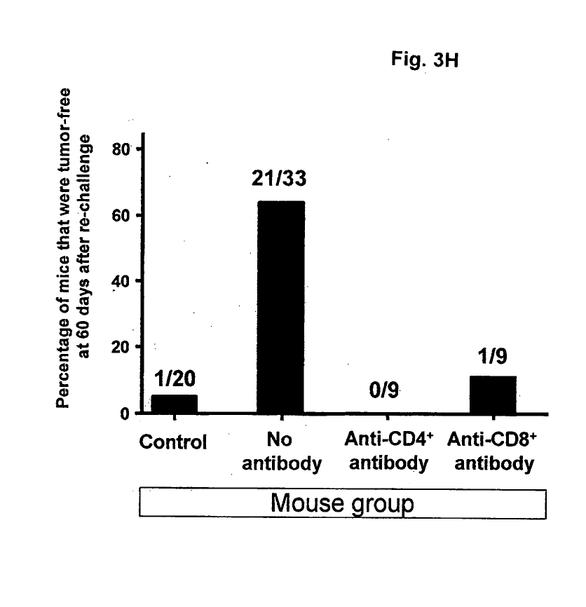

The following concerns the number of doses of Listeria given to the mice. “1×” means that the indicated Listeria strains were administered only at t=3 days post tumor implant (only one dose). “3×” means that the indicated Listeria strains were administered at t=3 days, 6 days, and 9 days. “5×” means that the indicated Listeria strains were administered at t=3 days, 6 days, 9 days, 12 days, and 15 days. The number of administered Listeria ΔactA cells was about 1×10⁷ colony forming units (CFU) while the number of Listeria ΔactAΔinlB given was about 2×10⁷ CFU (FIG. 3A).

The results were as follows. Where tumor-bearing mice received no Listeria, 50% of the mice died by 25 days, while 100% died by day 42. In contrast, mice treated with Listeria ΔactA or Listeria ΔactAΔinlB showed increased survival. For example, at t=25 days, all mice receiving either Listeria ΔactA or Listeria ΔactAΔinlB showed a survival rate of at least 90%. The survival rate was the greatest with Listeria ΔactA, where Listeria ΔactA was provided at 3× or 5× doses (FIG. 3A).

In a separate study (FIG. 3B), CT26 tumor cell-treated mice were given no Listeria (-▪-; squares); Listeria ΔactA (every three days, three doses in all) (-♦-; diamonds); Listeria ΔactA (weekly, three doses in all) (-Δ-; open triangles); Listeria ΔactAΔinlB (every three days, three doses in all) (-●-filled circles); or Listeria ΔactAΔinlB (weekly, three doses in all) (-∇-; inverted open triangles). The results demonstrated that with no treatment, all animals died before t=30 days, whereas Listeria-treatment resulted in survival of up to 50% of the animals at t=100 days (FIG. 1B). Again, the Listeria used to provide data for FIGS. 1A, B were not engineered to contain any nucleic acid encoding heterologous antigen.

In still another study (FIG. 3C), CT26 tumor cell-innoculated mice were treated as follows. Bacteria were grown on yeast broth with no glucose, where bacteria were administered i.v. Mice were given no Listeria (-♦-; diamonds); Listeria ΔactAΔinlB (3×10⁷ CFU, every three days, three doses in all) (-▪-; squares); Listeria ΔactAΔinlB (3×10⁵ CFU, every three days, three doses in all) (-▴-; filled triangles); Listeria ΔactAΔinlB (3×10³ CFU, every three days, three doses in all) (-●-; filled circles); Listeria ΔactAΔinlB (3×10⁷ CFU, weekly, three doses in all) (-□-; open squares); Listeria ΔactAΔinlB (3×10⁵ CFU, weekly, three doses in all) (-Δ-; open triangles); Listeria ΔactAΔinlB (3×10³ CFU, weekly, three doses in all) (-ο-; open circles). An observation that can be made is that, with no treatment, all of the animals died by t=30 days, while mice receiving Listeria ΔactAΔinlB (3×10⁷ CFU) weekly (-□-; open squares) had the greatest survival.

Studies of tumor-bearing mice treated with Listeria, where the Listeria was not engineered to express a heterologous antigen, were continued, where these continued studies included administration of cyclophosphamide (Cytoxan®; CTX) (FIGS. 3D and 3E). The day of CTX treatment (t=day 4) was held constant, while the day of Listeria administration was varied (FIG. 3D). When administered, CTX was provided at 50 mg/kg (i.p.). All doses of L. monocytogenes were 3×10⁷, where the bacteria were prepared by growing in yeast broth with no glucose. The following provides a legend to the figure: Data from mice with no treatment (-▪-; filled squares); treated with CTX only (day 4 injection) (-●-; filled circles); Listeria ΔactAΔinlB only (Listeria administered on days 3, 10, 17) (-▴-; filled triangles); CTX (day 4) with Listeria ΔactAΔinlB (Listeria administered on days 5, 12, and 19) (-ο-; open circles); CTX (day 4) with Listeria ΔactAΔinlB (Listeria administered on days 6, 13, and 20) (-□-; open. squares); CTX (day 4) with Listeria ΔactAΔinlB (Listeria administered on days 7, 14, 21) (-Δ-; open triangles); CTX (day 4) with Listeria ΔactAΔinlB (Listeria administered on days 8, 15, and 22) (-∇-; open inverted triangles); and CTX (day 4) with Listeria ΔactAΔinlB (Listeria administered on days 12, 19, and 26) (-⋄-; open diamonds). The results were as follows. With no treatment (no CTX; no Listeria), survival of the mice at about t=50 days was about 20% (-570 -; closed squares). With CTX only, survival was about 60% at t=50 days (-●-; filled circles). In some protocols that included both CTX and bacteria, survival was between 90-100% after t=60 days (Listeria administered at t=day 5, 6, or 7).

FIG. 3D demonstrates that administering CTX (at t=4 days) alone results in some increase in survival, and that administering CTX (at t=4 days) plus Listeria (Listeria administered at days 5, 12, and 19; Listeria administered at days 6, 13, and 20; or Listeria at days 7, 14, and 21) results in even greater survival.

The following demonstrates that CTX+Listeria can improve survival, and illustrates tests showing how long administration of this combination can be delayed and where the delated combination still improved survival.

FIG. 3E demonstrates combination therapy, and the effects of delaying combination therapy. In this figure, “combination therapy” means the combination of Listeria ΔactAΔinlB (not engineered to express any heterologous antigen) plus cyclophosphamide. Where no treatment was give, half the animals died by about t=32 days. When administered, CTX was provided at 50 mg/kg (i.p.). All doses of L. monocytogenes were 3×10⁷, where the bacteria were prepared by growing in yeast broth with no glucose.

Where the combination dose schedule was started at t=4 days (CTX at day 4 and Listeria at days 5, 12, and 19) (-∇-; open inverted triangles), near maximal survival was found, and here 90% of the animals were surviving at t=60 days. Where the combination dose schedule was delayed somewhat, and started at t=7 days (CTX at day 7 and Listeria at days 8, 15, and 22), about 90% of the animals were surviving at t=48 days, with about half surviving at t=53 days (-⋄-; open diamonds). With further delay in initiating combination therapy, and started at t=12 days (CTX at t=12 days and Listeria at days 13, 20, and 27), survival was relatively poor (-ο-; open circles) (FIG. 3E).

The experiments for which results are shown in FIGS. 3F, 3G, and 3H involve the use of depleting antibodies which, when injected in a mouse, deplete a predetermined type of immune cell, for example, CD8+T cells or NK cells.

The results shown in FIG. 3F provide insight into the mechanisms by which Listeria (not engineered to express any tumor antigen) improves survival to tumors in the absence of a second vaccine. (GVAX was not used in this particular experiment.)

The experimental methods for FIG. 3F were as follows: On Day 0, female Balb/c mice were implanted with 1×10⁵ CT26 cells via hemispleen surgery, and randomized into different treatment groups. CD4+and CD8+T cell and NK cell depletion was initiated one week prior to tumor cell implantation followed by two additional injections on Days 6 and 13 of the GK1.5 (anti-CD4), 2.43 (anti-CD8) and anti-AsialoGM (anti-NK) antibodies, respectively. Depletion of the respective lymphocyte population was confirmed by flow cytometry in separate cohorts of mice. Three weekly treatments with 3×10⁷ cfu of Lm ΔactAΔinlB were initiated on Day 3, except for the control, and mice were followed for survival.

FIG. 3F shows the percent survival of the mice inoculated with CT26 tumors, where the CT26-tumor cell inoculated mice were treated with Lm ΔactAΔinlB or with no Lm ΔactAΔinlB , as indicated. The treated mice either received no antibody or received antibodies that specifically deplete CD4⁺T cells; CD8⁺T cells; or NK cells, as indicated. The results demonstrated maximal, or near maximal, survival where mice received Lm ΔactAΔinlB after receiving no depleting antibodies; Lm ΔactAΔinlB after receiving anti-CD4⁺T cell antibodies; or Lm ΔactAΔinlB after receiving anti-CD8⁺T cell antibodies). In contrast, low survival occurred where Lm ΔactAΔinlB was not administered, or where Lm ΔactAΔinlB was administered to mice who had received anti-NK cell antibodies. These results indicate that following the initial inoculation with tumor cells, Lm-mediated stimulation of NK cells is of major importance for survival to tumors, whereas CD8⁺T cells and CD4⁺T cells are relatively unimportant to survival.

The following addresses the mechanisms by which Listeria (not engineered to express any tumor antigen), in combination with GM-CSF vaccine, improves survival to tumors. FIG. 3G reveals survival of mice to CT26 tumors, where CT26-tumor cell inoculated mice were treated with Listeria ΔactA plus GM-CSF vaccine, along with an agent that specifically depletes CD4⁺T cells (-▴-; GK1.5 antibody), CD8⁺T cells (-Δ-; 2.43 antibody), or NK cells (- [symbol] -; anti-asialo-GM1 antibody), or no other agent (-●-; no treatment, NT). Treatment with the indicated antibodies was for two weeks prior to implantation of intra-hepatic tumor cells. Antibody-dependent depletion of over 90% of CD4⁺T cells, CD8⁺T cells, or NK cells, was confirmed by flow cytometry analysis of liver and spleen from one or two animals from each group. The results demonstrate that maximal survival of tumor cell-bearing mice occurred where mice were treated with Listeria plus vaccine (-ο-) or with Listeria plus vaccine along with the CD4⁺T cell-depleting antibody (-▴-; GK1.5 antibody). In contrast, survival was poor (as poor as with no administered therapeutic agents) where tumor cell-bearing mice were treated with Listeria plus vaccine along with an antibody that depletes CD8⁺T cells or with an antibody that depletes NK cells.

In some embodiments, the present invention provides a method to improve survival to a cancer, by administering a Listeria plus attenuated tumor cells, where the attenuated tumor cells share antigenic properties with the cancer; and where the survival to the cancer is mediated by, and not limited to, NK cells and/or CD8⁺T cells. Moreover, the present invention also provides, in some embodiments, a method to improve survival to an infectious agent (e.g., virus, bacteria, parasite), by administering a Listeria plus attenuated infectious agent, where the attenuated infectious agent shares antigenic properties with the infectious agent, and where the survival to the infectious agent is mediated by, and not limited to, NK cells and/or CD8⁺T cells.

FIG. 3H shows the results of a depletion study where long term survivors that were previously injected with Lm ΔactAΔinlB following inoculation with CT26 tumor cells were re-challenged with CT26 tumor cells. Briefly, experimental mouse groups were inoculated with CT26 tumor cells (1×105 CT26 cells), via the hemispleen model, at t=0 days, and were subsequently injected with Lm ΔactAΔinlB (1×10⁷ bacteria/dose) at t=3, 10, and 17 days (three doses). At t >100 days, about 50-60% of the mice were still alive, and these were the long term survivors. To evaluate tumor specific T cell immunity, long-term survivors were rechallenged subcutaneously with CT26 cells (2×10⁵ CT26 cells).

Prior to the CT26 tumor cell rechallenge, anti-CD4 antibodies or anti-CD8 antibodies were administered to some of the long-term survivors. The anti-CD4 antibody and anti-CD8 antibodies used in the experiment were prepared at Cerus Corporation, Concord, Calif., although anti-CD4 antibodies and anti-CD8 antibodies suitable for depleting experiments are commercially available (e.g., Invitrogen, Carlsbad, Calif.; R & D Systems, Minneapolis, Minn.). The depleting antibodies were injected (0.25 mg injected, i.p.) eight, four, and one day prior to the CT26 cell re-challenge. T cell subsets depletion was confirmed by flow cytometry analysis. Survival of mice to the CT26 cell re-challenge was determined after waiting at least 60 days after the CT26 cell re-challenge dose.

At the time of the re-challenge of the experimental mice, naive mice (controls) were also inoculated with CT26 cells. The control mice had never been earlier exposed to either CT26 tumor cells or Lm ΔactAΔinlB , that is, they were naive for both CT26 cells and for Lm ΔactAΔinlB .

The results, shown in FIG. 3H, demonstrate that in the control group, only one out of 20 mice survived the CT26 cell re-challenge. In the experimental group (i.e., the long-term survivors), about two thirds of the mice (21 out of 33 mice) survived the tumor cell re-challenge. However, where experimental mice had also received either anti-CD4 antibody or anti-CD8 antibody, most of the mice died in response to the tumor cell re-challenge. These results demonstrate that Lm ΔactAΔinlB , an engineered bacterium that does not contain any nucleic acid encoding a tumor antigen, can stimulate long-term tumor-specific adaptive (memory) immune response, and that this long-term adaptive immune response was both CD4⁺T cell and CD8⁺T cell dependent.

IV. Listeria Did Not Provoke Toxic Effects in Regenerating Liver.

The following control study assessed the time course for recovery from partial hepatectomy (Table 4). Partial liver resection is commonly used in the treatment of liver tumors. The time course of recovery from partial hepatectomy was assessed by the release of hepatic enzymes (serum alanine aminotransferase (ALT); serum aspartate aminotransferase (AST)) (see, e.g., Nathwani, et al. (2005) Hepatology 41:380-383; Clavien, et al. (2003) Ann Surg. 238:843-850). Serum enzyme levels were found to reach a basal level by t=3 days after the partial hepatectomy (Table 4).

TABLE 4
Mean serum enzyme levels at intervals after partial hepatectomy.

Day

| | 0 | 1 | 2 | 3 | 4 | 5 |
|---|---|---|---|---|---|---|
| Mean | 4401 | 939 | 209 | 110 | 94 | 171 |
| AST | | | | | | |
| Mean | 5228 | 952 | 198 | 130 | 41 | 58 |
| ALT | | | | | | |

The following control study demonstrated that the LD₅₀ for Listeria is the same, or similar, in normal mice and in hemispleen mice. In normal mice, the LD₅₀ for Listeria ΔactA was 1.0×10⁸ bacteria (also expressed using the following terminology: 1.0e8), and for Listeria ΔactAΔinlB was 2 to 5×10⁸ bacteria. In the hemispleen mice, the LD₅₀ for Listeria ΔactA was 1.23×10⁸ bacteria (also expressed using the following terminology: 1.23e8), and for Listeria ΔactAΔinlB was greater than 1.49×10⁸ bacteria (Table 5).

Naive mice, or mice receiving a partial hepatectomy were titrated with Listeria ΔactA or with Listeria ΔactAΔinlB , to determine if the partial hepatectomy influenced Listeria toxicity (Table 5). At t=0 days, mice received no surgery, or a partial hepatectomy (about 40%). At t=3 days, all mice received the indicated amount of Listeria (Table 5).

TABLE 5
Survival of naive mice and partial hepatectomized
mice after Listeria challenge.

| | | Partial | Naive mice |
|---|---|---|---|
| | LD₅₀ | hepatectomized | (no partial |
| Listeria administered. | (Listeria dose) | mice | hepatectomy) |
| Listeria ΔactA | 5.52 × 10⁸ | 2/2 | 0/3 |
| Listeria ΔactA | 1.44 × 10⁸ | 3/3 | 0/3 |
| Listeria ΔactA | 6.29 × 10⁷ | 2/3 | 1/3 |
| Listeria ΔactA | 8.30 × 10⁶ | 0/3 | 3/3 |
| Listeria ΔactAΔinlB | 6.67 × 10⁸ | 2/2 | 0/3 |
| Listeria ΔactAΔinlB | 1.12 × 10⁸ | 3/3 | 0/3 |
| Listeria ΔactAΔinlB | 5.57 × 10⁷ | 1/3 | 0/3 |
| Listeria ΔactAΔinlB | 1.12 × 10⁷ | 1/2 | 3/3 |

V. Administration of Listeria Activates Immune Cells in the Liver.

L. monocytogenes was administered to mice followed by assessment of the in vivo modulation of immune response, as determined by extracting the immune cells from the liver and spleen, and by identifying these cells. Except where indicated, Listeria was administered to mice at t=0 hours, followed by sacrifice at t=24 hours. In the time course experiments, where indicated, mice were sacrificed at t=24 hours or at t=48 hours. Livers and spleens were homogenized and dispersed. Cells were washed twice with Hanks Balanced Salt Solution (HBSS), then blocked for 15 min on ice with 4% HAB and anti-CD16/32 antibody. HAB is “Hanks Azide Buffer,” which contains 1% bovine serum albumin, 0.1% sodium azide, and 1 mM EDTA.

Antibody specific for the cell marker of interest was added, and cells incubated 30 minutes on ice. Cells were washed three times, then suspended in 1% formaldehyde, and analyzed by Fluorescence Activated Cell Sorting (FACS). L. monocytogenes (ΔactA or ΔactAΔinlB ) was administered at an amount equivalent to zero LD₅₀ (HBSS only); 0.01 LD₅₀; 0.1 LD₅₀; or 0.25 LD₅₀. Table 6 discloses some of the parameters studied in the following experiments.

TABLE 6
Parameters measured in immune cells extracted from liver and spleen.

% NK cells compared to total leukocytes.
NK cell activation (CD69)
% NKT cells compared to total leukocytes.
NKT cell activation (CD69)
% T cells compared to total leukocytes.
% CD8⁺ T cells compared to total leukocytes.
% CD4⁺ T cells compared to total leukocytes.
CD4⁺ T cell activation (CD69)
CD8⁺ T cell activation (CD69)
% neutrophils compared to total leukocytes.
% of CD4⁺ T cells that are CD4⁺CD25⁺ T cells.
Time courses for changes in the % of NK cells and neutrophils.

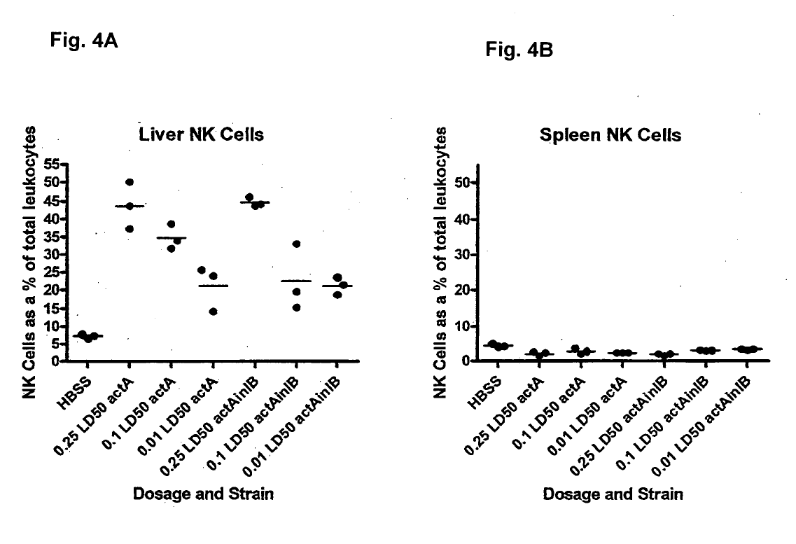

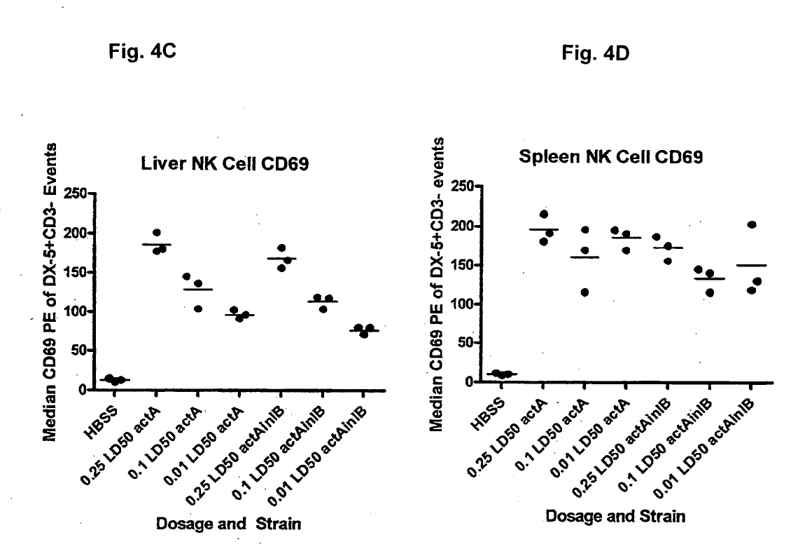

The results were as follows (FIGS. 4A to 4D). The percent of NK cells (% of total leukocytes) increased in the liver, with increasing doses of Listeria. With increasing doses, the percent of total leukocytes that was NK cells increased from about 7% (only HBSS administered, no bacteria), about 20% (dose of 0.01 LD₅₀); about 35% (0.1 LD₅₀); and about 44% (0.25 LD₅₀) (FIG. 4A). NK cell activation in the liver, as assessed by mean fluorescence intensity of expressed CD69, increased from about 10 (arbitrary units where value in absence of cells is zero) (HBSS only, no bacteria); to about 100 (0.01 LD₅₀); to about 130 (0.1 LD₅₀), to about 190 (0.25 LD₅₀) (FIG. 4C). The designation “only HBSS administered” means that no bacteria were administered, and that the data point represents a control value. FIGS. 4B and 4D disclose spleen data.

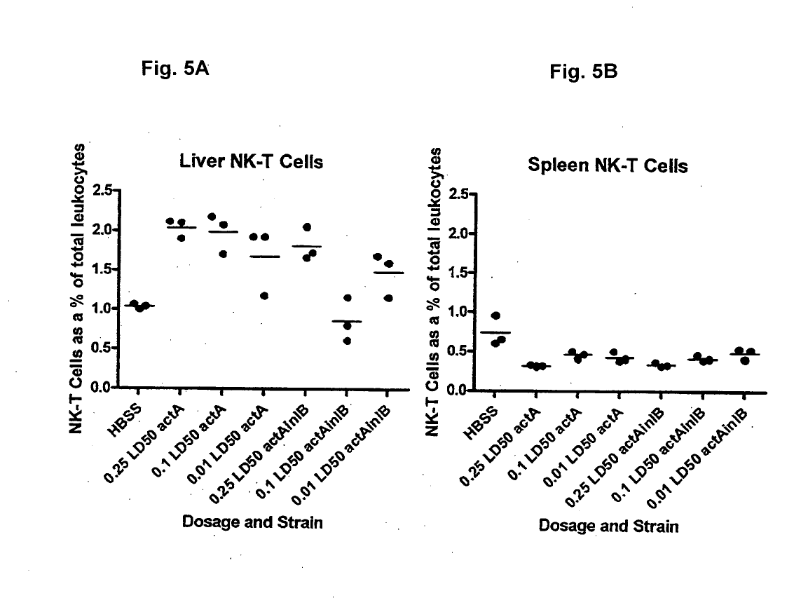

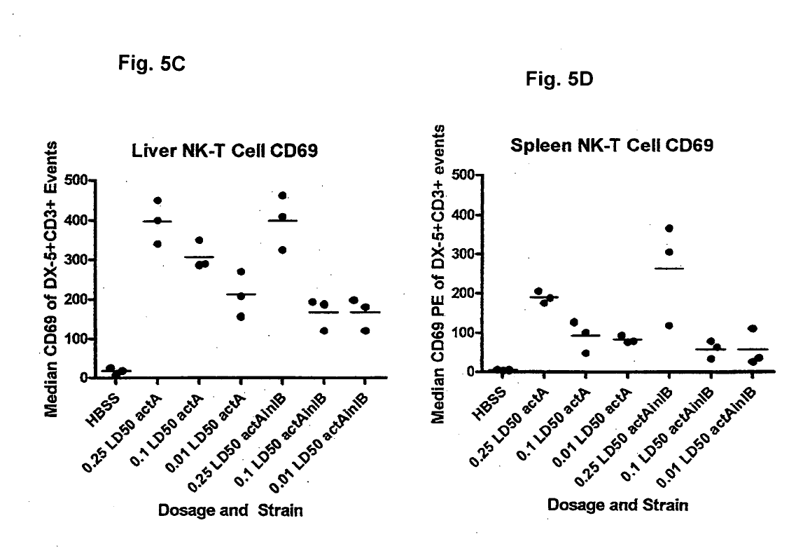

The following concerns NKT cells. Activation of NKT cells in the liver increased with administration of Listeria, where activation after giving Listeria ΔactA was about 5 (HBSS only, no bacteria); 200 (0.01 LD₅₀); 300 (0.1 LD₅₀); and 400 (0.25 LD₅₀) (FIGS. 5A and 5C). After administering the other deletion mutant of Listeria (Listeria ΔactAΔinlB ), maximal activation was also found with administration of 0.25 LD₅₀. (The term “maximal activation” means that maximal activation found with the indicated doses, and does not necessarily mean that higher doses cannot generate even higher states of activation.) (FIGS. 5A and 5C). FIGS. 5B and 5D reveal spleen data.

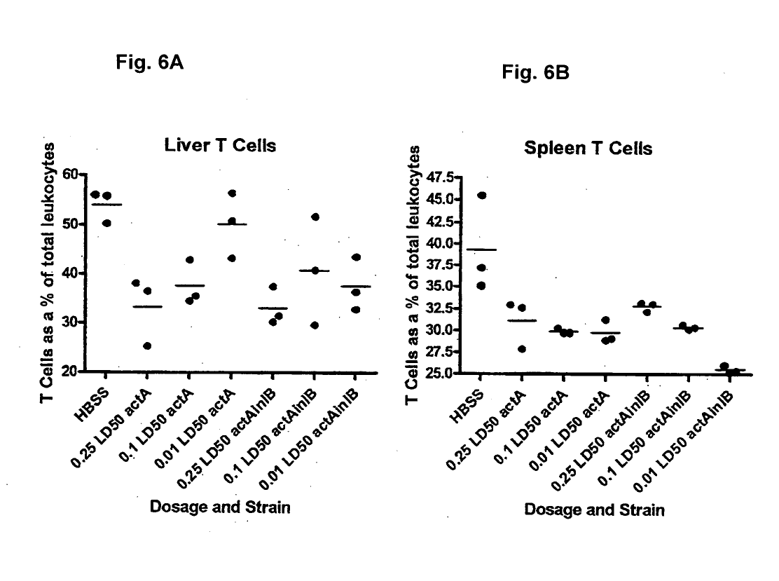

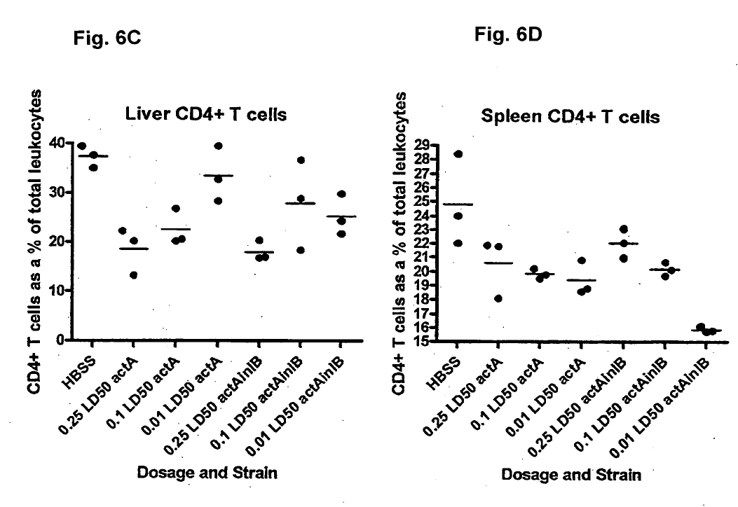

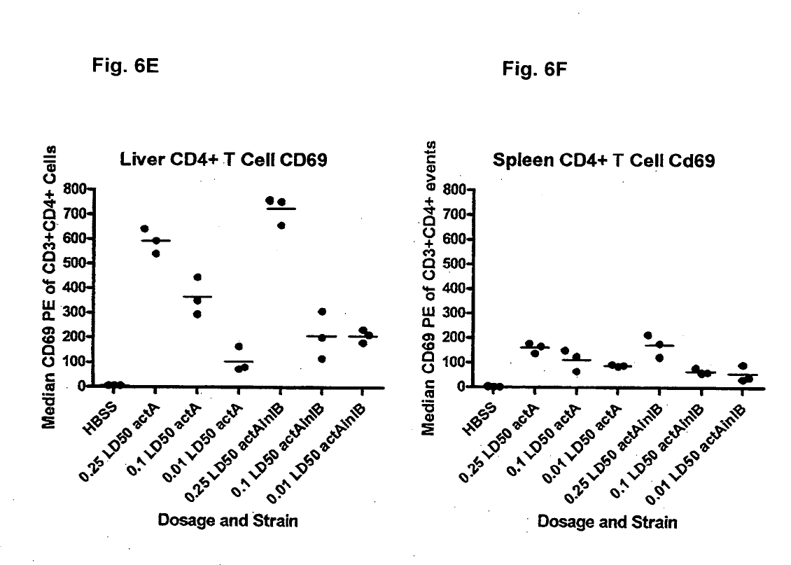

FIGS. 6A and 6B discloses results with total liver T cells.

The following concerns CD4⁺T cells in the liver (FIGS. 6C to 6F). After administering Listeria ΔactA, activation was about 0 (HBSS only, no bacteria), 100 (0.01 LD₅₀), 350 (0.1 LD₅₀), and 600 (0.25 LD₅₀). With administering the other Listeria strain, Listeria ΔactAΔinlB , maximal activation also occurred at the highest dose (FIGS. 6A, C, and E). FIGS. 6B, D, and F disclose spleen data.

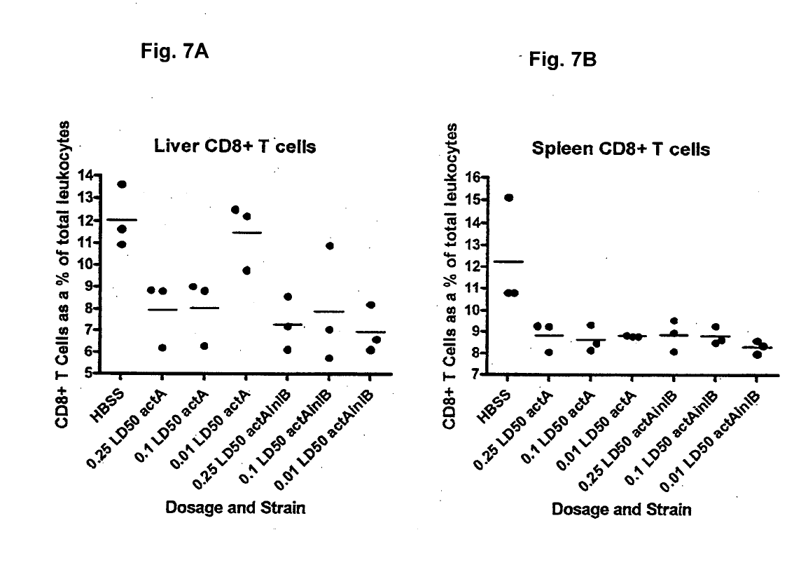

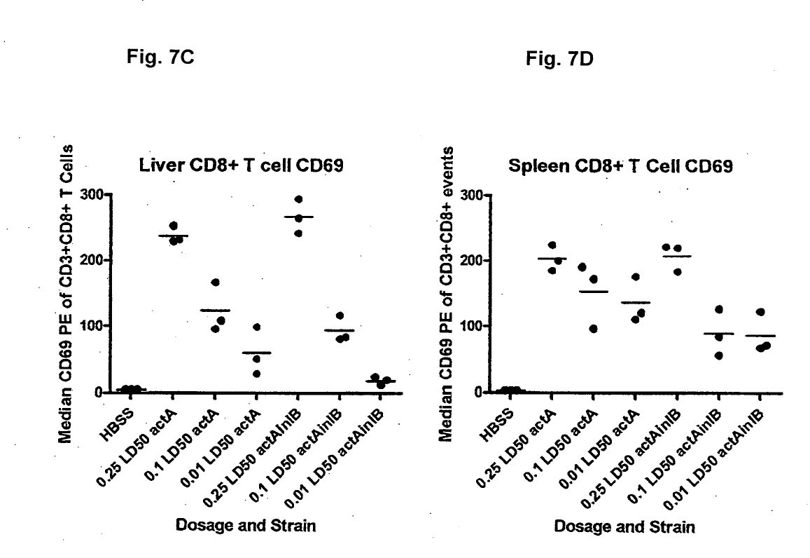

The following concerns CD8⁺T cells in the liver (FIGS. 7A to 7D). Activation of CD8⁺T cells in liver with Listeria ΔactA was about 0 (HBSS only, no bacteria), 60 (0.01 LD₅₀), 120 (0.1 LD₅₀), and 230 (0.25 LD₅₀). Administration of the other strain of Listeria, Listeria ΔactAΔinlB , produced a similar activation profile (FIGS. 7A and 7C. FIGS. 7B and 7D show spleen data.

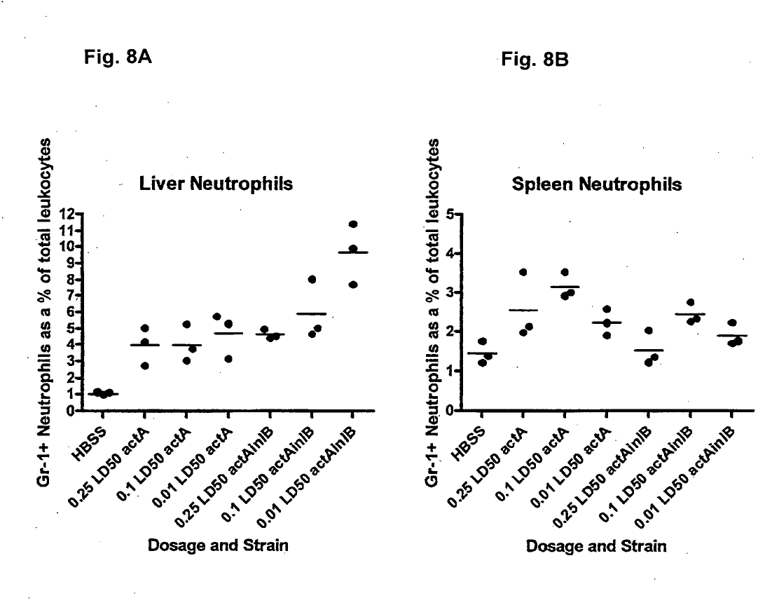

The following concerns neutrophils (FIGS. 8A and 8B). Liver neutrophils increased from about 1% (HBSS only, no bacteria) to about 4-5%, with all three doses of administered Listeria ΔactA. With administered Listeria ΔactAΔinlB , the neutrophils accounted for about 5-10% of the total leukocytes (FIGS. 8A). FIG. 8B shows spleen data.

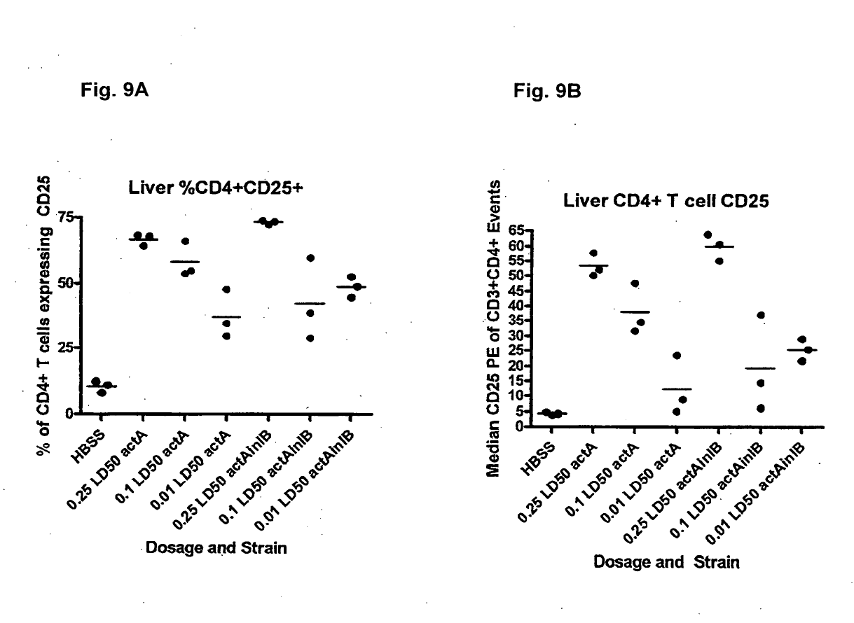

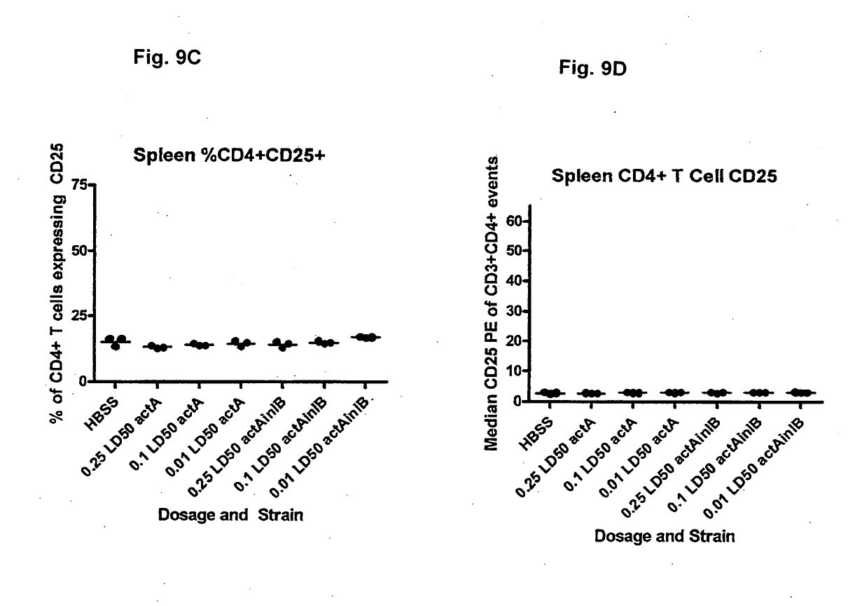

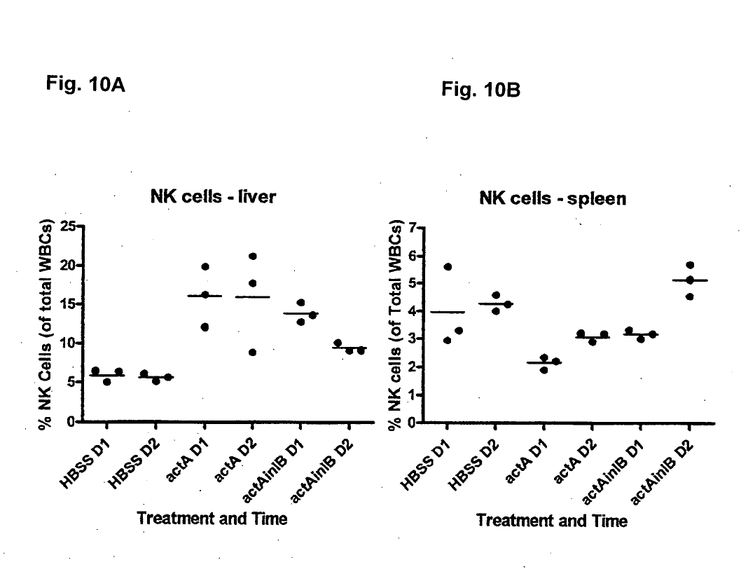

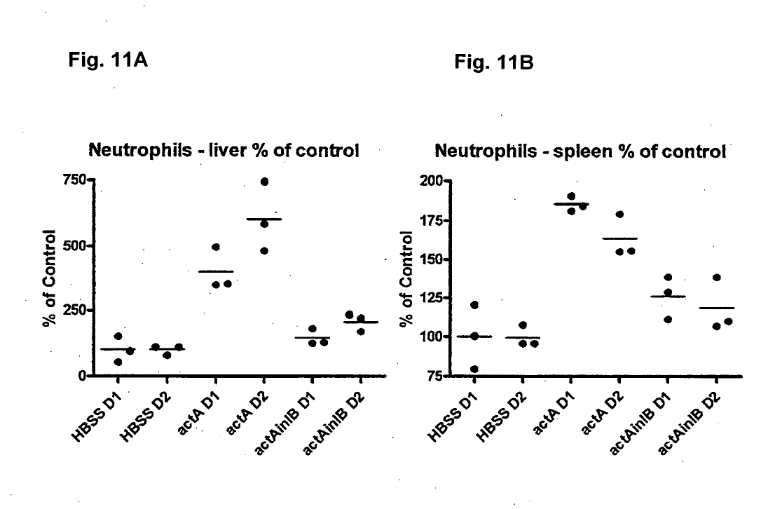

The presence of CD4⁺T cells expressing CD25 was also measured, as was the mean amount of CD25 expressed on individual cells (FIGS. 9A to 9D). CD25 expression was measured after administering Listeria ΔactA or Listeria ΔactAΔinlB . Data from liver CD4⁺T cells and spleen CD4⁺T cells are shown (FIGS. 9A to 9D).

The following concerns dendritic cells, that is, CD8+ alpha negative dendritic cells. Control mice were administered HBSS, while experimental mice were given L. monocytogenes ΔactA (expressing ova). The percentage of these dendritic cells, compared to all splenocytes, was determined over the course of several days. A goal of the present work was to determine the effect of administered Listeria on this dendritic cell population. (For assessing this goal, it is not expected to be relevant if the Listeria expresses ova.) Maturation of the DCs was also measured, as assessed by the markers CD80 and CD86. CD80 and CD86 are DC maturation markers (Gerosa, et al. (2005) J. Immunol. 174:727-734; Kubo, et al. (2004) J. Immunol. 173:7249-7258). For these dendritic cells, control treatment (HBSS salt solution) resulted in relatively constant percentage values (2.0% (day 1); 1.9% (day 2); 1.9% (day 4); 1.6% (day 7)). Experimental treatment (Listeria ΔactA ova) resulted in marked increases in the percent of this type of dendritic cell (3.4% (day 1); 7.3% (day 2); 2.0% (day 4); 1.9% (day 7)). Regarding the CD80 and CD86 markers, the following results were found. Control treatment (HBSS salt solution) of mice resulted in the following CD80 relative expression values for DCs isolated from the spleen: 105 (day 1); 78 (day 2); 91 (day 3), 53 (day 4). Experimental treatment (Listeria ΔactA ova) resulted in dramatic increases in these CD80 expression expression values, that is, on days one and two: 372 (day 1); 298 (day 2); 98 (day 3); 102 (day 7). The following data concern the other marker, CD86. Control treatment (HBSS salt solution) resulted in these CD86 expression values: 31 (day 1); 18 (day 2); 30 (day 4); and 30 (day 7). Experimental treatment provoked a dramatic increase in CD86 expression on days one and two: 257 (day 1); 80 (day 2); 38 (day 4); and 24 (day 7).

The above results, which concern populations of dendritic cells, and the maturation of dendritic cells, are important for immune response to tumors and infections, for a number of reasons. To give two examples, an administered Listeria that enhances DC populations or DC maturation is expected to enhance NK cell function and also to relieve the suppressive effects of regulatory T cells (see, e.g., Gerosa, et al. (2005) J. Immunol. 174:727-734; Kubo, et al. (2004) J. Immunol. 173:7249-7258).

VI. Time Course Studies with Administration of Attenuated Listeria, with Data Disclosing Stimulation of NK Cells and Neutrophils.

Mice were administered HBSS, Listeria ΔactA, or Listeria ΔactAΔinlB , and sacrificed 24 hours later (D1) or 48 hours later (D2), followed by determinations of the number of NK cells or neutrophils, as compared to the total number of leukocytes. Data from analysis of leukocytes recovered from the liver demonstrated that the percent of leukocytes occurring as NK cells was the same on both days (about 6%) with doses of HBSS, the same on both days (about 16%) with doses of Listeria ΔactA, and somewhat greater at t=24 hours (14%) than at t=48 hours (10%) after doses of the other Listeria1 strain, Listeria ΔactAΔinlB (FIG. 10A). FIG. 10B discloses spleen data.

Data from the analysis of neutrophils recovered from the liver demonstrated that in HBSS-administered mice, neutrophils accounted for about 0.2 to 0.8% of liver leukocytes. One day after administering Listeria ΔactA, neutrophils accounted for about 3% of the liver leukocytes, with lesser percent values found under the other conditions of the experiment (FIG. 11A). FIG. 11B discloses spleen data.

A separate study revealed that administering Lm ΔactAΔinlB to mice resulted in the in vivo generation of activated NK cells, where the activated NK cells showed an enhanced ability to kill YAC-1 cells, in vitro. YAC-1 cells are conventionally used as an NK cell target. C57BL/6 mice were injected with 3×10⁷ cfu of Lm ΔactAΔinlB , or with a negative control vehicle. After a delay of 24 h, 48 h, or 72 h, lymphocytes were harvested from the liver or spleen, and the harvested lymphocytes (contains NK cells) were mixed with chromium-labeled YAC-1 cells (the target cells), and then incubated for 4 h. With lymphocytes harvested at the 48 h time point, for example, liver NK cells produced about 50% lysis of the target cells (whereas only 3% target cell lysis occurred where lymphocytes were from vehicle-treated mice). With lymphocytes harvested at the 48 h time point, spleen NK cells produced about 30% lysis of the target cells (whereas only 7% lysis of target cells occurred where lymphocytes were from vehicle-treated mice). Thus, the methods of the invention provide for administering Lm for activating and/or increasing hepatic levels of NK cells, where the NK cells are effective at lysing target cells.

VII. Administering Listeria Increases Numbers of Immune Cells in the Liver (Time Course Studies).

The following discloses the time course of accumulation of various immune cells in the liver following administration of Listeria ΔactA. Concurrent work illustrates the influence, on immune cell accumulation, produced by administering only tumor cells engineered to express GM-CSF (GVAX), or produced by administering Listeria ΔactA together with GVAX.

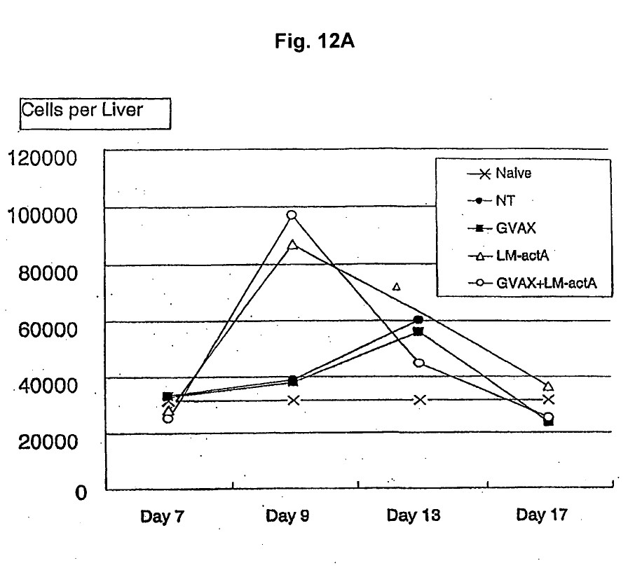

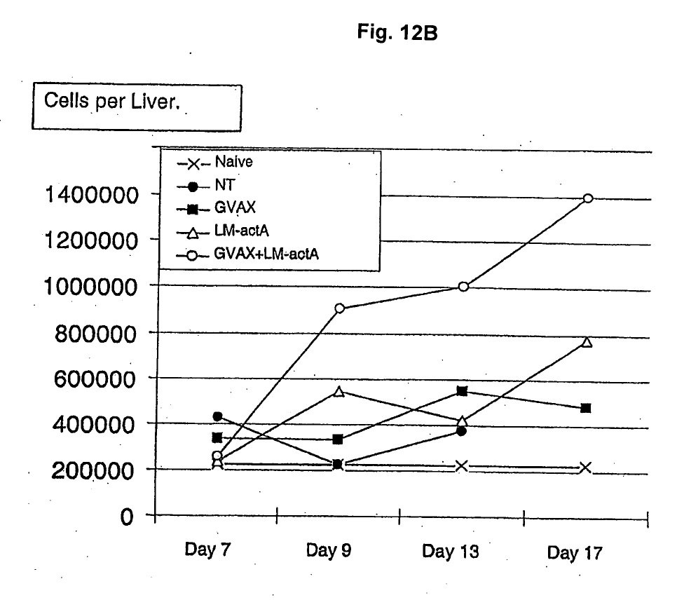

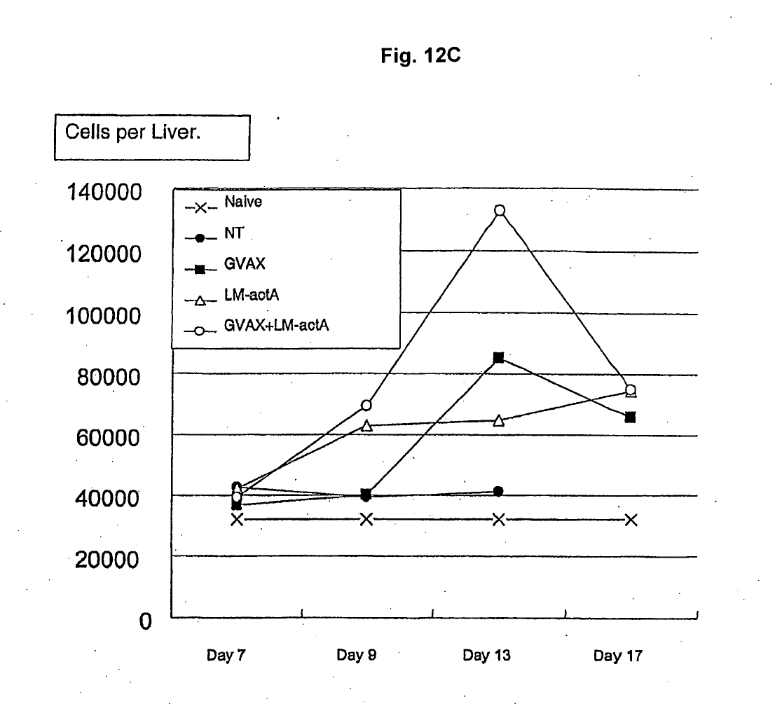

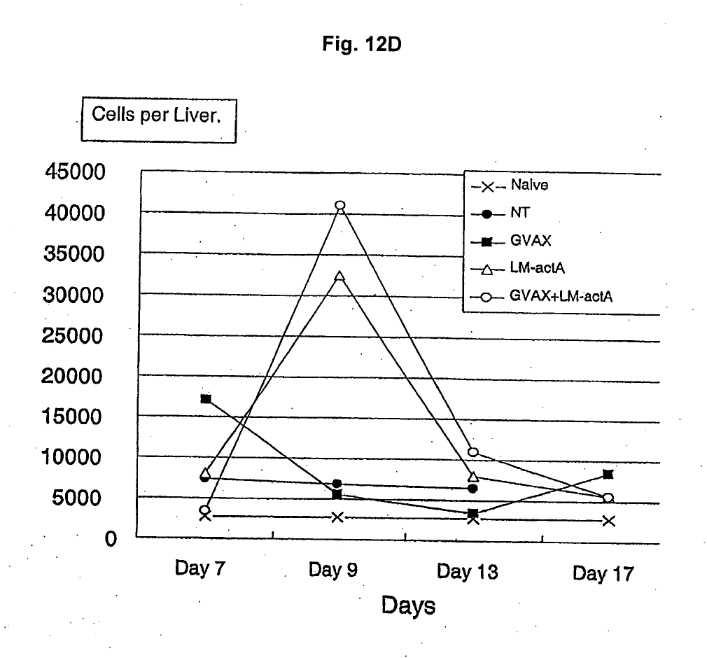

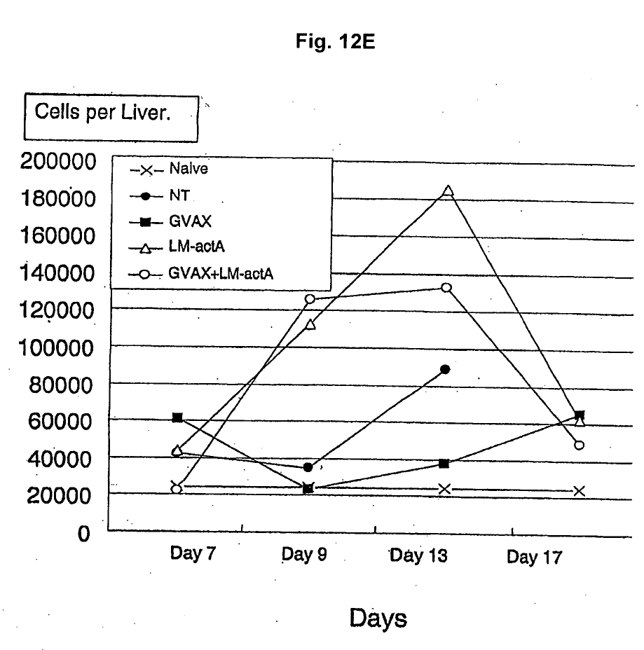

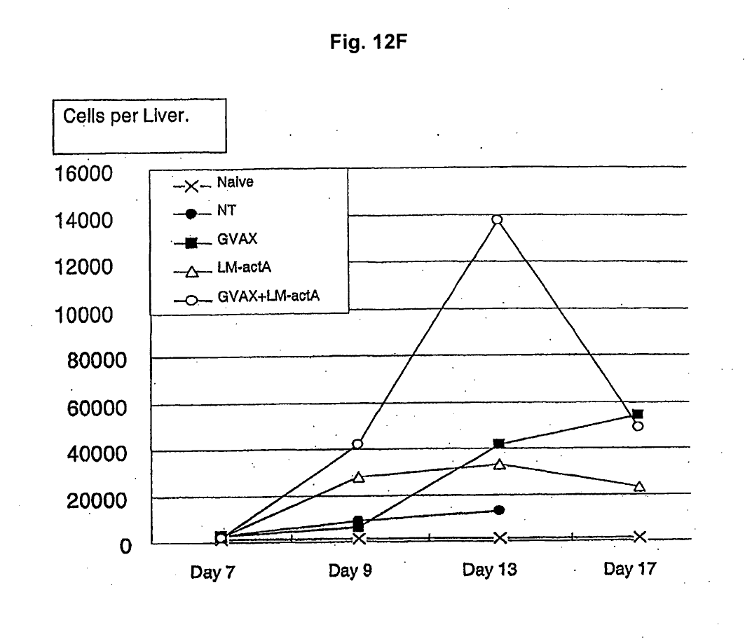

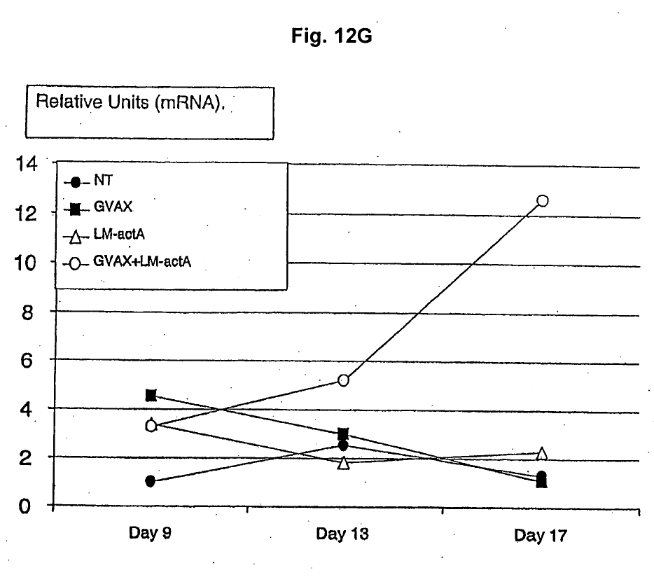

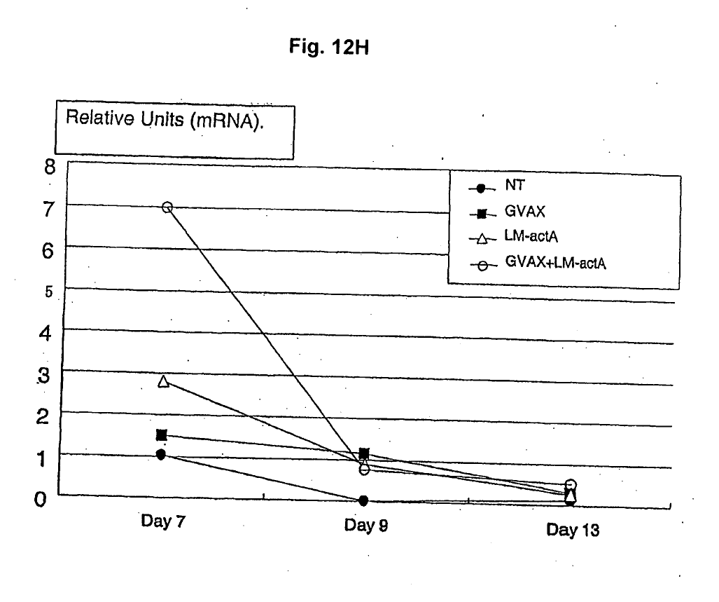

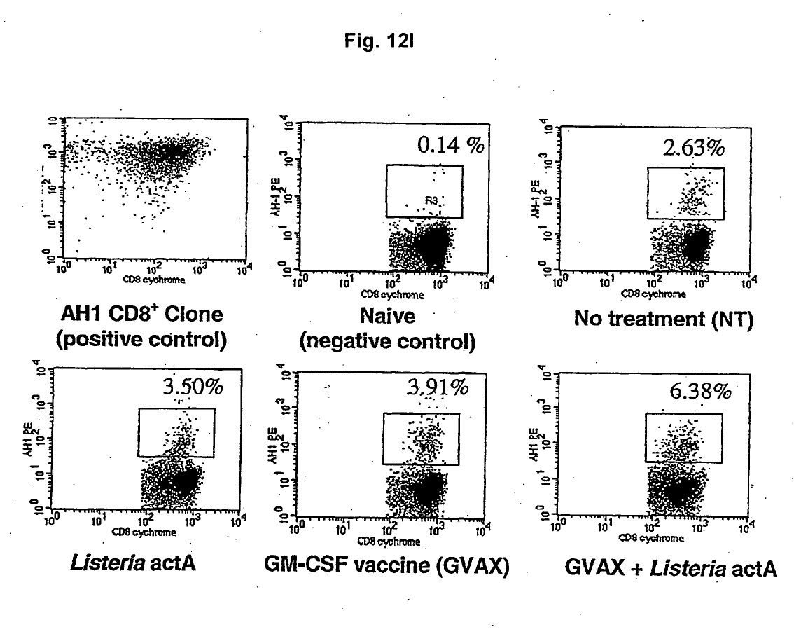

Balb/c mice were treated under the following conditions, followed by measuring the number of various immune cells in the liver. The treatments were:
(1) Naive mice (not administered any tumor cells);
(2) No treatment (NT) mice (administered tumor cells but not treated with Listeria and not treated with GVAX);
(3) Administered tumor cells and GVAX;
(4) Administered tumor cells and Listeria ΔactA (Lm-actA); and
(5) Administered tumor cells, GVAX, and Listeria ΔactA (Lm-actA).

Where Listeria ΔactA was given, the number of administered bacteria was 1×10⁷ CFU. The immune cells that were identified and counted were: NK cells (FIG. 12A); NKT cells (FIG. 12B); CD8⁺T cells (FIG. 12C); plasmacytoid dendritic cells (plasmacytoid DCs) (FIG. 12D); myeloid DCs (FIG. 12E); and tumor specific CD8⁺T cells (FIG. 12F). The activation state of tumor specific CD8⁺T cells (in the liver) was assessed by measuring expression of interferon-gamma (IFNgamma mRNA) (FIG. 12G). The activation state of NK cells (in the liver) was also assessed, where activation was assessed by measuring IFNgamma mRNA (FIG. 12H).

The results were as follows. Regarding the general baseline population range, the dendritic cells (DCs) in the liver tended occur at the lowest population ranges while NK cells, NKT cells, and CD8⁺T cells tended to occur at the highest population ranges. The baselines for all cell types was constant for the naive mice (FIGS. 12A-12F). When Listeria alone was administered to tumor-bearing mice, the NK cell population showed a peak at about t=9 days (FIG. 12A); the NKT cell population showed an increasing trend up to at least 17 days (FIG. 12B); CD8⁺T cells showed a steady increasing trend up to at least 17 days (FIG. 12C); plasmacytoid DCs showed a peak at about t=9 days (FIG. 12D); the myeloid DC population peaked at about t=13 days (FIG. 12E); while tumor-specific CD8⁺T cells peaked at about t=13 days (FIG. 12F).

GVAX alone increased the populations of all of the immune cells (FIGS. 12A-12F). Listeria in combination with GVAX revealed additive effects, or synergic effects, in the cases of NKT cells (FIG. 12B); CD8⁺T cells (FIG. 12C); plasmacytoid DCs (FIG. 12D); and tumor specific CD8⁺T cells (FIG. 12F).

The activation state of a number of immune cells was assessed, where assessment was by assays of interferon-gamma (IFN-gamma) mRNA. Assays for IFN-gamma mRNA expressed by tumor specific CD8⁺T cells revealed that the greatest increase in expression occurred with administration of both Listeria and GVAX to the mice (FIG. 12G). Assays for IFN-gamma mRNA expressed by NK cells also showed that the greatest increase in expression occurred with administration of both Listeria and GVAX to the mice (FIG. 12H). With regard to the mice receiving both Listeria and GVAX, a difference was noted in following IFN-gamma expression by the tumor specific CD8⁺T cells and NK cells, namely that expression by the CD8⁺T cells was highest at later time periods, while expression by the NK cells was highest at the earlier time periods (FIGS. 12G and H).

The following concerns FIG. 121. FIG. 121 shows analysis of CD8⁺T cells taken from livers of CT26 tumor cell-innoculated mice, where the mice had also been administered, e.g., various therapeutic agents. The therapeutic treatments, including controls, included no therapeutic treatment (NT); L. monocytogenes ΔactA; GM-CSF vaccine only (GVAX); and L. monocytogenes ΔactA plus GVAX. With no therapeutic treatment (NT), the percent of tumor antigen-specific CD8⁺T cells was 2.63%.

The results were as follows. With Listeria only, the percent of tumor antigen-specific CD8⁺T cells was higher (3.5%); with GVAX only, and the percent of tumor antigen-specific CD8⁺T cells was also higher (3.91%). But with Listeria plus GVAX the percent of expression of tumor antigen-specific CD8⁺T cells was much higher (6.38%), demonstrating synergy between the Listeria and the GM-CSF vaccine (FIG. 121).

In detail, the figure illustrates analysis of tumor-specific CD8⁺T cells that infiltrate the liver in treated mice with hepatic metastases. Specific flow cytometry plots on cells isolated from the livers of mice sacrificed on day 13 and stained with anti-CD8 (FITC) and L d-AH 1 tetramers (cychrome) are shown. Note that AH 1 is the immunodominant MHC class I-restricted tumor antigen recognized by CT-26-specific CD8⁺T cells. The study involved positive and negative controls (AHI-specific CD8⁺T cell clone as a positive control; and hepatic CD8+cells from naive non-tumor-bearing mice as a negative control). The data represent the results from the pooled and processed livers of three mice. Treatment with both CT-26/GM-CSF and Listeria ΔactA resulted in the highest level of hepatic AH 1-specific CD8⁺T cells.

VIII. Administering an Attenuated Tumor Cell Line that Expresses GM-CSF Increases Survival to Tumors, While Administering that Tumor Cell Line with Listeria ΔactA or Listeria ΔactAΔinlB Further Increases Survival to Tumors.

Tumor bearing mice were treated by administering: (1) Salt water only (HBSS); (2) A vaccine comprising a tumor cell line secreting a cytokine (CT26 cells expressing the cytokine GM-CSF) (GM-CSF vaccine); (3) The vaccine plus Listeria ΔactA; or (4) The vaccine plus Listeria ΔactAΔinlB .

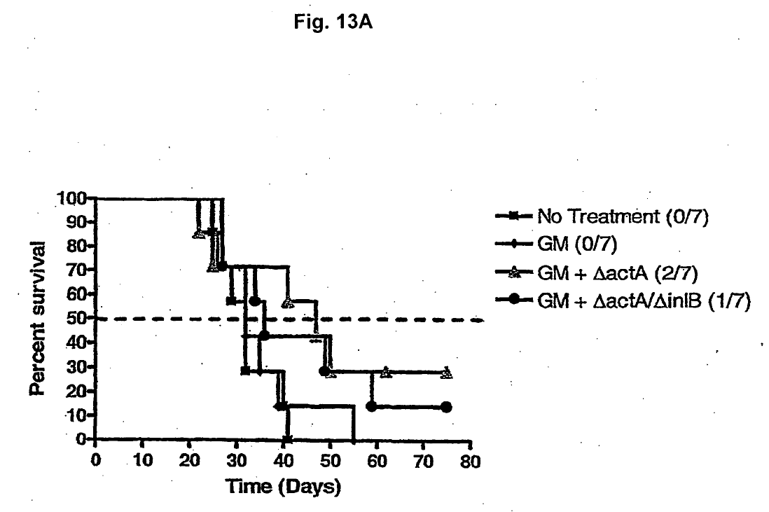

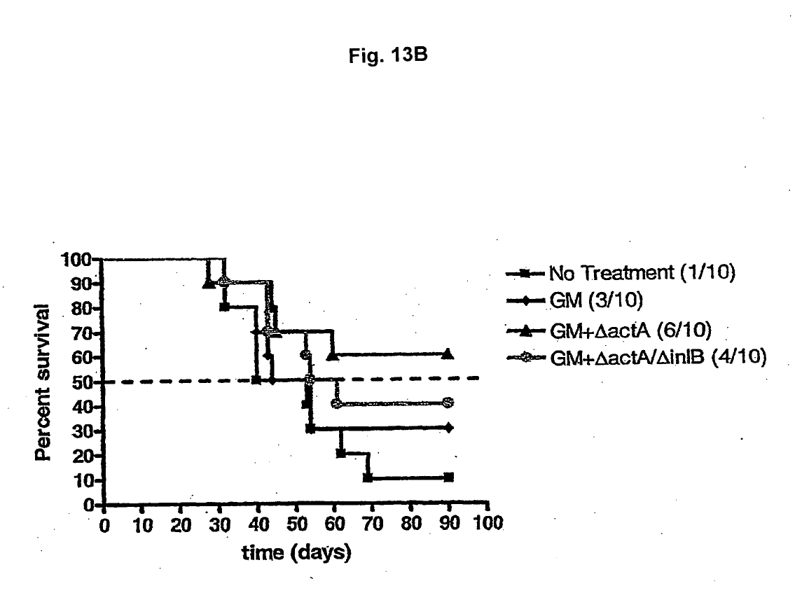

Tumor cells (1×10⁵ CT26 cells) in 0.05 ml HBSS were administered into the hemispleen, followed by a flush of 0.25 ml HBSS. Irradiated GM-CSF expressing CT26 cells (1×10⁶ cells) (also known as “vaccine”) were administered in 0.30 ml of HBSS, with 0.10 ml injection per site (subcutaneously; s.c.). Listeria was administered in amount equivalent to 0.1 LD₅₀, where administration was in 0.20 ml HBSS (i.p.) or in 0.10 ml HBSS (intravenously; i.v.). The time line for the various administrations during the course of the experiment was as follows: tumor (t=0 days); vaccine (t=3 days); vaccine plus Listeria (t=6 days); vaccine (t=13 days); and vaccine (t=21 days). Conditions of the experiment included no treatment (-▴-; filled squares); vaccine only (-⋄-; diamonds); vaccine plus Listeria ΔactA (-▴-; filled triangles); and vaccine plus Listeria ΔactAΔinlB (-●-; filled circles). CT26 tumor cells were administered at t=day zero, while GM-CSF vaccine was given at t=3 days, and Listeria provided at t=6 days. For FIGS. 13A and 13B, the Listeria dose was 1×10⁷ CFU.

FIG. 13A discloses the percent survival of the mice versus time (days) during the study. The results demonstrated that in the “no treatment” group, there were zero survivors by t=40 days, and that survival was somewhat greater in the vaccine only group, with zero survivors by t=55 days. The vaccine plus Listeria groups resulted in markedly enhanced survival, with about 28% survival at t=48 days in both vaccine plus Listeria ΔactA group and vaccine plus Listeria ΔactAΔinlB group, while at t=75 days, 28% survival was found in the vaccine plus Listeria ΔactA group, and about 15% survival in the vaccine plus Listeria ΔactAΔinlB group (FIG. 13A). FIG. 13B shows data from a repeated trial of the same experiment as above. Again, mice receiving no treatment showed the poorest survival, with only one mouse surviving at t=90 days. Again, mice receiving the GM-CSF vaccine with Listeria showed the best survival. Here, 6 out of 10 mice receiving the GM-CSF vaccine plus Listeria ΔactA still survived at t=90 days, and 4 out of 10 mice receiving the GM-CSF vaccine plus Listeria ΔactAΔinlB survived at t=90 days (FIG. 13B).

The present invention provides a method comprising administering an attenuated Listeria (e.g., L. monocytogenes ΔactA or L. monocytogenes ΔactAΔinlB ), with attenuated tumor cells (e.g. irradiated metastatic cells), where the cells had been engineered to express a cytokine, e.g., GM-CSF. In the present invention, the Listeria are not engineered to comprise any nucleic acid encoding any heterologous antigen, e.g., a tumor or infectious agent antigen. In another aspect of the present invention, the Listeria are engineered to comprise a nucleic acid encoding a heterologous antigen.

IX. Cyclophosphamide Increases Survival to Tumors.

Administering cyclophosphamide (CTX) increased survival of mice bearing tumors under each of these three conditions:
(1) Mice treated with GM-CSF vaccine only;
(2) Mice treated with GM-CSF vaccine plus Listeria ΔactA;
(3) Mice treated with GM-CSF vaccine plus Listeria ΔactAΔinlB .

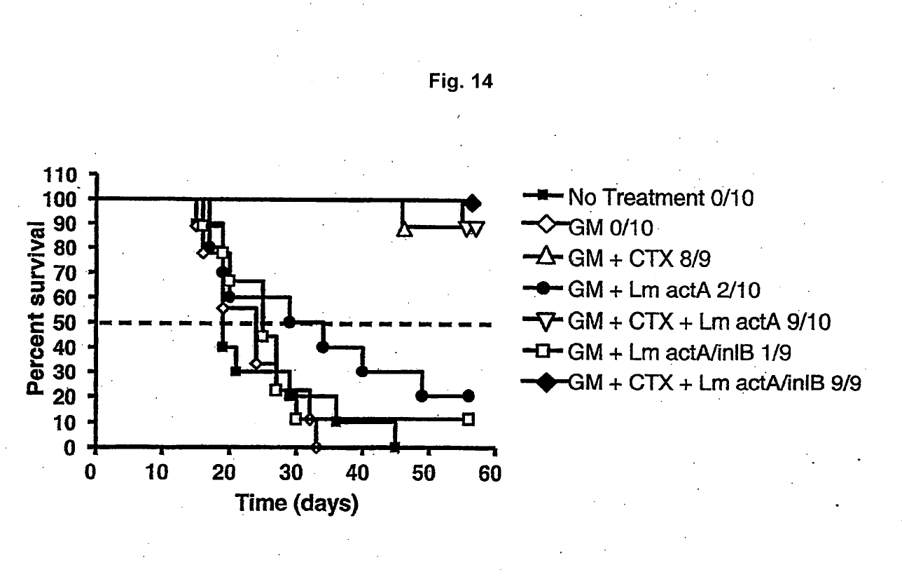

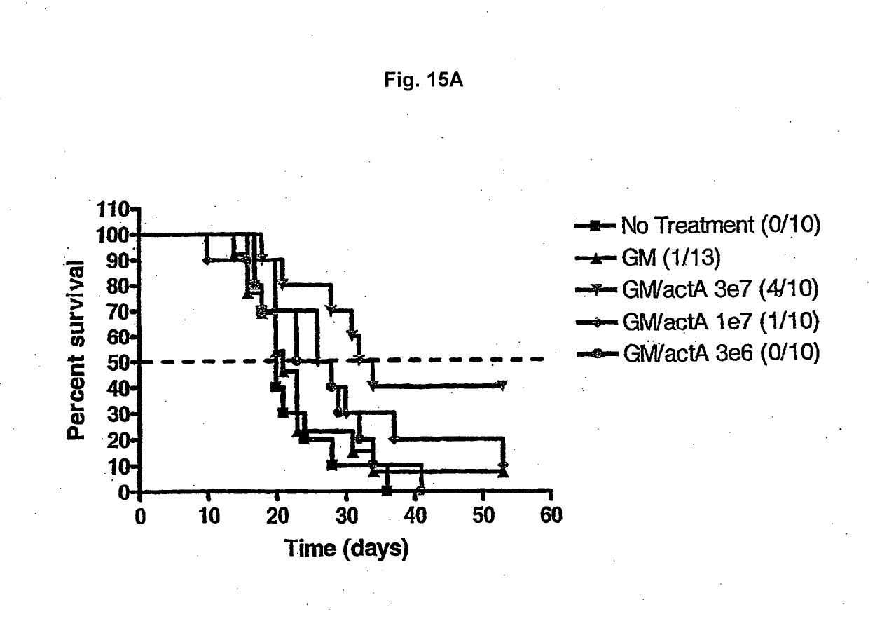

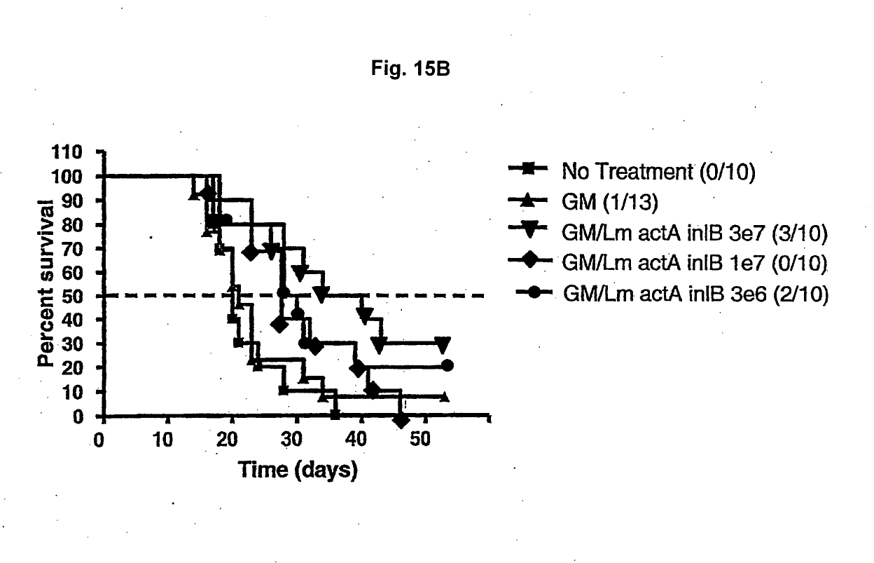

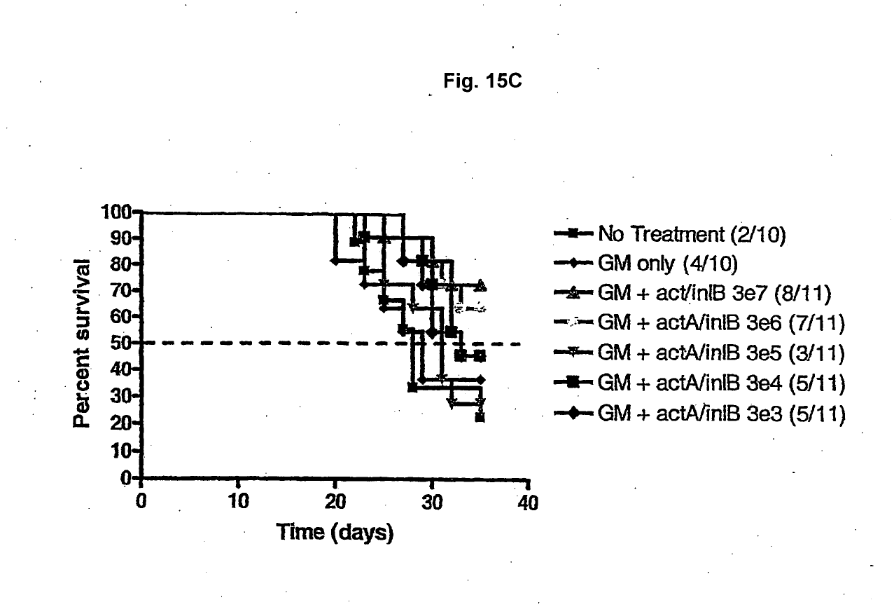

Mice were inoculated with CT26 tumor cells on day zero (FIG. 14). The dose of the CT26 tumor cells used to generate the tumors was 0.1 million cells. Therapeutic treatment was as follows: no treatment (-▪-; filled squares); treatment with GM-CSF vaccine only (-♦-; open diamonds); treatment with GM-CSF vaccine and cyclophosphamide (CTX) (-Δ-; open triangles); treatment with GM-CSF plus Listeria ΔactA (-●-; filled circles); treatment with GM-CSF, cyclophosphamide, and Listeria ΔactA (-V-; open inverted triangles); GM-CSF plus Listeria ΔactAΔinlB (-□-; open squares); or treatment with GM-CSF, cyclophosphamide, and Listeria ΔactAΔinlB (-♦-; filled diamonds) (FIG. 15). CTX was given at 100 mg CTX per kg body weight (intraperitoneally; i.p.). Cyclophosphamide was from Sigma (St. Louis, Mo.), and dissolved in HBSS before injecting in animals.

Tumor cells were administered at day zero. For this study, each mouse receiving the GM-CSF vaccine received three doses of the GM-CSF vaccine (at t=3, 15, and 31 days). Where cyclophosphamide was administered, there was only one dose, and it was given at t=day 2. Listeria ΔactA was administered at t=6, 19, and 34 days (1×10⁷ CFU). Listeria ΔactAΔinlB was also administered at the same days, and at the same dosage (t=6, 19, and 34 days (1×10⁷ CFU)) (FIG. 14).

Lowest rates of survival were found in the no treatment group, and in mice receiving GM-CSF vaccine only (FIG. 14). Mice treated with the GM-CSF vaccine plus Listeria ΔactA showed a marked increase in survival time, where about 30% survival was found at t=40 days. The following concerns groups receiving CTX. Where the GM-CSF vaccine was supplemented with CTX only, 90% survival was found at t=45 days. Greater rates of survival were found when the GM-CSF vaccine was supplemented with CTX plus Listeria . For example, when the GM-CSF vaccine was supplemented with CTX plus Listeria ΔactAΔinlB , survival at t=55 days was 100% (-♦-; filled diamonds) (FIG. 15).

The present invention provides a method comprising administering an attenuated Listeria (e.g., L. monocytogenes ΔactA or L. monocytogenes ΔactAΔinlB ), with attenuated tumor cells (e.g. irradiated metastatic cells), where the cells had been engineered to express a cytokine, e.g., GM-CSF, with an agent that inhibits action of T regulatory cells (e.g., CTX). In the present invention, the Listeria are not engineered to comprise any nucleic acid encoding any heterologous antigen, e.g., a tumor or infectious agent antigen.

X. Titrating Tumor-Bearing Mice with Listeria , with Constant Administration of Vaccine.

FIGS. 15A to 15C disclose results where various numbers of Listeria were administered to tumor-bearing mice (constant administration of vaccine). In detail, the work involved titrating CT26 cell-tumor bearing mice with Listeria ΔactA (constant GM-CSF vaccine treatment) or with Listeria ΔactAΔinlB (constant GM-CSF vaccine treatment).

In the following studies, tumor-bearing mice were “titrated” with various amounts of attenuated Listeria. In all cases, GM-CSF vaccine was administered on three days (at t=days 3, 17, and 31), and in all cases, Listeria ΔactA (or Listeria ΔactAΔinlB ) was administered on three days (at t=days 6, 20, and 34).

Mice were inoculated with CT26 tumor cells. Mice received either no treatment (-▪-; squares); GM-CSF vaccine only (-▴-; triangles); GM-CSF vaccine with 3×10⁷ Listeria (- V -; inverted triangles); GM-CSF vaccine with 1×10⁷ Listeria (-♦-; diamonds); or GM-CSF vaccine with 3×10⁶ Listeria (-●-; filled circles). FIG. 15A depicts results where the administered attenuated Listeria were deleted in only one virulence gene (Listeria ΔactA) (range of 3×10⁶ to 3×10⁷ bacteria), while FIG. 15B shows results with Listeria deleted in two different virulence genes (Listeria ΔactAΔinlB ) (range of 3×10⁶ to 3×10⁷ bacteria). FIG. 15C also depicts results with Listeria ΔactAΔinlB , where the bacteria were administered in the range of 3×10³ to 3×10⁷ bacteria.

Poorest survival rates were found in mice receiving no treatment or administered the GM-CSF vaccine only. Administration of Listeria, along with the GM-CSF vaccine improved survival, where the low and middle bacterial dose levels (3×10³ to 3×10⁵) appeared to provide similar improvement in survivals. Here, the dose of 3×10⁶ bacteria seemed to work as well as 1×10⁷ bacteria. Even better survival was found at the high dose (3×10⁷ bacteria). At the high bacterial dose (with GM-CSF vaccine), about 30-40% survival was found at t=53 days (FIGS. 15A and B).

FIG. 15C demonstrates that the highest survival rate was obtained with the highest level of administered bacteria (3×10⁷ bacteria; -▴-; triangle), where 70% survival was found at t=35 days. Survival was similar, or slightly lower, with administration of 3×10⁶ bacteria (-●-; filled circle). Still lower levels of survival were found with administration with lesser numbers of bacteria (3×10⁵ bacteria; -▾-; inverted triangle) (3×1 bacteria; -▪-; squares) (3×10³ bacteria; -♦-; diamonds). At one of the levels of administered bacteria (3×10⁵ bacteria; -▾-; triangles), survival was found to be somewhat better than the no treatment group, though survival was as low as the “no treatment” group at time periods after t=30 days. Results from the “no treatment” group (-▪-; squares) and GM-CSF vaccine only group (-♦-; diamonds) were as indicated.

The present invention provides a method of administering an attenuated Listeria (e.g., Listeria ΔactA or Listeria ΔactAΔinlB ) by way of a plurality of doses, and an attenuated tumor vaccine, by way of a plurality of doses. In one aspect, the attenuated tumor is engineered to contain a nucleic acid encoding a cytokine, e.g., GM-CSF. In another aspect, the attenuated tumor is not engineered to contain a nucleic acid encoding a cytokine.

XI. Listeria (Not Containing a Nucleic Acid Encoding a Tumor Antigen) Reduced Tumor Metastases to the Lung.

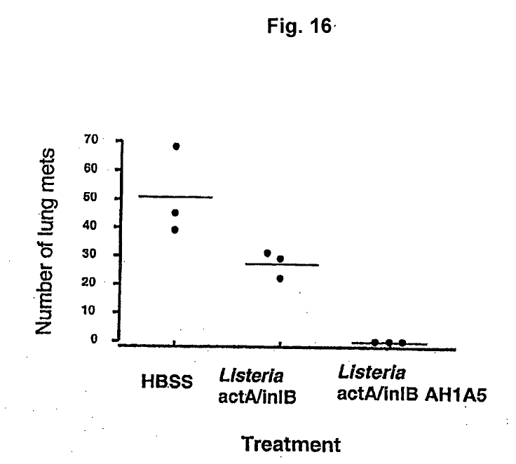

FIG. 16 shows data from lung tumors (not liver tumors). FIG. 16 discloses dose response curves, showing response of lung tumors to various doses of administered Listeria. The tumors arose from CT26 cells injected into the spleen. The figure discloses a control study, where tumor cell-innoculated mice were treated with salt solution (HBSS). Also shown are results from treatment with Listeria ΔactAΔinlB not containing any nucleic acid encoding a tumor antigen (1×10⁷ bacteria administered), and with Listeria ΔactAΔinlB engineered to containing a nucleic acid encoding a positive control tumor antigen (AH1-A5) (1×10⁷ bacteria administered), an epitope derived from gp100. With salt water treatment, there were about fifty lung metastases. With Listeria not engineered to express any tumor antigen, the number of lung metastases was cut in half (about 25-30 lung metastases). With Listeria engineered to express AH1-A5, there were essentially zero lung metastases (FIG. 16).

XII. Listeria (Not Engineered to Contain a Nucleic Acid Encoding a Tumor Antigen) Stimulates Long-Term Adaptive Immunity to Tumors.

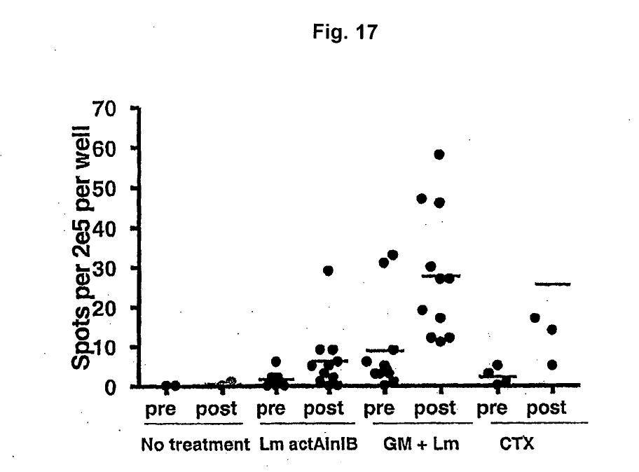

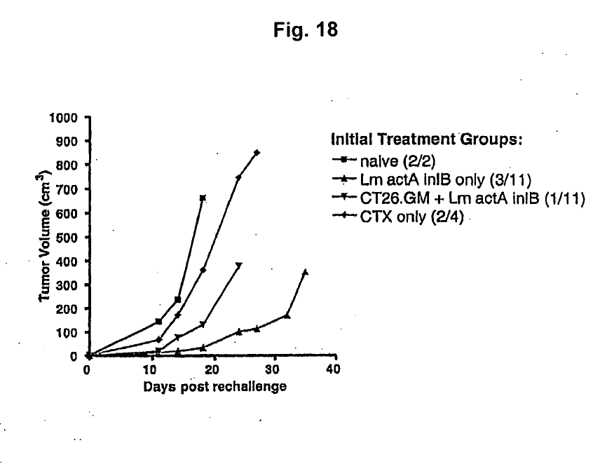

FIGS. 17 and 18 demonstrate that treating tumor-bearing mice with Listeria (Listeria not engineered to encode any heterologous antigen) stimulates adaptive immunity to the tumor, i.e., to antigens of the tumor. Mice were initially inoculated (t=0 days) with CT26 tumor cells by way of the hemispleen model, and then treated with:
(1) No treatment with any therapeutic agent (“naive mice”);
(2) Listeria ΔactAΔinlB (3 cycles of Listeria ΔactAΔinlB beginning at t=3 days after inoculation with the CT26 tumor cells. Administration of Listeria was once weekly for three weeks. The Listeria ΔactAΔinlB had not been engineered to express any tumor antigen;
(3) GM-CSF vaccine with Listeria ΔactAΔinlB (1 injection of Listeria ΔactAΔinlB at t=6 days). Administration of the GM-CSF vaccine was started three days after injecting the tumor cells in the hemispleen, that is, on days 3, 6, and 10; or
(4) Cyclophosphamide (CTX) (50 mg/kg).

At t=100 days (shortly before the re-challenge) and at t=107 days (post re-challenge), surviving mice in each group were assessed for long-term immunity (Elispot assays) to the immunodominant antigen of the CT26 cells (AH1 antigen). The first Elispot assay (pre re-challenge) served as a baseline assay for use in assessing adaptive immune response. The second Elispot assay (107 days; post re-challenge) was used to assess adaptive immune response. At t=102 days, all mice were inoculated with CT26 tumor cells by way of a subcutaneous re-challenge. The subcutaneous CT26 tumor cell re-challenge was with 2×10⁵ cells (twice the dose initially injected in the hemispleen). FIG. 17 demonstrates that the re-challenge with CT26 tumor cells:
(1) Failed to stimulate detectable anti-AH1-immunity in the group of mice that had never been treated with any therapeutic agent (the “no treatment” group);
(2) Produced a detectable, or modest, Elispot response in the mice that had originally received Listeria ΔactAΔinlB alone;
(3) Produced a stronger Elispot response in mice that had originally received both the GM-CSF vaccine and Listeria ΔactAΔinlB ; and
(4) Produced a moderate Elispot response in mice that had originally received only cyclophosphamide (CTX) (FIG. 17).

In short, the results demonstrate that treatment with either Listeria ΔactAΔinlB alone; GM-CSF vaccine and Listeria ΔactAΔinlB ; or cyclophosphamide (CTX) alone, can produce a long term effect on the immune system. The long term effect resulted in clearly detectable immune responses to the re-challenge.

Tumor volume was assessed in the days following the CT26 tumor cell re-challenge (FIG. 18). Tumors resulting from the subcutaneous injection presented as bumps under the skin. The dimensions of these tumors were measured topically. The results demonstrated that, in the days following the re-challenge, tumors arising from the re-challenge grew and increased in volume. However, tumor growth was the greatest in the animals that had never received any therapeutic agent, while tumor growth was significantly inhibited in animals that had initially been treated with the Listeria ΔactAΔinlB alone or with GM-CSF vaccine and Listeria ΔactAΔinlB (FIG. 18).

A number of the mice studied in the re-challenge experiment were found to be tumor-free. Regarding these tumor-free mice, the results demonstrated that none of the naive mice (no therapeutic treatment) (out of 2 naive mice in all) were tumor free following the re-challenge; about 50% of the CTX-only mice (out of 4 CTX-only mice in all) were tumor free; while about 75% of the Listeria ΔactAΔinlB only treated mice (out of 11 Listeria ΔactAΔinlB only mice in all) and about 90% of the GM-CSF vaccine plus Listeria ΔactAΔinlB -treated mice (out of 11 GM-CSF vaccine plus Listeria ΔactAΔinlB in all) were tumor free.

The following concerns tumors induced by MC38 cells, rather than CT26 cells. Separate studies with C57BL/6 mice inoculated with MC38 cells demonstrated that all control mice died by t=43 days, with half dying by about t=38 days. Experimental mice administered 3×10⁷ cfu Lm ΔactAΔinlB (doses at t=3, 10, and 17 days), survived to at least t=90 days. In the Lm ΔactAΔinlB -treated group, about half the mice had died by t=50 days, and about 80% had died by t=90 days. The above commentary on MC38 cells refers to a study where CTX was not administered. In short Lm ΔactAΔinlB improved survival to MC38 cells, without any administered CTX. As mentioned earlier, CT26 tumor cells are from Balb/c mice, whereas MC38 tumor cells are from C57Bl/6 mice, where Balb/c mice are Th2 type responders and C57Bl/6 mice are Th1 type responders.

The present invention provides a method comprising administration of a metabolically active Listeria for stimulating adaptive immunity (including long-term adaptive immunity; memory response; and recall response), e.g., to a tumor, cancer, infectious agent, viral, parasitic, or bacterial antigen. The invention encompasses the above method, further comprising administration of one or more of a cytokine, e.g., GM-CSF, an attenuated tumor, an attenuated tumor expressing the cytokine, or an inhibitor of Tregs, such as cyclophosphamide (CTX). In another aspect, the above invention comprises the above method, where the Listeria is not engineered to express a heterologous antigen, e.g., an antigen derived from a tumor cell, cancer cell, or infective agent.

Also provided is a method comprising administering a metabolically active attenuated Listeria for stimulating adaptive immunity (including long-term adaptive immunity; memory response; and recall response), e.g., to a tumor, cancer, infectious agent, viral, parasitic, or bacterial antigen. The invention encompasses the above method, further comprising administration of one or more of a cytokine, e.g., GM-CSF, an attenuated tumor, an attenuated tumor expressing the cytokine, or an inhibitor of Tregs, such as cyclophosphamide (CTX). In another aspect, the above invention comprises the above method, where the Listeria is not engineered to express a heterologous antigen, e.g., an antigen derived from a tumor cell, cancer cell, or infective agent.

XIII. Cytokines.

A. Mouse Cytokines

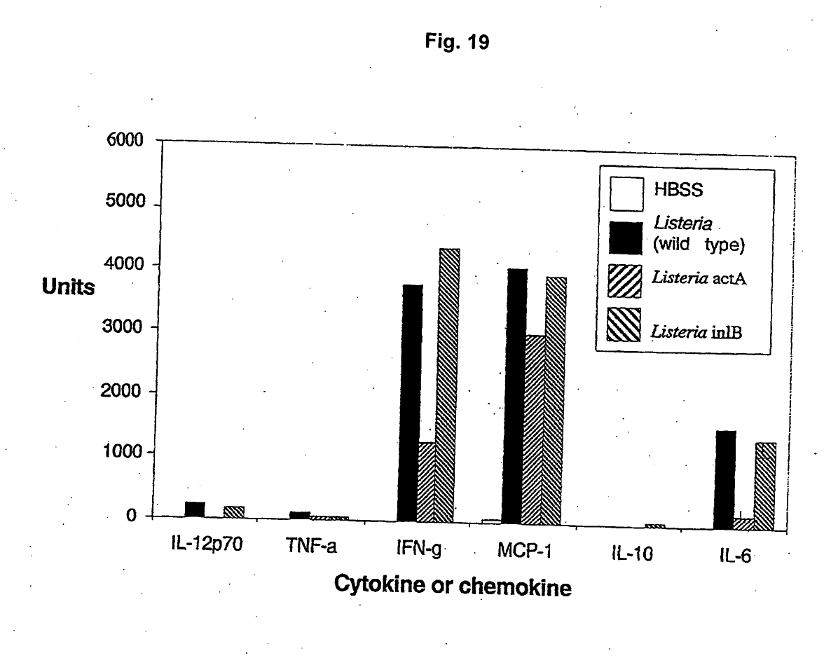

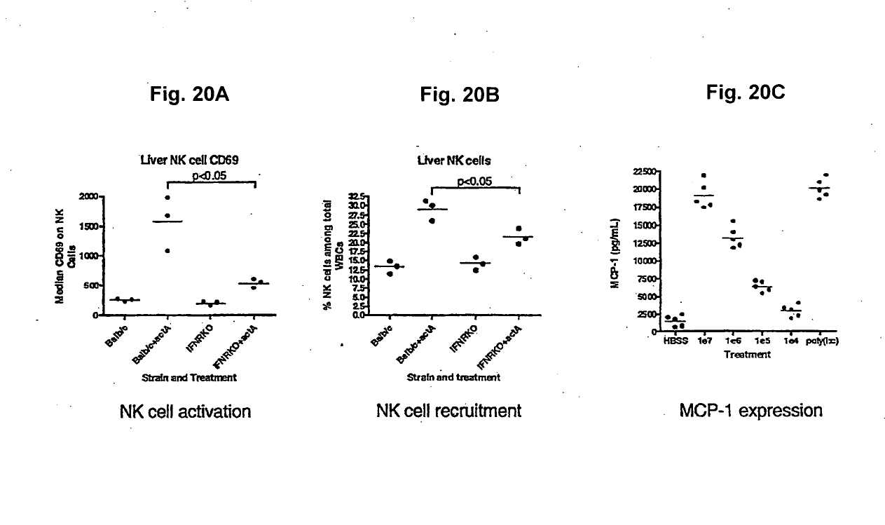

Listeria's influence on cytokine expression in mice is demonstrated in FIGS. 19 and FIGS. 20A, 20B, and 20C.

FIG. 19 demonstrates that administering Listeria stimulates the expression of a number of cytokines. Serum cytokine levels are shown, following a single intravenous administration of Listeria. Cohorts of mice (3 per group) were sampled for serum 24 hrs following a single intravenous administration of salt (HBSS), or of 0.1 LD₅₀ L. monocytogenes ΔactA, L. monocytogenes ΔinlB, or wild-type L. monocytogenes . The cytokines assayed were the p70 subunit of interleukin- 12 (IL-12); TNFalpha; IFNgamma; MCP-1; IL-10; and IL-6. Cytokine levels were determined using the Cytokine Bead Array (CBA) kit (BD Biosciences, San Jose, Calif.). Results are represented as mean +/−SD. The results demonstrated that wild type Listeria, Listeria ΔactA; and Listeria ΔinlB; stimulated expression of interferon-gamma; MCP-1; and IL-6. Of these three, administering wild type Listeria or Listeria ΔactA resulted in the most marked increases in expression of these cytokines.

The present invention provides a method for stimulating expression of IFN-gamma; MCP-1; IL-6; or both IFN-gamma and MCP-1; both IFN-gamma and IL-6; or both IL-6 and MCP-1; or all three of MCP-1, IL-6, and IFN-gamma, comprising administering Listeria ΔactA; Listeria ΔinlB; or attenuated mutant Listeria ΔactΔinlB.

Also provided is a method for stimulating MCP-1 dependent immune response; IFNgamma dependent immune response; or IL-6 dependent immune response, comprising administering Listeria ΔactA; Listeria ΔinlB; or attenuated mutant Listeria ΔactΔinlB. Moreover, what is provided is a method for stimulating an immune response dependent on both IFN-gamma and MCP-1; both IFN-gamma and IL-6; both MCP-1 and IL-6; or dependent on all three of IFN-gamma, MCP-1, and IL-6, comprising administering Listeria ΔactA; Listeria ΔinlB; or attenuated mutant Listeria ΔactΔinlB (FIG. 19).

The following concerns FIGS. 20A, 20B, and 20C. Listeria (not engineered to express any heterologous antigen) provoked the activation and recruitment of NK cells to the liver, where these effects were shown to be mediated by interferon-beta. The following demonstrates that IFN-alpha/beta signaling is required for activation and recruitment of NK cells to the liver in response to Listeria. Livers from 3 individual mice per experimental group were harvested 24 hrs. post single IV administration of 1×10⁷ c.f.u. of L. monocytogenes ΔactA. The harvested livers were processed, and the leukocyte population was counted by forward and side scatter with flow cytometry. The NK cell compartment was evaluated by counting cells that stained positive for both DX5 and/or CD69. The results demonstrated that, with Listeria administration, CD69 expression on NK cells increased from a basal level of about 250 (no Listeria) to about 1500 (yes Listeria) (FIG. 19A). This increase was markedly reduced where mice were IFN receptor knockout mice, thus demonstrating a role of interferon-alpha/beta in Listeria's influence on NK cells activation. Regarding NK cell recruitment, FIG. 19B demonstrates that the percent of NK cells among the total hepatic white blood cells increased from about 13% (no Listeria) to about 30% (yes Listeria), where this effect was reduced in the IFN receptor knockout mice.

In addition to assessing NK cell number, serum cytokine was measured, 24 hrs following a single IV administration of L. monocytogenes ΔactA/ΔinlB. Cohorts of five mice were given a single IV administration of L. monocytogenes ΔactAΔinlB at the dose indicated in the figure and serum was sampled 24 hrs later. The positive control for innate activation consisted of a single IV dose of 100 micrograms of poly I:C (FIG. 20C). The results demonstrate the dramatic effect of Listeria in increasing serum MCP-1. In detail, mice were titrated with Listeria ΔactAΔinlB , where the titration involved zero; 10,000; 0.1 million; 1 million; and 10 million administered bacteria. Again, the results demonstrate that Listeria stimulates an increase in MCP- 1 expression. Methods for assessing DX5 expression are available (see, e.g., Arase, et al. (2001) J. Immunol. 167:1141-1144).

Cytokine levels were measured in serum, where the serum was from blood harvested from mice at various times after administering Listeria or a toll-like receptor (TLR) agonist. The treatment groups were (1) Salt water (HBSS) treatment only (0.2 ml); (2) L. monocytogenes ΔactAΔinlB (1×10⁷ bacteria); (3) L. monocytogenes Δhly (deleted in the gene encoding listeriolysin) (3×10⁸ bacteria); (4) L. monocytogenes killed but metabolically active (KBMA) (3×10⁸ bacteria) (see, e.g. Brockstedt, et al. (2005) Nat. Medicine 11:853-860); (5) heat killed L. monocytogenes ΔactAΔinlB (3×10⁸ bacteria); (6) poly(I:C) (0.1 mg); or (7) CpG (0.1 mg). Peripheral blood was withdrawn at various times, and assessed for cytokine concentration (Mouse Cytokine/Chemokine LINCOplex® Kit Catalog #MCYTO-70K; Linco, St. Charles, Mo.; or BD® Cytometric Bead Array, San Jose, Calif.). CpG was CpG ODN 1826, purchased through Invivogen. Cytokine levels were measured on samples withdrawn at 2, 4, 8, 12, and 24 hours after administration of bacteria or TLR agonist.

The cytokines measured included granulocyte-colony stimulating factor (G-CSF); interferon-gamma (IFN-gamma); interleukin- 1 alpha (IL-1alpha); interleukin-6 (IL-6); interleukin- 10 (IL-10); interleukin- 12p70 (IL-12p70); interleukin-13 (IL-13); IP-10; KC (mouse ortholog of IL-8); MCP-1; MIP-1a; and TNF.

The following cytokines were also measured, where in the case of these cytokines, they were not detected in serum: IL-1beta; IL-2; IL-4; IL-5; IL-7; IL-9; IL-15; IL-17; and granulocyte-monocyte-colony stimulating factor (GM-CSF). In short, these cytokines were not detected under the recited conditions.

Table 7 discloses some of the results.

TABLE 7
Cytokine concentrations in mouse serum after administering Listeria,
poly(I:C), or CpG.

| | | | | | Group 5 | | |
|---|---|---|---|---|---|---|---|
| | | | | | Listeria | | |
| | | | | | ΔactA | | |
| | | Group 2 | Group 3 | Group 4 | ΔinlB | Group 6 | |
| | Group 1 | Listeria | Listeria | Listeria | (heat | Poly | Group 7 |
| | HBSS | ΔactAΔinlB | Δhly | (KBMA) | killed) | (I:C) | CpG |
| | Kinetics and cytokine concentration (pg/ml) | | | | | | |
| G-CSF | Basal | Linear | Early | Early | Early | Early rise | Early rise |
| | level | rise from | high rise | high rise | high rise | to 1200 pg/ml | to 3000 pg/ml |
| | (300-600 pg/ml). | 2-24 h, to | to 18,000 pg/ml | to | to 13,000 pg/ml | (2 h), with | (2 h), with |
| | | a peak of | (2 h), with | 25,000-100,000 pg/ml | (2 h), then | peak at | peak at |
| | | 15,000 pg/ml | peak at | (2-12 h), | gradual | 8-12 h | 8-12 h |
| | | (24 h). | 8-12 h | then | return to | (6,000 pg/ml), | (10,000 pg/ml), |
| | | | (20,000 pg/ml), | return to | basal at | and drop | and drop |
| | | | and | basal | 24 h. | to basal | to basal |
| | | | gradual | (24 h). | | (24 h). | (24 h). |
| | | | drop to | | | | |
| | | | basal | | | | |
| | | | (24 h). | | | | |
| IFN- | Basal | Near | Near | Near | Basal | Early rise | Increase |
| gamma | level | basal at | basal at | basal at | level. | to 15 pg/ml | detected |
| | (<0.05 pg/ml). | 2-4 h, | 2 h, with | 2 h, with | | (2 h) with | at 4 h (10 pg/ml) |
| | | with rise | rise at 4 h, | rise at 4 h, | | plateau | and 8 h |
| | | at 8 h, and | and low | and peak | | (25-30 pg/ml) | (23 pg/ml), |
| | | high | peak (65 pg/ml) | (760 pg/ml) | | at | with |
| | | peak | at | at | | 4-8 h, | decrease. |
| | | (2500 pg/ml) | 8 h, with | 8 h, with | | followed | |
| | | at | decrease. | decrease. | | by return | |
| | | 12 h. | | | | to near | |
| | | | | | | basal. | |
| IL-1alpha | Basal | Near | Early | Early | Early | Near | Early |
| | level (5 pg/ml). | basal at | increase | increase | increase | basal at | increase |
| | | 2 h, with | to 750 pg/ml | to 1200 pg/ml | to 700 pg/ml | 2 h, with | to 130 pg/ml |
| | | linear | (2 h), with | (2 h), with | (2 | plateau | (2 h), with |
| | | increase | peak at | peak at | and 4 h), | (170-300 pg/ml) | peak at |
| | | starting | 4 h (1000 pg/ml) | 8 h (1500 pg/ml) | and | at | 8 h (600 pg/ml) |
| | | from | and drop | and drop | gradual | 8-12 h, | and drop |
| | | 4-24 h to | towards | towards | drop | and basal | to basal |
| | | peak (900 pg/ml) | basal by | basal by | towards | at 24 h. | by 24 h. |
| | | at | 24 h. | 24 h. | basal by | | |
| | | 24 h. | | | 24 h. | | |
| IL-6 | Basal | Basal at | Early rise | Early | Early | Early rise | Early rise |
| | level | 2 h, with | (to 1000 pg/ml) | high rise | peak (750 pg/ml) | (7500 pg/ml) | (2000 pg/ml) |
| | (5-70 pg/ml). | increase | with peak | with peak | (2 h) with | at | at |
| | | starting at | at 2 h, | at 2 h | return to | 2 h, with | 2 h, with |
| | | 4 h, peak | with | (5000 pg/ml), | basal by | gradual | gradual |
| | | at 8 h | gradual | with | 4 h. | drop | drop |
| | | (1250 pg/ml), | return to | gradual | | (5000 pg/ml | (1000 pg/ml |
| | | and drop | basal at | drop | | at | at |
| | | to 200 pg/ml | 24 h. | (2500 pg/ml | | 4 h) to | 4 h) to |
| | | (24 h). | | at | | near basal | near basal |
| | | | | 4 h) to | | at 12 h. | at 12 h. |
| | | | | basal at | | | |
| | | | | 24 h. | | | |
| IL-10 | Basal | Basal at | Moderate | Moderate | Sporadic | Sporadic | Basal at |
| | level | 2 h, with | levels at | levels at | spikes in | spikes in | 2 h, with a |
| | (<1 pg/ml). | gradual | 2 h, 4 h, | 2 h and | the range | the range | peak at |
| | | increase, | 12 h, with | 12 h, with | of 14-35 pg/ml | of 14-80 pg/ml | 4 h (200 pg/ml), |
| | | starting at | a peak at | a peak at | found at | found at | and low |
| | | 8 h, to a | 8 h (250 pg/ml). | 4-8 h | 2 h and at | 2 h and at | plateau |
| | | peak at | Basal at | (200-250 pg/ml). | 8 h. | 8 h. Basal | from |
| | | 24 h (40 pg/ml). | 24 h. | Basal at | Basal at | at 4 h, | 8-24 h |
| | | | | 24 h. | 4 h, 12 h, | 12 h, 24 h. | (30-60 pg/ml). |
| | | | | | 24 h. | | |
| IL-12p70 | Basal | Basal at | Early rise | Early rise | Early rise | Early rise | Early rise |
| | level | 2 h, with | with a | to | with a | with a | with a |
| | (40-60 pg/ml). | gradual | peak of | 200-400 pg/ml | peak of | peak of | peak of |
| | | increase | 100-150 | found at | 180 at 2 h | 300 at 2 h | 1100 at |
| | | to a peak | at 2 h-8 h, | 2-4 h, | followed | followed | 2 h |
| | | of 550 pg/ml | followed | with a | by a | by a | followed |
| | | (12 h), | by a | peak of | steady | steady | by a |
| | | and | decrease | 1500 pg/ml | decrease | decrease | steady |
| | | decrease | to 65-75 pg/ml | (8 h), and | (basal at | (basal at | decrease, |
| | | to 250 pg/ml | (12-24 h). | drop to | 8-24 h). | 12-24 h). | reaching |
| | | (24 h). | | near basal | | | basal at |
| | | | | levels | | | 24 h. |
| | | | | (12 h-24 h). | | | |
| IL-13 | Basal | Basal | Basal | Early rise | Basal | Basal | Basal |
| | level | levels at | levels at | to about | levels at | levels at | levels at |
| | (3.2 pg/ml). | 2 h-24 h, | 2 h-24 h, | 80 pg/ml | 2 h-24 h, | 2 h-24 h, | 2 h-24 h, |
| | | but with | but with | (2 h), with | but with | but with | but with |
| | | sporadic | sporadic | near basal | sporadic | sporadic | sporadic |
| | | spikes (to | spikes (to | level at | spikes (to | spikes (to | spikes (to |
| | | about 250 pg/ml) | about 250 pg/ml) | 4 h, and | about 250 pg/ml) | about 200 pg/ml) | about 100 pg/ml) |
| | | at | at | peak to | at | at | at |
| | | 24 h in | 8 h in | 380 pg/ml | 12 h in | 4 h, 12 h, | 4 h and 8 h |
| | | some | some | (8 h), and | some | 24 h, in | in some |
| | | mice. | mice. | drop to | mice. | some | mice. |
| | | | | basal | | mice. | |
| | | | | (12 h, | | | |
| | | | | 24 h), | | | |
| | | | | with | | | |
| | | | | sporadic | | | |
| | | | | spikes at | | | |
| | | | | 12 h and | | | |
| | | | | 24 h. | | | |
| IP-10 | Basal | Early rise | Early rise | Early rise | Early rise | Early rise | Early rise |
| | level | at 2 h to | to 900 pg/ml | to 1000 pg/ml | to 820 pg/ml | to 1400 pg/ml | to 1000 pg/ml |
| | (<10 pg/ml). | 500 pg/ml, | (2 h) with | (2 h) with | at | at | at |
| | | with a | a plateau | plateau at | 2 h, with | 2 h, with | 2 h, with |
| | | peak | at this | this level | gradual | peak at | peak at |
| | | occurring | level to | continuing | drop, | 4 h (2100 pg/ml), | 4 h(1400 pg/ml), |
| | | at 8 h-24 h | 8 h, and | to 12 h | with near | and | and |
| | | (1400 pg/ml | decrease | with | basal | gradual | gradual |
| | | at | to 250 pg/ml | slight | levels at | drop (800 pg/ml | drop (400 pg/ml |
| | | 12 h). | (24 h). | drop to | 12 h and | at | at |
| | | | | 700 pg/ml | 24 h. | 24 h). | 24 h). |
| | | | | (24 h). | | | |
| KC | Basal | Early rise | Early | Early | Early rise | Early rise | Early rise |
| | level | to 500 pg/ml | high rise | high rise | to 1500 pg/ml | to 900 pg/ml | to 1100 pg/ml |
| | (<25 pg/ml). | (2 h) with | to 2000 pg/ml | to 5100 pg/ml | at | (2 h) with | (2 h) with |
| | | lower | (2 h) with | at | 2 h, with | near basal | drop to |
| | | levels at | maintained | 2 h, with | return to | levels | 500 pg/ml |
| | | 4 h-8 h | low levels | gradual | a maintained | (4 h-24 h). | (4 h), and |
| | | (200 pg/ml), | (<300 pg/ml) | drop, and | basal | | basal |
| | | increase | at | near basal | level at | | levels at |
| | | at 12 h | 4-12 h, | levels at | 4 h-8 h. | | 12 h-24 h. |
| | | (700 pg/ml) | and basal | 24 h. | | | |
| | | and drop | level at | | | | |
| | | at 24 h | 24 h. | | | | |
| | | (200 pg/ml). | | | | | |
| MCP-1 | Basal | Early rise | Early | Early | Early rise | Early | Early rise |
| | level | by 2 h | high rise | high rise | to 7000 pg/ml | high rise | to 10,000 pg/ml |
| | (100 pg/ml). | (3000 pg/ml) | to 9000 pg/ml | to 16,000 pg/ml, | (2 h), | to 29,000 pg/ml | (2 h), then |
| | | with peak | (2 h), with | with a | followed | (2 h), then | gradual |
| | | at 12 h | gradual | peak at | by sudden | gradual | drop to |
| | | (10,000 pg/ml) | decrease, | 4 h | drop at | drop | 5000 pg/ml |
| | | and maintained | and basal | (24,000 pg/ml), | 4 h, with | (10,000 pg/ml | (8 h) and |
| | | levels at | levels at | and | near basal | at | low levels |
| | | 24 h | 12 h and | gradual | levels | 8 h), and | (800 pg/ml) |
| | | (5500 pg/ml). | 24 h. | drop to | (8 h-12 h). | low levels | at |
| | | | | 1000 pg/ml | | (1000 pg/ml) | 12 h-24 h. |
| | | | | (24 h). | | at | |
| | | | | | | 12 h and | |
| | | | | | | 24 h. | |
| MIP-1a | Basal | Basal | Early rise | Early | Early rise | Early rise | Early rise |
| | level | level until | to 850 pg/ml | high rise | to 600 pg/ml | to 1800 pg/ml | to 1300-1500 |
| | (3.2 pg/ml). | 12 h, | at | to 3000 pg/ml | (2 h), with | (2 h) with | at 2-4 h, |
| | | where | 2 h, | (2 h), | drop to | gradual | with |
| | | level at | followed | with4500 pg/ml | near basal | drop | gradual |
| | | 24 h is | by drop | peak at | by 4 h. | towards | return to |
| | | 280 pg/ml. | to near | 4 h, then | | basal by | near basal |
| | | | basal by | drop. | | 8 h. | at 12 h. |
| | | | 8 h. | | | | |
| TNF | Basal | Slow | Early | Early | Early rise | Early rise | Plateau of |
| | level | increase | peak of | peak of | to 500 pg/ml | to 1500 pg/ml | 300-550 |
| | (3.2 pg/ml). | evident | 550 pg/ml | 1600 pg/ml | (2 h) with | (2 h) with | at 2-8 h, |
| | | by 2-4 h, | at 2 h, | by | return to | return | followed |
| | | with peak | with | 2 h, with | basal by | towards | by drp to |
| | | (250 pg/ml) | gradual | gradual | 8 h. | basal by | basal at |
| | | at | drop to | drop to | | 8 h. | 24 h. |
| | | 12 h. | basal by | basal by | | | |
| | | | 12 h. | 12 h. | | | |
| The present invention, in certain embodiments, provides methods of modulating, e.g., stimulating, expression of one or any combination of G-CSF; IFN-gamma; IL-1alpha; IL-6; IL-10; IL-12p70 (interleukin-12 is a heterodimeric cytokine of p40 and p35 subunits); IL-13; IP-10; KC; MCP-1; MIP-1a; TNF. Provided is a method of stimulating or inhibiting a condition or disorder that is dependent on, or is modulated by, one or any | | | | | | | |
| # combination of G-CSF; IFN-gamma; IL-1alpha; IL-6; IL-10; IL-12p70 (interleukin-12 is a heterodimeric cytokine of p40 and p35 subunits); IL-13; IP-10; KC; MCP-1; MIP-1a; TNF. | | | | | | | |

B. Monkey Cytokines

Cytokine expression was measured in non-human primates that were administered Lm ΔactAΔinlB . Cynomolgus monkeys, both male and female, were administered with vehicle, 1×10^{7, 3×10}⁸, or 1×10¹⁰ cfu of Lm ΔactAΔinlB . A total of 32 cynomolgous monkeys (16 per gender) were randomly assigned to the four dose groups.

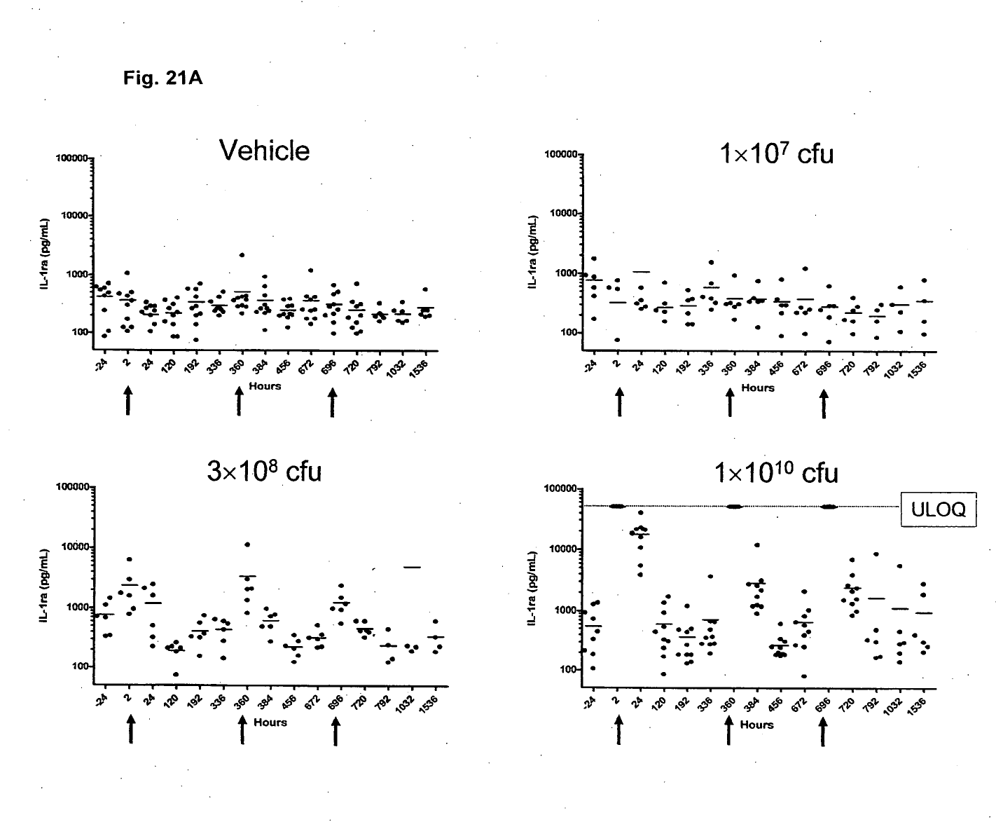

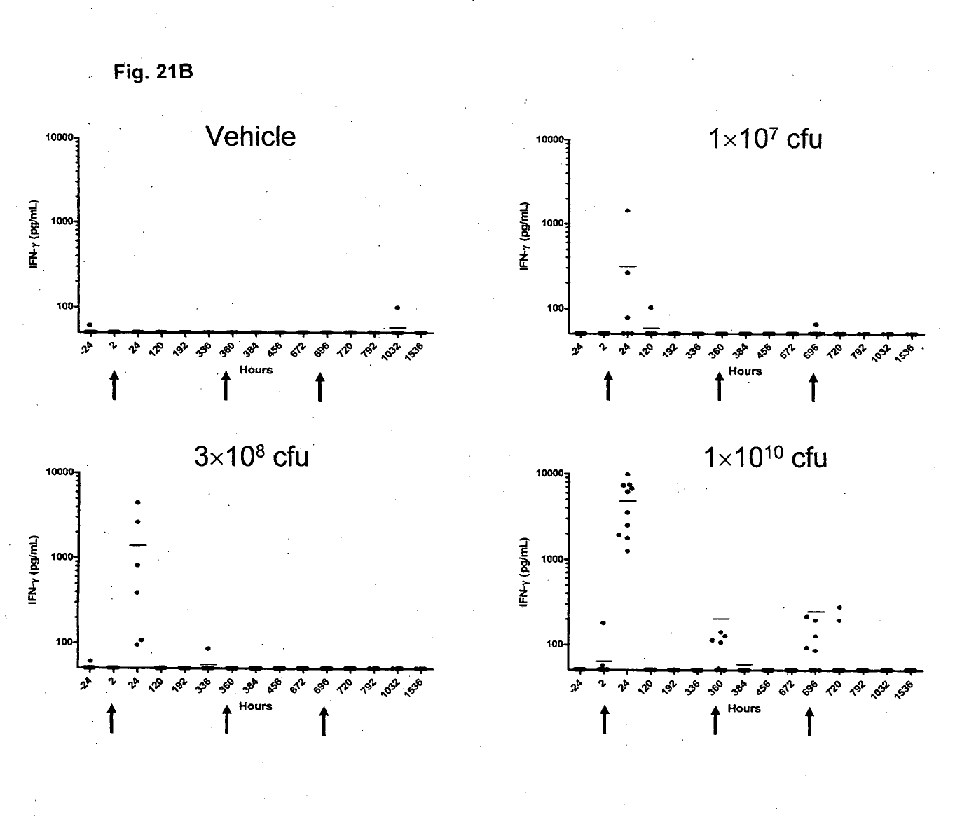

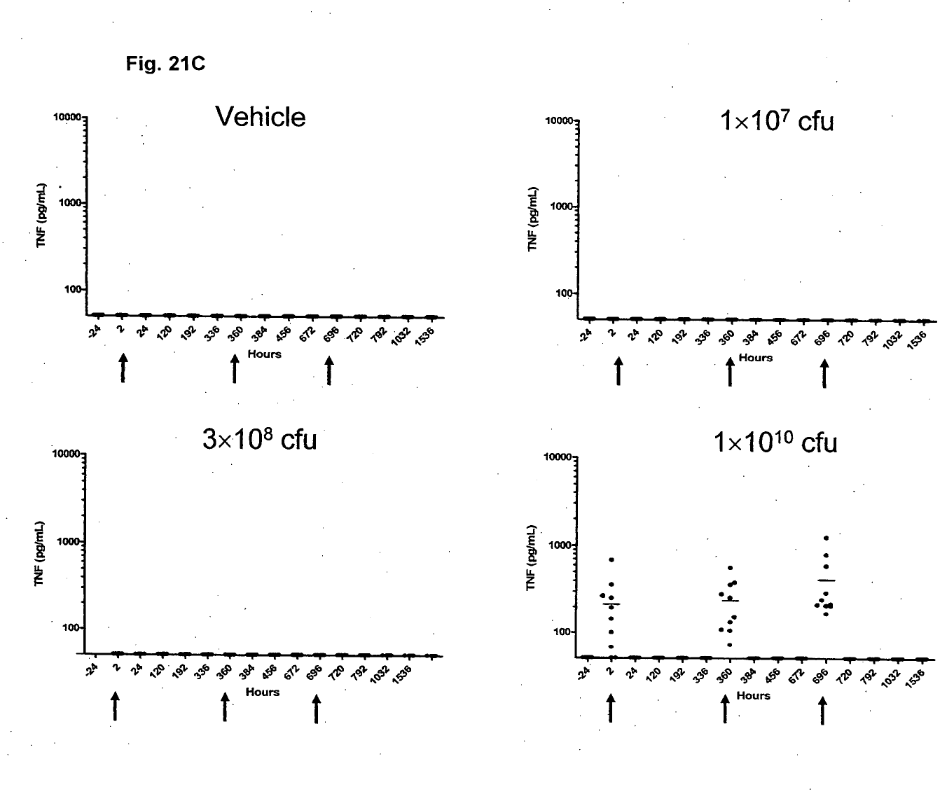

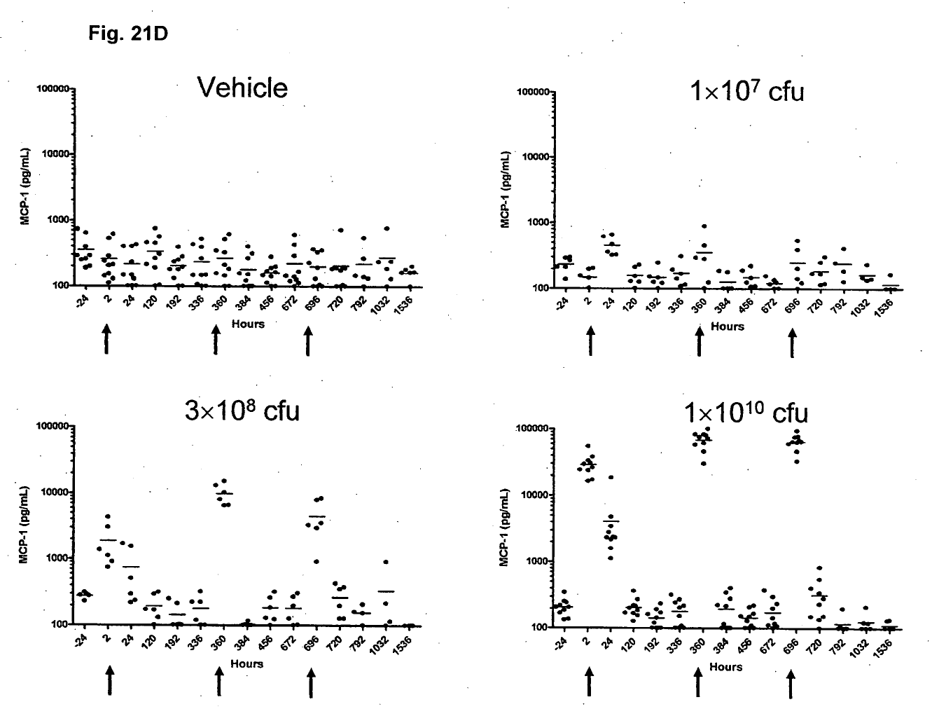

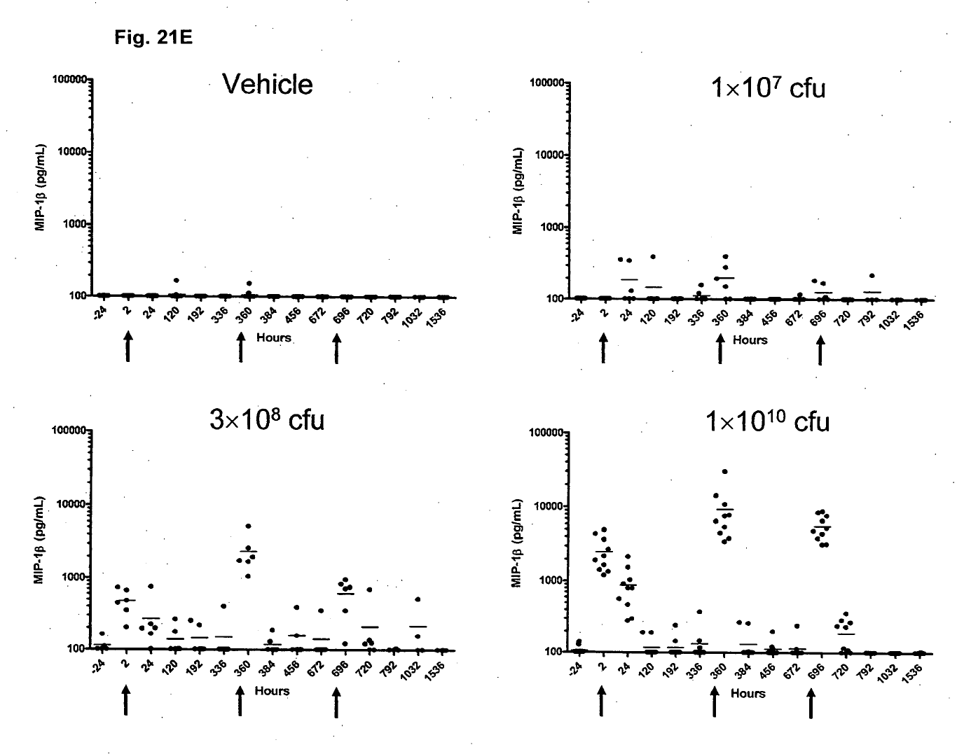

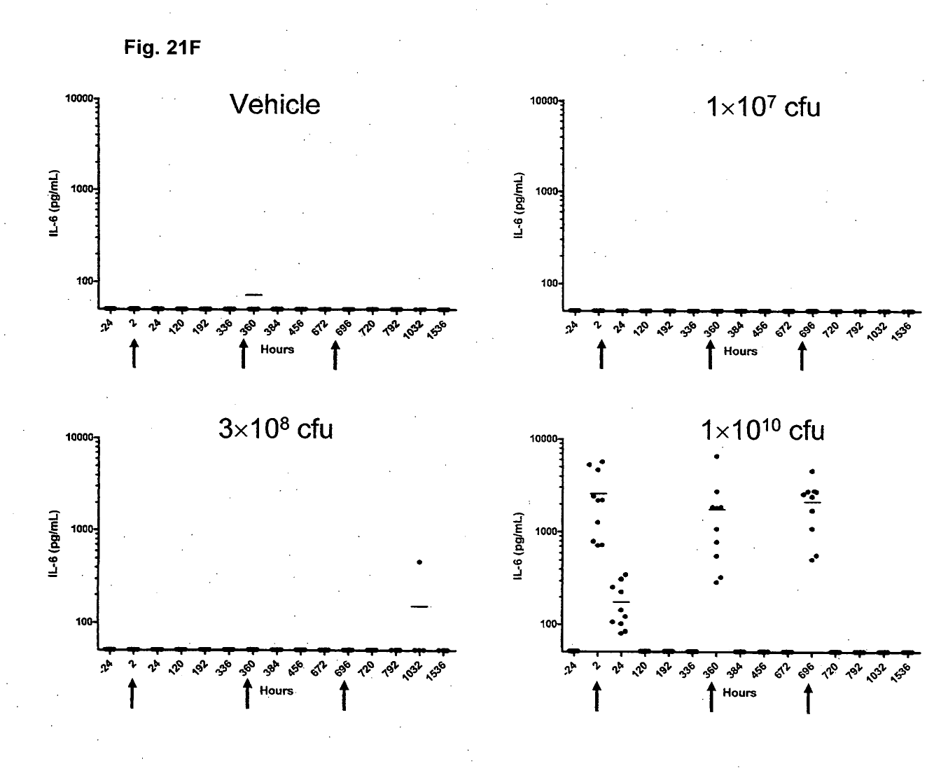

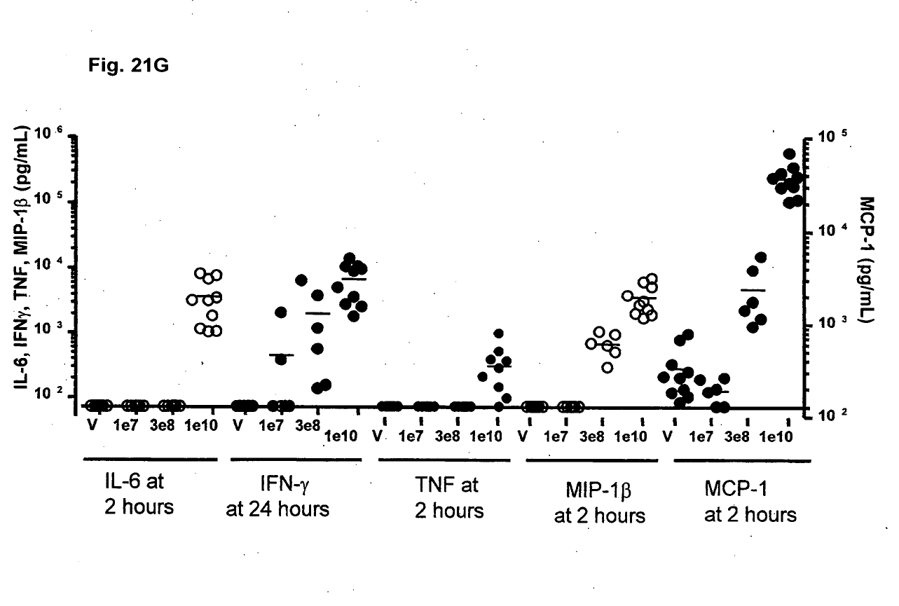

Administration was via a 30 minute (i.v.) infusion every week for five total doses. Serial serum and plasma samples were analyzed for the respective cytokines: IL-1Ralpha; IFNgamma; TNFalpha; MCP-1; MIP-1beta; and IL-6 (FIGS. 21A-F). FIG. 21G also shows cytokine expression by cynomolgus monkeys, and discloses cytokine expression following the first infusion of Lm ΔactAΔinlB . IL-6, IFNgamma, TNF, MIP-1beta, and MCP-1 were measured after the initial infusion, as indicated (FIG. 21G). Serum levels of each of these cytokines increased, specifically in response to Lm ΔactAΔinlB , where the increases all demonstrated a dependence on the dose.

XIV. Optimal Anti-Tumor Activity Requires Cytosolic Entry by Listeria monocytogenes.

Liver-specific CT-26 metastasis were established following the protocol described by Jain et al., Ann. Surg. Oncol. 10:810-820 (2003) with slight modifications. CT26 is an N-nitroso-N-methylurethane-induced murine colon adenocarcinoma cell line derived from Balb/c mice. Cells were maintained in culture in Dulbecco modified Eagle medium (DMEM) supplemented with 10% fetal bovine serum (FBS) and penicillin/streptomycin (50 U/ml).

On Day 0, female Balb/c mice were implanted with 1×10⁵ CT26 cells via hemispleen surgery. Briefly, Balb/c mice were anesthetized via isoflurane and a left flank incision was made to expose the spleen. The spleen was divided into two hemispleens by using two medium-size Horizon titanium surgical clips (Weck Closure Systems, Research Triangle Park, N.C.) leaving the vascular pedicles intact. Using a 27-gauge needle, 10⁵ viable CT-26 cells were injected into one half of the spleen. The CT-26 tumor cells then flow into the splenic and portal veins and deposit in the liver. The vascular pedicle draining the cancer-contaminated hemispleen was ligated and the CT-26-contaminated hemispleen was excised, leaving a functional hemispleen free of tumor cells.

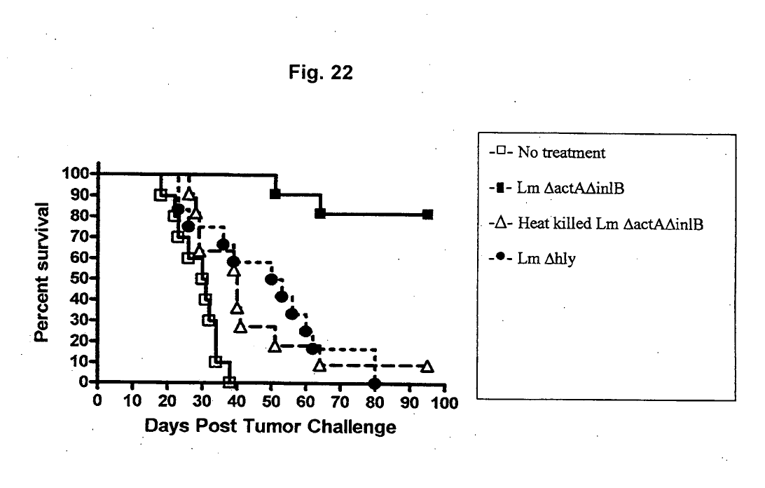

To understand the necessity for bacterial entry into the cytosol, tumor bearing mice were immunized with either live Lm ΔactAΔinlB , heat-killed (HK) Lm ΔactAΔinlB , or L. monocytogenes unable to produce LLO (Δhly, unable to escape the phagocytic vacuole). The Listeria were diluted in HBSS to the appropriate concentration and administered intravenously into the mice in a final volume of 100 or 200 μl. Balb/c mice bearing 3 day established hepatic metastasis were treated with Lm ΔactAΔinlB (3e7 cfu), heat-killed Lm ΔactAΔinlB (3e8 cfu), or Δhly Lm (3e8 cfu). The vaccinations were given on day 3, 10, and 17. The percent survival is shown in FIG. 22 for each group (n=6-10 mice per group).

Both HK-Lm ΔactAΔinlB and LLO-deficient L. monocytogenes significantly prolonged the median survival (MST 40 and 52 days respectively) relative to untreated controls (MST 31 days), although a majority of the animals succumbed to tumor burden. This is in striking contrast to mice that were treated with Lm ΔactAΔinlB where 80% of Lm ΔactAΔinlB treated mice remained tumor free for the duration of the study (FIG. 22). These results indicate that optimal Lm-induced anti-tumor activity requires cytosolic entry.

Many modifications and variations of this invention, as will be apparent to one of ordinary skill in the art, can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

A Listeria monocytogenes ΔactAΔinlB strain was deposited with the American Type Culture Collection (ATCC), 10801 University Blvd., Manassas, Va. 20110-2209, United States of America (P.O. Box 1549, Manassas, Va., 20108, United States of America), on Oct. 3, 2003, under the provisions of the Budapest Treaty on the International Recognition of the Deposit of Microorganisms for the Purposes of Patent Procedure, and designated with accession number PTA-5562. Another Listeria monocytogenes strain, an ΔactAAuvrAB strain, was also deposited with the ATCC on Oct. 3, 2003, under the provisions of the Budapest Treaty on the International Recognition of the Deposit of Microorganisms for the Purposes of Patent Procedure, and designated with accession number PTA-5563.

What is claimed is:

1. A method for stimulating an immune response against a cancerous or infectious condition in a mammal having the condition, comprising administering to the mammal effective amounts of a Listeria and:
a. an antibody that specifically binds to an antigen of the condition; or
b. a binding compound derived from the antigen-binding site of an antibody that specifically binds to an antigen of the condition and also specifically binds to an immune cell that mediates antibody-dependent cell cytotoxicity (ADCC),
wherein the combination of the Listeria and the antibody, or binding compound, is effective in stimulating the response.

2. The method of claim 1, wherein the Listeria and the antibody, or binding compound, are administered simultaneously.

3. The method of claim 1, wherein the Listeria and the antibody, or binding compound, are not administered simultaneously.

4. The method of claim 1, wherein the Listeria is attenuated.

5. The method of claim 1, wherein the binding compound derived from the antigen-binding site of an antibody further comprises an Fc region, or an Fc region derivative.

6. The method of claim 5, wherein the Fc region derivative has one or both of:
a. enhanced affinity for an activating receptor expressed by the cell that mediates ADCC; or
b. decreased affinity for an inhibiting receptor expressed by the cell that mediates ADCC.

7. The method of claim 5, wherein the Fc region derivative comprises an IgG1 Fc region that contains one or more of the mutations:
a. S298A;
b. E333A; or
c. K334A,
wherein the mutation is useful in mediating increased activation of the cell that mediates ADCC.

8. The method of claim 1, wherein the binding compound comprises:
a. a bispecific antibody, and wherein the first binding site of the bispecific antibody specifically binds to the antigen of the condition and the second binding site of the bispecific antibody specifically binds to the immune cell that mediates ADCC; or
b. a peptide mimetic of an antibody that specifically binds to the antigen of the condition.

9. The method of claim 1, wherein the Listeria is metabolically active and is essentially incapable of one or more of:
a. forming colonies;
b. replicating; or
c. dividing.

10. The method of claim 1, wherein the Listeria is essentially metabolically inactive.

11. The method of claim 1, wherein the attenuated Listeria is attenuated in one or more of:
a. growth;
b. cell-to-cell spread;
c. binding to or entry into a cell;
d. replication; or
e. DNA repair.

12. The method of claim 1, wherein the Listeria is attenuated by one or more of:
a. an actA mutation;
b. an inlB mutation;
c. a uvrA mutation;
d. a uvrB mutation;
e. a uvrC mutation;
f. a nucleic acid targeted compound; or
g. a uvrAB mutation and a nucleic acid targeting compound.

13. The method of claim 12, wherein the nucleic acid targeting compound is a psoralen.

14. The method of claim 1, wherein the condition comprises one or more of a tumor, cancer, or pre-cancerous disorder.

15. The method of claim 1, wherein the condition comprises an infection.

16. The method of claim 1, wherein the condition comprises an infection by one or more of:
a. hepatitis B;
b. hepatitis C;
c. human immunodeficiency virus (HIV);
d. cytomegalovirus (CMV);
e. Epstein-Barr virus (EBV); or
f. leishmaniasis.

17. The method of claim 1, wherein the condition is of the liver.

18. The method of claim 1, wherein the immune response is against a cell of the condition.

19. The method of claim 1, wherein the immune response comprises an innate immune response.

20. The method of claim 1, wherein the immune response comprises an adaptive immune response.

21. The method of claim 1, wherein the mammal is human.

22. The method of claim 1, wherein the Listeria is Listeria monocytogenes.

23. The method of claim 1, wherein the Listeria comprises a nucleic acid encoding a heterologous antigen.

24. The method of claim 1, wherein the attenuated Listeria is one reagent, and the antibody, or the binding compound, is a second reagent, further comprising administering a third reagent to the mammal.

25. The method of claim 24, wherein the third reagent comprises one or more of:
a. an agonist or antagonist of a cytokine;
b. an inhibitor of a T regulatory cell (Treg); or
c. cyclophosphamide (CTX).

26. The method of claim 1, wherein the immune response comprises activation of, or an inflammation by, one or any combination of:
a. an NK cell;
b. an NKT cell;
c. a dendritic cell (DC);
d. a monocyte or macrophage;
e. a neutrophil;
f. a toll-like receptor (TLR); or
g. a nucleotide-binding oligomerization domain protein (NOD protein), as compared with immune response in the absense of the administering of the effective amount of the Listeria.

27. The method of claim 1, wherein the immune response comprises increased expression of one or any combination of:
a. CD69;
b. interferon-gamma (IFNgamma);
c. interferon-alpha (IFNalpha) or interferon-beta (IFNbeta);
d. interleukin-12 (IL-12);
e. monocyte chemoattractant protein (MCP-1); or
f. interleukin-6 (IL-6),
as compared with expression in the absence of the administering of the effective amount of the Listeria.

28. The method of claim 1, wherein the stimulating comprises:
a. an increase in percent of NK cells in a population of hepatic leukocytes in the mammal, compared to the percent without the administering of the Listeria; or
b. an increase in expression of an activation marker by a hepatic NK cell, compared to the expression without the administering of the Listeria.

29. The method of claim 28 wherein the increase in the percent of NK cells is at least:
a. 5%;
b. 10%;
c. 15%;
d. 20%; or
e. 25%,
greater than compared to the percent without the administering of the attenuated Listeria.

30. The method of claim 1, wherein the administered Listeria is one or both of:
a. not administered orally to the mammal; or
b. administered to the mammal as a composition that is at least 99% free of other types of bacteria.

31. A method for treating a cancerous or infectious condition in a mammal having the condition, comprising administering to the mammal effective amounts of a Listeria with:
a. an antibody that specifically binds to an antigen of the condition; or
b. a binding compound derived from an antibody that specifically binds to an antigen of the condition and also specifically binds to an immune cell that mediates ADCC,
wherein the combination of the Listeria and the antibody, or binding compound, is effective in ameliorating or reducing the condition.

32. The method of claim 31, wherein the Listeria and the antibody, or binding compound, are administered simultaneously.

33. The method of claim 31, wherein the Listeria and the antibody, or binding compound, are not administered simultaneously.

34. The method of claim 31, wherein the Listeria is attenuated.

35. The method of claim 31, wherein the binding compound derived from the antigen-binding site of an antibody further comprises an Fc region, or an Fc region derivative.

36. The method of claim 35, wherein the Fc region derivative has one or both of:
a. enhanced affinity for an activating receptor expressed by the cell that mediates ADCC; or
b. decreased affinity for an inhibiting receptor expressed by the cell that mediates ADCC.

37. The method of claim 35, wherein the Fc region derivative comprises an an IgG1 Fc region that contains one or more of the mutations:
a. S298A;
b. E333A; or
c. K334A,
wherein the mutation is useful in mediating increased activation of the cell that mediates ADCC.

38. The method of claim 31, wherein the binding compound comprises:
a. a bispecific antibody, wherein the first binding site of the bispecific antibody specifically binds to the antigen of the condition and the second binding site of the bispecific antibody specifically binds to the immune cell that mediates ADCC; or
b. a peptide mimetic of an antibody that specifically binds to the antigen of the condition.

39. The method of claim 31, wherein the Listeria is metabolically active and is essentially incapable of one or more of:
a. forming colonies;
b. replicating; or
c. dividing.

40. The method of claim 31, wherein the Listeria is essentially metabolically inactive.

41. The method of claim 31, wherein the Listeria is attenuated in one or more of:
a. growth;
b. cell-to-cell spread;
c. binding to or entry into a cell;
d. replication; or
e. DNA repair.

42. The method of claim 31, wherein the Listeria is attenuated by one or more of:
a. an actA mutation;
b. an inlB mutation;
c. a uvrA mutation;
d. a uvrB mutation;
e. a uvrC mutation;
f. a nucleic acid targeting compound; or
g. a uvrAB mutation and a nucleic acid targeting compound.

43. The method of claim 42, wherein the nucleic acid targeting compound is a psoralen.

44. The method of claim 31, wherein the condition comprises a cancer, tumor, or pre-cancerous disorder.

45. The method of claim 31, wherein the condition comprises an infection.

46. The method of claim 31, wherein the condition comprises an infection by one or more of:
a. hepatitis B;
b. hepatitis C;
c. human immunodeficiency virus (HIV);
d. cytomegalovirus (CMV);
e. Epstein-Barr virus (EBV); or
f. leishmaniasis.

47. The method of claim 31, wherein the condition is of the liver.

48. The method of claim 31, wherein the immune response is against a cell of the condition.

49. The method of claim 31, wherein the treating results in a stimulated innate immune response.

50. The method of claim 31, wherein the treating results in a stimulated adaptive immune response.

51. The method of claim 31, wherein the mammal is human.

52. The method of claim 31, wherein the Listeria is Listeria monocytogenes.

53. The method of claim 31, wherein the Listeria comprises a nucleic acid encoding a heterologous antigen.

54. The method of claim 31, wherein the Listeria is a first reagent, and the antibody or the binding compound is a second reagent, further comprising administering a third reagent to the mammal.

55. The method of claim 54, wherein the third reagent comprises one or more of:
a. an agonist or antagonist of a cytokine;
b. an inhibitor of a T regulatory cell (Treg); or
c. cyclophosphamide (CTX).

56. The method of claim 31, wherein the treating results in activation of, or inflammation by, one or any combination, of:
a. an NK cell;
b. an NKT cell;
c. a dendritic cell (DC);
d. a monocyte or macrophage;
e. a neutrophil; or
f. a toll-like receptor (TLR) or nucleotide-binding oligomerization domain (NOD) protein, as compared with immune response in the absence of the administering of the effective amount of the Listeria.

57. The method of claim 31, wherein the treating results in increased expression of one or any combination of:
a. CD69;
b. interferon-gamma (IFNgamma);
c. interferon-alpha (IFNalpha) or interferon-beta (IFNbeta);
d. interleukin-12 (IL-12);
e. monocyte chemoattractant protein (MCP-1); or
f. interleukin-6 (IL-6),
as compared with expression in the absence of the administering of the effective amount of the Listeria.

58. The method of claim 31, wherein the treating results in:
a. an increase in percent of NK cells in hepatic leukocytes in the mammal, compared to the percent without the administering of the Listeria; or
b. an increase in expression of an activation marker by a hepatic NK cell, compared to the expression without the administering of the Listeria.

59. The method of claim 58 wherein the increase in percent of NK cells is at least:
a. 5%;
b. 10%;
c. 15%;
d. 20%; or
e. 25%,
greater than compared to the percent without administering the Listeria.

60. The method of claim 31, wherein the treating increases survival of the mammal, as determined by comparison to a suitable control mammal having the condition and not administered with the Listeria, antibody, or binding compound.

61. The method of claim 31, wherein the condition comprises one or more of cancer cells, tumors, or an infectious agent, and wherein the treating reduces one or more of the:
a. number of tumors or cancer cells;
b. tumor mass; or
c. titer of the infectious agent,
in the mammal.

62. The method of claim 31, wherein the administered Listeria is one or both of:
a. not administered orally to the mammal; or
b. administered to the mammal as a composition that is at least 99% free of other types of bacteria.

63. A kit for for use in the methods of claim 1 or claim 31 comprising:
(a) a composition comprised of Listeria ; and
(b) a composition comprised of (i) an antibody that specifically binds to an antigen of the condition, or (ii) a binding compound derived from an antigen binding-site of an antibody that specifically binds to an antigen of the condition and also specifically binds to an immune cell that mediates ADCC;
and optionally containing instructions for use of the compositions, wherein the compositions are packaged in suitable containers.
